SEQUENCE LISTING (409 sequences)

SEQ ID NO: 1 - protein, 10 aa, Artificial Sequence
  Description of Artificial Sequence: Synthetic peptide
GFDIQDTYMH

SEQ ID NO: 2 - protein, 17 aa, Artificial Sequence
  Description of Artificial Sequence: Synthetic peptide
RIDPASGHTKYDPKFQV

SEQ ID NO: 3 - protein, 17 aa, Artificial Sequence
  Description of Artificial Sequence: Synthetic peptide
RIEPASGHIKYDPKFQG

SEQ ID NO: 4 - protein, 17 aa, Artificial Sequence
  Description of Artificial Sequence: Synthetic peptide
RIDPASGHIKYDPKFQG

SEQ ID NO: 5 - protein, 17 aa, Artificial Sequence
  Description of Artificial Sequence: Synthetic peptide
RIEPASGHIKYDPKFQV

SEQ ID NO: 6 - protein, 7 aa, Artificial Sequence
  Description of Artificial Sequence: Synthetic peptide
SGGLPDV

SEQ ID NO: 7 - protein, 9 aa, Artificial Sequence
  Description of Artificial Sequence: Synthetic peptide
ARSGGLPDV

SEQ ID NO: 8 - protein, 7 aa, Artificial Sequence
  Description of Artificial Sequence: Synthetic peptide
SGGLPDW

SEQ ID NO: 9 - protein, 9 aa, Artificial Sequence
  Description of Artificial Sequence: Synthetic peptide
ARSGGLPDW

SEQ ID NO: 10 - protein, 10 aa, Artificial Sequence
  Description of Artificial Sequence: Synthetic peptide
RASSSVSYMY

SEQ ID NO: 11 - protein, 7 aa, Artificial Sequence
  Description of Artificial Sequence: Synthetic peptide
ATSNLAS

SEQ ID NO: 12 - protein, 9 aa, Artificial Sequence
  Description of Artificial Sequence: Synthetic peptide
QQWEGNPRT

SEQ ID NO: 13 - protein, 9 aa, Artificial Sequence
  Description of Artificial Sequence: Synthetic peptide
QQWKGNPRT

SEQ ID NO: 14 - protein, 9 aa, Artificial Sequence
  Description of Artificial Sequence: Synthetic peptide
QQWSGNPRT

SEQ ID NO: 15 - protein, 9 aa, Artificial Sequence
  Description of Artificial Sequence: Synthetic peptide
QQWSRNPRT

SEQ ID NO: 16 - protein, 10 aa, Artificial Sequence
  Description of Artificial Sequence: Synthetic peptide
  MOD_RES (3)..(3): D or E
  MOD_RES (4)..(4): I, L, P, or V
  MOD_RES (5)..(5): G, Q, S, or V
  MOD_RES (7)..(7): A, S, or T
  MOD_RES (8)..(8): F or Y
  MOD_RES (9)..(9): I, L, or M
GFXXXDXXXH

SEQ ID NO: 17 - protein, 10 aa, Artificial Sequence
  Description of Artificial Sequence: Synthetic peptide
  MOD_RES (3)..(3): D or E
  MOD_RES (4)..(4): I, P, or V
  MOD_RES (5)..(5): G, Q, S, or V
  MOD_RES (8)..(8): F or Y
  MOD_RES (9)..(9): I or M
GFXXXDTXXH

SEQ ID NO: 18 - protein, 17 aa, Artificial Sequence
  Description of Artificial Sequence: Synthetic peptide
  MOD_RES (2)..(2): I or L
  MOD_RES (3)..(3): D or E
  MOD_RES (5)..(5): A or E
  MOD_RES (6)..(6): G or S
  MOD_RES (7)..(7): A or G
  MOD_RES (9)..(9): I, L, T, or V
  MOD_RES (11)..(11): I, L, M, S, T, V, or Y
  MOD_RES (12)..(12): D, I, N, R, or S
  MOD_RES (16)..(16): Q or R
  MOD_RES (17)..(17): A, D, E, G, H, K, L, M, N, P, R, S, T, or V
RXXPXXXHXKXXPKFXX

SEQ ID NO: 19 - protein, 7 aa, Artificial Sequence
  Description of Artificial Sequence: Synthetic peptide
  MOD_RES (1)..(1): L or S
  MOD_RES (2)..(2): A or G
  MOD_RES (4)..(4): A, L, or M
  MOD_RES (6)..(6): D or E
  MOD_RES (7)..(7): K, M, Q, R, S, T, V, or W
XXGXPXX

SEQ ID NO: 20 - protein, 7 aa, Artificial Sequence
  Description of Artificial Sequence: Synthetic peptide
  MOD_RES (4)..(4): L or M
  MOD_RES (7)..(7): E, I, K, L, M, Q, T, V, W, or Y
SGGXPDX

SEQ ID NO: 21 - protein, 10 aa, Artificial Sequence
  Description of Artificial Sequence: Synthetic peptide
  MOD_RES (1)..(1): G, R, or W
  MOD_RES (7)..(7): I or S
  MOD_RES (8)..(8): F or Y
  MOD_RES (9)..(9): L or M
  MOD_RES (10)..(10): R or Y
XASSSVXXXX

SEQ ID NO: 22 - protein, 7 aa, Artificial Sequence
  Description of Artificial Sequence: Synthetic peptide
  MOD_RES (2)..(2): K or T
  MOD_RES (3)..(3): E, P, or S
  MOD_RES (4)..(4): L, N, or P
  MOD_RES (6)..(6): A or T
AXXXLXS

SEQ ID NO: 23 - protein, 9 aa, Artificial Sequence
  Description of Artificial Sequence: Synthetic peptide
  MOD_RES (1)..(1): Q or N
  MOD_RES (4)..(4): D, E, H, N, Q, or S
  MOD_RES (5)..(5): A or G
  MOD_RES (6)..(6): D, F, K, N, R, S, or T
XQWXXXPRT

SEQ ID NO: 24 - protein, 9 aa, Artificial Sequence
  Description of Artificial Sequence: Synthetic peptide
  MOD_RES (1)..(1): H, N, Q, or S
  MOD_RES (3)..(3): F, H, I, P, R, S, W, or Y
  MOD_RES (4)..(4): D, E, H, N, Q, S, or V
  MOD_RES (5)..(5): A, D, G, Q, or S
  MOD_RES (6)..(6): D, F, H, K, L, M, N, Q, R, S, or T
  MOD_RES (9)..(9): T or S
XQXXXXPRX

SEQ ID NO: 25 - protein, 8 aa, Artificial Sequence
  Description of Artificial Sequence: Synthetic peptide
GFTFSSYW

SEQ ID NO: 26 - protein, 8 aa, Artificial Sequence
  Description of Artificial Sequence: Synthetic peptide
GGSFTGFY

SEQ ID NO: 27 - protein, 10 aa, Artificial Sequence
  Description of Artificial Sequence: Synthetic peptide
  MOD_RES (3)..(3): P, S, D, Q, or N
  MOD_RES (5)..(5): T or R
  MOD_RES (6)..(6): N, T, Y, or H
GYXFXXYGIS

SEQ ID NO: 28 - protein, 10 aa, Artificial Sequence
  Description of Artificial Sequence: Synthetic peptide
GYDFTYYGIS

SEQ ID NO: 29 - protein, 7 aa, Artificial Sequence
  Description of Artificial Sequence: Synthetic peptide
SRSYYWG

SEQ ID NO: 30 - protein, 7 aa, Artificial Sequence
  Description of Artificial Sequence: Synthetic peptide
TSNMGVV

SEQ ID NO: 31 - protein, 5 aa, Artificial Sequence
  Description of Artificial Sequence: Synthetic peptide
LYGMN

SEQ ID NO: 32 - protein, 5 aa, Artificial Sequence
  Description of Artificial Sequence: Synthetic peptide
NYGMN

SEQ ID NO: 33 - protein, 10 aa, Artificial Sequence
  Description of Artificial Sequence: Synthetic peptide
GYTFTSSWMH

SEQ ID NO: 34 - protein, 5 aa, Artificial Sequence
  Description of Artificial Sequence: Synthetic peptide
SYDIN

SEQ ID NO: 35 - protein, 5 aa, Artificial Sequence
  Description of Artificial Sequence: Synthetic peptide
SYFWS

SEQ ID NO: 36 - protein, 5 aa, Artificial Sequence
  Description of Artificial Sequence: Synthetic peptide
GYYWN

SEQ ID NO: 37 - protein, 8 aa, Artificial Sequence
  Description of Artificial Sequence: Synthetic peptide
GFTFSTYG

SEQ ID NO: 38 - protein, 5 aa, Artificial Sequence
  Description of Artificial Sequence: Synthetic peptide
TYGMS

SEQ ID NO: 39 - protein, 8 aa, Artificial Sequence
  Description of Artificial Sequence: Synthetic peptide
IKEDGSEK

SEQ ID NO: 40 - protein, 7 aa, Artificial Sequence
  Description of Artificial Sequence: Synthetic peptide
INHRGNT

SEQ ID NO: 41 - protein, 17 aa, Artificial Sequence
  Description of Artificial Sequence: Synthetic peptide
  MOD_RES (4)..(4): T, P, S, or A
  MOD_RES (8)..(8): N, G, V, K, or A
  MOD_RES (9)..(9): T or K
  MOD_RES (10)..(10): H or N
  MOD_RES (13)..(13): Q or R
  MOD_RES (14)..(14): K or M
  MOD_RES (15)..(15): L or H
WISXYNGXXXYAXXXQG

SEQ ID NO: 42 - protein, 17 aa, Artificial Sequence
  Description of Artificial Sequence: Synthetic peptide
WISTYNGNTHYARMLQG

SEQ ID NO: 43 - protein, 16 aa, Artificial Sequence
  Description of Artificial Sequence: Synthetic peptide
SIYYNGRTYYNPSLKS

SEQ ID NO: 44 - protein, 16 aa, Artificial Sequence
  Description of Artificial Sequence: Synthetic peptide
HILWDDREYSNPALKS

SEQ ID NO: 45 - protein, 17 aa, Artificial Sequence
  Description of Artificial Sequence: Synthetic peptide
WINTYTGEPTYADDFKG

SEQ ID NO: 46 - protein, 8 aa, Artificial Sequence
  Description of Artificial Sequence: Synthetic peptide
IHPNSGGT

SEQ ID NO: 47 - protein, 10 aa, Artificial Sequence
  Description of Artificial Sequence: Synthetic peptide
  MOD_RES (8)..(8): N or Y
WLNPNSGXTG

SEQ ID NO: 48 - protein, 16 aa, Artificial Sequence
  Description of Artificial Sequence: Synthetic peptide
YIYYSGNTKYNPSLKS

SEQ ID NO: 49 - protein, 16 aa, Artificial Sequence
  Description of Artificial Sequence: Synthetic peptide
EINHAGNTNYNPSLKS

SEQ ID NO: 50 - protein, 8 aa, Artificial Sequence
  Description of Artificial Sequence: Synthetic peptide
ISGTGRTT

SEQ ID NO: 51 - protein, 17 aa, Artificial Sequence
  Description of Artificial Sequence: Synthetic peptide
WMNTYSGVTTYADDFKG

SEQ ID NO: 52 - protein, 15 aa, Artificial Sequence
  Description of Artificial Sequence: Synthetic peptide
AREDYDSYYKYGMDV

SEQ ID NO: 53 - protein, 13 aa, Artificial Sequence
  Description of Artificial Sequence: Synthetic peptide
ASPFYDFWSGSDY

SEQ ID NO: 54 - protein, 15 aa, Artificial Sequence
  Description of Artificial Sequence: Synthetic peptide
  MOD_RES (8)..(8): S or A
  MOD_RES (9)..(9): Y or P
  MOD_RES (15)..(15): V, A, or G
ENYYGSGXXRGGMDX

SEQ ID NO: 55 - protein, 15 aa, Artificial Sequence
  Description of Artificial Sequence: Synthetic peptide
ENYYGSGAYRGGMDV

SEQ ID NO: 56 - protein, 11 aa, Artificial Sequence
  Description of Artificial Sequence: Synthetic peptide
EDYGDYGAFDI

SEQ ID NO: 57 - protein, 14 aa, Artificial Sequence
  Description of Artificial Sequence: Synthetic peptide
MSRNYYGSSYVMDY

SEQ ID NO: 58 - protein, 10 aa, Artificial Sequence
  Description of Artificial Sequence: Synthetic peptide
DTAMDYAMAY

SEQ ID NO: 59 - protein, 13 aa, Artificial Sequence
  Description of Artificial Sequence: Synthetic peptide
DYGKYGDYYAMDY

SEQ ID NO: 60 - protein, 14 aa, Artificial Sequence
  Description of Artificial Sequence: Synthetic peptide
ARGDYYGYVSWFAY

SEQ ID NO: 61 - protein, 10 aa, Artificial Sequence
  Description of Artificial Sequence: Synthetic peptide
EVPETAAFEY

SEQ ID NO: 62 - protein, 10 aa, Artificial Sequence
  Description of Artificial Sequence: Synthetic peptide
ETGSYYGFDY

SEQ ID NO: 63 - protein, 12 aa, Artificial Sequence
  Description of Artificial Sequence: Synthetic peptide
GYCRSTTCYFDY

SEQ ID NO: 64 - protein, 14 aa, Artificial Sequence
  Description of Artificial Sequence: Synthetic peptide
TKERGDYYYGVFDY

SEQ ID NO: 65 - protein, 13 aa, Artificial Sequence
  Description of Artificial Sequence: Synthetic peptide
EGYVFDDYYATDY

SEQ ID NO: 66 - protein, 12 aa, Artificial Sequence
  Description of Artificial Sequence: Synthetic peptide
QSILYSSNNKNY

SEQ ID NO: 67 - protein, 11 aa, Artificial Sequence
  Description of Artificial Sequence: Synthetic peptide
QSLVHSDGNTY

SEQ ID NO: 68 - protein, 11 aa, Artificial Sequence
  Description of Artificial Sequence: Synthetic peptide
RASQSVSSYLA

SEQ ID NO: 69 - protein, 11 aa, Artificial Sequence
  Description of Artificial Sequence: Synthetic peptide
RASQGISSALA

SEQ ID NO: 70 - protein, 10 aa, Artificial Sequence
  Description of Artificial Sequence: Synthetic peptide
SASSSVNYMH

SEQ ID NO: 71 - protein, 16 aa, Artificial Sequence
  Description of Artificial Sequence: Synthetic peptide
KSSQNIVHSDGNTYLE

SEQ ID NO: 72 - protein, 16 aa, Artificial Sequence
  Description of Artificial Sequence: Synthetic peptide
RSSQSIVHSNGNTYLD

SEQ ID NO: 73 - protein, 6 aa, Artificial Sequence
  Description of Artificial Sequence: Synthetic peptide
QNINVL

SEQ ID NO: 74 - protein, 14 aa, Artificial Sequence
  Description of Artificial Sequence: Synthetic peptide
  MOD_RES (10)..(10): G or A
  MOD_RES (11)..(11): L, S, or Q
  MOD_RES (14)..(14): H or L
TSSSSDIGAXXGVX

SEQ ID NO: 75 - protein, 11 aa, Artificial Sequence
  Description of Artificial Sequence: Synthetic peptide
RASQSINNYLN

SEQ ID NO: 76 - protein, 12 aa, Artificial Sequence
  Description of Artificial Sequence: Synthetic peptide
RASQSVRSSYLA

SEQ ID NO: 77 - protein, 6 aa, Artificial Sequence
  Description of Artificial Sequence: Synthetic peptide
QTISSW

SEQ ID NO: 78 - protein, 16 aa, Artificial Sequence
  Description of Artificial Sequence: Synthetic peptide
RSSQNIVHSDGNTYLE

SEQ ID NO: 79 - protein, 3 aa, Artificial Sequence
  Description of Artificial Sequence: Synthetic peptide
WAS

SEQ ID NO: 80 - protein, 3 aa, Artificial Sequence
  Description of Artificial Sequence: Synthetic peptide
KIS

SEQ ID NO: 81 - protein, 7 aa, Artificial Sequence
  Description of Artificial Sequence: Synthetic peptide
DASNRAT

SEQ ID NO: 82 - protein, 7 aa, Artificial Sequence
  Description of Artificial Sequence: Synthetic peptide
DASSLES

SEQ ID NO: 83 - protein, 7 aa, Artificial Sequence
  Description of Artificial Sequence: Synthetic peptide
STSNLAS

SEQ ID NO: 84 - protein, 7 aa, Artificial Sequence
  Description of Artificial Sequence: Synthetic peptide
KVSNRFS

SEQ ID NO: 85 - protein, 3 aa, Artificial Sequence
  Description of Artificial Sequence: Synthetic peptide
KAS

SEQ ID NO: 86 - protein, 7 aa, Artificial Sequence
  Description of Artificial Sequence: Synthetic peptide
GYYNRPS

SEQ ID NO: 87 - protein, 7 aa, Artificial Sequence
  Description of Artificial Sequence: Synthetic peptide
AASSLQS

SEQ ID NO: 88 - protein, 7 aa, Artificial Sequence
  Description of Artificial Sequence: Synthetic peptide
GASSRAT

SEQ ID NO: 89 - protein, 3 aa, Artificial Sequence
  Description of Artificial Sequence: Synthetic peptide
AAS

SEQ ID NO: 90 - protein, 9 aa, Artificial Sequence
  Description of Artificial Sequence: Synthetic peptide
QQYYSTPFT

SEQ ID NO: 91 - protein, 9 aa, Artificial Sequence
  Description of Artificial Sequence: Synthetic peptide
MQATQFPLT

SEQ ID NO: 92 - protein, 9 aa, Artificial Sequence
  Description of Artificial Sequence: Synthetic peptide
QQRSNWPWT

SEQ ID NO: 93 - protein, 9 aa, Artificial Sequence
  Description of Artificial Sequence: Synthetic peptide
QQFNSYPLT

SEQ ID NO: 94 - protein, 8 aa, Artificial Sequence
  Description of Artificial Sequence: Synthetic peptide
HQWNNYGT

SEQ ID NO: 95 - protein, 9 aa, Artificial Sequence
  Description of Artificial Sequence: Synthetic peptide
FQGSHVPLT

SEQ ID NO: 96 - protein, 9 aa, Artificial Sequence
  Description of Artificial Sequence: Synthetic peptide
QQGQSYPYT

SEQ ID NO: 97 - protein, 10 aa, Artificial Sequence
  Description of Artificial Sequence: Synthetic peptide
  MOD_RES (3)..(3): Y, W, or F
QSXDGTLSAL

SEQ ID NO: 98 - protein, 9 aa, Artificial Sequence
  Description of Artificial Sequence: Synthetic peptide
QQSYSTPRT

SEQ ID NO: 99 - protein, 8 aa, Artificial Sequence
  Description of Artificial Sequence: Synthetic peptide
QQYGSSPT

SEQ ID NO: 100 - protein, 8 aa, Artificial Sequence
  Description of Artificial Sequence: Synthetic peptide
QQYHRSWT

SEQ ID NO: 101 - protein, 116 aa, Artificial Sequence
  Description of Artificial Sequence: Synthetic polypeptide
QVQLVQSGAEVKKPGASVKVSCKASGFDIQDTYMHWVKQRPGQGLEWMGRIDPASGHTKYDPKFQVRVTITRDTSTSTVYMELSSLRSEDTAVYYCARSGGLPDVWGQGTTVTVSS

SEQ ID NO: 102 - protein, 116 aa, Artificial Sequence
  Description of Artificial Sequence: Synthetic polypeptide
QVQLVQSGAEVKKPGASVKVSCKASGFDIQDTYMHWVKQRPGQGLEWMGRIDPASGHTKYDPKFQVRVTMTRDTSTSTAYLELSSLRSEDTAVYYCARSGGLPDVWGQGTTVTVSS

SEQ ID NO: 103 - protein, 116 aa, Artificial Sequence
  Description of Artificial Sequence: Synthetic polypeptide
EVQLVQSGAEVKKPGASVKVSCKASGFDIQDTYMHWVRQRPGQGLEWIGRIDPASGHTKYDPKFQVRATITTDTSTSTAYLELSSLRSEDTAVYYCARSGGLPDVWGQGTTVTVSS

SEQ ID NO: 104 - protein, 116 aa, Artificial Sequence
  Description of Artificial Sequence: Synthetic polypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSS

SEQ ID NO: 105 - protein, 116 aa, Artificial Sequence
  Description of Artificial Sequence: Synthetic polypeptide
QVQLVQSGAEVKKPGASVKVSCKASGFDIQDTYMHWVKQRPGQGLEWMGRIDPASGHTKYDPKFQVRATITRDTSTSTAYLELSSLRSEDTAVYYCARSGGLPDVWGQGTTVTVSS

SEQ ID NO: 106 - protein, 116 aa, Artificial Sequence
  Description of Artificial Sequence: Synthetic polypeptide
QVQLVQSGAEVKKPGASVKVSCKASGFDIQDTYMHWVRQRPGQGLEWMGRIDPASGHTKYDPKFQVRVTITTDTSTSTVYMELSSLRSEDTAVYYCARSGGLPDVWGQGTTVTVSS

SEQ ID NO: 107 - protein, 116 aa, Artificial Sequence
  Description of Artificial Sequence: Synthetic polypeptide
QVQLVQSGAEVKKPGASVKVSCKASGFDIQDTYMHWVRQRPGQGLEWIGRIDPASGHTKYDPKFQVRATITTDTSTSTAYLELSSLRSEDTAVYYCARSGGLPDVWGQGTTVTVSS

SEQ ID NO: 108 - protein, 116 aa, Artificial Sequence
  Description of Artificial Sequence: Synthetic polypeptide
QVQLVQSGAEVKKPGASVKVSCKASGFDIQDTYMHWVKQRPGQGLEWIGRIDPASGHTKYDPKFQVRATITTDTSTSTAYLELSSLRSEDTAVYYCARSGGLPDVWGQGTTVTVSS

SEQ ID NO: 109 - protein, 116 aa, Artificial Sequence
  Description of Artificial Sequence: Synthetic polypeptide
EVQLVQSGAEVKKPGASVKVSCKASGFDIQDTYMHWVKQRPGQGLEWIGRIDPASGHTKYDPKFQVRATITTDTSTSTAYLELSSLRSEDTAVYYCARSGGLPDVWGQGTTVTVSS

SEQ ID NO: 110 - protein, 116 aa, Artificial Sequence
  Description of Artificial Sequence: Synthetic polypeptide
QVQLVQSGAEVKKPGASVKVSCKASGFDIQDTYMHWVRQRPGQGLEWMGRIDPASGHTKYDPKFQVRVTITTDTSTSTAYLELSSLRSEDTAVYYCARSGGLPDVWGQGTTVTVSS

SEQ ID NO: 111 - protein, 116 aa, Artificial Sequence
  Description of Artificial Sequence: Synthetic polypeptide
QVQLVQSGAEVKKPGASVKVSCKASGFDIQDTYMHWVRQRPGQGLEWMGRIDPASGHTKYDPKFQVRVTITRDTSTSTAYLELSSLRSEDTAVYYCARSGGLPDVWGQGTTVTVSS

SEQ ID NO: 112 - protein, 116 aa, Artificial Sequence
  Description of Artificial Sequence: Synthetic polypeptide
QVQLVQSGAEVKKPGASVKVSCKASGFDIQDTYMHWVRQRPGQGLEWIGRIDPASGHTKYDPKFQVRVTITRDTSTSTAYLELSSLRSEDTAVYYCARSGGLPDVWGQGTTVTVSS

SEQ ID NO: 113 - protein, 116 aa, Artificial Sequence
  Description of Artificial Sequence: Synthetic polypeptide
QVQLVQSGAEVKKPGASVKVSCKASGFDIQDTYMHWVRQRPGQGLEWIGRIDPASGHTKYDPKFQVRATITRDTSTSTAYLELSSLRSEDTAVYYCARSGGLPDVWGQGTTVTVSS

SEQ ID NO: 114 - protein, 116 aa, Artificial Sequence
  Description of Artificial Sequence: Synthetic polypeptide
QVQLVQSGAEVKKPGASVKVSCKASGFDIQDTYMHWVKQRPGQGLEWMGRIDPASGHTKYDPKFQVRVTITRDTSTSTAYLELSSLRSEDTAVYYCARSGGLPDVWGQGTTVTVSS

SEQ ID NO: 115 - protein, 116 aa, Artificial Sequence
  Description of Artificial Sequence: Synthetic polypeptide
QVQLVQSGAEVKKPGASVKVSCKASGFDIQDTYMHWVKQRPGQGLEWIGRIDPASGHTKYDPKFQVRVTITTDTSTSTAYLELSSLRSEDTAVYYCARSGGLPDVWGQGTTVTVSS

SEQ ID NO: 116 - protein, 116 aa, Artificial Sequence
  Description of Artificial Sequence: Synthetic polypeptide
EVQLVQSGAEVKKPGASVKVSCKASGFDIQDTYMHWVKQAPGQGLEWMGRIEPASGHIKYDPKFQGRVTMTRDTSTSTVYMELSSLRSEDTAVYYCARSGGLPDWWGQGTTVTVSS

SEQ ID NO: 117 - protein, 116 aa, Artificial Sequence
  Description of Artificial Sequence: Synthetic polypeptide
QVQLVQSGAEVKKPGASVKVSCKASGFDIQDTYMHWVKQRPGQGLEWMGRIEPASGHIKYDPKFQGRVTMTRDTSTSTVYMELSSLRSEDTAVYYCARSGGLPDWWGQGTTVTVSS

SEQ ID NO: 118 - protein, 116 aa, Artificial Sequence
  Description of Artificial Sequence: Synthetic polypeptide
QVQLVQSGAEVKKPGASVKVSCKASGFDIQDTYMHWVKQRPGQGLEWMGRIDPASGHTKYDPKFQVRVTITTDTSTSTAYLELSSLRSEDTAVYYCARSGGLPDVWGQGTTVTVSS

SEQ ID NO: 119 - protein, 116 aa, Artificial Sequence
  Description of Artificial Sequence: Synthetic polypeptide
QVQLVQSGAEVKKPGASVKVSCKASGFDIQDTYMHWVKQRPGQGLEWMGRIDPASGHTKYDPKFQVRVTITRDTSTSTAYMELSSLRSEDTAVYYCARSGGLPDVWGQGTTVTVSS

SEQ ID NO: 120 - protein, 116 aa, Artificial Sequence
  Description of Artificial Sequence: Synthetic polypeptide
QVQLVQSGAEVKKPGASVKVSCKASGFDIQDTYMHWVKQRPGQGLEWIGRIDPASGHTKYDPKFQVRVTITRDTSTSTAYLELSSLRSEDTAVYYCARSGGLPDVWGQGTTVTVSS

SEQ ID NO: 121 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIEPASGHIKYDPKFQVRVTMTRDTSTSTVYMELSSLRSEDTAVYYCARSGGLPDWWGQGTTVTVSS

SEQ ID NO: 122 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIEPASGHIKYDPKFQVRATMTRDTSTSTVYMELSSLRSEDTAVYYCARSGGLPDWWGQGTTVTVSS

SEQ ID NO: 123 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIEPASGHIKYDPKFQVRVTITRDTSTSTVYMELSSLRSEDTAVYYCARSGGLPDWWGQGTTVTVSS

SEQ ID NO: 124 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIEPASGHIKYDPKFQVRATITRDTSTSTVYMELSSLRSEDTAVYYCARSGGLPDWWGQGTTVTVSS

SEQ ID NO: 125 - protein, 116 aa, Artificial Sequence
  Description of Artificial Sequence: Synthetic polypeptide
QVQLVQSGAEVKKPGASVKVSCKASGFDIQDTYMHWVKQAPGQGLEWMGRIEPASGHIKYDPKFQGRVTMTRDTSTSTVYMELSSLRSEDTAVYYCARSGGLPDWWGQGTTVTVSS

SEQ ID NO: 126 - protein, 116 aa, Artificial Sequence
  Description of Artificial Sequence: Synthetic polypeptide
QVQLVQSGAEVKKPGASVKVSCKASGFDIQDTYMHWVKQAPGQGLEWMGRIDPASGHIKYDPKFQGRVTMTRDTSTSTVYMELSSLRSEDTAVYYCARSGGLPDWWGQGTTVTVSS

SEQ ID NO: 127 - protein, 116 aa, Artificial Sequence
  Description of Artificial Sequence: Synthetic polypeptide
QVQLVQSGAEVKKPGASVKVSCKASGFDIQDTYMHWVKQAPGQGLEWMGRIEPASGHIKYDPKFQGRATMTRDTSTSTVYMELSSLRSEDTAVYYCARSGGLPDWWGQGTTVTVSS

SEQ ID NO: 128 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIEPASGHIKYDPKFQGRATMTRDTSTSTVYMELSSLRSEDTAVYYCARSGGLPDWWGQGTTVTVSS

SEQ ID NO: 129 - protein, 116 aa, Artificial Sequence
  Description of Artificial Sequence: Synthetic polypeptide
EVQLVQSGAEVKKPGASVKVSCKASGFDIQDTYMHWVRQAPGQGLEWMGRIEPASGHIKYDPKFQGRATMTRDTSTSTVYMELSSLRSEDTAVYYCARSGGLPDWWGQGTTVTVSS

SEQ ID NO: 130 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIDPASGHIKYDPKFQGRATMTRDTSTSTVYMELSSLRSEDTAVYYCARSGGLPDWWGQGTTVTVSS

SEQ ID NO: 131 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIEPASGHIKYDPKFQGRATITTDTSTSTVYMELSSLRSEDTAVYYCARSGGLPDWWGQGTTVTVSS

SEQ ID NO: 132 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIEPASGHIKYDPKFQGRATMTRDTSTSTAYMELSSLRSEDTAVYYCARSGGLPDWWGQGTTVTVSS

SEQ ID NO: 133 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIEPASGHIKYDPKFQGRVTMTRDTSTSTAYMELSSLRSEDTAVYYCARSGGLPDWWGQGTTVTVSS

SEQ ID NO: 134 - protein, 116 aa, Artificial Sequence
  Description of Artificial Sequence: Synthetic polypeptide
EVQLVQSGAEVKKPGASVKVSCKASGFDIQDTYMHWVRQAPGQGLEWMGRIEPASGHIKYDPKFQGRVTMTRDTSTSTAYMELSSLRSEDTAVYYCARSGGLPDWWGQGTTVTVSS

SEQ ID NO: 135 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIDPASGHIKYDPKFQGRVTMTRDTSTSTAYMELSSLRSEDTAVYYCARSGGLPDWWGQGTTVTVSS

SEQ ID NO: 136 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIEPASGHIKYDPKFQGRVTMTRDTSTSTVYMELSSLRSEDTAVYYCARSGGLPDWWGQGTTVTVSS

SEQ ID NO: 137 - protein, 116 aa, Artificial Sequence
  Description of Artificial Sequence: Synthetic polypeptide
DVQLVQSGAEVKKPGASVKVSCKASGFDIQDTYMHWVRQAPGQGLEWMGRIEPASGHIKYDPKFQGRVTMTRDTSTSTVYMELSSLRSEDTAVYYCARSGGLPDWWGQGTTVTVSS

SEQ ID NO: 138 - protein, 116 aa, Artificial Sequence
  Description of Artificial Sequence: Synthetic polypeptide
QVQLVQSGAEVKKPGASVKVSCKASGFDIQDTYMHWVKQAPGQGLEWIGRIDPASGHTKYDPKFQVRATITTDTSTSTAYLELSSLRSEDTAVYYCARSGGLPDVWGQGTTVTVSS

SEQ ID NO: 139 - protein, 116 aa, Artificial Sequence
  Description of Artificial Sequence: Synthetic polypeptide
QVQLVQSGAEVKKPGASVKVSCKASGFDIQDTYMHWVRQAPGQGLEWIGRIDPASGHTKYDPKFQVRATITTDTSTSTAYLELSSLRSEDTAVYYCARSGGLPDVWGQGTTVTVSS

SEQ ID NO: 140 - protein, 116 aa, Artificial Sequence
  Description of Artificial Sequence: Synthetic polypeptide
QVQLVQSGAEVKKPGASVKVSCKASGFDIQDTYMHWVKQRPGQGLEWMGRIDPASGHTKYDPKFQVRATITTDTSTSTAYLELSSLRSEDTAVYYCARSGGLPDVWGQGTTVTVSS

SEQ ID NO: 141 - protein, 116 aa, Artificial Sequence
  Description of Artificial Sequence: Synthetic polypeptide
QVQLVQSGAEVKKPGASVKVSCKASGFDIQDTYMHWVKQRPGQGLEWIGRIDPASGHTKYDPKFQVRATITTDTSTSTVYLELSSLRSEDTAVYYCARSGGLPDVWGQGTTVTVSS

SEQ ID NO: 142 - protein, 116 aa, Artificial Sequence
  Description of Artificial Sequence: Synthetic polypeptide
QVQLVQSGAEVKKPGASVKVSCKASGFDIQDTYMHWVKQRPGQGLEWIGRIDPASGHTKYDPKFQVRATITTDTSTSTAYMELSSLRSEDTAVYYCARSGGLPDVWGQGTTVTVSS

SEQ ID NO: 143 - protein, 116 aa, Artificial Sequence
  Description of Artificial Sequence: Synthetic polypeptide
QVQLVQSGAEVKKPGASVKVSCKASGFDIQDTYMHWVKQRPGQGLEWIGRIDPASGHTKYDPKFQVRATITTDTSTSTVYMELSSLRSEDTAVYYCARSGGLPDVWGQGTTVTVSS

SEQ ID NO: 144 - protein, 116 aa, Artificial Sequence
  Description of Artificial Sequence: Synthetic polypeptide
QVQLVQSGAEVKKPGASVKVSCKASGFDIQDTYMHWVKQRPGQGLEWIGRIDPASGHTKYDPKFQVRATMTTDTSTSTAYLELSSLRSEDTAVYYCARSGGLPDVWGQGTTVTVSS

SEQ ID NO: 145 - protein, 116 aa, Artificial Sequence
  Description of Artificial Sequence: Synthetic polypeptide
QVQLVQSGAEVKKPGASVKVSCKASGFDIQDTYMHWVKQRPGQGLEWIGRIDPASGHTKYDPKFQVRATITRDTSTSTAYLELSSLRSEDTAVYYCARSGGLPDVWGQGTTVTVSS

SEQ ID NO: 146 - protein, 116 aa, Artificial Sequence
  Description of Artificial Sequence: Synthetic polypeptide
QVQLVQSGAEVKKPGASVKVSCKASGFDIQDTYMHWVKQRPGQGLEWIGRIDPASGHTKYDPKFQVRVTMTTDTSTSTAYLELSSLRSEDTAVYYCARSGGLPDVWGQGTTVTVSS

SEQ ID NO: 147 - protein, 116 aa, Artificial Sequence
  Description of Artificial Sequence: Synthetic polypeptide
QVQLVQSGAEVKKPGASVKVSCKASGFDIQDTYMHWVKQRPGQGLEWIGRIDPASGHTKYDPKFQVRATMTRDTSTSTAYLELSSLRSEDTAVYYCARSGGLPDVWGQGTTVTVSS

SEQ ID NO: 148 - protein, 116 aa, Artificial Sequence
  Description of Artificial Sequence: Synthetic polypeptide
QVQLVQSGAEVKKPGASVKVSCKASGFDIQDTYMHWVKQRPGQGLEWIGRIDPASGHTKYDPKFQVRVTMTRDTSTSTAYLELSSLRSEDTAVYYCARSGGLPDVWGQGTTVTVSS

SEQ ID NO: 149 - protein, 116 aa, Artificial Sequence
  Description of Artificial Sequence: Synthetic polypeptide
DVQLVQSGAEVKKPGASVKVSCKASGFDIQDTYMHWVKQRPGQGLEWIGRIDPASGHTKYDPKFQVRATITTDTSTSTAYLELSSLRSEDTAVYYCARSGGLPDVWGQGTTVTVSS

SEQ ID NO: 150 - protein, 116 aa, Artificial Sequence
  Description of Artificial Sequence: Synthetic polypeptide
QVQLVQSGAEVKKPGASVKVSCKASGFDIQDTYMHWVRQRPGQGLEWMGRIEPASGHIKYDPKFQGRVTMTRDTSTSTVYMELSSLRSEDTAVYYCARSGGLPDWWGQGTTVTVSS

SEQ ID NO: 151 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIDPASGHIKYDPKFQGRVTMTRDTSTSTVYMELSSLRSEDTAVYYCARSGGLPDWWGQGTTVTVSS

SEQ ID NO: 152 - protein, 116 aa, Artificial Sequence
  Description of Artificial Sequence: Synthetic polypeptide
QVQLVQSGAEVKKPGASVKVSCKASGFDIQDTYMHWVRQAPGQGLEWIGRIEPASGHIKYDPKFQGRVTMTRDTSTSTVYMELSSLRSEDTAVYYCARSGGLPDWWGQGTTVTVSS

SEQ ID NO: 153 - protein, 116 aa, Artificial Sequence
  Description of Artificial Sequence: Synthetic polypeptide
QVQLVQSGAEVKKPGASVKVSCKASGFDIQDTYMHWVRQAPGQGLEWIGRIDPASGHIKYDPKFQGRVTMTRDTSTSTVYMELSSLRSEDTAVYYCARSGGLPDWWGQGTTVTVSS

SEQ ID NO: 154 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIEPASGHVKYDPKFQGRVTMTRDTSTSTVYMELSSLRSEDTAVYYCARSGGLPDWWGQGTTVTVSS

SEQ ID NO: 155 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIEPASGHIKYDPKFQGRVTITRDTSTSTVYMELSSLRSEDTAVYYCARSGGLPDWWGQGTTVTVSS

SEQ ID NO: 156 - protein, 116 aa, Artificial Sequence
  Description of Artificial Sequence: Synthetic polypeptide
EVQLVQSGAEVKKPGASVKVSCKASGFDIQDTYMHWVRQAPGQGLEWMGRIEPASGHIKYDPKFQGRVTMTRDTSTSTVYMELSSLRSEDTAVYYCARSGGLPDWWGQGTTVTVSS

SEQ ID NO: 157 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIEPASGHIKYDPKFQGRVTMTTDTSTSTVYMELSSLRSEDTAVYYCARSGGLPDWWGQGTTVTVSS

SEQ ID NO: 158 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIEPASGHIKYDPKFQGRVTITTDTSTSTVYMELSSLRSEDTAVYYCARSGGLPDWWGQGTTVTVSS

SEQ ID NO: 159 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIEPASGHTKYDPKFQGRVTMTRDTSTSTVYMELSSLRSEDTAVYYCARSGGLPDWWGQGTTVTVSS

SEQ ID NO: 160 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIEPASGHIKYDPKFQGRATITRDTSTSTVYMELSSLRSEDTAVYYCARSGGLPDWWGQGTTVTVSS

SEQ ID NO: 161 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIEPASGHIKYDPKFQGRVTMTRDTSTSTVYLELSSLRSEDTAVYYCARSGGLPDWWGQGTTVTVSS

SEQ ID NO: 162 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIEPASGHIKYDPKFQGRVTMTRDTSTSTAYLELSSLRSEDTAVYYCARSGGLPDWWGQGTTVTVSS

SEQ ID NO: 163 - protein, 116 aa, Artificial Sequence
  Description of Artificial Sequence: Synthetic polypeptide
QVQLVQSGAEVKKPGASVKVSCKASGFDIQDTYMHWVRQRPGQGLEWMGRIDPASGHTKYDPKFQVRATITTDTSTSTAYLELSSLRSEDTAVYYCARSGGLPDVWGQGTTVTVSS

SEQ ID NO: 164 - protein, 116 aa, Artificial Sequence
  Description of Artificial Sequence: Synthetic polypeptide
QVQLVQSGAEVKKPGASVKVSCKASGFDIQDTYMHWVRQRPGQGLEWIGRIDPASGHTKYDPKFQVRVTITTDTSTSTAYLELSSLRSEDTAVYYCARSGGLPDVWGQGTTVTVSS

SEQ ID NO: 165 - protein, 116 aa, Artificial Sequence
  Description of Artificial Sequence: Synthetic polypeptide
QVQLVQSGAEVKKPGASVKVSCKASGFDIQDTYMHWVRQRPGQGLEWMGRIDPASGHTKYDPKFQVRVTITTDTSTSTVYLELSSLRSEDTAVYYCARSGGLPDVWGQGTTVTVSS

SEQ ID NO: 166 - protein, 116 aa, Artificial Sequence
  Description of Artificial Sequence: Synthetic polypeptide
QVQLVQSGAEVKKPGASVKVSCKASGFDIQDTYMHWVRQRPGQGLEWMGRIDPASGHTKYDPKFQVRVTITTDTSTSTAYMELSSLRSEDTAVYYCARSGGLPDVWGQGTTVTVSS

SEQ ID NO: 167 - protein, 116 aa, Artificial Sequence
  Description of Artificial Sequence: Synthetic polypeptide
QVQLVQSGAEVKKPGASVKVSCKASGFDIQDTYMHWVRQRPGQGLEWMGRIDPASGHTKYDPKFQVRVTMTTDTSTSTAYLELSSLRSEDTAVYYCARSGGLPDVWGQGTTVTVSS

SEQ ID NO: 168 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIDPASGHIKYDPKFQGRATITTDTSTSTVYMELSSLRSEDTAVYYCARSGGLPDWWGQGTTVTVSS

SEQ ID NO: 169 - protein, 116 aa, Artificial Sequence
  Description of Artificial Sequence: Synthetic polypeptide
EVQLVQSGAEVKKPGASVKVSCKASGFDIQDTYMHWVRQAPGQGLEWMGRIEPASGHIKYDPKFQGRATITTDTSTSTVYMELSSLRSEDTAVYYCARSGGLPDWWGQGTTVTVSS

SEQ ID NO: 170 - protein, 116 aa, Artificial Sequence
  Description of Artificial Sequence: Synthetic polypeptide
EVQLQQSGAELVKPGASVKLSCTASGFDIQDTYMHWVKQRPEQGLEWIGRIDPASGHTKYDPKFQVKATITTDTSSNTAYLQLSSLTSEDTAVYYCSRSGGLPDVWGAGTTVTVSS

SEQ ID NO: 171 - protein, 116 aa, Artificial Sequence
  Description of Artificial Sequence: Synthetic polypeptide
QVHLQQSGPELVKPGASVKLSCKASGYTFTKYDINWVRQRPEQGLEWIGWIFPGDGRTDYNEKFKGKATLTTDKSSSTAYMEVSRLTSEDSAVYFCARYGPAMDYWGQGTSVTVAS

SEQ ID NO: 172 - protein, 116 aa, Artificial Sequence
  Description of Artificial Sequence: Synthetic polypeptide
QVQLVQSGAEVKKPGASVKVSCKASGFDICDTYMHWVKQRPGQGLEWIGRIDPASGHTKYDPKFQVRATITTDTSTSTAYLELSSLRSEDTAVYYCARSGGLPDVWGQGTTVTVSS

SEQ ID NO: 173 - protein, 116 aa, Artificial Sequence
  Description of Artificial Sequence: Synthetic polypeptide
QVQLVQSGAEVKKPGASVKLSCKASGFDICDTYMHWVRQRPGQGLEWIGRIDPASGHTKYDPKFQVRATMTTDTSTSTVYLELSSLRSEDTAVYYCARSGGLPDVWGQGTTVTVSS

SEQ ID NO: 174 - protein, 116 aa, Artificial Sequence
  Description of Artificial Sequence: Synthetic polypeptide
QVQLVQSGAEVVKPGASVKLSCKASGFDICDTYMHWVRQRPGQGLEWMGRIDPASGHTKYDPKFQVRVTMTTDTSTSTVYLELSSLRSEDTAVYYCARSGGLPDVWGQGTTVTVSS

SEQ ID NO: 175 - protein, 116 aa, Artificial Sequence
  Description of Artificial Sequence: Synthetic polypeptide
QVQLVQSGAEVKKPGASVKVSCKASGFDICDTYMHWVKQRPGQGLEWIGRIDPASGHTKYDPKFQVRATITRDTSTSTAYLELSSLRSEDTAVYYCSRSGGLPDVWGQGTTVTVSS

SEQ ID NO: 176 - protein, 116 aa, Artificial Sequence
  Description of Artificial Sequence: Synthetic polypeptide
QVQLVQSGAEVKKPGASVKVSCKASGFDICDTYMHWVKQAPGQGLEWMGRIDPASGHTKYDPKFQVRATMTTDTSTSTAYLELSSLRSEDTAVYYCSRSGGLPDVWGQGTTVTVSS

SEQ ID NO: 177 - protein, 116 aa, Artificial Sequence
  Description of Artificial Sequence: Synthetic polypeptide
QVQLVQSGAEVKKPGASVKLSCKASGFDIQDTYMHWVRQAPGQGLEWMGRIDPASGHTKYDPKFQVRVTMTTDTSTSTVYMELSSLRSEDTAVYYCSRSGGLPDVWGQGTTVTVSS

SEQ ID NO: 178 - protein, 116 aa, Artificial Sequence
  Description of Artificial Sequence: Synthetic polypeptide
QVQLVQSGAEVKKPGASVKLSCKASGFDIQDTYMHWVRQAPGQGLEWMGRIDPASGHTKYDPKFQVRVTMTRDTSTSTVYMELSSLRSEDTAVYYCSRSGGLPDVWGQGTTVTVSS

SEQ ID NO: 179 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIDPASGHTKYDPKFQVRVTMTRDTSTSTVYMELSSLRSEDTAVYYCARSGGLPDVWGQGTTVTVSS

SEQ ID NO: 180 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIDPASGHTKYDPKFQVRVTMTRDTSTSTVYMELSSLRSEDTAVYYCARSGGLPDKWGQGTTVTVSS

SEQ ID NO: 181 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIDPASGHTKYDPKFQVRVTMTRDTSTSTVYMELSSLRSEDTAVYYCARSGGLPDMWGQGTTVTVSS

SEQ ID NO: 182 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIDPASGHTKYDPKFQVRVTMTRDTSTSTVYMELSSLRSEDTAVYYCARSGGLPDQWGQGTTVTVSS

SEQ ID NO: 183 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIDPASGHTKYDPKFQVRVTMTRDTSTSTVYMELSSLRSEDTAVYYCARSGGLPDWWGQGTTVTVSS

SEQ ID NO: 184 - protein, 116 aa, Artificial Sequence
  Description of Artificial Sequence: Synthetic polypeptide
  MOD_RES (28)..(28): D or E
  MOD_RES (29)..(29): I, P, or V
  MOD_RES (30)..(30): G, Q, S, or V
  MOD_RES (33)..(33): F or Y
  MOD_RES (34)..(34): I or M
  MOD_RES (102)..(102): L or M
  MOD_RES (105)..(105): E, I, K, L, M, Q, T, V, W, or Y
QVQLVQSGAEVKKPGASVKVSCKASGFXXXDTXXHWVRQAPGQGLEWMGRIDPASGHTKYDPKFQVRVTMTRDTSTSTVYMELSSLRSEDTAVYYCARSGGXPDXWGQGTTVTVSS

SEQ ID NO: 185 - protein, 116 aa, Artificial Sequence
  Description of Artificial Sequence: Synthetic polypeptide
  MOD_RES (28)..(28): D or E
  MOD_RES (29)..(29): I, P, or V
  MOD_RES (30)..(30): G, Q, S, or V
  MOD_RES (33)..(33): F or Y
  MOD_RES (34)..(34): I or M
  MOD_RES (102)..(102): L or M
  MOD_RES (105)..(105): E, I, K, L, M, Q, T, V, W, or Y
QVQLVQSGAEVKKPGASVKVSCKASGFXXXDTXXHWVRQAPGQGLEWMGRIDPASGHTKYDPKFQVRVTMTRDTSTSTVYMELSSLRSEDTAVYYCSRSGGXPDXWGQGTTVTVSS

SEQ ID NO: 186 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIEPASGHIKYSPKFQGRVTMTRDTSTSTVYMELSSLRSEDTAVYYCARSGGLPDWWGQGTTVTVSS

SEQ ID NO: 187 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIEPASGHVKYSPKFQVRVTMTRDTSTSTVYMELSSLRSEDTAVYYCARSGGLPDWWGQGTTVTVSS

SEQ ID NO: 188 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIEPASGHVKYDPKFQTRVTMTRDTSTSTVYMELSSLRSEDTAVYYCARSGGLPDWWGQGTTVTVSS

SEQ ID NO: 189 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIDPASGHIKYDPKFQKRVTMTRDTSTSTVYMELSSLRSEDTAVYYCARSGGLPDMWGQGTTVTVSS

SEQ ID NO: 190 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIDPASGHVKIDPKFQVRVTMTRDTSTSTVYMELSSLRSEDTAVYYCARSGGLPDMWGQGTTVTVSS

SEQ ID NO: 191 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIDPASGHLKYDPKFQVRVTMTRDTSTSTVYMELSSLRSEDTAVYYCARSGGLPDMWGQGTTVTVSS

SEQ ID NO: 192 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIDPASGHLKYDPKFQRRVTMTRDTSTSTVYMELSSLRSEDTAVYYCARSGGLPDMWGQGTTVTVSS

SEQ ID NO: 193 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIDPASGHLKYDPKFQNRVTMTRDTSTSTVYMELSSLRSEDTAVYYCARSGGLPDKWGQGTTVTVSS

SEQ ID NO: 194 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIEPASGHLKYDPKFQERVTMTRDTSTSTVYMELSSLRSEDTAVYYCARSGGLPDKWGQGTTVTVSS

SEQ ID NO: 195 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIDPASGHLKYDPKFQGRVTITRDTSASTAYMELSSLRSEDTAVYYCARSGGLPDMWGQGTTVTVSS

SEQ ID NO: 196 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIDPASGHLKYDPKFQGRVTITRDTSASTVYMELSSLRSEDTAVYYCARSGGLPDMWGQGTTVTVSS

SEQ ID NO: 197 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIDPASGHTKYDPKFQGRATITTDTSASTAYLQLSSLRSEDTAVYYCARSGGLPDVWGQGTTVTVSS

SEQ ID NO: 198 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIDPASGHTKYDPKFQVRATITTDTSASTAYLQLSSLRSEDTAVYYCARSGGLPDFWGQGTTVTVSS

SEQ ID NO: 199 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIDPASGHTKYDPKFQGRATITTDTSASTAYLQLSSLRSEDTAVYYCARSGGLPDFWGQGTTVTVSS

SEQ ID NO: 200 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIDPASGHTKYDPKFQGRATITTDTSASTAYLQLSSLRSEDTAVYYCARSGGLPDLWGQGTTVTVSS

SEQ ID NO: 201 - protein, 106 aa, Artificial Sequence
  Description of Artificial Sequence: Synthetic polypeptide
EIVLTQSPGTLSLSPGERATLSCRASSSVSYMYWYQQKPGQAPRPLIYATSNLASGIPDRFSGSGSGTDFTLTISRLEPEDFAVYYCQQWEGNPRTFGGGTKLEIK

SEQ ID NO: 202 - protein, 106 aa, Artificial Sequence
  Description of Artificial Sequence: Synthetic polypeptide
EIVLTQSPGTLSLSPGERATLSCRASSSVSYMYWYQQKPGQAPRLLIYATSNLASGIPDRFSGSGSGTDFTLTISRLEPEDFAVYYCQQWEGNPRTFGGGTKLEIK

SEQ ID NO: 203 - protein, 106 aa, Artificial Sequence
  Description of Artificial Sequence: Synthetic polypeptide
EIVLTQSPGTLSLSPGERATLSCRASSSVSYMYWYQQKPGQAPRLLIYATSNLASGIPDRFSGSGSGTDFTLTISRLEPEDFAVYYCQQWSGNPRTFGGGTKLEIK

SEQ ID NO: 204 - protein, 106 aa, Artificial Sequence
  Description of Artificial Sequence: Synthetic polypeptide
EIVLTQSPGTLSLSPGERATLSCRASSSVSYMYWYQQKPGQAPRLLIYATSNLASGIPDRFSGSGSGTDFTLTISRLEPEDFAVYYCQQWKGNPRTFGGGTKLEIK

SEQ ID NO: 205 - protein, 106 aa, Artificial Sequence
  Description of Artificial Sequence: Synthetic polypeptide
EIVLTQSPGTLSLSPGERATLSCRASSSVSYMYWYQQKPGQAPRPWIYATSNLASGIPDRFSGSGSGTDFTLTISRLEPEDFAVYYCQQWEGNPRTFGGGTKLEIK

SEQ ID NO: 206 - protein, 106 aa, Artificial Sequence
  Description of Artificial Sequence: Synthetic polypeptide
EIVLTQSPGTLSLSPGERATLSCRASSSVSYMYWYQQKPGQAPRPLIYATSNLASGIPDRFSGSGSGTDFTLTISRLEPEDFAVYYCQQWKGNPRTFGGGTKLEIK

SEQ ID NO: 207 - protein, 106 aa, Artificial Sequence
  Description of Artificial Sequence: Synthetic polypeptide
EIVLTQSPGTLSLSPGERATLSCRASSSVSYMYWYQQKPGQAPRLLIYATSNLASGIPDRFSGSGSGTDFTLTISRLEPEDFAVYYCQQWSRNPRTFGGGTKLEIK

SEQ ID NO: 208 - protein, 106 aa, Artificial Sequence
  Description of Artificial Sequence: Synthetic polypeptide
EIVLTQSPGTLSASPGERATMSCRASSSVSYMYWYQQKPGQAPRPWIYATSNLASGVPDRFSGSGSGTDYTLTISRVEPEDFAVYYCQQWSGNPRTFGGGTKLEIK

SEQ ID NO: 209 - protein, 106 aa, Artificial Sequence
  Description of Artificial Sequence: Synthetic polypeptide
EIVLTQSPGTLSLSPGERATLSCRASSSVSYMYWYQQKPGQAPRLLIYATSNLASGVPDRFSGSGSGTDFTLTISRLEPEDFAVYYCQQWEGNPRTFGGGTKLEIK

SEQ ID NO: 210 - protein, 106 aa, Artificial Sequence
  Description of Artificial Sequence: Synthetic polypeptide
EIVLTQSPGTLSASPGERATLSCRASSSVSYMYWYQQKPGQAPRPWIYATSNLASGVPDRFSGSGSGTDYTLTISRVEPEDFAVYYCQQWSGNPRTFGGGTKLEIK

SEQ ID NO: 211 - protein, 106 aa, Artificial Sequence
  Description of Artificial Sequence: Synthetic polypeptide
EIVLTQSPGTLSASPGERATMSCRASSSVSYMYWYQQKPGQAPRLLIYATSNLASGVPDRFSGSGSGTDYTLTISRVEPEDFAVYYCQQWSGNPRTFGGGTKLEIK

SEQ ID NO: 212 - protein, 106 aa, Artificial Sequence
  Description of Artificial Sequence: Synthetic polypeptide
EIVLTQSPGTLSASPGERATMSCRASSSVSYMYWYQQKPGQAPRPWIYATSNLASGVPDRFSGSGSGTDFTLTISRVEPEDFAVYYCQQWSGNPRTFGGGTKLEIK

SEQ ID NO: 213 - protein, 106 aa, Artificial Sequence
  Description of Artificial Sequence: Synthetic polypeptide
EIVLTQSPGTLSASPGERATMSCRASSSVSYMYWYQQKPGQAPRPWIYATSNLASGVPDRFSGSGSGTDYTLTISRLEPEDFAVYYCQQWSGNPRTFGGGTKLEIK

SEQ ID NO: 214 - protein, 106 aa, Artificial Sequence
  Description of Artificial Sequence: Synthetic polypeptide
EIVLTQSPGTLSASPGERATMSCRASSSVSYMYWYQQKPGQAPRPWIYATSNLASGVPDRFSGSGSGTDFTLTISRLEPEDFAVYYCQQWSGNPRTFGGGTKLEIK

SEQ ID NO: 215 - protein, 106 aa, Artificial Sequence
  Description of Artificial Sequence: Synthetic polypeptide
EIVLTQSPGTLSASPGERATMSCRASSSVSYMYWYQQKPGQAPRPWIYATSNLASGIPDRFSGSGSGTDYTLTISRVEPEDFAVYYCQQWSGNPRTFGGGTKLEIK

SEQ ID NO: 216 - protein, 106 aa, Artificial Sequence
  Description of Artificial Sequence: Synthetic polypeptide
EIVLTQSPGTLSLSPGERATLSCRASSSVSYMYWYQQKPGQAPRLLIYATSNLASGIPDRFSGSGSGTDYTLTISRLEPEDFAVYYCQQWEGNPRTFGGGTKLEIK

SEQ ID NO: 217 - protein, 106 aa, Artificial Sequence
  Description of Artificial Sequence: Synthetic polypeptide
EIVLTQSPGTLSLSPGERATLSCRASSSVSYMYWYQQKPGQAPRLLIYATSNLASGIPDRFSGSGSGTDFTLTISRVEPEDFAVYYCQQWEGNPRTFGGGTKLEIK

SEQ ID NO: 218 - protein, 106 aa, Artificial Sequence
  Description of Artificial Sequence: Synthetic polypeptide
EIVLTQSPGTLSLSPGERATLSCRASSSVSYMYWYQQKPGQAPRLLIYATSNLASGIPDRFSGSGSGTDYTLTISRVEPEDFAVYYCQQWEGNPRTFGGGTKLEIK

SEQ ID NO: 219 - protein, 106 aa, Artificial Sequence
  Description of Artificial Sequence: Synthetic polypeptide
EIVLTQSPGTLSASPGERATLSCRASSSVSYMYWYQQKPGQAPRLLIYATSNLASGIPDRFSGSGSGTDFTLTISRLEPEDFAVYYCQQWEGNPRTFGGGTKLEIK

SEQ ID NO: 220 - protein, 106 aa, Artificial Sequence
  Description of Artificial Sequence: Synthetic polypeptide
EIVLTQSPGTMSLSPGERATLSCRASSSVSYMYWYQQKPGQAPRLLIYATSNLASGIPDRFSGSGSGTDFTLTISRLEPEDFAVYYCQQWEGNPRTFGGGTKLEIK

SEQ ID NO: 221 - protein, 106 aa, Artificial Sequence
  Description of Artificial Sequence: Synthetic polypeptide
QIVLSQSPAILSASPGEKVTMTCRASSSVSYMYWYQQKPGSSPKPWIYATSNLASGVPDRFSGSGSGTSYSLTISRVEAEDAATYYCQQWSGNPRTFGGGTKLEIK

SEQ ID NO: 222 - protein, 131 aa, Artificial Sequence
  Description of Artificial Sequence: Synthetic polypeptide
MKLPVRLLVLMFWIPASSSDVLMTQTPLSLPVSLGDQASISCRSSQTIVHSNGDTYLDWFLQKPGQSPKLLIYKVSNRFSGVPDRFSGSGSGTDFTLKISRVEAEDLGVYYCFQGSHVPYTFGGGTKLEIK

SEQ ID NO: 223 - protein, 106 aa, Artificial Sequence
  Description of Artificial Sequence: Synthetic polypeptide
EIVLTQSPGTLSLSPGERATMSCRASSSVSYMYWYQQKPGQAPRPWIYATSNLASGIPDRFSGSGSGTDFTLTISRVEPEDFAVYYCQQWSGNPRTFGGGTKLEIK

SEQ ID NO: 224 - protein, 106 aa, Artificial Sequence
  Description of Artificial Sequence: Synthetic polypeptide
EIVLTQSPGTLSLSPGERVTMSCRASSSVSYMYWYQQKPGQAPRPWIYATSNLASGVPDRFSGSGSGTDFTLTISRVEPEDFAVYYCQQWSGNPRTFGGGTKVEIK

SEQ ID NO: 225 - protein, 106 aa, Artificial Sequence
  Description of Artificial Sequence: Synthetic polypeptide
EIVLTQSPGTLSASPGERVTLSCRASSSVSYMYWYQQKPGQAPRPWIYATSNLASGVPDRFSGSGSGTDFTLTISRLEPEDFAVYYCQQWSGNPRTFGGGTKLEIK

SEQ ID NO: 226 - protein, 106 aa, Artificial Sequence
  Description of Artificial Sequence: Synthetic polypeptide
EIVLTQSPGTLSASPGERVTMSCRASSSVSYMYWYQQKPGQAPRLLIYATSNLASGVPDRFSGSGSGTDFTLTISRVEPEDFAVYYCQQWSGNPRTFGGGTKLEIK

SEQ ID NO: 227 - protein, 106 aa, Artificial Sequence
  Description of Artificial Sequence: Synthetic polypeptide
EIVLTQSPGTLSLSPGERVTMSCRASSSVSYMYWYQQKPGQAPRPWIYATSNLASGVPDRFSGSGSGTDYTLTISRLEPEDFAVYYCQQWSGNPRTFGGGTKLEIK

SEQ ID NO: 228 - protein, 106 aa, Artificial Sequence
  Description of Artificial Sequence: Synthetic polypeptide
EIVLTQSPGTLSLSPGERATLSCRASSSVSYMYWYQQKPGQAPRLLIYATSNLASGVPDRFSGSGSGTDYTLTISRLEPEDFAVYYCQQWSGNPRTFGGGTKLEIK

SEQ ID NO: 229 - protein, 106 aa, Artificial Sequence
  Description of Artificial Sequence: Synthetic polypeptide
EIVLTQSPGTLSLSPGERATLSCRASSSVSYMYWYQQKPGQAPRLLIYATSNLASGIPDRFSGSGSGTDFTLTISRLEPEDFAVYYCQQWDGNPRTFGGGTKLEIK

SEQ ID NO: 230 - protein, 106 aa, Artificial Sequence
  Description of Artificial Sequence: Synthetic polypeptide
EIVLTQSPGTLSLSPGERATLSCRASSSVSYMYWYQQKPGQAPRLLIYATSNLASGIPDRFSGSGSGTDFTLTISRLEPEDFAVYYCQQWHGNPRTFGGGTKLEIK

SEQ ID NO: 231 - protein, 106 aa, Artificial Sequence
  Description of Artificial Sequence: Synthetic polypeptide
EIVLTQSPGTLSLSPGERATLSCRASSSVSYMYWYQQKPGQAPRLLIYATSNLASGIPDRFSGSGSGTDFTLTISRLEPEDFAVYYCQQWNGNPRTFGGGTKLEIK

SEQ ID NO: 232 - protein, 106 aa, Artificial Sequence
  Description of Artificial Sequence: Synthetic polypeptide
EIVLTQSPGTLSLSPGERATLSCRASSSVSYMYWYQQKPGQAPRLLIYATSNLASGIPDRFSGSGSGTDFTLTISRLEPEDFAVYYCQQWQGNPRTFGGGTKLEIK

SEQ ID NO: 233 - protein, 106 aa, Artificial Sequence
  Description of Artificial Sequence: Synthetic polypeptide
  MOD_RES (88)..(88): Q or N
  MOD_RES (91)..(91): D, E, H, N, Q, or S
  MOD_RES (92)..(92): A or G
  MOD_RES (93)..(93): D, F, K, N, R, S, or T
EIVLTQSPGTLSLSPGERATLSCRASSSVSYMYWYQQKPGQAPRLLIYATSNLASGIPDRFSGSGSGTDFTLTISRLEPEDFAVYYCXQWXXXPRTFGGGTKLEIK

SEQ ID NO: 234 - protein, 106 aa, Artificial Sequence
  Description of Artificial Sequence: Synthetic polypeptide
EIVLTQSPGTLSLSPGERATLSCGASSSVSYMYWYQQKPGQAPRLLIYATSNLASGIPDRFSGSGSGTDFTLTISRLEPEDFAVYYCQQWEGNPRTFGGGTKLEIK

SEQ ID NO: 235 - protein, 106 aa, Artificial Sequence
  Description of Artificial Sequence: Synthetic polypeptide
EIVLTQSPGTLSLSPGERATLSCRASSSVSYMYWYQQKPGQAPRLLIYATSNLASGIPDRFSGSGSGTDFTLTISRLEPEDFAVYYCSQWSGNPRTFGGGTKLEIK

SEQ ID NO: 236 - protein, 106 aa, Artificial Sequence
  Description of Artificial Sequence: Synthetic polypeptide
EIVLTQSPGTLSLSPGERATLSCRASSSVSYMYWYQQKPGQAPRLLIYATSNLASGIPDRFSGSGSGTDFTLTISRLEPEDFAVYYCQQWSGNPRSFGGGTKLEIK

SEQ ID NO: 237 - protein, 113 aa, Artificial Sequence
  Description of Artificial Sequence: Synthetic polypeptide
DIVMTQSPDSLAVSLGERATINCKSSQSILYSSNNKNYLAWYQQKPGQPPKLLIYWASTRESGVPDRFSGSGSGTDFTLTISSLQAEDVSVYYCQQYYSTPFTFGPGTKVDIK

SEQ ID NO: 238 - protein, 112 aa, Artificial Sequence
  Description of Artificial Sequence: Synthetic polypeptide
DIMLTQTPLTSPVTLGQPASISCKSSQSLVHSDGNTYLSWLQQRPGQPPRLLFYKISNRFSGVPDRFSGSGAGTDFTLKISRVEAEDVGVYYCMQATQFPLTFGGGTKVEIK

SEQ ID NO: 239 - protein, 107 aa, Artificial Sequence
  Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 240 - protein, 214 aa, Artificial Sequence
  Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 241 - protein, 107 aa, Artificial Sequence [sequence omitted]

SEQ ID NO: 242 - protein, 106 aa, Artificial Sequence
  Description of Artificial Sequence: Synthetic polypeptide
DIQLTQSPSFLSASVGDRVTITCSASSSVNYMHWYQQKPGKAPKLLIYSTSNLASGVPSRFSGSGSGTEFTLTISSLQPEDFATYYCHQWNNYGTFGQGTKVEIKR

SEQ ID NO: 243 - protein, 113 aa, Artificial Sequence
  Description of Artificial Sequence: Synthetic polypeptide
DVVMTQSPLSLPVTLGQPASISCKSSQNIVHSDGNTYLEWFQQRPGQSPRRLIYKVSNRFSGVPDRFSGSGSGTDFTLKISRVEAEDVGVYYCFQGSHVPLTFGGGTKVEIKR

SEQ ID NO: 244 - protein, 113 aa, Artificial Sequence
  Description of Artificial Sequence: Synthetic polypeptide
DVVMTQSPLSLPVTLGQPASISCKSSQNIVHSDGNTYLEWFQQRPGQSPRRLIYKVSNRFSGVPDRFSGSGSGTDFTLKISRVEAEDVGVYYCFQGSHVPLTFGQGTKVEIKR

SEQ ID NO: 245 - protein, 113 aa, Artificial Sequence
  Description of Artificial Sequence: Synthetic polypeptide
DVVMTQTPLSLPVTPGEPASISCKSSQNIVHSDGNTYLEWYLQKPGQSPQLLIYKVSNRFSGVPDRFSGSGSGTDFTLKISRVEAEDLGVYYCFQGSHVPLTFGGGTKVEIKR

SEQ ID NO: 246 - protein, 113 aa, Artificial Sequence
  Description of Artificial Sequence: Synthetic polypeptide
DVVMTQTPLSLPVSLGDQASISCKSSQNIVHSDGNTYLEWYLQKPGQSPKVLIYKVSNRFSGVPDRFSGSGSGTDFTLKISRVEAEDLGVYYCFQGSHVPLTFGGGTKVEIKR

SEQ ID NO: 247 - protein, 113 aa, Artificial Sequence
  Description of Artificial Sequence: Synthetic polypeptide
DVVMTQSPLSLPVTLGQPASISCRSSQSIVHSNGNTYLDWFQQRPGQSPRRLIYKVSNRFSGVPDRFSGSGSGTDFTLKISRVEAEDVGVYYCFQGSHVPLTFGGGTKVEIKR

SEQ ID NO: 248 - protein, 113 aa, Artificial Sequence
  Description of Artificial Sequence: Synthetic polypeptide
DVVMTQSPLSLPVTLGQPASISCRSSQSIVHSNGNTYLDWFQQRPGQSPRRLIYKVSNRFSGVPDRFSGSGSGTDFTLKISRVEAEDVGVYYCFQGSHVPLTFGQGTKVEIKR

SEQ ID NO: 249 - protein, 113 aa, Artificial Sequence
  Description of Artificial Sequence: Synthetic polypeptide
DVVMTQTPLSLPVTPGEPASISCRSSQSIVHSNGNTYLDWYLQKPGQSPQLLIYKVSNRFSGVPDRFSGSGSGTDFTLKISRVEAEDLGVYYCFQGSHVPLTFGGGTKVEIKR

SEQ ID NO: 250 - protein, 113 aa, Artificial Sequence
  Description of Artificial Sequence: Synthetic polypeptide
DVVMTQTPLSLPVSLGDQASISCRSSQSIVHSNGNTYLDWYLQKPGQSPKVLIYKVSNRFSGVPDRFSGSGSGTDFTLKINRVEAEDLGVYFCFQGSHVPLTFGGGTKLEIKR

SEQ ID NO: 251 - protein, 107 aa, Artificial Sequence
  Description of Artificial Sequence: Synthetic polypeptide
DIQMNQSPSSLSASLGDTITITCHASQNINVLLSWYQQKPGNIPKLLIYKASNLHTGVPSRFSGSGSGTGFTFTISSLQPEDIATYYCQQGQSYPYTFGGGTKLEIK

SEQ ID NO: 252 - protein, 107 aa, Artificial Sequence
  Description of Artificial Sequence: Synthetic polypeptide
DIQMTQSPSSLSASVGDRVTITCQASQDISNYLNWYQQKPGKAPKLLIYDASNLETGVPSRFSGSGSGTDFTFTISSLQPEDIATYYCQQYDNLPYTFGQGTKLEIK

SEQ ID NO: 253 - protein, 107 aa, Artificial Sequence
  Description of Artificial Sequence: Synthetic polypeptide
DIQMTQSPSSLSASVGDRVTITCQASQNINVLLNWYQQKPGKAPKLLIYKASNLHTGVPSRFSGSGSGTDFTFTISSLQPEDIATYYCQQGQSYPYTFGQGTKLEIK

SEQ ID NO: 254 - protein, 107 aa, Artificial Sequence
  Description of Artificial Sequence: Synthetic polypeptide
DIQMNQSPSSLSASVGDRVTITCQASQNINVLLSWYQQKPGKAPKLLIYKASNLHTGVPSRFSGSGSGTDFTFTISSLQPEDIATYYCQQGQSYPYTFGQGTKLEIK

SEQ ID NO: 255 - protein, 111 aa, Artificial Sequence
  Description of Artificial Sequence: Synthetic polypeptide
  MOD_RES (32)..(33): Any amino acid
  MOD_RES (36)..(36): Any amino acid
  MOD_RES (93)..(93): Any amino acid
QSVLTQPPSVSGAPGQRVTISCTSSSSDIGAXXGVXWYQQLPGTAPKLLIEGYYNRPSGVPDRFSGSKSGTSASLTITGLLPEDEGDYYCQSXDGTLSALFGGGTKLTVLG

SEQ ID NO: 256 - protein, 111 aa, Artificial Sequence
  Description of Artificial Sequence: Synthetic polypeptide
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSWDGTLSALFGGGTKLTVLG

SEQ ID NO: 257 - protein, 111 aa, Artificial Sequence
  Description of Artificial Sequence: Synthetic polypeptide
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSYDGTLSALFGGGTKLTVLG

SEQ ID NO: 258 - protein, 111 aa, Artificial Sequence
  Description of Artificial Sequence: Synthetic polypeptide
QSVLTQPPSVSGAPGQRVTISCTSSSSDIGAALGVHWYQQLPGTAPKLLIEGYYNRPSGVPDRFSGSKSGTSASLTITGLLPEDEGDYYCQSWDGTLSALFGGGTKLTVLG

SEQ ID NO: 259 - protein, 111 aa, Artificial Sequence
  Description of Artificial Sequence: Synthetic polypeptide
QSVLTQPPSVSGAPGQRVTISCTSSSSDIGAGSGVHWYQQLPGTAPKLLIEGYYNRPSGVPDRFSGSKSGTSASLTITGLLPEDEGDYYCQSWDGTLSALFGGGTKLTVLG

SEQ ID NO: 260 - protein, 111 aa, Artificial Sequence
  Description of Artificial Sequence: Synthetic polypeptide
QSVLTQPPSVSGAPGQRVTISCTSSSSDIGAGQGVHWYQQLPGTAPKLLIEGYYNRPSGVPDRFSGSKSGTSASLTITGLLPEDEGDYYCQSWDGTLSALFGGGTKLTVLG

SEQ ID NO: 261 - protein, 111 aa, Artificial Sequence
  Description of Artificial Sequence: Synthetic polypeptide
QSVLTQPPSVSGAPGQRVTISCTSSSSDIGAGLGVLWYQQLPGTAPKLLIEGYYNRPSGVPDRFSGSKSGTSASLTITGLLPEDEGDYYCQSWDGTLSALFGGGTKLTVLG

SEQ ID NO: 262 - protein, 111 aa, Artificial Sequence
  Description of Artificial Sequence: Synthetic polypeptide
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSFDGTLSALFGGGTKLTVLG

SEQ ID NO: 263 - protein, 107 aa, Artificial Sequence
  Description of Artificial Sequence: Synthetic polypeptide
DIQMTQSPSSLSASVGDRVTITCRASQSINNYLNWYQQRPGKAPKLLIYAASSLQSGVPSRFSGSGSGTDFTLTISSLQPGDFATYYCQQSYSTPRTFGQGTKLEIK

SEQ ID NO: 264 - protein, 107 aa, Artificial Sequence
  Description of Artificial Sequence: Synthetic polypeptide
EIVLTQSPGTLSLSPGERATLSCRASQSVRSSYLAWYQQKPGQAPRLLIYGASSRATGIPDRFSGSGSGTDFTLTISRLEPEDFAVYYCQQYGSSPTFGQGTRLEIK

SEQ ID NO: 265 - protein, 106 aa, Artificial Sequence
  Description of Artificial Sequence: Synthetic polypeptide
DIQMTQSPSTLSASVGDRVTITCRASQTISSWLAWYQQTPEKAPKLLIYAASNLQSGVPSRFSGSGSGTEFTLTISSLQPDDFATYYCQQYHRSWTFGQGTKVEIT

SEQ ID NO: 266 - protein, 112 aa, Artificial Sequence
  Description of Artificial Sequence: Synthetic polypeptide
DVLMTQTPLSLPVSLGDQASISCRSSQNIVHSDGNTYLEWYLQKPGQSPKLLIYKVSNRFSGVPDRFSGSGSGTDFTLKISRVEAEDLGIYYCFQGSHVPLTFGAGTKLELK

SEQ ID NO: 267 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIDPASGHSKYDPKFQVRATITTDTSASTAYLQLSSLRSEDTAVYYCARSGGLPDFWGQGTTVTVSS

SEQ ID NO: 268 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIDPASGHYKYDPKFQVRATITTDTSASTAYLQLSSLRSEDTAVYYCARSGGLPDFWGQGTTVTVSS

SEQ ID NO: 269 - protein, 122 aa, Artificial Sequence
  Description of Artificial Sequence: Synthetic polypeptide
EVQLVESGGGLVQPGGSLRLSCAVSGFTFSSYWMSWVRQAPGKGLEWVANIKEDGSEKNYVDSVKGRFTLSSDNAKNSLYLQMNSLRAEDTAVYYCAREDYDSYYKYGMDVWGQGTAVIVSS

SEQ ID NO: 270 - protein, 119 aa, Artificial Sequence
  Description of Artificial Sequence: Synthetic polypeptide
QVQLQQWGAGLLKPSETLSLTCAVYGGSFTGFYWSWIRQPPGKGLEWIGEINHRGNTNYNPSLKSRVTMSVDTSKNQFSLNMISVTAADTAMYFCASPFYDFWSGSDYWGQGTLVTVSS

SEQ ID NO: 271 - protein, 124 aa, Artificial Sequence
  Description of Artificial Sequence: Synthetic polypeptide
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSS

SEQ ID NO: 272 - protein, 453 aa, Artificial Sequence
  Description of Artificial Sequence: Synthetic polypeptide
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 273 - protein, 121 aa, Artificial Sequence
  Description of Artificial Sequence: Synthetic polypeptide
QLQLQESGPGLVKPSETLSLTCTVSGGSISSRSYYWGWIRQPPGKGLEWIGSIYYNGRTYYNPSLKSRVTISVDTSKNQFSLKLSSVTAADTAVYYCAREDYGDYGAFDIWGQGTMVTVSS

SEQ ID NO: 274 - protein, 124 aa, Artificial Sequence
  Description of Artificial Sequence: Synthetic polypeptide
QVTLKESGPALVKPTQTLTLTCTFSGFSLSTSNMGVVWIRQPPGKALEWLAHILWDDREYSNPALKSRLTISKDTSKNQVVLTMTNMDPVDTATYYCARMSRNYYGSSYVMDYWGQGTLVTVSS

SEQ ID NO: 275 - protein, 119 aa, Artificial Sequence
  Description of Artificial Sequence: Synthetic polypeptide
QVQLVQSGSELKKPGASVKVSCKASGYTFTLYGMNWVRQAPGQGLEWMGWINTYTGEPTYADDFKGRFVFSLDTSVSTAYLQISSLKAEDTAVYYCARDTAMDYAMAYWGQGTLVTVSS

SEQ ID NO: 276 - protein, 119 aa, Artificial Sequence
  Description of Artificial Sequence: Synthetic polypeptide
QVQLVQSGSELKKPGASVKVSCKASGYTFTLYGMNWVKQAPGKGLKWMGWINTYTGEPTYADDFKGRFVFSLDTSVSTAYLQISSLKAEDTAVYFCARDTAMDYAMAYWGQGTLVTVSS

SEQ ID NO: 277 - protein, 122 aa, Artificial Sequence
  Description of Artificial Sequence: Synthetic polypeptide
QVQLVQSGSELKKPGASVKVSCKASGYTFTNYGMNWVRQAPGQGLEWMGWINTYTGEPTYADDFKGRFVFSLDTSVSTAYLQISSLKAEDTAVYYCARDYGKYGDYYAMDYWGQGTLVTVSS

SEQ ID NO: 278 - protein, 122 aa, Artificial Sequence
  Description of Artificial Sequence: Synthetic polypeptide
QVQLVQSGSELKKPGASVKVSCKASGYTFTNYGMNWVRQAPGKGLKWMGWINTYTGEPTYADDFKGRFVFSLDTSVSTAYLQISSLKAEDTAVYFCARDYGKYGDYYAMDYWGQGTLVTVSS

SEQ ID NO: 279 - protein, 119 aa, Artificial Sequence
  Description of Artificial Sequence: Synthetic polypeptide
QVQLQQPGSVLVRPGASVKVSCKASGYTFTSSWMHWAKQRPGQGLEWIGEIHPNSGGTNYNEKFKGKATVDTSSSTAYVDLSSLTSEDSAVYYCARGDYYGYVSWFAYWGQGTLVTVSS

SEQ ID NO: 280 - protein, 121 aa, Artificial Sequence
  Description of Artificial Sequence: Synthetic polypeptide
QVQLVQSGAEVKKPGASVKVSCKASGYTFTSSWMHWARQAPGQGLEWIGEIHPNSGGTNYAQKFQGRATLTVDTSSSTAYMELSRLRSDDTAVYYCARGDYYGYVSWFAYWGQGTLVTVSS

SEQ ID NO: 281 - protein, 121 aa, Artificial Sequence
  Description of Artificial Sequence: Synthetic polypeptide
QVQLVQSGAEVKKPGASVKVSCKASGYTFTSSWMHWARQAPGQGLEWIGEIHPNSGGTNYAQKFQGRATMTVDTSISTAYMELSRLRSDDTAVYYCARGDYYGYVSWFAYWGQGTLVTVSS

SEQ ID NO: 282 - protein, 121 aa, Artificial Sequence
  Description of Artificial Sequence: Synthetic polypeptide
QVQLVQSGAEVKKPGASVKVSCKASGYTFTSSWMHWARQAPGQGLEWIGEIHPNSGGTNYAQKFQGRVTMTVDTSISTAYMELSRLRSDDTAVYYCARGDYYGYVSWFAYWGQGTLVTVSS

SEQ ID NO: 283 - protein, 121 aa, Artificial Sequence
  Description of Artificial Sequence: Synthetic polypeptide
QVQLVQSGAEVKKPGASVKVSCKASGYTFTSSWMHWARQAPGQGLEWMGEIHPNSGGTNYAQKFQGRVTMTVDTSISTAYMELSRLRSDDTAVYYCARGDYYGYVSWFAYWGQGTLVTVSS

SEQ ID NO: 284 - protein, 119 aa, Artificial Sequence
  Description of Artificial Sequence: Synthetic polypeptide
QVQLVQSGAEVKKPGASVKVSCKASGYTFTSYDINWVRQAPGQGLEWMGWLNPNSGNTGYAQKFQGRVTMTADRSTSTAYMELSSLRSEDTAVYYCAREVPETAAFEYWGQGTLVTVSS

SEQ ID NO: 285 - protein, 119 aa, Artificial Sequence
  Description of Artificial Sequence: Synthetic polypeptide
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSS

SEQ ID NO: 286 - protein, 118 aa, Artificial Sequence
  Description of Artificial Sequence: Synthetic polypeptide
QVQLQESGPGLVKPSETLSLTCTVSGGSISSYFWSWIRQPPGKGLEWIGYIYYSGNTKYNPSLKSRVTISIDTSKNQFSLKLSSVTAADTAVYYCARETGSYYGFDYWGQGTLVTVSS

SEQ ID NO: 287 - protein, 120 aa, Artificial Sequence
  Description of Artificial Sequence: Synthetic polypeptide
QVQLQQWGAGLLKPSETLSLTCAVHGGSFSGYYWNWIRQPPGKGLEWIGEINHAGNTNYNPSLKSRVTISLDTSKNQFSLTLTSVTAADTAVYYCARGYCRSTTCYFDYWGQGTLVTVSS

SEQ ID NO: 288 - protein, 121 aa, Artificial Sequence
  Description of Artificial Sequence: Synthetic polypeptide
EVQLLESGGGLVQPGKSLRLSCAVSGFTFSTYGMNWVRQAPGKGLEWVSSISGTGRTTYHADSVQGRFTVSRDNSKNILYLQMNSLRADDTAVYFCTKERGDYYYGVFDYWGQGTLVTVSS

SEQ ID NO: 289 - protein, 122 aa, Artificial Sequence
  Description of Artificial Sequence: Synthetic polypeptide
QIQLVQSGPELKKPGETVKISCKASGYTFTTYGMSWVKQAPGKGLKWMGWMNTYSGVTTYADDFKGRFAFSLETSASTAYMQIDNLKNEDTATYFCAREGYVFDDYYATDYWGQGTSVTVSS

SEQ ID NO: 290-300 - skipped (11 entries)

SEQ ID NO: 301 - protein, 124 aa, Artificial Sequence
  Description of Artificial Sequence: Synthetic polypeptide
  MOD_RES (1)..(1): A, R, N, D, C, Q, E, G, H, I, L, K, M, F, P, S, T, W, Y, or V
  MOD_RES (26)..(35): This region may encompass one of the following sequences: “GFDIQDTYMH” or “GFTFSSYW” or “GGSFTGFY” or “GYDFTYYGIS” or “SRSYYWG” or “TSNMGVV” or “LYGMN” or “NYGMN” or “GYTFTSSWMH” or “SYDIN” or “SYFWS” or “GYYWN” or “GFTFSTYG” or “TYGMS”
  MOD_RES (38)..(38): A, R, N, D, C, Q, E, G, H, I, L, K, M, F, P, S, T, W, Y, or V
  MOD_RES (40)..(40): A, R, N, D, C, Q, E, G, H, I, L, K, M, F, P, S, T, W, Y, or V
  MOD_RES (48)..(48): A, R, N, D, C, Q, E, G, H, I, L, K, M, F, P, S, T, W, Y, or V
  MOD_RES (50)..(66): This region may encompass one of the following sequences: “RIDPASGHTKYDPKFQV” or “RIEPASGHIKYDPKFQG” or “RIDPASGHIKYDPKFQG” or “RIEPASGHIKYDPKFQV” or “IKEDGSEK” or “INHRGNT” or “WISTYNGNTHYARMLQG” or “SIYYNGRTYYNPSLKS”
  MOD_RES (50)..(66): CONT. FROM ABOVE: or “HILWDDREYSNPALKS” or “WINTYTGEPTYADDFKG” or “IHPNSGGT” or “YIYYSGNTKYNPSLKS” or “EINHAGNTNYNPSLKS” or “ISGTGRTT” or “WMNTYSGVTTYADDFKG”
  MOD_RES (68)..(68): A, R, N, D, C, Q, E, G, H, I, L, K, M, F, P, S, T, W, Y, or V
  MOD_RES (70)..(70): A, R, N, D, C, Q, E, G, H, I, L, K, M, F, P, S, T, W, Y, or V
  MOD_RES (72)..(72): A, R, N, D, C, Q, E, G, H, I, L, K, M, F, P, S, T, W, Y, or V
  MOD_RES (79)..(79): A, R, N, D, C, Q, E, G, H, I, L, K, M, F, P, S, T, W, Y, or V
  MOD_RES (81)..(81): A, R, N, D, C, Q, E, G, H, I, L, K, M, F, P, S, T, W, Y, or V
  MOD_RES (99)..(113): This region may encompass one of the following sequences: “SGGLPDV” or “ARSGGLPDV” or “SGGLPDW” or “ARSGGLPDW” or “AREDYDSYYKYGMDV” or “ASPFYDFWSGSDY” or “ENYYGSGAYRGGMDV” or “EDYGDYGAFDI” or “MSRNYYGSSYVMDY” or “DTAMDYAMAY” or
  MOD_RES (99)..(113): CONT. FROM ABOVE: “DYGKYGDYYAMDY” or “ARGDYYGYVSWFAY” or “EVPETAAFEY” or “ETGSYYGFDY” or “GYCRSTTCYFDY” or “TKERGDYYYGVFDY” or “EGYVFDDYYATDY”
XVQLVQSGAEVKKPGASVKVSCKASXXXXXXXXXXWVXQXPGQGLEWXGXXXXXXXXXXXXXXXXXRXTXTXDTSTSTXYXELSSLRSEDTAVYYCARXXXXXXXXXXXXXXXWGQGTTVTVSS

SEQ ID NO: 302 - protein, 122 aa, Artificial Sequence
  Description of Artificial Sequence: Synthetic polypeptide
  MOD_RES (1)..(1): A, R, N, D, C, Q, E, G, H, I, L, K, M, F, P, S, T, W, Y, or V
  MOD_RES (26)..(35): This region may encompass one of the following sequences: “GFDIQDTYMH” or “GFTFSSYW” or “GGSFTGFY” or “GYDFTYYGIS” or “SRSYYWG” or “TSNMGVV” or “LYGMN” or “NYGMN” or “GYTFTSSWMH” or “SYDIN” or “SYFWS” or “GYYWN” or “GFTFSTYG” or “TYGMS”
  MOD_RES (38)..(38): A, R, N, D, C, Q, E, G, H, I, L, K, M, F, P, S, T, W, Y, or V
  MOD_RES (40)..(40): A, R, N, D, C, Q, E, G, H, I, L, K, M, F, P, S, T, W, Y, or V
  MOD_RES (48)..(48): A, R, N, D, C, Q, E, G, H, I, L, K, M, F, P, S, T, W, Y, or V
  MOD_RES (50)..(66): This region may encompass one of the following sequences: “RIDPASGHTKYDPKFQV” or “RIEPASGHIKYDPKFQG” or “RIDPASGHIKYDPKFQG” or “RIEPASGHIKYDPKFQV” or “IKEDGSEK” or “INHRGNT” or “WISTYNGNTHYARMLQG” or “SIYYNGRTYYNPSLKS” or “HILWDDREYSNPALKS”
  MOD_RES (50)..(66): CONT. FROM ABOVE: or “WINTYTGEPTYADDFKG” or “IHPNSGGT” or “YIYYSGNTKYNPSLKS” or “EINHAGNTNYNPSLKS” or “ISGTGRTT” or “WMNTYSGVTTYADDFKG”
  MOD_RES (68)..(68): A, R, N, D, C, Q, E, G, H, I, L, K, M, F, P, S, T, W, Y, or V
  MOD_RES (70)..(70): A, R, N, D, C, Q, E, G, H, I, L, K, M, F, P, S, T, W, Y, or V
  MOD_RES (72)..(72): A, R, N, D, C, Q, E, G, H, I, L, K, M, F, P, S, T, W, Y, or V
  MOD_RES (79)..(79): A, R, N, D, C, Q, E, G, H, I, L, K, M, F, P, S, T, W, Y, or V
  MOD_RES (81)..(81): A, R, N, D, C, Q, E, G, H, I, L, K, M, F, P, S, T, W, Y, or V
  MOD_RES (97)..(111): This region may encompass one of the following sequences: “SGGLPDV” or “ARSGGLPDV” or “SGGLPDW” or “ARSGGLPDW” or “AREDYDSYYKYGMDV” or “ASPFYDFWSGSDY” or “ENYYGSGAYRGGMDV” or “EDYGDYGAFDI” or “MSRNYYGSSYVMDY” or “DTAMDYAMAY” or
  MOD_RES (97)..(111): CONT. FROM ABOVE: “DYGKYGDYYAMDY” or “ARGDYYGYVSWFAY” or “EVPETAAFEY”or “ETGSYYGFDY” or “GYCRSTTCYFDY” or “TKERGDYYYGVFDY” or “EGYVFDDYYATDY”
XVQLVQSGAEVKKPGASVKVSCKASXXXXXXXXXXWVXQXPGQGLEWXGXXXXXXXXXXXXXXXXXRXTXTXDTSTSTXYXELSSLRSEDTAVYYCXXXXXXXXXXXXXXXWGQGTTVTVSS

SEQ ID NO: 303 - protein, 112 aa, Artificial Sequence
  Description of Artificial Sequence: Synthetic polypeptide
  MOD_RES (24)..(39): This region may encompass one of the following sequences: “RASSSVSYMY” or “QSILYSSNNKNY” or “QSLVHSDGNTY” or “RASQSVSSYLA” or “RASQGISSALA” or “SASSSVNYMH” or “KSSQNIVHSDGNTYLE” or “RSSQSIVHSNGNTYLD” or “QNINVL” or “RASQSINNYLN” or “RASQSVRSSYLA”
  MOD_RES (24)..(39): CONT. FROM ABOVE: or “QTISSW” or “RSSQNIVHSDGNTYLE”
  MOD_RES (51)..(51): A, R, N, D, C, Q, E, G, H, I, L, K, M, F, P, S, T, W, Y, or V
  MOD_RES (52)..(52): A, R, N, D, C, Q, E, G, H, I, L, K, M, F, P, S, T, W, Y, or V
  MOD_RES (55)..(61): This region may encompass one of the following sequences: “ATSNLAS” or “WAS” or “KIS” or “DASNRAT” or “DASSLES” or “STSNLAS” or “KVSNRFS” or “KAS” or “GYYNRPS” or “AASSLQS” or “GASSRAT” or “AAS”
  MOD_RES (94)..(102): This region may encompass one of the following sequences: “QQWEGNPRT” or “QQWKGNPRT” or “QQWSGNPRT” or “QQWSRNPRT” or “QQYYSTPFT” or “MQATQFPLT” or “QQRSNWPWT” or “QQFNSYPLT” or “HQWNNYGT” or “FQGSHVPLT” or “QQGQSYPYT” or “QQSYSTPRT” or
  MOD_RES (94)..(102): CONT. FROM ABOVE: “QQYGSSPT” or “QQYHRSWT”
EIVLTQSPGTLSLSPGERATLSCXXXXXXXXXXXXXXXXWYQQKPGQAPRXXIYXXXXXXXGIPDRFSGSGSGTDFTLTISRLEPEDFAVYYCXXXXXXXXXFGGGTKLEIK

SEQ ID NO: 304 - protein, 25 aa, Artificial Sequence [sequence omitted]

SEQ ID NO: 305 - protein, 14 aa, Artificial Sequence
  Description of Artificial Sequence: Synthetic peptide
WVKQRPGQGLEWMG

SEQ ID NO: 306 - protein, 32 aa, Artificial Sequence
  Description of Artificial Sequence: Synthetic polypeptide
RVTITRDTSTSTVYLELSSLRSEDTAVYYCAR

SEQ ID NO: 307 - protein, 30 aa, Artificial Sequence
  Description of Artificial Sequence: Synthetic polypeptide
RVTITRDTSTSTVYLELSSLRSEDTAVYYC

SEQ ID NO: 308 - protein, 11 aa, Artificial Sequence
  Description of Artificial Sequence: Synthetic peptide
WGQGTTVTVSS

SEQ ID NO: 309 - protein, 23 aa, Artificial Sequence
  Description of Artificial Sequence: Synthetic peptide
EIVLTQSPGTLSLSPGERATLSC

SEQ ID NO: 310 - protein, 15 aa, Artificial Sequence
  Description of Artificial Sequence: Synthetic peptide
WYQQKPGQAPRPLIY

SEQ ID NO: 311 - protein, 32 aa, Artificial Sequence
  Description of Artificial Sequence: Synthetic polypeptide
GIPDRFSGSGSGTDFTLTISRLEPEDFAVYYC

SEQ ID NO: 312 - protein, 10 aa, Artificial Sequence
  Description of Artificial Sequence: Synthetic peptide
FGGGTKLEIK

SEQ ID NO: 313 - protein, 14 aa, Artificial Sequence
  Description of Artificial Sequence: Synthetic peptide
WVRQRPGQGLEWIG

SEQ ID NO: 314 - protein, 32 aa, Artificial Sequence
  Description of Artificial Sequence: Synthetic polypeptide
RATITTDTSTSTAYLELSSLRSEDTAVYYCAR

SEQ ID NO: 315 - protein, 30 aa, Artificial Sequence
  Description of Artificial Sequence: Synthetic polypeptide
RATITTDTSTSTAYLELSSLRSEDTAVYYC

SEQ ID NO: 316 - protein, 98 aa, Artificial Sequence
  Description of Artificial Sequence: Synthetic polypeptide
QVQLVQSGAEVKKPGASVKVSCKASGYTFNSYYMHWVRQAPGQGLEWMGIINPSGGSTSYAQKFQGRVTMTRDTSTSTVYMELSSLRSEDTAVYYCAR

SEQ ID NO: 317 - protein, 96 aa, Artificial Sequence
  Description of Artificial Sequence: Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 318 - skipped

SEQ ID NO: 319 - protein, 107 aa, Artificial Sequence [sequence omitted]

SEQ ID NO: 320 - protein, 330 aa, Artificial Sequence [sequence omitted]

SEQ ID NO: 321 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 322 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 323 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 324 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 325 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 326 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 327 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 328 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 329 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 330 - protein, 329 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 331 - protein, 329 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 332 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 333 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 334 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 335 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 336 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 337 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 338 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 339 - protein, 329 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 340 - protein, 329 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 341 - protein, 329 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 342 - protein, 329 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 343 - protein, 329 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 344 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 345 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 346 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 347 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 348 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 349 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 350 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 351 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 352 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 353 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 354 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 355 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 356 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 357 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 358 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 359 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 360 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 361 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 362 - protein, 327 aa, Artificial Sequence
  Description of Artificial Sequence: Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 363 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 364 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEEFGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 365 - protein, 327 aa, Artificial Sequence
  Description of Artificial Sequence: Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 366 - protein, 327 aa, Artificial Sequence
  Description of Artificial Sequence: Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 367 - protein, 327 aa, Artificial Sequence
  Description of Artificial Sequence: Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 368 - protein, 446 aa, Artificial Sequence
  Description of Artificial Sequence: Synthetic polypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 369 - protein, 446 aa, Artificial Sequence
  Description of Artificial Sequence: Synthetic polypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 370 - protein, 446 aa, Artificial Sequence
  Description of Artificial Sequence: Synthetic polypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 371 - protein, 446 aa, Artificial Sequence
  Description of Artificial Sequence: Synthetic polypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 372 - protein, 446 aa, Artificial Sequence
  Description of Artificial Sequence: Synthetic polypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 373 - protein, 446 aa, Artificial Sequence
  Description of Artificial Sequence: Synthetic polypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 374 - protein, 446 aa, Artificial Sequence
  Description of Artificial Sequence: Synthetic polypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 375 - protein, 446 aa, Artificial Sequence
  Description of Artificial Sequence: Synthetic polypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEEFGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 376 - protein, 446 aa, Artificial Sequence
  Description of Artificial Sequence: Synthetic polypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 377 - protein, 446 aa, Artificial Sequence
  Description of Artificial Sequence: Synthetic polypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 378 - protein, 443 aa, Artificial Sequence
  Description of Artificial Sequence: Synthetic polypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 379 - protein, 443 aa, Artificial Sequence
  Description of Artificial Sequence: Synthetic polypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 380 - protein, 443 aa, Artificial Sequence
  Description of Artificial Sequence: Synthetic polypeptide
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 381 - protein, 213 aa, Artificial Sequence
  Description of Artificial Sequence: Synthetic polypeptide
EIVLTQSPGTLSLSPGERATLSCRASSSVSYMYWYQQKPGQAPRPLIYATSNLASGIPDRFSGSGSGTDFTLTISRLEPEDFAVYYCQQWEGNPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 382 - protein, 14 aa, Artificial Sequence
  Description of Artificial Sequence: Synthetic peptide
WVRQAPGQGLEWMG

SEQ ID NO: 383 - protein, 30 aa, Artificial Sequence
  Description of Artificial Sequence: Synthetic polypeptide
RVTMTRDTSTSTVYMELSSLRSEDTAVYYC

SEQ ID NO: 384 - protein, 30 aa, Artificial Sequence
  Description of Artificial Sequence: Synthetic polypeptide
RATITTDTSASTAYLQLSSLRSEDTAVYYC

SEQ ID NO: 385 - protein, 30 aa, Artificial Sequence
  Description of Artificial Sequence: Synthetic polypeptide
RVTITRDTSASTVYMELSSLRSEDTAVYYC

SEQ ID NO: 386 - protein, 30 aa, Artificial Sequence
  Description of Artificial Sequence: Synthetic polypeptide
RVTITRDTSASTAYMELSSLRSEDTAVYYC

SEQ ID NO: 387 - protein, 32 aa, Artificial Sequence
  Description of Artificial Sequence: Synthetic polypeptide
RVTMTRDTSTSTVYMELSSLRSEDTAVYYCSR

SEQ ID NO: 388 - protein, 32 aa, Artificial Sequence
  Description of Artificial Sequence: Synthetic polypeptide
RVTMTRDTSTSTVYMELSSLRSEDTAVYYCAR

SEQ ID NO: 389 - protein, 15 aa, Artificial Sequence
  Description of Artificial Sequence: Synthetic peptide
WYQQKPGQAPRLLIY

SEQ ID NO: 390 - protein, 32 aa, Artificial Sequence
  Description of Artificial Sequence: Synthetic polypeptide
GVPDRFSGSGSGTDYTLTISRLEPEDFAVYYC

SEQ ID NO: 391 - protein, 6 aa, Artificial Sequence
  Description of Artificial Sequence: Synthetic 6xHis tag
HHHHHH

SEQ ID NO: 392-400 - skipped (9 entries)

SEQ ID NO: 401 - DNA, 51 bp, Homo sapiens
ctaagatgacattaatctctgtatgrctccttggtacagttcttttccaca

SEQ ID NO: 402 - DNA, 51 bp, Homo sapiens
acataagtgggaagtagatcaagacrtgcttataattacattcttgtggtt

SEQ ID NO: 403 - DNA, 51 bp, Homo sapiens
tgagccagggcacagcctcccctggrgagctctgggaaagtgggcagcaat

SEQ ID NO: 404 - DNA, 51 bp, Homo sapiens
gaaactgtagactttgcttaaaaagrgtctcatatcattttcaaaatagac

SEQ ID NO: 405 - DNA, 51 bp, Homo sapiens
attctagaatgactggaatacccagmaaactgggtaagaatcatgtgttct

SEQ ID NO: 406 - DNA, 51 bp, Homo sapiens
ctgtgtgcggcagcatttacgtaggrggtacagacagcagcctttcactta

SEQ ID NO: 407 - DNA, 51 bp, Homo sapiens
cagggctcagaactgtccgggtcccrtcagtgttggggcggaagaggaaga

SEQ ID NO: 408 - DNA, 51 bp, Homo sapiens
tccctctaaacactgctttcactgcrttccataagattgttttttgttgtt

SEQ ID NO: 409 - DNA, 51 bp, Homo sapiens
gattataagtaacatgtgtttattgrttttttttaaaaaagggtaaatatc

(19) United States
(12) Patent Application Publication
(10) Pub. No.: US 2024/0209103 A1
(43) Pub. Date: Jun. 27, 2024

(54) PATIENT SELECTION METHODS AND KITS FOR THERAPIES TARGETING TL1A

(71) Applicant: CEDARS-SINAI MEDICAL CENTER, Los Angeles, CA (US)

(72) Inventors: Alka POTDAR, Los Angeles, CA (US); Janine BILSBOROUGH, Adelaide (AU); Dermot P. MCGOVERN, Los Angeles, CA (US); Dalin LI, Los Angeles, CA (US)

(21) Appl. No.: 18/557,551

(22) PCT Filed: Apr. 27, 2022
(86) PCT No.: PCT/US2022/026514

§ 371 Date: Oct. 26, 2023

Related U.S. Application Data
(60) Provisional application No. 63/181,114, filed on Apr. 28, 2021.

(51) Int. Cl. C07K 16/28; A61P 1/12; A61P 37/06; G01N 33/68; G16B 20/00

(52) U.S. Cl. CPC C07K 16/2875; A61P 1/12; A61P 37/06; G01N 33/6893; G16B 20/00; G01N 2333/70575; G01N 2800/065; G01N 2800/52

(57) ABSTRACT

A Provided herein are methods and compositions for patient selection and therapies targeting TL1A. In particular, provided is a method of treating moderate to severely active Crohn's disease (CD) or ulcerative colitis (UC) in a subject, the method comprising: administering a therapeutically effective amount of an inhibitor of Tumor necrosis factor-like cytokine 1A (TL1A) activity or expression to a subject with moderately to severely active CD or UC that has been determined have a polygenetic risk score (PRS) in the 75^{th} percentile, which is indicative of high fold-change of TL1A expression relative to a cut-off fold-change value. Calculating a PRS comprises providing genomic data comprising one or more genotypes (e.g. rs11221332, rs7134599, rs6062496, rs4246905, rs7468800, rs1569328, rs2284553, rs6062504, and rs7556897) of the subject associated with high TL1A fold-change relative to an index or a control.

### CROSS-REFERENCE

This application claims the benefit of U.S. Provisional Application No. 63/181,114, filed Apr. 28, 2021, which application is incorporated herein by reference in its entirety.

### INCORPORATION BY REFERENCE OF SEQUENCE LISTING

The instant application contains a Sequence Listing which has been submitted electronically in ASCII format and is hereby incorporated by reference in its entirety. Said ASCII copy, created on Apr. 14, 2022, is named 56884-795_601_SL.txt and is 463,276 bytes in size.

### SUMMARY

In certain aspects, disclosed herein is a method of treating moderate to severely active Crohn's disease (CD) or ulcerative colitis (UC) in a subject, the method comprising: administering a therapeutically effective amount of an inhibitor of Tumor necrosis factor-like cytokine 1A (TL1A) activity or expression to a subject with moderately to severely active CD or UC that has been determined have a polygenetic risk score (PRS) in the 75^{th} percentile, which is indicative of high fold-change of TL1A expression relative to a cut-off fold-change value. In some embodiments, a high TL1A fold-change is at least about 2 standard deviations above the mean of the index or control. In some embodiments, calculating a PRS comprises providing genomic data comprising one or more genotypes of the subject, wherein the one or more genotypes is associated with high TL1A fold-change relative to an index or a control. In some embodiments, the method comprises selecting, from a database, one or more genetic variants corresponding to: the one or more genotypes of the subject, or a predetermined genetic variant in a linkage disequilibrium (LD) therewith, wherein the one or more genetic variants comprises rs11221332, rs7134599, rs6062496, rs4246905, rs7468800, rs1569328, rs2284553, rs6062504, and rs7556897; In some embodiments, the method comprises calculating a genetic risk score for the subject, based, at least in part, on the one or more selected genetic variants. In some embodiments, LD is defined by (i) a D′ value of at least 0.80 or (ii) a D′ value of 0 and an R² value of at least about 0.90. In some embodiments, the predetermined genetic variant is selected in (b) based least in part, on an association with case versus control. In some embodiments, the association with case comprises association with inflammatory bowel disease, Crohn's disease, or ulcerative colitis. In some embodiments, the index or control is a population of subjects with inflammatory disease, a fibrostenotic disease, or a fibrotic disease. In some embodiments, the index or control is a population of subjects without inflammatory disease, a fibrostenotic disease, or a fibrotic disease. In some embodiments, the anti-TL1A inhibitor is an anti-TL1A antibody. In some embodiments, the anti-TL1A antibody is selected from the group consisting of an anti-TL1A antibody comprising a HCDR1 as set forth by SEQ ID NOS: 27 or 28, a HCDR2 as set forth by SEQ ID NOS: 41 or 42, a HCDR3 as set forth by SEQ ID NOS: 54 or 55, a LCDR1 as set forth by SEQ ID NO: 68, a LCDR2 as set forth by SEQ ID NO: 81, and a LCDR3 as set forth by SEQ ID NO: 92; an anti-TL1A antibody comprises a HCDR1 as set forth by SEQ ID NO: 1, a HCDR2 as set forth by SEQ ID NO: 2, a HCDR3 as set forth by SEQ ID NO: 6, a LCDR1 as set forth by SEQ ID NO: 10, a LCDR2 as set forth by SEQ ID NO: 11, and a LCDR3 as set forth by SEQ ID NO: 12; an anti-TL1A antibody comprises a HCDR1 as set forth by SEQ ID NO: 16 or 17, a HCDR2 as set forth by SEQ ID NO: 18, a HCDR3 as set forth by SEQ ID NO: 19 or 20, a LCDR1 as set forth by SEQ ID NO: 21, a LCDR2 as set forth by SEQ ID NO: 22, and a LCDR3 as set forth by SEQ ID NO: 23 or 24; an anti-TL1A antibody comprises a HCDR1 as set forth by SEQ ID NO: 25, a HCDR2 as set forth by SEQ ID NO: 39, a HCDR3 as set forth by SEQ ID NO: 52, a LCDR1 as set forth by SEQ ID NO: 66, a LCDR2 as set forth by SEQ ID NO: 79, and a LCDR3 as set forth by SEQ ID NO: 90; and an anti-TL1A antibody comprises a HCDR1 as set forth by SEQ ID NO: 30, a HCDR2 as set forth by SEQ ID NO: 44, a HCDR3 as set forth by SEQ ID NO: 57, a LCDR1 as set forth by SEQ ID NO: 70, a LCDR2 as set forth by SEQ ID NO: 83, and a LCDR3 as set forth by SEQ ID NO: 94.

In certain aspects, disclosed herein is a computer-implemented method of analyzing genomic data, the method comprising: providing genomic data comprising one or more genotypes of the subject, wherein the one or more genotypes is associated with high Tumor necrosis factor-like cytokine 1A (TL1A) fold-change relative to an index or a control; selecting one or more genetic variants corresponding to: the one or more genotypes of the subject, or a predetermined genetic variant in a linkage disequilibrium (LD) therewith, wherein the one or more genetic variants comprises rs11221332, rs7134599, rs6062496, rs4246905, rs7468800, rs1569328, rs2284553, rs6062504, and rs7556897; calculating a genetic risk score for the subject, based, at least in part, on the one or more genetic variants selected in (b); predicting high TL1A fold-change in the subject based, least in part, on the genetic risk score calculated in (c). In some embodiments, LD is defined by (i) a D′ value of at least 0.80 or (ii) a D′ value of 0 and an R² value of at least about 0.80. In some embodiments, the predetermined genetic variant is selected in (b) based least in part, on an association with case versus control. In some embodiments, the association with case comprises association with inflammatory bowel disease, Crohn's disease, or ulcerative colitis. In some embodiments, the index or control is a population of subjects with inflammatory disease, a fibrostenotic disease, or a fibrotic disease. In some embodiments, the index or control is a population of subjects without inflammatory disease, a fibrostenotic disease, or a fibrotic disease. In some embodiments, a high TL1A fold-change is at least about 2 standard deviations above the mean of the index or control. In some embodiments, the method comprises predetermining that the predetermined genetic variant is associated with TL1A fold-change. In some embodiments, the method comprises predetermining that the predetermined genetic variant is implicated in the TL1A pathway. In some embodiments, the predetermined genetic variant is selected based on phasing unphased genotype data from the subject to generate a subject-specific phased haplotypes based on the clinical condition of the subject. In some embodiments, selecting the predetermined genetic variant comprises: (a) imputing subject-specific genotypes not present in the phased subject-specific phased haplotypes using phased haplotype data from a reference group that has the same clinical phenotypes as the subject; and (b) selecting the predetermined genetic variant from the imputed subject-specific genotypes. In some embodiments, the clinical phenotype comprises fibrosis, fibrostenosis, stricturing and/or penetrating disease, obstructive disease, mrUC, refractory CD, or perianal CD. In some embodiments, the clinical phenotype comprises the presence of perianal Crohn's disease. In some embodiments, the treatment is selected from the list consisting of administration of thiopurine, administration of methotrexate, administration of a biologic, and a surgery. In some embodiments, high TL1A fold-change is associated with the subject being at risk for developing, a non-response or loss-of-response to a standard therapy comprising glucocorticosteriods, anti-TNF therapy, anti-a4-b7 therapy, anti-IL12p40 therapy, or a combination thereof.

In certain aspects, disclosed herein is a method of treating moderate to severely active Crohn's disease (CD) or ulcerative colitis (UC) in a subject, the method comprising: administering a therapeutically effective amount of an inhibitor of Tumor necrosis factor-like cytokine 1A (TL1A) activity or expression to a subject with moderately to severely active CD or UC that has been determined to have high fold-change of TL1A expression relative to a cut-off fold-change value, when the high fold-change of the TL1A expression is measured by: (a) stimulating peripheral blood mononuclear cells (PBMCs) obtained from the subject with immune complex; (b) measuring a TL1A expression level at a first time point and a second time point, wherein the second time point is later than the first time point; and (c) calculating the fold-change of the TL1A expression by dividing the TL1A expression level at the second time point by the TL1A expression level at the first time point. In some embodiments, the cut-off value is derived from a control sample obtained from a subject that does not have the moderately to severely active CD or UC. In some embodiments, the cut-off value is derived from the mean plus two times standard deviation for a population. In some embodiments, the first time point is 6 hours after treating the sample of PMBCs with sera derived from the subject. In some embodiments, the second time point is 24 hours after treating the sample of PMBCs with sera derived from the subject. In some embodiments, the second time point is 48 hours after treating the sample of PMBCs with sera derived from the subject. In some embodiments, the second time point is 72 hours after treating the sample of PMBCs with sera derived from the subject. In some embodiments, the subject has an inflammatory disease or condition. In some embodiments, the inflammatory disease or condition comprises inflammatory bowel disease, Crohn's disease, obstructive Crohn's disease, or ulcerative colitis. In some embodiments, the Crohn's disease is ileal, ileocolonic, or colonic Crohn's disease. In some embodiments, the subject has, or is at risk for developing, a non-response or loss-of-response to a standard therapy comprising glucocorticosteriods, anti-TNF therapy, anti-a4-b7 therapy, anti-IL12p40 therapy, or a combination thereof. In some embodiments, the method comprises administering an anti-TL1A antibody. In some embodiments, the anti-TL1A inhibitor is an anti-TL1A antibody. In some embodiments, the anti-TL1A antibody is selected from the group consisting of an anti-TL1A antibody comprising a HCDR1 as set forth by SEQ ID NOS: 27 or 28, a HCDR2 as set forth by SEQ ID NOS: 41 or 42, a HCDR3 as set forth by SEQ ID NOS: 54 or 55, a LCDR1 as set forth by SEQ ID NO: 68, a LCDR2 as set forth by SEQ ID NO: 81, and a LCDR3 as set forth by SEQ ID NO: 92; an anti-TL1A antibody comprises a HCDR1 as set forth by SEQ ID NO: 1, a HCDR2 as set forth by SEQ ID NO: 2, a HCDR3 as set forth by SEQ ID NO: 6, a LCDR1 as set forth by SEQ ID NO: 10, a LCDR2 as set forth by SEQ ID NO: 11, and a LCDR3 as set forth by SEQ ID NO: 12; an anti-TL1A antibody comprises a HCDR1 as set forth by SEQ ID NO: 16 or 17, a HCDR2 as set forth by SEQ ID NO: 18, a HCDR3 as set forth by SEQ ID NO: 19 or 20, a LCDR1 as set forth by SEQ ID NO: 21, a LCDR2 as set forth by SEQ ID NO: 22, and a LCDR3 as set forth by SEQ ID NO: 23 or 24; an anti-TL1A antibody comprises a HCDR1 as set forth by SEQ ID NO: 25, a HCDR2 as set forth by SEQ ID NO: 39, a HCDR3 as set forth by SEQ ID NO: 52, a LCDR1 as set forth by SEQ ID NO: 66, a LCDR2 as set forth by SEQ ID NO: 79, and a LCDR3 as set forth by SEQ ID NO: 90; and an anti-TL1A antibody comprises a HCDR1 as set forth by SEQ ID NO: 30, a HCDR2 as set forth by SEQ ID NO: 44, a HCDR3 as set forth by SEQ ID NO: 57, a LCDR1 as set forth by SEQ ID NO: 70, a LCDR2 as set forth by SEQ ID NO: 83, and a LCDR3 as set forth by SEQ ID NO: 94.

In certain aspects, disclosed herein is a method of inhibiting or reducing Tumor necrosis factor-like cytokine 1A (TL1A) activity or expression in a subject, the method comprising: obtaining peripheral blood mononuclear cells (PBMCs) from the subject; bringing the PMBCs into contact with immune-complex under conditions sufficient to produce TL1A by the PBMCs; measuring TL1A expression at a first time point; measuring TL1A expression at a second time point, wherein the second time point is later in time than the first time point; calculating the fold-change of TL1A expression between the first and the second time point; and administering to the subject a therapeutically effective amount of an inhibitor of TL1A activity or expression. In some embodiments, measuring the TL1A expression at the first time point in (c) is performed 6 hours after the contacting in (b). In some embodiments, measuring the TL1A expression at the second time point in (d) is performed 24 hours after the contacting in (b). In some embodiments, measuring the TL1A expression at the second time point in (d) is performed 48 hours after the contacting in (b). In some embodiments, measuring the TL1A expression at the second time point in (d) is performed 72 hours after the contacting in (b). In some embodiments, the subject has an inflammatory disease, a fibrostenotic disease, or a fibrotic disease. In some embodiments, the inflammatory disease, a fibrostenotic disease, or a fibrotic disease comprises inflammatory bowel disease, Crohn's disease, obstructive Crohn's disease, or ulcerative colitis. In some embodiments, the Crohn's disease is ileal, ileocolonic, or colonic Crohn's disease. In some embodiments, the subject has, or is at risk for developing, a non-response or loss-of-response to a standard therapy comprising glucocorticosteriods, anti-TNF therapy, anti-a4-b7 therapy, anti-IL12p40 therapy, or a combination thereof. In some embodiments, the inhibitor of TL1A expression or activity comprises an anti-TL1A antibody. In some embodiments, the anti-TL1A antibody is selected from the group consisting of an anti-TL1A antibody comprising a HCDR1 as set forth by SEQ ID NOS: 27 or 28, a HCDR2 as set forth by SEQ ID NOS: 41 or 42, a HCDR3 as set forth by SEQ ID NOS: 54 or 55, a LCDR1 as set forth by SEQ ID NO: 68, a LCDR2 as set forth by SEQ ID NO: 81, and a LCDR3 as set forth by SEQ ID NO: 92; an anti-TL1A antibody comprises a HCDR1 as set forth by SEQ ID NO: 1, a HCDR2 as set forth by SEQ ID NO: 2, a HCDR3 as set forth by SEQ ID NO: 6, a LCDR1 as set forth by SEQ ID NO: 10, a LCDR2 as set forth by SEQ ID NO: 11, and a LCDR3 as set forth by SEQ ID NO: 12; an anti-TL1A antibody comprises a HCDR1 as set forth by SEQ ID NO: 16 or 17, a HCDR2 as set forth by SEQ ID NO: 18, a HCDR3 as set forth by SEQ ID NO: 19 or 20, a LCDR1 as set forth by SEQ ID NO: 21, a LCDR2 as set forth by SEQ ID NO: 22, and a LCDR3 as set forth by SEQ ID NO: 23 or 24; an anti-TL1A antibody comprises a HCDR1 as set forth by SEQ ID NO: 25, a HCDR2 as set forth by SEQ ID NO: 39, a HCDR3 as set forth by SEQ ID NO: 52, a LCDR1 as set forth by SEQ ID NO: 66, a LCDR2 as set forth by SEQ ID NO: 79, and a LCDR3 as set forth by SEQ ID NO: 90; and an anti-TL1A antibody comprises a HCDR1 as set forth by SEQ ID NO: 30, a HCDR2 as set forth by SEQ ID NO: 44, a HCDR3 as set forth by SEQ ID NO: 57, a LCDR1 as set forth by SEQ ID NO: 70, a LCDR2 as set forth by SEQ ID NO: 83, and a LCDR3 as set forth by SEQ ID NO: 94.

In certain aspects, disclosed herein is a method of selecting a subject for treatment, the method comprising obtaining peripheral blood mononuclear cells (PBMCs) from the subject bringing the PMBCs into contact with immune-complex under conditions sufficient to produce Tumor necrosis factor-like cytokine 1A (TL1A) by the PBMCs; measuring TL1A expression at a first time point; measuring TL1A expression at a second time point; calculating the fold-change of TL1A expression between the first and the second time point; and selecting the subject for treatment with an inhibitor of TL1A activity or expression, provided the fold-change of TL1A is high relative to an index or a control. In some embodiments, measuring the TL1A expression at the first time point in (c) is performed 6 hours after the contacting in (b). In some embodiments, measuring the TL1A expression at the second time point in (d) is performed 24 hours after the contacting in (b). In some embodiments, measuring the TL1A expression at the second time point in (d) is performed 48 hours after the contacting in (b). In some embodiments, measuring the TL1A expression at the second time point in (d) is performed 72 hours after the contacting in (b). In some embodiments, the subject has an inflammatory disease, a fibrostenotic disease, or a fibrotic disease. In some embodiments, the inflammatory disease, a fibrostenotic disease, or a fibrotic disease comprises inflammatory bowel disease, Crohn's disease, obstructive Crohn's disease, or ulcerative colitis. In some embodiments, the Crohn's disease is ileal, ileocolonic, or colonic Crohn's disease. In some embodiments, the subject has, or is at risk for developing, a non-response or loss-of-response to a standard therapy comprising glucocorticosteriods, anti-TNF therapy, anti-a4-b7 therapy, anti-IL12p40 therapy, or a combination thereof. In some embodiments, the inhibitor of TL1A expression or activity comprises an anti-TL1A antibody. In some embodiments, the anti-TL1A antibody is selected from the group consisting of an anti-TL1A antibody comprising a HCDR1 as set forth by SEQ ID NOS: 27 or 28, a HCDR2 as set forth by SEQ ID NOS: 41 or 42, a HCDR3 as set forth by SEQ ID NOS: 54 or 55, a LCDR1 as set forth by SEQ ID NO: 68, a LCDR2 as set forth by SEQ ID NO: 81, and a LCDR3 as set forth by SEQ ID NO: 92; an anti-TL1A antibody comprises a HCDR1 as set forth by SEQ ID NO: 1, a HCDR2 as set forth by SEQ ID NO: 2, a HCDR3 as set forth by SEQ ID NO: 6, a LCDR1 as set forth by SEQ ID NO: 10, a LCDR2 as set forth by SEQ ID NO: 11, and a LCDR3 as set forth by SEQ ID NO: 12; an anti-TL1A antibody comprises a HCDR1 as set forth by SEQ ID NO: 16 or 17, a HCDR2 as set forth by SEQ ID NO: 18, a HCDR3 as set forth by SEQ ID NO: 19 or 20, a LCDR1 as set forth by SEQ ID NO: 21, a LCDR2 as set forth by SEQ ID NO: 22, and a LCDR3 as set forth by SEQ ID NO: 23 or 24; an anti-TL1A antibody comprises a HCDR1 as set forth by SEQ ID NO: 25, a HCDR2 as set forth by SEQ ID NO: 39, a HCDR3 as set forth by SEQ ID NO: 52, a LCDR1 as set forth by SEQ ID NO: 66, a LCDR2 as set forth by SEQ ID NO: 79, and a LCDR3 as set forth by SEQ ID NO: 90; and an anti-TL1A antibody comprises a HCDR1 as set forth by SEQ ID NO: 30, a HCDR2 as set forth by SEQ ID NO: 44, a HCDR3 as set forth by SEQ ID NO: 57, a LCDR1 as set forth by SEQ ID NO: 70, a LCDR2 as set forth by SEQ ID NO: 83, and a LCDR3 as set forth by SEQ ID NO: 94.

In certain aspects, disclosed herein is a method of sample preparation, the method comprising: obtaining peripheral blood mononuclear cells (PBMCs) from the subject bringing the PMBCs into contact with immune-complex under conditions sufficient to produce Tumor necrosis factor-like cytokine 1A (TL1A) by the PBMCs; measuring TL1A expression at a first time point; measuring TL1A expression at a second time point; and calculating the fold-change of TL1A between the first and the second time point; wherein a high fold-change of TL1A relative to an index or control indicates that the subject has a moderate to severely active form of CD or UC. In some embodiments, measuring the TL1A expression at the first time point in (c) is performed 6 hours after the contacting in (b). In some embodiments, measuring the TL1A expression at the second time point in (d) is performed 24 hours after the contacting in (b). In some embodiments, measuring the TL1A expression at the second time point in (d) is performed 48 hours after the contacting in (b). In some embodiments, measuring the TL1A expression at the second time point in (d) is performed 72 hours after the contacting in (b). In some embodiments, the Crohn's disease is ileal, ileocolonic, or colonic Crohn's disease.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a workflow according to an embodiment of the present disclosure for processing a biological sample obtained from a subject to inform the selection of a therapeutic agent to treat a disease or a condition of the subject.

FIG. 2 shows a computer-implemented workflow according to an embodiment of the present disclosure for generating an electronic report to a user, such as a physician, comprising a genetic risk score of a subject based on an analysis of genotype data from the subject.

FIG. 3 shows a computer system that is programmed or otherwise configured to implement methods provided herein.

FIGS. 4A-4C show the association of TL1A fold-change with the Crohn's Disease polygenic risk score (PRS). FIG. 4A compares the TL1A fold-change from 6 hours to 18 hours with the PRS. FIG. 4B compares the TL1A fold-change from 6 hours to 24 hours with the PRS. FIG. 4C compares the TL1A fold-change from 6 hours to 72 hours with the PRS.

FIGS. 5A-5C show the association of TL1A fold-change with the Inflammatory Bowel Disease polygenic risk score (PRS). FIG. 5A compares the TL1A fold-change from 6 hours to 18 hours with the PRS. FIG. 5B compares the TL1A fold-change from 6 hours to 24 hours with the PRS. FIG. 5C compares the TL1A fold-change from 6 hours to 72 hours with the PRS.

FIG. 6 depicts a computer-implemented method of analyzing genomic data.

### DETAILED DESCRIPTION

Provided herein are methods for the treatment, analysis, characterization, and diagnosis of a disease or condition. Also provided are methods of selection for treatment of a disease or condition. The methods disclosed herein provide for the calculation of a polygenic risk score. In some embodiments, the polygenic risk score described herein can be used as a surrogate for the levels of TL1A fold-change in a subject. In some embodiments, the levels of TL1A fold-change in a subject are used to provide information about the prognosis of a disease or disorder.

High levels of TL1A fold-change between two time points are associated with worse clinical parameters in patients with certain inflammatory conditions such as inflammatory bowel disease (IBD). This indicates that a subject with high TL1A fold-change may be a good candidate for treatment with second-line therapies, such as an inhibitor of TL1A expression or activity. Disclosed herein are polygenic risk scores that are predictive of high levels of TL1A fold-change in subjects with IBD or Crohn's disease (CD). As demonstrated in the examples of this disclosure, these subjects having a high PRS (e.g. in the 75^{th} percentile) were found to also have high levels of TL1A fold-change. Thus, without being bound by any particular theory, a high PRS disclosed herein can be used as a proxy for detecting high levels of TL1A fold-change in a subject, making them a a good candidate for treatment with an inhibitor of TL1A activity or expression. The genotypes disclosed herein that were used in calculating the PRS are significantly associated with incidences of disease versus control (IBD v. non-IBD, CD v. non-CD) and are at genetic loci involved in the TL1A-mediated inflammatory pathways. Thus, without being bound by any particular theory, a high CD-PRS and IBD-PRS disclosed herein are predictive of high TL1A fold-change in subjects with CD and IBD, respectively, based on a genetic association with biological relevance. Moreover, high TL1A-fold change was discovered to be strongly correlated with increased disease severity in these subjects, which suggests that a high PRS is also indicative of increased disease severity of the CD or IBD in these subjects. Accordingly a high PRS disclosed herein is predictive of an increased chance of developing a severe form of disease (e.g., IBD, CD) and responding positively to a therapeutic strategy blocking TL1A activity or expression to treat the disease. Conversely, a low PRS disclosed herein is predictive of an increased chance of not developing a severe form of the disease and may also indicate that the subject will not respond positively to a therapeutic strategy blocking TL1A activity or expression to treat the disease.

### Subject

The subject disclosed herein can be a mammal, such as for example a mouse, rat, guinea pig, rabbit, non-human primate, or farm animal. In some embodiments, the subject is human. In some embodiments, the subject is suffering from a symptom related to a disease or condition disclosed herein (e.g., abdominal pain, cramping, diarrhea, rectal bleeding, fever, weight loss, fatigue, loss of appetite, dehydration, and malnutrition, anemia, or ulcers). In some embodiments, the subject is a pediatric subject. For instance, a human subject may be 17 years old or younger. In some embodiments, the subject is an adult subject. For instance, a human subject may be between 18 and 64 years old. In some embodiments, the subject is an elderly subject. For instance, a human subject may be 65 years old or older. In some embodiments, the subject is female. In some embodiments, the subject is male.

In some embodiments, the subject is susceptible to, or is inflicted with, thiopurine toxicity, or a disease caused by thiopurine toxicity (such as pancreatitis or leukopenia). The subject may experience, or is suspected of experiencing, non-response or loss-of-response to a standard treatment (e.g., anti-TNF therapy, anti-a4-b7 therapy (vedolizumab), anti-IL12p40 therapy (ustekinumab), Thalidomide, a glucocorticosteriod, or Cytoxin). In some embodiments, the subject may be currently receiving one or more of the standard therapies disclosed herein as a “first-line” therapy, such as the anti-TNF therapy and a glucocorticosteriods. In some embodiments, the subject is in need of a “second-line” therapy disclosed herein (e.g., anti-TL1A antibody) because the subject is either not responding to an induction of the standard therapy or has lost response to the standard therapy during a treatment course or regimen.

### Disease or Condition

The disease or condition disclosed herein may be an inflammatory disease, a fibrostenotic disease, or a fibrotic disease. In some embodiments, the disease or the condition is a TL1A-mediated disease or condition. The term, “TL1A-mediated disease or condition” refers to a disease or a condition pathology or pathogenesis that is driven, at least in part, by TL1A signaling. In some embodiments, the disease or the condition is immune-mediated disease or condition, such as those mediated by TL1A. In some embodiments, the disease or condition is Crohn's disease or ulcerative colitis. In some embodiments, the Crohn's disease or ulcerative colitis is moderate to severe Crohn's disease or ulcerative colitis.

In some embodiments the disease or the condition is an inflammatory disease or disorder that is mediated, at least in part, by TL1A signaling. Non-limiting examples of inflammatory disease include, allergy, ankylosing spondylitis, asthma, atopic dermatitis, autoimmune diseases or disorders, cancer, celiac disease, chronic obstructive pulmonary disease (COPD), chronic peptic ulcer, cystic fibrosis, diabetes (e.g., type 1 diabetes and type 2 diabetes), glomerulonephritis, gout, hepatitis (e.g., active hepatitis), an immune-mediated disease or disorder, inflammatory bowel disease (IBD) such as Crohn's disease and ulcerative colitis, myositis, osteoarthritis, pelvic inflammatory disease (PID), multiple sclerosis, neurodegenerative diseases of aging, periodontal disease (e.g., periodontitis), preperfusion injury transplant rejection, psoriasis, pulmonary fibrosis, rheumatic disease, scleroderma, sinusitis, tuberculosis.

In some embodiments, the disease or the condition is an autoimmune disease that is mediated, at least in part, by TL1A signaling. Non-limiting examples of autoimmune disease or disorder include Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Vogt-Koyanagi-Harada Disease.

In some embodiments, the disease or the condition is a cancer that is mediated, at least in part, by TL1A signaling. Non-limiting examples of cancers include Adenoid Cystic Carcinoma, Adrenal Gland Cancer, Amyloidosis, Anal Cancer, Ataxia-Telangiectasia, Atypical Mole Syndrome, Basal Cell Carcinoma, Bile Duct Cancer, Birt Hogg Dube Syndrome, Bladder Cancer, Bone Cancer, Brain Tumor, Breast Cancer, Breast Cancer in Men, Carcinoid Tumor, Cervical Cancer, Colorectal Cancer, Ductal Carcinoma, Endometrial Cancer, Esophageal Cancer, Gastric Cancer, Gastrointestinal Stromal Tumor (GIST), HER2-Positive Breast Cancer, Islet Cell Tumor, Juvenile Polyposis Syndrome, Kidney Cancer, Laryngeal Cancer, Leukemia—Acute Lymphoblastic Leukemia, Leukemia—Acute Lymphocytic (ALL), Leukemia—Acute Myeloid AML, Leukemia—Adult, Leukemia—Childhood, Leukemia—Chronic Lymphocytic (CLL), Leukemia—Chronic Myeloid (CML), Liver Cancer, Lobular Carcinoma, Lung Cancer, Lung Cancer—Small Cell (SCLC), Lung Cancer—Non-small Cell (NSCLC), Lymphoma—Hodgkin's, Lymphoma—Non-Hodgkin's, Malignant Glioma, Melanoma, Meningioma, Multiple Myeloma, Myelodysplastic Syndrome (MDS), Nasopharyngeal Cancer, Neuroendocrine Tumor, Oral Cancer, Osteosarcoma, Ovarian Cancer, Pancreatic Cancer, Pancreatic Neuroendocrine Tumors, Parathyroid Cancer, Penile Cancer, Peritoneal Cancer, Peutz-Jeghers Syndrome, Pituitary Gland Tumor, Polycythemia Vera, Prostate Cancer, Renal Cell Carcinoma, Retinoblastoma, Salivary Gland Cancer, Sarcoma, Sarcoma—Kaposi, Skin Cancer, Small Intestine Cancer, Stomach Cancer, Testicular Cancer, Thymoma, Thyroid Cancer, Uterine (Endometrial) Cancer, Vaginal Cancer, and Wilms' Tumor.

In some embodiments, the disease or the condition is an inflammatory bowel disease, such as Crohn's disease (CD) or ulcerative colitis (UC). A subject may suffer from fibrosis, fibrostenosis, or a fibrotic disease, either isolated or in combination with an inflammatory disease. In some cases, the CD is severe CD. The severe CD may result from inflammation that has led to the formation of scar tissue in the intestinal wall (fibrostenosis) and/or swelling. In some cases, the severe CD is characterized by the presence of fibrotic and/or inflammatory strictures. The strictures may be determined by computed tomography enterography (CTE), and magnetic resonance imaging enterography (MRE). The disease or condition may be characterized as refractory, which in some cases, means the disease is resistant to a standard treatment (e.g., anti-TNFα therapy). Non-limiting examples of standard treatment include glucocorticosteriods, anti-TNF therapy, anti-a4-b7 therapy (vedolizumab), anti-IL12p40 therapy (ustekinumab), Thalidomide, and Cytoxin.

### Calculating a Genetic Risk Score

In some embodiments, described herein are methods of calculating a genetic risk score for a subject. The genetic variants disclosed herein comprise SNVs, indels, and/or CNVs. Each genetic variant comprises units of risk used to calculate a genetic risk score. In some instances, a unit of risk within an SNV comprises the risk allele. In some instances, a unit of risk within an indel comprises the insertion or deletion. In some instances, a unit of risk within a CNV comprises an increase or a decrease in a number of copies of a gene or segment of a gene as compared to a wild-type copy number. Many methods of calculating a genetic risk score may be used to calculate the genetic risk score of the subject according to the present methods and systems.

Disclosed herein, in some embodiments, are methods of calculating a genetic risk score of a subject. In some instances, the units of risk within an SNV (e.g. risk allele), an Indel (e.g. insertion or deletion), and/or CNV (e.g. copy number), may be assigned an arbitrary numerical value. In a non-limiting example of calculating a genetic risk score involving SNVs, a homozygous genotype for a risk allele within a SNV (RR) is assigned a numerical value 2; a heterozygous genotype for a risk allele within a SN is assigned a numerical value 1; a genotype that is nonrisk (N) is assigned a numerical value 0. Next, each polymorphism is weighted according to the strength of their association with the phenotype of the disease or condition. The weighted sum of each numerical value for all individual SNVs are added together, and divided by the total number of genetic variants used in the model, to generate a raw score for the subject. The same calculations are performed for each individual belonging to the subject group, thereby generating a range of raw scores. In some embodiments, the subject group comprises individuals with the same disease or condition as the subject. The distribution of the genetic risk score is normally distributed within the population.

In certain aspects, disclosed herein is a method of calculating a genetic risk score, the method comprising providing genomic data comprising one or more genotypes of the subject, wherein the one or more genotypes is associated with high TL1A fold-change relative to an index or a control; selecting, from a database, one or more genetic variants corresponding to: the one or more genotypes of the subject, or a predetermined genetic variant in a linkage disequilibrium (LD) therewith; and calculating a genetic risk score for the subject, based, at least in part, on the one or more genetic variants. In some embodiments, the method further comprises weighting the genotypes at the one or more genetic variants. In some embodiments, a larger weight is given to genotypes that have the strongest genetic association with a disease of interest, such as CD or IBD. In some embodiments, a larger weight is given to genotypes that have the strongest genetic association with tissue expression of TL1A (e.g., cis-eQTL). In some embodiments, a larger weight is gen to genotypes at one or more genetic variants at the TNFSF15 gene. In certain embodiments, the genetic risk score is a polygenetic risk score (PRS). In some embodiments, linkage disequilibrium comprises a D′ value of at least 0.70, 0.75, 0.80, 0.85, 0.90, 0.95, or 0.99. In some embodiments, linkage disequilibrium comprises a D′ value of 0 and a R² value of at least 0.7, 0.75, 0.80, 0.85, 0.90, 0.95 or 0.99.

In certain aspects, disclosed herein is a method of calculating a genetic risk score, the method comprising: providing genomic data comprising one or more genotypes of the subject, wherein the one or more genotypes is associated with high TL1A fold-change relative to an index or a control; detecting the one or more genetic variants or a predetermined genetic variant in a linkage disequilibrium (LD) therewith; and calculating a genetic risk score for the subject, based, at least in part, on the one or more genetic variants or the predetermined genetic variant that were detected. In some embodiments, the method further comprises weighting the genotypes at the one or more genetic variants. In some embodiments, a larger weight is given to genotypes that have the strongest genetic association with a disease of interest, such as CD or IBD. In some embodiments, a larger weight is given to genotypes that have the strongest genetic association with tissue expression of TL1A (e.g., cis-eQTL). In some embodiments, a larger weight is gen to genotypes at one or more genetic variants at the TNFSF15 gene. In certain embodiments, the genetic risk score is a polygenetic risk score (PRS). In some embodiments, linkage disequilibrium comprises a D′ value of at least 0.70, 0.75, 0.80, 0.85, 0.90, 0.95, or 0.99. In some embodiments, linkage disequilibrium comprises a D′ value of 0 and a R² value of at least 0.7, 0.75, 0.80, 0.85, 0.90, 0.95 or 0.99.

In certain embodiments, the one or more genotypes is associated with high TL1A fold-change relative to an index or control. In certain embodiments, the predetermined genetic variant is selected based at least in part on an association with case (e.g., IBD, CD) versus control (e.g., non-IBD). In certain embodiments, the association with case comprises association with inflammatory disease, a Crohn's disease, or ulcerative colitis. In some embodiments, the association with control comprises an association with non-IBD. In certain embodiments, the index or control is obtained from a subject or a population of subjects with inflammatory disease, a fibrostenotic disease, or a fibrotic disease. In certain embodiments, the index or control is obtained from a subject or a population of subjects without inflammatory disease, a fibrostenotic disease, or a fibrotic disease.

In some embodiments, identifying the predetermined genetic variant comprising estimating a haplotype (also referred to herein “haplotype phasing”). In some embodiments, haplotype phasing is performed using an algorithm, such as, expectation-maximization algorithm, hidden Markov model (HMM), PHASE (v2.1.1), fastPHASE, BEAGLE, IMPUTE2, MaCH, SHAPEIT1, HAPI-UR, or SHAPEIT2. In certain embodiments, the predetermined genetic variant is imputed using phased haplotype data from a reference group that has the same clinical phenotypes as the subject.

### Genotypes

Disclosed herein are genotypes used to generate a polygenic risk score. In some embodiments, the genotypes that may be detected in a sample obtained from a subject by analyzing the genetic material in the sample. In some embodiments, the subject may be human. In some embodiments, the genetic material is obtained from a subject having a disease or condition disclosed herein. In some cases, the genetic material is obtained from blood, serum, plasma, sweat, hair, tears, urine, and other techniques. In some cases, the genetic material is obtained from a biopsy, e.g., from the intestinal track of the subject.

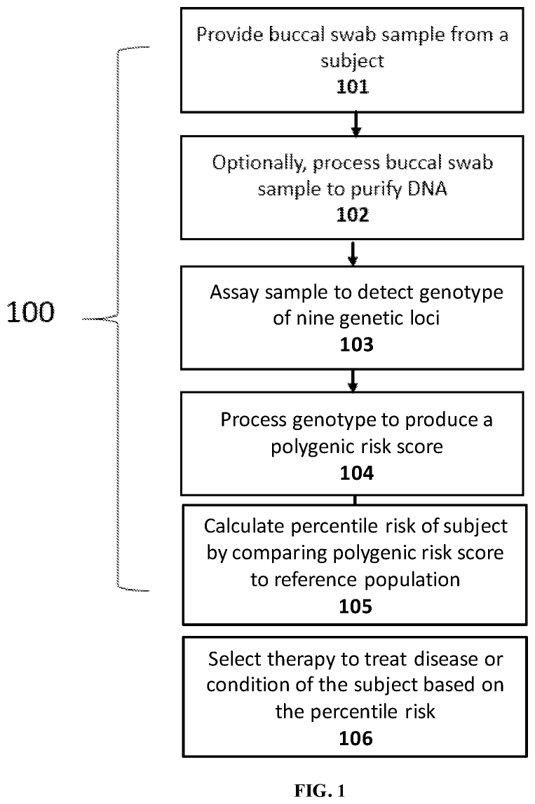

Referring to FIG. 1, the methods, systems and kits of the present disclosure involve, in some embodiments, the steps of providing a buccal swab sample from a subject 101, optionally purifying DNA from the sample by processing the sample 102, assaying the optionally processed sample to detect genotypes of nine genetic loci in the sample 103, processing the genotypes to produce a polygenic risk score (PRS) 104, calculating the percentile risk of the subject by comparing the PRS to a reference population 105 and selecting a therapy to treat a disease or disorder of the subject based on the percentile 106.

The genotypes described herein are detected using suitable genotyping devices (e.g., array, sequencing). In some embodiments, a sample is obtained from the subject or patient indirectly or directly. In some embodiments, the sample may be obtained by the subject. In other instances, the sample may be obtained by a healthcare professional, such as a nurse or physician. The sample may be derived from virtually any biological fluid or tissue containing genetic information, such as blood.

The genotypes of the present disclosure comprise genetic material that is deoxyribonucleic acid (DNA). In some embodiments, the genotype comprises a denatured DNA molecule or fragment thereof. In some embodiments, the genotype comprises DNA selected from: genomic DNA, viral DNA, mitochondrial DNA, plasmid DNA, amplified DNA, circular DNA, circulating DNA, cell-free DNA, or exosomal DNA. In some embodiments, the DNA is single-stranded DNA (ssDNA), double-stranded DNA, denaturing double-stranded DNA, synthetic DNA, and combinations thereof. The circular DNA may be cleaved or fragmented.

The genotypes disclosed herein comprise at least one polymorphism located at a gene or genetic locus described herein. In some embodiments, the gene or genetic locus is selected from the group consisting of ETS Proto-oncogene 1 (ETS1), Interferon Gamma (IFNG), TNF Receptor Superfamily Member 6b (TNFRSF6B), TNF Superfamily Member 15 (TNFSF15), TNF Superfamily Member 8 (TNFSF8), U2 (novel transcript AF111167.2), Fos Proto-oncogene (FOS), Interferon Gamma Receptor 2 (IFNGR2), Zinc Finger CCH-Type and G-Patch Domain Containing (ZGPAT), Solute Carrier Family 19 Member 3 (SLC19A3), and C—C Motif Chemokine Ligand 20 (CC120). In some embodiments, the gene of genetic locus comprises a gene or genetic locus provided in Table 10. The genotypes disclosed herein are, in some cases, a haplotype. In some embodiments, the genotype comprises a particular polymorphism, a polymorphism in linkage disequilibrium (LD) therewith, or a combination thereof. In some cases, LD is defined by an r2 of at least or about 0.70, 0.75, 0.80, 0.85, 0.90, or 1.0. The genotypes disclosed herein can comprise at least or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more polymorphisms. In some embodiments, the genotypes disclosed herein comprise a combination of 9 polymorphisms, such as those provided in Table 10.

The polymorphisms described herein can be a single nucleotide polymorphism, or an indel (insertion/deletion). In some embodiments, the polymorphism is an insertion or a deletion of at least one nucleobase (e.g., an indel). In some embodiments, the genotype may comprise a copy number variation (CNV), which is a variation in a number of a nucleic acid sequence between individuals in a given population. In some embodiments, the CNV comprises at least or about two, three, four, five, six, seven, eight, nine, ten, twenty, thirty, forty or fifty nucleic acid molecules. In some embodiments, the genotype is heterozygous. In some embodiments, the genotype is homozygous.

Disclosed herein, in the following embodiments, are genotypes disclosed herein: (1) A genotype comprising at least one polymorphism at a gene or genetic locus. (2) The genotype of embodiment 1 comprising a polymorphism provided in Table 10. (3) The genotype of embodiments 1-2 that is heterozygous. (4) The genotype of embodiments 1-2 that is homozygous. (5) The genotype of embodiments 1-4, wherein the genotype comprises at least two polymorphisms. (6) The genotype of embodiments 1-4, wherein the genotype comprises at least three polymorphisms. (7) The genotype of embodiments 1-4, wherein the genotype comprises at least four polymorphisms. (8) The genotype of embodiments 1-4, wherein the genotype comprises at least five polymorphisms. (9) The genotype of embodiments 1-4, wherein the genotype comprises at least six polymorphisms. (10) The genotype of embodiments 1-4, wherein the genotype comprises at least seven polymorphisms. (11) The genotype of embodiments 1-4, wherein the genotype comprises at least eight polymorphisms. (12) The genotype of embodiments 1-4, wherein the genotype comprises at least nine polymorphisms. (13) The genotype of embodiment 1, comprising a polymorphism in linkage disequilibrium with a polymorphism provided in Table 10. (14) The genotype of embodiment 13, wherein LD is defined by (i) a D′ value of at least about 0.70, or (ii) a D′ value of 0 and an r2 value of at least about 0.70. (15) The genotype of embodiment 13, wherein LD is defined by (i) a D′ value of at least about 0.80, or (ii) a D′ value of 0 and an r2 value of at least about 0.80. (16) The genotype of embodiment 13, wherein LD is defined by (i) a D′ value of at least about 0.90, or (ii) a D′ value of 0 and an r2 value of at least about 0.90. (17) The genotype of embodiment 13, wherein LD is defined by (i) a D′ value of at least about 0.95, or (ii) a D′ value of 0 and an r2 value of at least about 0.95. (18) The genotype of embodiments 1-17, wherein the gene or genetic locus is selected from the group consisting of ETS Proto-oncogene 1 (ETS1), Interferon Gamma (IFNG), TNF Receptor Superfamily Member 6b (TNFRSF6B), TNF Superfamily Member 15 (TNFSF15), TNF Superfamily Member 8 (TNFSF8), U2, Fos Proto-oncogene (FOS), Interferon Gamma Receptor 2 (IFNGR2), Zinc Finger CCH-Type and G-Patch Domain Containing (ZGPAT), Solute Carrier Family 19 Member 3 (SLC19A3), and C—C Motif Chemokine Ligand 20 (CC120). (19) The genotype of embodiments 5-6, wherein the genotype comprises at least two polymorphisms selected from rs11221332, rs7134599, rs6062496, rs4246905, rs7468800, rs1569328, rs2284553, rs6062504, and rs7556897. (20) The genotype of embodiments 5-6, wherein the genotype comprises at least three polymorphisms selected from rs11221332, rs7134599, rs6062496, rs4246905, rs7468800, rs1569328, rs2284553, rs6062504, and rs7556897. (21) The genotype of embodiments 5-6, wherein the genotype comprises at least four polymorphisms selected from rs11221332, rs7134599, rs6062496, rs4246905, rs7468800, rs1569328, rs2284553, rs6062504, and rs7556897. (22) The genotype of embodiments 5-6, wherein the genotype comprises at least five polymorphisms selected from rs11221332, rs7134599, rs6062496, rs4246905, rs7468800, rs1569328, rs2284553, rs6062504, and rs7556897. (23) The genotype of embodiments 5-6, wherein the genotype comprises at least six polymorphisms selected from rs11221332, rs7134599, rs6062496, rs4246905, rs7468800, rs1569328, rs2284553, rs6062504, and rs7556897. (24) The genotype of embodiments 5-6, wherein the genotype comprises at least seven polymorphisms selected from rs11221332, rs7134599, rs6062496, rs4246905, rs7468800, rs1569328, rs2284553, rs6062504, and rs7556897. (25) The genotype of embodiments 5-6, wherein the genotype comprises at least eight polymorphisms selected from rs11221332, rs7134599, rs6062496, rs4246905, rs7468800, rs1569328, rs2284553, rs6062504, and rs7556897. (26) The genotype of embodiments 5-6, wherein the genotype comprises at least nine polymorphisms selected from rs11221332, rs7134599, rs6062496, rs4246905, rs7468800, rs1569328, rs2284553, rs6062504, and rs7556897.

In some embodiments, disclosed herein provide genotypes that are associated with, and therefore indicative of, a subject having or being susceptible to developing a particular disease or condition, or a subclinical phenotype thereof. In addition, the genotypes disclosed herein are associated with an increase TNFSF15 (TL1A) fold-change. Thus, the genotypes are indicative that the subject will have a positive therapeutic response to an inhibitor of TL1A activity or expression. Table 10 provides polymorphisms associated with, and therefore predictive of an increase in TNSFF15 (TL1A) fold-change.

### TL1A Fold-Change

In certain aspects, disclosed herein are methods related to predicting or calculating a fold-change of TL1A expression. In some embodiments, fold-change of TL1A is the change in expression of TL1A obtained from a subject at a first time point as compared with the expression of TL1a obtained from the subject at a second time point, where the second time point is later in time than the first time point. In certain aspects, the methods described herein comprise: obtaining peripheral blood mononuclear cells (PBMCs) from the subject; bringing the PMBCs into contact with immune-complex under conditions sufficient to produce TL1A by the PBMCs; measuring TL1A expression at a first time point; measuring TL1A expression at a second time point; and calculating the fold-change of TL1A between the first and the second time point.

In certain aspects, calculating the fold-change of TL1A expression comprises dividing the TL1A expression level at the second time point by the TL1A expression level at the first time point.

In certain embodiments, the first time point is at least 6 hours, 18 hours, 24 hours, 48 hours, or 72 hours after contacting the PMBCs with an immune complex. In certain embodiments, the first time point is at least 6 hours after contacting the PBMCs with the immune complex. In certain embodiments, the first time point is at least 18 hours after contacting the PBMCs with the immune complex. In certain embodiments, the first time point is at least 24 hours after contacting the PBMCs with the immune complex. In certain embodiments, the first time point is at least 48 hours after contacting the PBMCs with the immune complex. In certain embodiments, the first time point is at least 72 hours after contacting the PBMCs with the immune complex. In certain embodiments, the second time point is at least 6 hours, 18 hours, 24 hours, 48 hours, or 72 hours after contacting the PMBCs with an immune complex. In certain embodiments, the second time point is at least 6 hours after contacting the PBMCs with the immune complex. In certain embodiments, the second time point is at least 18 hours after contacting the PBMCs with the immune complex. In certain embodiments, the second time point is at least 24 hours after contacting the PBMCs with the immune complex. In certain embodiments, the second time point is at least 48 hours after contacting the PBMCs with the immune complex. In certain embodiments, the second time point is at least 72 hours after contacting the PBMCs with the immune complex.

In certain embodiments, the TL1A fold-change comprises a high TL1A fold-change. In certain embodiments, a high TL1A fold-change comprises a TL1A fold-change that is at least equal to the mean plus one standard deviation relative to an index or control population. In certain embodiments, a high TL1A fold-change comprises a TL1A fold-change that is at least equal to the mean plus two standard deviations relative to an index or control population. In certain embodiments, a high TL1A fold-change comprises a TL1A fold-change that is at least equal to the mean plus three standard deviations relative to an index or control population.

In certain embodiments, the method further comprises selecting the subject for treatment with an inhibitor of TL1A activity of expression, provided the fold-change of TL1A is high relative to an index or control. In some embodiments, the method further comprises administering a therapeutically effective amount of an inhibitor of TL1A activity or expression. In certain embodiments, a high fold-change of TL1A relative to an index or control indicates that the subject has a moderate to severely active form of CD or UC.

In certain embodiments, the one or more genotypes is associated with high TL1A fold-change relative to an index or control. In certain embodiments, the genetic risk score is associated with high TL1A fold-change relative to an index or control. In certain embodiments, the polygenic risk score is associated with high TL1A fold-change relative to an index or control. In certain embodiments, a polygenic risk score in the 75^{th} percentile is associated with high TL1A fold-change relative to an index or control. In certain embodiments, a polygenic risk score in the 75^{th} percentile is associated with a moderate to sever form of Crohn's disease or ulcerative colitis. In certain embodiments, a polygenic risk score can be used to diagnose a patient with a moderate to severe form of Crohn's disease or ulcerative colitis. In certain embodiments, a polygenic risk score can be used to select a patient with Crohn's disease or ulcerative colitis for treatment with an inhibitor of TL1A activity or expression.

In certain embodiments, the predetermined genetic variant is selected based at least in part on an association with case versus control. In certain embodiments, the association with case comprises association with inflammatory disease, a Crohn's disease, or ulcerative colitis. In certain embodiments, the index or control is a population of subjects with inflammatory disease, a fibrostenotic disease, or a fibrotic disease. In certain embodiments, the index or control is a population of subjects without inflammatory disease, a fibrostenotic disease, or a fibrotic disease.

### Methods

Methods disclosed herein comprise methods for detection of a genotype in a subject and methods of treating a subject. In some embodiments, methods further comprise calculating a genetic risk score disclosed herein, which is predictive of high TL1A fold-change in the subject. In some embodiments, the methods describe methods for characterizing the treatment of a subject. In some embodiments, the methods comprise methods of monitoring treatment. In some embodiments, the methods comprise methods of selecting a subject for treatment. In some embodiments, the method comprise methods of inhibiting or reducing TL1A activity or expression in a subject. In some embodiments, the subject has an inflammatory, fibrotic or fibrostenotic disease.

### Methods of Detection

Methods disclosed herein for detecting a genotype in a sample from a subject comprise analyzing the genetic material in the sample to detect at least one of a presence, an absence, and a quantity of a nucleic acid sequence encompassing the genotype of interest. In some embodiments, the sample is assayed to measure a presence, absence or quantity of at least three polymorphisms. In some embodiments, the sample is assayed to measure a presence, absence, or quantity of at least four polymorphisms. In some embodiments, the sample is assayed to measure a presence, absence, or quantity of at least five polymorphisms. In some embodiments, at least three genotypes are detected, using the methods described herein.

In some cases, the nucleic acid sequence comprises DNA. In some embodiments, the nucleic acid sequence comprises a denatured DNA molecule or fragment thereof. In some embodiments, the nucleic acid sequence comprises DNA selected from: genomic DNA, viral DNA, mitochondrial DNA, plasmid DNA, amplified DNA, circular DNA, circulating DNA, cell-free DNA, complementary DNA (cDNA), or exosomal DNA. In some embodiments, the DNA is single-stranded DNA (ssDNA), double-stranded DNA, denaturing double-stranded DNA, synthetic DNA, and combinations thereof. The circular DNA may be cleaved or fragmented. In some embodiments, the nucleic acid sequence comprises RNA. In some embodiments, the nucleic acid sequence comprises fragmented RNA. In some embodiments, the nucleic acid sequence comprises partially degraded RNA. In some embodiments, the nucleic acid sequence comprises a microRNA or portion thereof. In some embodiments, the nucleic acid sequence comprises an RNA molecule or a fragmented RNA molecule (RNA fragments) selected from: a microRNA (miRNA), a pre-miRNA, a pri-miRNA, a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (lncRNA), a small nuclear RNA (snRNA), a circulating RNA, a cell-free RNA, an exosomal RNA, a vector-expressed RNA, an RNA transcript, a synthetic RNA, and combinations thereof.

Nucleic acid-based detection techniques that may be useful for the methods herein include quantitative polymerase chain reaction (qPCR), gel electrophoresis, immunochemistry, in situ hybridization such as fluorescent in situ hybridization (FISH), cytochemistry, and next generation sequencing. In some embodiments, the methods involve TaqMan™ qPCR, which involves a nucleic acid amplification reaction with a specific primer pair, and hybridization of the amplified nucleic acids with a hydrolysable probe specific to a target nucleic acid.

In some embodiments, the methods involve hybridization and/or amplification assays that include, but are not limited to, Southern or Northern analyses, polymerase chain reaction analyses, and probe arrays. Non-limiting amplification reactions include, but are not limited to, qPCR, self-sustained sequence replication, transcriptional amplification system, Q-Beta Replicase, rolling circle replication, or any other nucleic acid amplification. As discussed, reference to qPCR herein includes use of TaqMan™ methods. An additional hybridization assay includes the use of nucleic acid probes conjugated or otherwise immobilized on a bead, multi-well plate, or other substrate, wherein the nucleic acid probes are configured to hybridize with a target nucleic acid sequence of a genotype provided herein.

In some embodiments, detecting the presence or absence of a genotype comprises sequencing genetic material from the subject. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modern sequencing technologies such as Illumina sequencing (e.g., Solexa), Roche 454 sequencing, Ion torrent sequencing, and SOLiD sequencing. In some cases, next-generation sequencing involves high-throughput sequencing methods. Additional sequencing methods may also be employed.

In some embodiments, a number of nucleotides that are sequenced are at least 5, 10, 15, 20, 25, 30, 35, 40, 45, 50, 100, 150, 200, 300, 400, 500, 2000, 4000, 6000, 8000, 10000, 20000, 50000, 100000, or more than 100000 nucleotides. In some embodiments, the number of nucleotides sequenced is in a range of about 1 to about 100000 nucleotides, about 1 to about 10000 nucleotides, about 1 to about 1000 nucleotides, about 1 to about 500 nucleotides, about 1 to about 300 nucleotides, about 1 to about 200 nucleotides, about 1 to about 100 nucleotides, about 5 to about 100000 nucleotides, about 5 to about 10000 nucleotides, about 5 to about 1000 nucleotides, about 5 to about 500 nucleotides, about 5 to about 300 nucleotides, about 5 to about 200 nucleotides, about 5 to about 100 nucleotides, about 10 to about 100000 nucleotides, about 10 to about 10000 nucleotides, about 10 to about 1000 nucleotides, about 10 to about 500 nucleotides, about 10 to about 300 nucleotides, about 10 to about 200 nucleotides, about 10 to about 100 nucleotides, about 20 to about 100000 nucleotides, about 20 to about 10000 nucleotides, about 20 to about 1000 nucleotides, about 20 to about 500 nucleotides, about 20 to about 300 nucleotides, about 20 to about 200 nucleotides, about 20 to about 100 nucleotides, about 30 to about 100000 nucleotides, about 30 to about 10000 nucleotides, about 30 to about 1000 nucleotides, about 30 to about 500 nucleotides, about 30 to about 300 nucleotides, about 30 to about 200 nucleotides, about 30 to about 100 nucleotides, about 50 to about 100000 nucleotides, about 50 to about 10000 nucleotides, about 50 to about 1000 nucleotides, about 50 to about 500 nucleotides, about 50 to about 300 nucleotides, about 50 to about 200 nucleotides, or about 50 to about 100 nucleotides.

In some embodiments, probes comprise a nucleic acid sequence of at least 10 contiguous nucleic acids provided in any one of SEQ ID NOS: 401-409 including the nucleobase indicated with a non-nucleobase letter (e.g., R, N, S), or a reverse complement thereof. In some embodiments, the probes may be used to detect the polymorphisms provided in Table 10, wherein the probe comprises a nucleic acid sequence of at least 10 contiguous nucleic acids provided in a corresponding SEQ ID NO or reverse complement thereof, the 10 contiguous nucleic acids comprising the “risk allele” also provided in Table 10 at a nucleoposition indicated with the non-nucleobase letter, or reverse complement thereof. In some embodiments, the probe comprises at least 70%, 80%, 85%, 90%, 91%, 92%, 93%, 94%, 95%, 96%, 97%, 98% or 99% sequence identity to any one of SEQ ID NOS: 401-409 or its reverse complement. In some embodiments, forward and reverse primers are used to amplify the target nucleic acid sequence. Forward and reverse primers may comprise a nucleic acid sequence flanking the risk allele provided in Table 10 corresponding to the nucleic acid sequence provided in any one of SEQ ID NOS: 401-409 or a reverse complement thereof.

Examples of molecules that are utilized as probes include, but are not limited to, RNA and DNA. In some embodiments, the term “probe” with regards to nucleic acids, refers to any molecule that is capable of selectively binding to a specifically intended target nucleic acid sequence. In some embodiments, probes are specifically designed to be labeled, for example, with a radioactive label, a fluorescent label, an enzyme, a chemiluminescent tag, a colorimetric tag, or other labels or tags. In some embodiments, the fluorescent label comprises a fluorophore. In some embodiments, the fluorophore is an aromatic or heteroaromatic compound. In some embodiments, the fluorophoreis a pyrene, anthracene, naphthalene, acridine, stilbene, benzoxazole, indole, benzindole, oxazole, thiazole, benzothiazole, canine, carbocyanine, salicylate, anthranilate, xanthenes dye, coumarin. In some examples, xanthene dyes include, e.g., fluorescein and rhodamine dyes. Fluorescein and rhodamine dyes include, but are not limited to 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N; N′-tetramethyl-6-carboxyrhodamine (TAMRA), 6-carboxy-X-rhodamine (ROX). Suitable fluorescent probes also include the naphthylamine dyes that have an amino group in the alpha or beta position. For example, naphthylamino compounds include 1-dimethylaminonaphthyl-5-sulfonate, 1-anilino-8-naphthalene sulfonate and 2-p-toluidinyl-6-naphthalene sulfonate, 5-(2′-aminoethyl)aminonaphthalene-1-sulfonic acid (EDANS). In some examples, coumarins include, e.g., 3-phenyl-7-isocyanatocoumarin; acridines, such as 9-isothiocyanatoacridine and acridine orange; N-(p-(2-benzoxazolyl)phenyl) maleimide; cyanines, such as, e.g., indodicarbocyanine 3 (Cy3), indodicarbocyanine 5 (Cy5), indodicarbocyanine 5.5 (Cy5.5), 3-(-carboxy-pentyl)-3′-ethyl-5,5′-dimethyloxacarbocyanine (CyA); 1H, 5H, 11H, 15H-Xantheno[2,3, 4-ij: 5,6, 7-i′j′]diquinolizin-18-ium, 9-[2 (or 4)-[[[6-[2,5-dioxo-1-pyrrolidinyl)oxy]-6-oxohexyl]amino]sulfonyl]-4 (or 2)-sulfophenyl]-2,3, 6,7, 12,13, 16,17-octahydro-inner salt (TR or Texas Red); or BODIPY™ dyes. In some cases, the probe comprises FAM as the dye label.

In some embodiments, primers and/or probes described herein for detecting a target nucleic acid are used in an amplification reaction. In some embodiments, the amplification reaction is qPCR. In an example, a qPCR is a method employing a TaqMan™ assay. In some embodiments, qPCR comprises using an intercalating dye. Examples of intercalating dyes include SYBR green I, SYBR green II, SYBR gold, ethidium bromide, methylene blue, Pyronin Y, DAPI, acridine orange, Blue View or phycoerythrin. In some embodiments, the intercalating dye is SYBR.

In some embodiments, a number of amplification cycles for detecting a target nucleic acid in an amplification assay is about 5 to about 30 cycles. In some embodiments, the number of amplification cycles for detecting a target nucleic acid is at least about 5 cycles. In some embodiments, the number of amplification cycles for detecting a target nucleic acid is at most about 30 cycles. In some embodiments, the number of amplification cycles for detecting a target nucleic acid is about 5 to about 10, about 5 to about 15, about 5 to about 20, about 5 to about 25, about 5 to about 30, about 10 to about 15, about 10 to about 20, about 10 to about 25, about 10 to about 30, about 15 to about 20, about 15 to about 25, about 15 to about 30, about 20 to about 25, about 20 to about 30, or about 25 to about 30 cycles.

In one aspect, the methods provided herein for determining the presence, absence, and/or quantity of a nucleic acid sequence comprise an amplification reaction such as qPCR. In one embodiment, genetic material is obtained from a sample of a subject, e.g., a sample of blood or serum. In certain embodiments where nucleic acids are extracted, the nucleic acids are extracted using any technique that does not interfere with subsequent analysis. In certain embodiments, this technique uses alcohol precipitation using ethanol, methanol, or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example is the GenElute Bacterial Genomic DNA Kit available from Sigma Aldrich. In certain embodiments, after extraction the nucleic acid is stored in water, Tris buffer, or Tris-EDTA buffer before subsequent analysis. In an embodiment, the nucleic acid material is extracted in water. In some cases, extraction does not comprise nucleic acid purification.

In an example qPCR assay, the nucleic acid sample is combined with primers and probes specific for a target nucleic acid that may or may not be present in the sample, and a DNA polymerase. An amplification reaction is performed with a thermal cycler that heats and cools the sample for nucleic acid amplification, and illuminates the sample at a specific wavelength to excite a fluorophore on the probe and detect the emitted fluorescence. For TaqMan™ methods, the probe may be a hydrolysable probe comprising a fluorophore and quencher that is hydrolyzed by DNA polymerase when hybridized to a target nucleic acid. In some cases, the presence of a target nucleic acid is determined when the number of amplification cycles to reach a threshold value is less than 30, 29, 28, 27, 26, 25, 24, 23, 22, 21, or 20 cycles.

In some embodiments, the target nucleic acid is at least 10 contiguous nucleic acid molecules of SEQ ID NO: 1 comprising a non-reference allele at nucleoposition 26 within SEQ ID NO: 1. In some embodiments, the target nucleic acid is at least 10 contiguous nucleic acid molecules of SEQ ID NO: 1 comprising a “G” or an “A” allele at nucleoposition 26 within SEQ ID NO: 1. In some embodiments, detecting the at least 10 contiguous nucleic acid molecules comprising a “G” or an “A” allele at nucleoposition 26 within SEQ ID NO: 1 is sufficient to detect the polymorphism at rs11221332.

In some embodiments, the target nucleic acid is at least 10 contiguous nucleic acid molecules of SEQ ID NO: 2 comprising a non-reference allele at nucleoposition 26 within SEQ ID NO: 2. In some embodiments, the target nucleic acid is at least 10 contiguous nucleic acid molecules of SEQ ID NO: 2 comprising a “G” or an “A” allele at nucleoposition 26 within SEQ ID NO: 2. In some embodiments, detecting the at least 10 contiguous nucleic acid molecules comprising a “G” or an “A” allele at nucleoposition 26 within SEQ ID NO: 2 is sufficient to detect the polymorphism at rs7134599.

In some embodiments, the target nucleic acid is at least 10 contiguous nucleic acid molecules of SEQ ID NO: 3 comprising a non-reference allele at nucleoposition 26 within SEQ ID NO: 3. In some embodiments, the target nucleic acid is at least 10 contiguous nucleic acid molecules of SEQ ID NO: 3 comprising an “A” or a “G” allele at nucleoposition 26 within SEQ ID NO: 3. In some embodiments, detecting the at least 10 contiguous nucleic acid molecules comprising an “A” or a “G” allele at nucleoposition 26 within SEQ ID NO: 3 is sufficient to detect the polymorphism at rs6062496.

In some embodiments, the target nucleic acid is at least 10 contiguous nucleic acid molecules of SEQ ID NO: 4 comprising a non-reference allele at nucleoposition 26 within SEQ ID NO: 4. In some embodiments, the target nucleic acid is at least 10 contiguous nucleic acid molecules of SEQ ID NO: 4 comprising a “G” or an “A” allele at nucleoposition 26 within SEQ ID NO: 4. In some embodiments, detecting the at least 10 contiguous nucleic acid molecules comprising a “G” or an “A” allele at nucleoposition 26 within SEQ ID NO: 4 is sufficient to detect the polymorphism at rs4246905.

In some embodiments, the target nucleic acid is at least 10 contiguous nucleic acid molecules of SEQ ID NO: 5 comprising a non-reference allele at nucleoposition 26 within SEQ ID NO: 5. In some embodiments, the target nucleic acid is at least 10 contiguous nucleic acid molecules of SEQ ID NO: 5 comprising a “C” or an “A” allele at nucleoposition 26 within SEQ ID NO: 5. In some embodiments, detecting the at least 10 contiguous nucleic acid molecules comprising a “C” or an “A” allele at nucleoposition 26 within SEQ ID NO: 5 is sufficient to detect the polymorphism at rs7468800.

In some embodiments, the target nucleic acid is at least 10 contiguous nucleic acid molecules of SEQ ID NO: 6 comprising a non-reference allele at nucleoposition 26 within SEQ ID NO: 6. In some embodiments, the target nucleic acid is at least 10 contiguous nucleic acid molecules of SEQ ID NO: 6 comprising a “G” or an “A” allele at nucleoposition 26 within SEQ ID NO: 6. In some embodiments, detecting the at least 10 contiguous nucleic acid molecules comprising a “G” or an “A” allele at nucleoposition 26 within SEQ ID NO: 6 is sufficient to detect the polymorphism at rs1569328.

In some embodiments, the target nucleic acid is at least 10 contiguous nucleic acid molecules of SEQ ID NO: 7 comprising a non-reference allele at nucleoposition 26 within SEQ ID NO: 7. In some embodiments, the target nucleic acid is at least 10 contiguous nucleic acid molecules of SEQ ID NO: 7 comprising a “G” or an “A” allele at nucleoposition 26 within SEQ ID NO: 7. In some embodiments, detecting the at least 10 contiguous nucleic acid molecules comprising a “G” or an “A” allele at nucleoposition 26 within SEQ ID NO: 7 is sufficient to detect the polymorphism at rs2284553.

In some embodiments, the target nucleic acid is at least 10 contiguous nucleic acid molecules of SEQ ID NO: 8 comprising a non-reference allele at nucleoposition 26 within SEQ ID NO: 8. In some embodiments, the target nucleic acid is at least 10 contiguous nucleic acid molecules of SEQ ID NO: 8 comprising a “G” or an “A” allele at nucleoposition 26 within SEQ ID NO: 8. In some embodiments, detecting the at least 10 contiguous nucleic acid molecules comprising a “G” or an “A” allele at nucleoposition 26 within SEQ ID NO: 8 is sufficient to detect the polymorphism at rs6062504.

In some embodiments, the target nucleic acid is at least 10 contiguous nucleic acid molecules of SEQ ID NO: 9 comprising a non-reference allele at nucleoposition 26 within SEQ ID NO: 9. In some embodiments, the target nucleic acid is at least 10 contiguous nucleic acid molecules of SEQ ID NO: 9 comprising an “A” or a “G” allele at nucleoposition 26 within SEQ ID NO: 9. In some embodiments, detecting the at least 10 contiguous nucleic acid molecules comprising an “A” or a “G” allele at nucleoposition 26 within SEQ ID NO: 9 is sufficient to detect the polymorphism at rs7556897.

In some embodiments, one target nucleic acid (e.g., a polymorphism) is detected with the methods disclosed herein. In some embodiments, at least 2, 3, 4, 5, 6, 7, 8, 9, or 10 target nucleic acids are detected. In some embodiments, the at least 2, 3, 4, 5, 6, 7, 8, 9, or 10 target nucleic acids are detected in a single multiplexed assay.

To practice the methods and systems provided herein, genetic material may be extracted from a sample obtained from a subject, e.g., a sample of blood or serum. In certain embodiments where nucleic acids are extracted, the nucleic acids are extracted using any technique that does not interfere with subsequent analysis. In certain embodiments, this technique uses alcohol precipitation using ethanol, methanol or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example is the GenElute Bacterial Genomic DNA Kit available from Sigma Aldrich. In certain embodiments, after extraction the nucleic acid is stored in water, Tris buffer, or Tris-EDTA buffer before subsequent analysis. In an example embodiment, the nucleic acid material is extracted in water. In some cases, extraction does not comprise nucleic acid purification. In certain embodiments, RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland).

In some embodiments, methods of detecting a presence, absence, or level of a target protein (e.g., biomarker) in the sample obtained from the subject involve detecting protein activity or expression. In some embodiments, the target protein is TL1A, or a binding partner of TL1A such as Death Domain Receptor 3 (DcR3). A target protein may be detected by use of an antibody-based assay, where an antibody specific to the target protein is utilized. In some embodiments, antibody-based detection methods utilize an antibody that binds to any region of target protein. In one example, the method of analysis comprises performing an enzyme-linked immunosorbent assay (ELISA). The ELISA assay may be a sandwich ELISA or a direct ELISA. Another example method of analysis comprises a single molecule array, e.g., Simoa. Other examples of methods of detection include immunohistochemistry and lateral flow assay. Other examples of methods for detecting target protein include, but are not limited to, gel electrophoresis, capillary electrophoresis, high performance liquid chromatography (HPLC), thin layer chromatography (TLC), hyperdiffusion chromatography, and the like, or various immunological methods such as fluid or gel precipitation reactions, immunodiffusion (single or double), immunoelectrophoresis, radioimmunoassay (RIA), immunofluorescent assays, and Western blotting. In some embodiments, antibodies, or antibody fragments, are used in methods such as Western blots or immunofluorescence techniques to detect the expressed proteins. The antibody or protein can be immobilized on a solid support for Western blots and immunofluorescence techniques. Suitable solid phase supports or carriers include any support capable of binding an antigen or an antibody. In some embodiments, supports or carriers include glass, polystyrene, polypropylene, polyethylene, dextran, nylon, amylases, natural and modified celluloses, polyacrylamides, gabbros, and magnetite.

In some cases, a target protein may be detected by detecting binding between the target protein and a binding partner of the target protein. Non-limiting examples of binding partners to TL1A include DcR3, and Tumor necrosis factor receptor superfamily member 25 (TNR25). In some embodiments, methods of analysis of protein-protein binding comprise performing an assay in vivo or in vitro, or ex vivo. In some embodiments, the method of analysis comprises an assay such as a co-immunoprecipitation (co-IP), pull-down, crosslinking protein interaction analysis, labeled transfer protein interaction analysis, or Far-western blot analysis, FRET based assay, including, for example FRET-FLIM, a yeast two-hybrid assay, BiFC, or split luciferase assay.

Disclosed herein are methods of detecting a presence or a level of one or more serological markers in a sample obtained from a subject. In some embodiments, the one or more serological markers comprises anti-Saccharomyces cerevisiae antibody (ASCA), an anti-neutrophil cytoplasmic antibody (ANCA), antibody against E. coli outer membrane porin protein C (anti-OmpC), anti-chitin antibody, pANCA antibody, anti-I2 antibody, and anti-Cbir1 flagellin antibody. In some embodiments, the antibodies comprises immunoglobulin A (IgA), immunoglobulin G (IgG), immunoglobulin E (IgE), or immunoglobulin M (IgM), immunoglobulin D (IgD), or a combination thereof. Any suitable method for detecting a target protein or biomarker disclosed herein may be used to detect a presence, absence, or level of a serological marker. In some embodiments, the presence or the level of the one or more serological markers is detected using an enzyme-linked immunosorbent assay (ELISA), a single molecule array (Simoa), immunohistochemistry, internal transcribed spacer (ITS) sequencing, or any combination thereof. In some embodiments, the ELISA is a fixed leukocyte ELISA. In some embodiments, the ELISA is a fixed neutrophil ELISA. A fixed leukocyte or neutrophil ELISA may be useful for the detection of certain serological markers, such as those described in Saxon et al., A distinct subset of antineutrophil cytoplasmic antibodies is associated with inflammatory bowel disease, J. Allergy Clin. Immuno. 86:2; 202-210 (August 1990). In some embodiments, ELISA units (EU) are used to measure positivity of a presence or level of a serological marker (e.g., seropositivity), which reflects a percentage of a standard or reference value. In some embodiments, the standard comprises pooled sera obtained from well-characterized patient population (e.g., diagnosed with the same disease or condition the subject has, or is suspected of having) reported as being seropositive for the serological marker of interest. In some embodiments, the control or reference value comprises 10, 20, 30, 40, 50, 60, 70, 80, 90, or 100 EU. In some embodiments, a quartile sum scores are calculated using, for example, the methods reported in Landers C J, Cohavy O, Misra R. et al., Selected loss of tolerance evidenced by Crohn's disease-associated immune responses to auto- and microbial antigens. Gastroenterology (2002)123:689-699.

### Methods of Treatment

Disclosed herein are methods of treating a subject, the methods comprising administering a therapeutically effective amount of an inhibitor of Tumor necrosis factor-like cytokine 1A (TL1A) activity or expression to a subject. In some embodiments, the subject has moderate to severely active CD or UC. In some embodiments, the subject has been determined to have high fold-change of TL1A expression relative to a cut-off fold-change value, using the methods described herein. In some embodiments, the inhibitor of TL1A activity or expression is an anti-TL1A antibody. Also disclosed herein are methods of generating antibodies and pharmaceutical compositions comprising an inhibitor of TL1A activity or expression.

In some embodiments, the inhibitor of TL1A activity or expression is effective to inhibit TL1A-DR3 binding. In some embodiments, the inhibitor of TL1A activity or expression comprises an allosteric modulator of TL1A. An allosteric modulator of TL1A may indirectly influence the effects TL1A on DR3, or TR6/DcR3 on TL1A or DR3. The inhibitor of TL1A activity or expression may be a direct inhibitor or indirect inhibitor. Non-limiting examples of an inhibitor of TL1A expression include RNA to protein TL1A translation inhibitors, antisense oligonucleotides targeting the TNFSF15 mRNA (such as miRNAs, or siRNA), epigenetic editing (such as targeting the DNA-binding domain of TNFSF15, or post-translational modifications of histone tails and/or DNA molecules). Non-limiting examples of an inhibitor of TL1A activity include antagonists to the TL1A receptors, (DR3 and TR6/DcR3), antagonists to TL1A antigen, and antagonists to gene expression products involved in TL1A mediated disease. Antagonists as disclosed herein, may include, but are not limited to, an anti-TL1A antibody, an anti-TL1A-binding antibody fragment, or a small molecule. The small molecule may be a small molecule that binds to TL1A or DR3. The anti-TL1A antibody may be monoclonal or polyclonal. The anti-TL1A antibody may be humanized or chimeric. The anti-TL1A antibody may be a fusion protein. The anti-TL1A antibody may be a blocking anti-TL1A antibody. A blocking antibody blocks binding between two proteins, e.g., a ligand and its receptor. Therefore, a TL1A blocking antibody includes an antibody that prevents binding of TL1A to DR3 and/or TR6/DcR3 receptors. In a non-limiting example, the TL1A blocking antibody binds to DR3. In another example, the TL1A blocking antibody binds to DcR3. In some cases, the TL1A antibody is an anti-TL1A antibody that specifically binds to TL1A.

### Anti-TL1A Antibodies

In one aspect, provided herein are antibodies that bind to TL1A. In some embodiments, an antibody comprises an antigen-binding fragment that refers to a portion of an antibody having antigenic determining variable regions of an antibody. Examples of antigen-binding fragments include, but are not limited to Fab, Fab′, F(ab′)₂, and Fv fragments, linear antibodies, single chain antibodies, and multispecific antibodies formed from antibody fragments. In some embodiments, an antibody refers to an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. In some embodiments, an antibody includes intact polyclonal antibodies, intact monoclonal antibodies, antibody fragments (such as Fab, Fab′, F(ab′)₂, and Fv fragments), single chain Fv (scFv) mutants, a CDR-grafted antibody, multispecific antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different subunit structures and three-dimensional configurations. Antibodies can be naked or conjugated to other molecules such as toxins, radioisotopes, etc.

In some embodiments, a humanized antibody refers to forms of non-human (e.g., murine) antibodies having specific immunoglobulin chains, chimeric immunoglobulins, or fragments thereof that contain minimal non-human (e.g., murine) sequences. In a non-limiting example, a humanized antibody comprises less than about 40% non-human sequence in the variable region. In some cases, a humanized antibody comprises less than about 20% non-human sequence in a full-length antibody sequence. In a further non-limiting example, a humanized antibody comprises less than about 20% non-human sequence in the framework region of each of the heavy chain and light chain variable regions. For instance, the humanized antibody comprises less than about 20%, 19%, 18%, 17%, 16%, 15%, 14%, 13%, 12%, 11%, 10%, 9%, 8%, 7%, 6%, 5%, 4%, 3%, 2%, or 1% non-human sequence in the framework region of each of the heavy chain and light chain variable regions. As another example, the humanized antibody comprises about or less than about 15, 14, 13, 12, 11, 10, 9, 8, 7, 6, 5, 4, 3, 2, or 1 non-human sequences in the framework region of each of the heavy chain and light chain variable regions. In some cases, humanized antibodies are human immunoglobulinsin which residues from the complementarity determining region (CDR) are replaced by residues from the CDR of a non-human species (e.g., mouse, rat, rabbit, hamster) that have the desired specificity, affinity, and capability. These humanized antibodies may contain one or more non-human species mutations, e.g., the heavy chain comprises about 1, 2, 3, 4, 5, 6, 7, 8, 9, 10, 11, 12, 13, 14, or 15 non-human species mutations in the framework region, and the light chain comprises about 1, 2, 3, 4, 5, 6, 7, 8, 9, 10, 11, 12, 13, 14, or 15 non-human species mutations in the framework region. The humanized heavy chain variable domain may comprise IGHV1-46*02 framework with no or fewer than about 10, 9, 8, 7, 6, 5, 4, 3, 2, or 1 amino acid mutations. The humanized light chain variable domain may comprise IGKV3-20 framework with no or fewer than about 10, 9, 8, 7, 6, 5, 4, 3, 2, or 1 amino acid mutations.

In some embodiments, chimeric antibodies refer to antibodies wherein the sequence of the immunoglobulin molecule is derived from two or more species. As a non-limiting example, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid eliciting an immune response in that species.

The terms “complementarity determining region,” and “CDR,” which are synonymous with “hypervariable region” or “HVR,” refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain embodiments, the CDRs of the antibodies described herein can be defined by a method selected from Kabat, Chothia, IMGT, Aho, AbM, or combinations thereof.

In some embodiments, an antibody that specifically binds to a protein indicates that the antibody reacts or associates more frequently, more rapidly, with greater duration, with greater affinity, or with some combination of the above to the protein than with alternative substances, including unrelated proteins.

In some embodiments, the terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as fusion with another polypeptide and/or conjugation, e.g., with a labeling component. Also included within the definition are, for example, polypeptides containing one or more analogs of an amino acid (for example, unnatural amino acids, etc.), as well as other modifications.

In some embodiments, a protein such as an antibody described herein comprises a hydrophobic amino acid. Non-limiting examples of hydrophobic amino acids include glycine (Gly), proline (Pro), phenylalanine (Phe), alanine (Ala), isoleucine (Ile), leucine (Leu), and valine (Val). In some embodiments, a protein such as an antibody described herein comprises a hydrophilic amino acid. Non-limiting examples of hydrophilic amino acids include serine (Ser), threonine (Thr), aspartic acid (Asp), glutamic acid (Glu), cysteine (Cys), asparagine (Asn), glutamine (Gln), arginine (Arg), and histidine (His). In some embodiments, a protein such as an antibody described herein comprises an amphipathic amino acid. Non-limiting examples of amphipathic amino acids include lysine (Lys), tryptophan (Trp), tyrosine (Tyr), and methionine (Met). In some embodiments, a protein such as an antibody described herein comprises an aliphatic amino acid. Non-limiting examples of aliphatic amino acids include alanine (Ala), isoleucine (Ile), leucine (Leu) and valine (Val). In some embodiments, a protein such as an antibody described herein comprises an aromatic amino acid. Non-limiting examples of aromatic amino acids include phenylalanine (Phe), tryptophan (Trp), and tyrosine (Tyr). In some embodiments, a protein such as an antibody described herein comprises an acidic amino acid. Non-limiting examples of acidic amino acids include aspartic acid (Asp) and glutamic acid (Glu). In some embodiments, a protein such as an antibody described herein comprises a basic amino acid. Non-limiting examples of basic amino acids include arginine (Arg), histidine (His), and lysine (Lys). In some embodiments, a protein such as an antibody described herein comprises a hydroxylic amino acid. Non-limiting examples of hydroxylic amino acids include serine (Ser) and threonine (Thr). In some embodiments, a protein such as an antibody described herein comprises a sulfur-containing amino acid. Non-limiting examples of sulfur-containing amino acids include cysteine (Cys) and methionine (Met). In some embodiments, a protein such as an antibody described herein comprises an amidic amino acid. Non-limiting examples of amidic amino acids include asparagine (Asn) and glutamine (Gln).

In some embodiments, “polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as, but not limited to methylated nucleotides and their analogs or non-nucleotide components. Modifications to the nucleotide structure may be imparted before or after assembly of the polymer. A polynucleotide may be further modified after polymerization, such as by conjugation with a labeling component.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program can be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In some embodiments, the term “about” means within 10% of the stated amount. For instance, an antibody variable region comprising about 80% identity to a reference variable region may comprise 72% to 88% identity to the reference variable region.

In certain aspects, antibodies are described herein that specifically bind to TL1A (Entrez Gene: 9966; UniProtKB: O95150). In some embodiments, the antibodies specifically bind to soluble TL1A. In some embodiments, the antibodies specifically bind to membrane bound TL1A. In some embodiments, an anti-TL1A antibody is provided having a heavy chain comprising four heavy chain framework regions (HCFR) and three heavy chain complementarity-determining regions (HCDR): HCFR1, HCDR1, HCFR2, HCDR2, HCFR3, HCDR3, and HCFR4; and a light chain comprising four light chain framework regions (LCFR) and three light chain complementarity-determining regions (LCDR): LCFR1, LCDR1, LCFR2, LCDR2, LCFR3, LCDR3, and LCFR4. An anti-TL1A antibody may comprise any region provided herein, for example, as provided in the tables, the examples, and the sequences.

### Anti-TL1A CDRs

In certain embodiments, an anti-TL1A antibody comprises a HCDR1 as set forth by SEQ ID NO: 1. In certain embodiments, an anti-TL1A antibody comprises a HCDR2 as set forth by any one of SEQ ID NOS: 2-5. In certain embodiments, an anti-TL1A antibody comprises a HCDR3 as set forth by any one of SEQ ID NOS: 6-9. In certain embodiments, an anti-TL1A antibody comprises a LCDR1 as set forth by SEQ ID NO: 10. In certain embodiments, an anti-TL1A antibody comprises a LCDR2 as set forth by SEQ ID NO: 11. In certain embodiments, an anti-TL1A antibody comprises a LCDR3 as set forth by any one of SEQ ID NOS: 12-15. In a non-limiting example, an anti-TL1A antibody comprises a HCDR1 as set forth by SEQ ID NO: 1, a HCDR2 as set forth by SEQ ID NO: 2, a HCDR3 as set forth by SEQ ID NO: 6, a LCDR1 as set forth by SEQ ID NO: 10, a LCDR2 as set forth by SEQ ID NO: 11, and a LCDR3 as set forth by SEQ ID NO: 12.

In certain embodiments, an anti-TL1A antibody comprises a HCDR1 as set forth by SEQ ID NO: 16 or 17. In certain embodiments, an anti-TL1A antibody comprises a HCDR2 as set forth by SEQ ID NO: 18. In certain embodiments, an anti-TL1A antibody comprises a HCDR3 as set forth by SEQ ID NO: 19 or 20. In certain embodiments, an anti-TL1A antibody comprises a LCDR1 as set forth by SEQ ID NO: 21. In certain embodiments, an anti-TL1A antibody comprises a LCDR2 as set forth by SEQ ID NO: 22. In certain embodiments, an anti-TL1A antibody comprises a LCDR3 as set forth by SEQ ID NO: 23 or 24.

In certain embodiments, an anti-TL1A antibody comprises a HCDR1 as set forth by any one of SEQ ID NOS: 25-38. In certain embodiments, an anti-TL1A antibody comprises a HCDR2 as set forth by any one of SEQ ID NOS: 39-51. In certain embodiments, an anti-TL1A antibody comprises a HCDR3 as set forth by any one of SEQ ID NOS: 52-65. In certain embodiments, an anti-TL1A antibody comprises a LCDR1 as set forth by any one of SEQ ID NOS: 66-78. In certain embodiments, an anti-TL1A antibody comprises a LCDR2 as set forth by any one of SEQ ID NOS: 79-89. In certain embodiments, an anti-TL1A antibody comprises a LCDR3 as set forth by any one of SEQ ID NOS: 90-100. In some cases, the anti-TL1A antibody comprises a HCDR1, HCDR2, HCDR3, LCDR1, LCDR2, and LCDR3 of antibody M1 (e.g., SEQ ID NOS: 25, 39, 52, 66, 79, and 90). In some cases, the anti-TL1A antibody comprises a HCDR1, HCDR2, HCDR3, LCDR1, LCDR2, and LCDR3 of antibody M2 (e.g., SEQ ID NOS: 26, 40, 53, 67, 80, and 91). In some cases, the anti-TL1A antibody comprises a HCDR1, HCDR2, HCDR3, LCDR1, LCDR2, and LCDR3 of antibody M3 (e.g., SEQ ID NOS: 27 or 28, 41 or 42, 54 or 55, 68, 81, and 92). In some cases, the anti-TL1A antibody comprises a HCDR1, HCDR2, HCDR3, LCDR1, LCDR2, and LCDR3 of antibody M4 (e.g., SEQ ID NOS: 29, 43, 56, 69, 82, and 93). In some cases, the anti-TL1A antibody comprises a HCDR1, HCDR2, HCDR3, LCDR1, LCDR2, and LCDR3 of antibody M5 (e.g., SEQ ID NOS: 30, 44, 57, 70, 83, and 94). In some cases, the anti-TL1A antibody comprises a HCDR1, HCDR2, HCDR3, LCDR1, LCDR2, and LCDR3 of antibody M6 (e.g., SEQ ID NOS: 31 or 32, 45, 58 or 59, 71 or 72, 84, and 95). In some cases, the anti-TL1A antibody comprises a HCDR1, HCDR2, HCDR3, LCDR1, LCDR2, and LCDR3 of antibody M7 (e.g., SEQ ID NOS: 33, 46, 60, 73, 85, and 96). In some cases, the anti-TL1A antibody comprises a HCDR1, HCDR2, HCDR3, LCDR1, LCDR2, and LCDR3 of antibody M8 (e.g., SEQ ID NOS: 34, 47, 61, 74, 86, and 97). In some cases, the anti-TL1A antibody comprises a HCDR1, HCDR2, HCDR3, LCDR1, LCDR2, and LCDR3 of antibody M9 (e.g., SEQ ID NOS: 35, 48, 62, 75, 87, and 98). In some cases, the anti-TL1A antibody comprises a HCDR1, HCDR2, HCDR3, LCDR1, LCDR2, and LCDR3 of antibody M10 (e.g., SEQ ID NOS: 36, 49, 63, 76, 88, and 99). In some cases, the anti-TL1A antibody comprises a HCDR1, HCDR2, HCDR3, LCDR1, LCDR2, and LCDR3 of antibody M11 (e.g., SEQ ID NOS: 37, 50, 64, 77, 89, and 100). In some cases, the anti-TL1A antibody comprises a HCDR1, HCDR2, HCDR3, LCDR1, LCDR2, and LCDR3 of antibody M12 (e.g., SEQ ID NOS: 38, 51, 65, 78, 84, and 95).

In certain embodiments, an anti-TL1A antibody comprises a HCDR1, HCDR2, HCDR3, LCDR1, LCDR2, and LCDR3 selected from Table 1.

TABLE 1
Example CDR amino acid sequences

| SEQ | | |
|---|---|---|
| ID NO | Description | Sequence |
| 1 | HCDR1 | GFDIQDTYMH |
| 2 | HCDR2a | RIDPASGHTKYDPKFQV |
| 3 | HCDR2b | RIEPASGHIKYDPKFQG |
| 4 | HCDR2c | RIDPASGHIKYDPKFQG |
| 5 | HCDR2d | RIEPASGHIKYDPKFQV |
| 6 | HCDR3a | SGGLPDV |
| 7 | HCDR3b | ARSGGLPDV |
| 8 | HCDR3c | SGGLPDW |
| 9 | HCDR3d | ARSGGLPDW |
| 10 | LCDR1 | RASSSVSYMY |
| 11 | LCDR2 | ATSNLAS |
| 12 | LCDR3a | QQWEGNPRT |
| 13 | LCDR3b | QQWKGNPRT |
| 14 | LCDR3c | QQWSGNPRT |
| 15 | LCDR3d | QQWSRNPRT |
| 16 | P HCDR1 | GFX₁X₂X₃DX₄X₅X₆H; X1 = D or E; |
| | | X2 = I, L, P, or V; X3 = G, Q, |
| | | S, or V; X4 = A, S, T; X5 = F |
| | | or Y; X6 = I, L, or M |
| 17 | P HCDR1 | GFX₁X₂X₃DTX₄X₅H; X1 = D, OR E; |
| | | X2 = I, P, OR V; X3 = G, Q, S, |
| | | OR V; X4 = F, OR Y; X5 = I, OR |
| | | M |
| 18 | P HCDR2 | RX₁X₂PX₃X₄X₅HX₆KX₇X₈PKFX₉X₁₀; X1 = |
| | | I or L; X2 = D or E; X3 = A or |
| | | E; X4 = G or S; X5 = A or G; |
| | | X6 = I, L, T, or V; X7 = I, L, |
| | | M, S, T, V, or Y; X8 = D, I, N, |
| | | R, or S; X9 = Q or R; X10 = A, |
| | | D, E, G, H, K, L, M, N, P, R, |
| | | S, T, or V |
| 19 | P HCDR3 | X₁X₂GX₃PX₄X₅; X1 = L or S; X2 = |
| | | A or G; X3 = A, L, or M; X4 = |
| | | D or E; X5 = K, M, Q, R, S, T, |
| | | V, or W |
| 20 | P HCDR3 | SGGX₁PDX₂; X₁ = L, OR M; X₂ = |
| | | E, I, K, L, M, Q, T, V, W, |
| | | OR Y |
| 21 | P LCDR1 | X₁ASSSVX₂X₃X₄X₅; X1 = G, R, or |
| | | W; X2 = I or S X3 = F or Y; |
| | | X4 = L or M; X5 = R or Y |
| 22 | P LCDR2 | AX₁X₂X₃LX₄S; X1 = K or T; |
| | | X2 = E, P, or S; X3 = L, N, |
| | | or P; X4 = A or T |
| 23 | P LCDR3 | X₁QWX₂X₃X₄PRT; X₁ = Q, OR N; |
| | | X₂ = D, E, H, N, Q, OR S; |
| | | X₃ = A, OR G; X₄ = D, F, K, N, |
| | | R, S, OR T |
| 24 | P LCDR3 | X₁QX₂X₃X₄X₅PRX₆; X1 = H, N, Q, |
| | | or S; X2 = F, H, I, P, R, S, |
| | | W, or Y; X3 = D, E, H, N, Q, |
| | | S, or V; X4 = A, D, G, Q, or |
| | | S; X5 = D, F, H, K, L, M, N, |
| | | Q, R, S, or T; X6 = T or S |
| 25 | M1 HCDR1 | GFTFSSYW |
| 26 | M2 HCDR1 | GGSFTGFY |
| 27 | M3 HCDR1 | GY(X1)F(X2)(X3)YGIS; X1 = P, |
| | | S, D, Q, N; X2 = T, R; X3 = |
| | | N, T, Y, H |
| 28 | M3 HCDR1 | GYDFTYYGIS |
| 29 | M4 HCDR1 | SRSYYWG |
| 30 | M5 HCDR1 | TSNMGVV |
| 31 | M6 HCDR1 | LYGMN |
| 32 | M6 HCDR1 | NYGMIN |
| 33 | M7 HCDR1 | GYTFTSSWMH |
| 34 | M8 HCDR1 | SYDIN |
| 35 | M9 HCDR1 | SYFWS |
| 36 | M10 HCDR1 | GYYWN |
| 37 | M11 HCDR1 | GFTFSTYG |
| 38 | M12 HCDR1 | TYGMS |
| 39 | M1 HCDR2 | IKEDGSEK |
| 40 | M2 HCDR2 | INHRGNT |
| 41 | M3 HCDR2 | WIS(X1)YNG(X2)(X3)(X4)YA(X5) |
| | | (X6)(X7)QG; X1 = T, P, S, A; |
| | | X2 = N, G, V, K, A; X3 = T, K; |
| | | X4 = H, N; X5 = Q, R; X6 = K, |
| | | M; X7 = L, H |
| 42 | M3 HCDR2 | WISTYNGNTHYARMLQG |
| 43 | M4 HCDR2 | SIYYNGRTYYNPSLKS |
| 44 | M5 HCDR2 | HILWDDREYSNPALKS |
| 45 | M6 HCDR2 | WINTYTGEPTYADDFKG |
| 46 | M7 HCDR2 | IHPNSGGT |
| 47 | M8 HCDR2 | WLNPNSGXTG; X = N, Y |
| 48 | M9 HCDR2 | YIYYSGNTKYNPSLKS |
| 49 | M10 HCDR2 | EINHAGNTNYNPSLKS |
| 50 | M11 HCDR2 | ISGTGRTT |
| 51 | M12 HCDR2 | WMNTYSGVTTYADDFKG |
| 52 | M1 HCDR3 | AREDYDSYYKYGMDV |
| 53 | M2 HCDR3 | ASPFYDFWSGSDY |
| 54 | M3 HCDR3 | ENYYGSG(X1)(X2)RGGMD(X3); |
| | | X1 = S, A; X2 = Y, P; |
| | | X3 = V, A, G |
| 55 | M3 HCDR3 | ENYYGSGAYRGGMDV |
| 56 | M4 HCDR3 | EDYGDYGAFDI |
| 57 | M5 HCDR3 | MSRNYYGSSYVMDY |
| 58 | M6 HCDR3 | DTAMDYAMAY |
| 59 | M6 HCDR3 | DYGKYGDYYAMDY |
| 60 | M7 HCDR3 | ARGDYYGYVSWFAY |
| 61 | M8 HCDR3 | EVPETAAFEY |
| 62 | M9 HCDR3 | ETGSYYGFDY |
| 63 | M10 HCDR3 | GYCRSTTCYFDY |
| 64 | M11 HCDR3 | TKERGDYYYGVFDY |
| 65 | M12 HCDR3 | EGYVFDDYYATDY |
| 66 | M1 LCDR1 | QSILYSSNNKNY |
| 67 | M2 LCDR1 | QSLVHSDGNTY |
| 68 | M3 LCDR1 | RASQSVSSYLA |
| 69 | M4 LCDR1 | RASQGISSALA |
| 70 | M5 LCDR1 | SASSSVNYMH |
| 71 | M6 LCDR1 | KSSQNIVHSDGNTYLE |
| 72 | M6 LCDR1 | RSSQSIVHSNGNTYLD |
| 73 | M7 LCDR1 | QNINVL |
| 74 | M8 LCDR1 | TSSSSDIGA(X1)(X2)GV(X3); X1 = |
| | | G, A; X2 = L, S, Q; X3 = H, L |
| 75 | M9 LCDR1 | RASQSINNYLN |
| 76 | M10 LCDR1 | RASQSVRSSYLA |
| 77 | M11 LCDR1 | QTISSW |
| 78 | M12 LCDR1 | RSSQNIVHSDGNTYLE |
| 79 | M1 LCDR2 | WAS |
| 80 | M2 LCDR2 | KIS |
| 81 | M3 LCDR2 | DASNRAT |
| 82 | M4 LCDR2 | DASSLES |
| 83 | M5 LCDR2 | STSNLAS |
| 84 | M6 & M12 | KVSNRFS |
| | LCDR2 | |
| 85 | M7 LCDR2 | KAS |
| 86 | M8 LCDR2 | GYYNRPS |
| 87 | M9 LCDR2 | AASSLQS |
| 88 | M10 LCDR2 | GASSRAT |
| 89 | M11 LCDR2 | AAS |
| 90 | M1 LCDR3 | QQYYSTPFT |
| 91 | M2 LCDR3 | MQATQFPLT |
| 92 | M3 LCDR3 | QQRSNWPWT |
| 93 | M4 LCDR3 | QQFNSYPLT |
| 94 | M5 LCDR3 | HQWNNYGT |
| 95 | M6 & M12 | FQGSHVPLT |
| | LCDR3 | |
| 96 | M7 LCDR3 | QQGQSYPYT |
| 97 | M8 LCDR3 | QSXDGTLSAL; X = Y, W, F |
| 98 | M9 LCDR3 | QQSYSTPRT |
| 99 | M10 LCDR3 | QQYGSSPT |
| 100 | M11 LCDR3 | QQYHRSWT |

In certain embodiments, an anti-TL1A antibody comprises the CDRs set forth in antibody A, B, C, D, E, F, G, H, I, A2, B2, C2, D2, E2, F2, G2, H2, or I2 of Table 2.

TABLE 2
CDR sequences from example anti-TL1A antibodies

| | Heavy Chain CDR | Light Chain CDR |
|---|---|---|
| | SEQ ID NOS | SEQ ID NOS |
| Antibody | (CDR1, CDR2, CDR3) | (CDR1, CDR2, CDR3) |
| A | 1, 2, 6 | 10, 11, 12 |
| B | 1, 3, 8 | 10, 11, 12 |
| C | 1, 4, 8 | 10, 11, 12 |
| D | 1, 2, 6 | 10, 11, 13 |
| E | 1, 2, 6 | 10, 11, 14 |
| F | 1, 5, 8 | 10, 11, 12 |
| G | 1, 5, 8 | 10, 11, 13 |
| H | 1, 3, 8 | 10, 11, 13 |
| A2 | 1, 2, 7 | 10, 11, 12 |
| B2 | 1, 3, 9 | 10, 11, 12 |
| C2 | 1, 4, 9 | 10, 11, 12 |
| D2 | 1, 2, 7 | 10, 11, 13 |
| E2 | 1, 2, 7 | 10, 11, 14 |
| F2 | 1, 5, 9 | 10, 11, 12 |
| G2 | 1, 5, 9 | 10, 11, 13 |
| H2 | 1, 3, 9 | 10, 11, 13 |
| I | 1, 5, 8 | 10, 11, 15 |
| I2 | 1, 5, 9 | 10, 11, 15 |

In certain embodiments, an anti-TL1A antibody comprises the heavy chain CDRs set forth in an antibody selected from Table 3.

TABLE 3
Example heavy chain variable region sequences

| SEQ | | |
|---|---|---|
| ID | | |
| NO | Description | Sequence |
| 101 | 217 VH, 158 VH | QVQLVQSGAEVKKPGASVKVSCKASGFDIQDTYMHWVKQRPGQGL |
| | | EWMGRIDPASGHTKYDPKFQVRVTITRDTSTSTVYMELSSLRSEDTA |
| | | VYYCARSGGLPDVWGQGTTVTVSS |
| 102 | 220 VH, 160 VH | QVQLVQSGAEVKKPGASVKVSCKASGFDIQDTYMHWVKQRPGQGL |
| | | EWMGRIDPASGHTKYDPKFQVRVTMTRDTSTSTAYLELSSLRSEDTA |
| | | VYYCARSGGLPDVWGQGTTVTVSS |
| 103 | 223 VH, 200 | EVQLVQSGAEVKKPGASVKVSCKASGFDIQDTYMHWVRQRPGQGL |
| | VH, 194 VL, 206 | EWIGRIDPASGHTKYDPKFQVRATITTDTSTSTAYLELSSLRSEDTAV |
| | VH | YYCARSGGLPDVWGQGTTVTVSS |
| 104 | 219 VH, 157 VH | QVQLVQSGAEVKKPGASVKVSCKASGFDIQDTYMHWVKQRPGQGL |
| | | EWMGRIDPASGHTKYDPKFQVRVTITRDTSTSTVYLELSSLRSEDTAV |
| | | YYCARSGGLPDVWGQGTTVTVSS |
| 105 | 221 VH, 125 VH | QVQLVQSGAEVKKPGASVKVSCKASGFDIQDTYMHWVKQRPGQGL |
| | | EWMGRIDPASGHTKYDPKFQVRATITRDTSTSTAYLELSSLRSEDTAV |
| | | YYCARSGGLPDVWGQGTTVTVSS |
| 106 | 213 VH, 162 VH | QVQLVQSGAEVKKPGASVKVSCKASGFDIQDTYMHWVRQRPGQGL |
| | | EWMGRIDPASGHTKYDPKFQVRVTITTDTSTSTVYMELSSLRSEDTA |
| | | VYYCARSGGLPDVWGQGTTVTVSS |
| 107 | 212 VH, 100 | QVQLVQSGAEVKKPGASVKVSCKASGFDIQDTYMHWVRQRPGQGL |
| | VH, 181 VH, 34 | EWIGRIDPASGHTKYDPKFQVRATITTDTSTSTAYLELSSLRSEDTAV |
| | VH, 79 VH | YYCARSGGLPDVWGQGTTVTVSS |
| 108 | 107 VH, 211 | QVQLVQSGAEVKKPGASVKVSCKASGFDIQDTYMHWVKQRPGQGL |
| | VH, 15 VH, 30 | EWIGRIDPASGHTKYDPKFQVRATITTDTSTSTAYLELSSLRSEDTAV |
| | VH, 29 VH, 48 | YYCARSGGLPDVWGQGTTVTVSS |
| | VH, 49 VH, 50 | |
| | VH, 51 VH, 52 | |
| | VH, 56 VH | |
| 109 | 205 VH, 199 | EVQLVQSGAEVKKPGASVKVSCKASGFDIQDTYMHWVKQRPGQGL |
| | VH, 55 VH, 193 | EWIGRIDPASGHTKYDPKFQVRATITTDTSTSTAYLELSSLRSEDTAV |
| | VH | YYCARSGGLPDVWGQGTTVTVSS |
| 110 | 129 VH, 139 | QVQLVQSGAEVKKPGASVKVSCKASGFDIQDTYMHWVRQRPGQGL |
| | VH, 140 VH, | EWMGRIDPASGHTKYDPKFQVRVTITTDTSTSTAYLELSSLRSEDTAV |
| | 215 VH | YYCARSGGLPDVWGQGTTVTVSS |
| 111 | 214 VH, 128 | QVQLVQSGAEVKKPGASVKVSCKASGFDIQDTYMHWVRQRPGQGL |
| | VH, 141 VH, | EWMGRIDPASGHTKYDPKFQVRVTITRDTSTSTAYLELSSLRSEDTAV |
| | 142 VH, 144 VH | YYCARSGGLPDVWGQGTTVTVSS |
| 112 | 216 VH, 123 VH | QVQLVQSGAEVKKPGASVKVSCKASGFDIQDTYMHWVRQRPGQGL |
| | | EWIGRIDPASGHTKYDPKFQVRVTITRDTSTSTAYLELSSLRSEDTAV |
| | | YYCARSGGLPDVWGQGTTVTVSS |
| 113 | 122 VH | QVQLVQSGAEVKKPGASVKVSCKASGFDIQDTYMHWVRQRPGQGL |
| | | EWIGRIDPASGHTKYDPKFQVRATITRDTSTSTAYLELSSLRSEDTAV |
| | | YYCARSGGLPDVWGQGTTVTVSS |
| 114 | 222 VH, 126 VH | QVQLVQSGAEVKKPGASVKVSCKASGFDIQDTYMHWVKQRPGQGL |
| | | EWMGRIDPASGHTKYDPKFQVRVTITRDTSTSTAYLELSSLRSEDTAV |
| | | YYCARSGGLPDVWGQGTTVTVSS |
| 115 | 188 VH, 41 VH, | QVQLVQSGAEVKKPGASVKVSCKASGFDIQDTYMHWVKQRPGQGL |
| | 102 VH | EWIGRIDPASGHTKYDPKFQVRVTITTDTSTSTAYLELSSLRSEDTAV |
| | | YYCARSGGLPDVWGQGTTVTVSS |
| 116 | 203 VH, 197 | EVQLVQSGAEVKKPGASVKVSCKASGFDIQDTYMHWVKQAPGQGL |
| | VH, 209 VH, | EWMGRIEPASGHIKYDPKFQGRVTMTRDTSTSTVYMELSSLRSEDTA |
| | 224 VH | VYYCARSGGLPDWWGQGTTVTVSS |
| 117 | 147 VH, 112 | QVQLVQSGAEVKKPGASVKVSCKASGFDIQDTYMHWVKQRPGQGL |
| | VH, 59 VH | EWMGRIEPASGHIKYDPKFQGRVTMTRDTSTSTVYMELSSLRSEDTA |
| | | VYYCARSGGLPDWWGQGTTVTVSS |
| 118 | 127 VH | QVQLVQSGAEVKKPGASVKVSCKASGFDIQDTYMHWVKQRPGQGL |
| | | EWMGRIDPASGHTKYDPKFQVRVTITTDTSTSTAYLELSSLRSEDTAV |
| | | YYCARSGGLPDVWGQGTTVTVSS |
| 119 | 159 VH, 218 VH | QVQLVQSGAEVKKPGASVKVSCKASGFDIQDTYMHWVKQRPGQGL |
| | | EWMGRIDPASGHTKYDPKFQVRVTITRDTSTSTAYMELSSLRSEDTA |
| | | VYYCARSGGLPDVWGQGTTVTVSS |
| 120 | 103 VH, 45 VH, | QVQLVQSGAEVKKPGASVKVSCKASGFDIQDTYMHWVKQRPGQGL |
| | 167 VH, 187 VH | EWIGRIDPASGHTKYDPKFQVRVTITRDTSTSTAYLELSSLRSEDTAV |
| | | YYCARSGGLPDVWGQGTTVTVSS |
| 121 | 64 VH, 148 VH, | QVQLVQSGAEVKKPGASVKVSCKASGFDIQDTYMHWVRQAPGQGL |
| | 97 VH, 114 VH, | EWMGRIEPASGHIKYDPKFQVRVTMTRDTSTSTVYMELSSLRSEDTA |
| | 130 VH, 133 | VYYCARSGGLPDWWGQGTTVTVSS |
| | VH, 137 VH, | |
| | 155 VH | |
| 122 | 67 VH, 138 VH, | QVQLVQSGAEVKKPGASVKVSCKASGFDIQDTYMHWVRQAPGQGL |
| | 115 VH, 149 | EWMGRIEPASGHIKYDPKFQVRATMTRDTSTSTVYMELSSLRSEDTA |
| | VH, 134 VH, 98 | VYYCARSGGLPDWWGQGTTVTVSS |
| | VH, 156 VH | |
| 123 | 68 VH, 99 VH, | QVQLVQSGAEVKKPGASVKVSCKASGFDIQDTYMHWVRQAPGQGL |
| | 116 VH | EWMGRIEPASGHIKYDPKFQVRVTITRDTSTSTVYMELSSLRSEDTAV |
| | | YYCARSGGLPDWWGQGTTVTVSS |
| 124 | 94 VH, 113 VH, | QVQLVQSGAEVKKPGASVKVSCKASGFDIQDTYMHWVRQAPGQGL |
| | 151 VH, 78 VH | EWMGRIEPASGHIKYDPKFQVRATITRDTSTSTVYMELSSLRSEDTAV |
| | | YYCARSGGLPDWWGQGTTVTVSS |
| 125 | 110 VH, 58 VH, | QVQLVQSGAEVKKPGASVKVSCKASGFDIQDTYMHWVKQAPGQGL |
| | 136 VH, 146 | EWMGRIEPASGHIKYDPKFQGRVTMTRDTSTSTVYMELSSLRSEDTA |
| | VH, 154 VH | VYYCARSGGLPDWWGQGTTVTVSS |
| 126 | 169 VH, 175 VH | QVQLVQSGAEVKKPGASVKVSCKASGFDIQDTYMHWVKQAPGQGL |
| | | EWMGRIDPASGHIKYDPKFQGRVTMTRDTSTSTVYMELSSLRSEDTA |
| | | VYYCARSGGLPDWWGQGTTVTVSS |
| 127 | 173 VH, 179 VH | QVQLVQSGAEVKKPGASVKVSCKASGFDIQDTYMHWVKQAPGQGL |
| | | EWMGRIEPASGHIKYDPKFQGRATMTRDTSTSTVYMELSSLRSEDTA |
| | | VYYCARSGGLPDWWGQGTTVTVSS |
| 128 | 96 VH, 132 VH, | QVQLVQSGAEVKKPGASVKVSCKASGFDIQDTYMHWVRQAPGQGL |
| | 65 VH, 150 VH | EWMGRIEPASGHIKYDPKFQGRATMTRDTSTSTVYMELSSLRSEDTA |
| | | VYYCARSGGLPDWWGQGTTVTVSS |
| 129 | 196 VH, 202 | EVQLVQSGAEVKKPGASVKVSCKASGFDIQDTYMHWVRQAPGQGL |
| | VH, 208 VH | EWMGRIEPASGHIKYDPKFQGRATMTRDTSTSTVYMELSSLRSEDTA |
| | | VYYCARSGGLPDWWGQGTTVTVSS |
| 130 | 172 VH, 178 VH | QVQLVQSGAEVKKPGASVKVSCKASGFDIQDTYMHWVRQAPGQGL |
| | | EWMGRIDPASGHIKYDPKFQGRATMTRDTSTSTVYMELSSLRSEDTA |
| | | VYYCARSGGLPDWWGQGTTVTVSS |
| 131 | 75 VH, 72 VH, | QVQLVQSGAEVKKPGASVKVSCKASGFDIQDTYMHWVRQAPGQGL |
| | 95 VH, 152 VH | EWMGRIEPASGHIKYDPKFQGRATITTDTSTSTVYMELSSLRSEDTAV |
| | | YYCARSGGLPDWWGQGTTVTVSS |
| 132 | 174 VH, 180 VH | QVQLVQSGAEVKKPGASVKVSCKASGFDIQDTYMHWVRQAPGQGL |
| | | EWMGRIEPASGHIKYDPKFQGRATMTRDTSTSTAYMELSSLRSEDTA |
| | | VYYCARSGGLPDWWGQGTTVTVSS |
| 133 | 109 VH, 91 VH, | QVQLVQSGAEVKKPGASVKVSCKASGFDIQDTYMHWVRQAPGQGL |
| | 135 VH, 145 | EWMGRIEPASGHIKYDPKFQGRVTMTRDTSTSTAYMELSSLRSEDTA |
| | VH, 153 VH | VYYCARSGGLPDWWGQGTTVTVSS |
| 134 | 198 VH, 204 | EVQLVQSGAEVKKPGASVKVSCKASGFDIQDTYMHWVRQAPGQGL |
| | VH, 210 VH | EWMGRIEPASGHIKYDPKFQGRVTMTRDTSTSTAYMELSSLRSEDTA |
| | | VYYCARSGGLPDWWGQGTTVTVSS |
| 135 | 170 VH, 176 VH | QVQLVQSGAEVKKPGASVKVSCKASGFDIQDTYMHWVRQAPGQGL |
| | | EWMGRIDPASGHIKYDPKFQGRVTMTRDTSTSTAYMELSSLRSEDTA |
| | | VYYCARSGGLPDWWGQGTTVTVSS |
| 136 | 31 VH, 85 VH, | QVQLVQSGAEVKKPGASVKVSCKASGFDIQDTYMHWVRQAPGQGL |
| | 86 VH, 87 VH, | EWMGRIEPASGHIKYDPKFQGRVTMTRDTSTSTVYMELSSLRSEDTA |
| | 88 VH, 89 VH, | VYYCARSGGLPDWWGQGTTVTVSS |
| | 90 VH, 143 VH | |
| 137 | 32 VH, 33 VH | DVQLVQSGAEVKKPGASVKVSCKASGFDIQDTYMHWVRQAPGQGL |
| | | EWMGRIEPASGHIKYDPKFQGRVTMTRDTSTSTVYMELSSLRSEDTA |
| | | VYYCARSGGLPDWWGQGTTVTVSS |
| 138 | 35 VH, 182 VH | QVQLVQSGAEVKKPGASVKVSCKASGFDIQDTYMHWVKQAPGQGL |
| | | EWIGRIDPASGHTKYDPKFQVRATITTDTSTSTAYLELSSLRSEDTAV |
| | | YYCARSGGLPDVWGQGTTVTVSS |
| 139 | 36 VH, 81 VH, | QVQLVQSGAEVKKPGASVKVSCKASGFDIQDTYMHWVRQAPGQGL |
| | 104 VH, 165 | EWIGRIDPASGHTKYDPKFQVRATITTDTSTSTAYLELSSLRSEDTAV |
| | VH, | YYCARSGGLPDVWGQGTTVTVSS |
| 140 | 37 VH, 82 VH, | QVQLVQSGAEVKKPGASVKVSCKASGFDIQDTYMHWVKQRPGQGL |
| | 101 VH, 183 VH | EWMGRIDPASGHTKYDPKFQVRATITTDTSTSTAYLELSSLRSEDTAV |
| | | YYCARSGGLPDVWGQGTTVTVSS |
| 141 | 38 VH, 190 VH | QVQLVQSGAEVKKPGASVKVSCKASGFDIQDTYMHWVKQRPGQGL |
| | | EWIGRIDPASGHTKYDPKFQVRATITTDTSTSTVYLELSSLRSEDTAV |
| | | YYCARSGGLPDVWGQGTTVTVSS |
| 142 | 39 VH, 191 VH | QVQLVQSGAEVKKPGASVKVSCKASGFDIQDTYMHWVKQRPGQGL |
| | | EWIGRIDPASGHTKYDPKFQVRATITTDTSTSTAYMELSSLRSEDTAV |
| | | YYCARSGGLPDVWGQGTTVTVSS |
| 143 | 40 VH, 105 VH, | QVQLVQSGAEVKKPGASVKVSCKASGFDIQDTYMHWVKQRPGQGL |
| | 192 VH | EWIGRIDPASGHTKYDPKFQVRATITTDTSTSTVYMELSSLRSEDTAV |
| | | YYCARSGGLPDVWGQGTTVTVSS |
| 144 | 42 VH, 83 VH, | QVQLVQSGAEVKKPGASVKVSCKASGFDIQDTYMHWVKQRPGQGL |
| | 186 VH | EWIGRIDPASGHTKYDPKFQVRATMTTDTSTSTAYLELSSLRSEDTAV |
| | | YYCARSGGLPDVWGQGTTVTVSS |
| 145 | 43 VH, 184 VH | QVQLVQSGAEVKKPGASVKVSCKASGFDIQDTYMHWVKQRPGQGL |
| | | EWIGRIDPASGHTKYDPKFQVRATITRDTSTSTAYLELSSLRSEDTAV |
| | | YYCARSGGLPDVWGQGTTVTVSS |
| 146 | 44 VH, 53 VH, | QVQLVQSGAEVKKPGASVKVSCKASGFDIQDTYMHWVKQRPGQGL |
| | 166 VH, 189 VH | EWIGRIDPASGHTKYDPKFQVRVTMTTDTSTSTAYLELSSLRSEDTAV |
| | | YYCARSGGLPDVWGQGTTVTVSS |
| 147 | 46 VH, 168 VH, | QVQLVQSGAEVKKPGASVKVSCKASGFDIQDTYMHWVKQRPGQGL |
| | 185 VH | EWIGRIDPASGHTKYDPKFQVRATMTRDTSTSTAYLELSSLRSEDTAV |
| | | YYCARSGGLPDVWGQGTTVTVSS |
| 148 | 47 VH | QVQLVQSGAEVKKPGASVKVSCKASGFDIQDTYMHWVKQRPGQGL |
| | | EWIGRIDPASGHTKYDPKFQVRVTMTRDTSTSTAYLELSSLRSEDTAV |
| | | YYCARSGGLPDVWGQGTTVTVSS |
| 149 | 54 VH | DVQLVQSGAEVKKPGASVKVSCKASGFDIQDTYMHWVKQRPGQGL |
| | | EWIGRIDPASGHTKYDPKFQVRATITTDTSTSTAYLELSSLRSEDTAV |
| | | YYCARSGGLPDVWGQGTTVTVSS |
| 150 | 57 VH, 111 VH | QVQLVQSGAEVKKPGASVKVSCKASGFDIQDTYMHWVRQRPGQGL |
| | | EWMGRIEPASGHIKYDPKFQGRVTMTRDTSTSTVYMELSSLRSEDTA |
| | | VYYCARSGGLPDWWGQGTTVTVSS |
| 151 | 60 VH, 117 VH | QVQLVQSGAEVKKPGASVKVSCKASGFDIQDTYMHWVRQAPGQGL |
| | | EWMGRIDPASGHIKYDPKFQGRVTMTRDTSTSTVYMELSSLRSEDTA |
| | | VYYCARSGGLPDWWGQGTTVTVSS |
| 152 | 61 VH | QVQLVQSGAEVKKPGASVKVSCKASGFDIQDTYMHWVRQAPGQGL |
| | | EWIGRIEPASGHIKYDPKFQGRVTMTRDTSTSTVYMELSSLRSEDTAV |
| | | YYCARSGGLPDWWGQGTTVTVSS |
| 153 | 62 VH, 118 VH | QVQLVQSGAEVKKPGASVKVSCKASGFDIQDTYMHWVRQAPGQGL |
| | | EWIGRIDPASGHIKYDPKFQGRVTMTRDTSTSTVYMELSSLRSEDTAV |
| | | YYCARSGGLPDWWGQGTTVTVSS |
| 154 | 63 VH, 120 VH | QVQLVQSGAEVKKPGASVKVSCKASGFDIQDTYMHWVRQAPGQGL |
| | | EWMGRIEPASGHVKYDPKFQGRVTMTRDTSTSTVYMELSSLRSEDT |
| | | AVYYCARSGGLPDWWGQGTTVTVSS |
| 155 | 66 VH | QVQLVQSGAEVKKPGASVKVSCKASGFDIQDTYMHWVRQAPGQGL |
| | | EWMGRIEPASGHIKYDPKFQGRVTITRDTSTSTVYMELSSLRSEDTAV |
| | | YYCARSGGLPDWWGQGTTVTVSS |
| 156 | 69 VH, 108 VH | EVQLVQSGAEVKKPGASVKVSCKASGFDIQDTYMHWVRQAPGQGL |
| | | EWMGRIEPASGHIKYDPKFQGRVTMTRDTSTSTVYMELSSLRSEDTA |
| | | VYYCARSGGLPDWWGQGTTVTVSS |
| 157 | 70 VH, 73 VH | QVQLVQSGAEVKKPGASVKVSCKASGFDIQDTYMHWVRQAPGQGL |
| | | EWMGRIEPASGHIKYDPKFQGRVTMTTDTSTSTVYMELSSLRSEDTA |
| | | VYYCARSGGLPDWWGQGTTVTVSS |
| 158 | 71 VH, 74 VH | QVQLVQSGAEVKKPGASVKVSCKASGFDIQDTYMHWVRQAPGQGL |
| | | EWMGRIEPASGHIKYDPKFQGRVTITTDTSTSTVYMELSSLRSEDTAV |
| | | YYCARSGGLPDWWGQGTTVTVSS |
| 159 | 76 VH, 119 VH | QVQLVQSGAEVKKPGASVKVSCKASGFDIQDTYMHWVRQAPGQGL |
| | | EWMGRIEPASGHTKYDPKFQGRVTMTRDTSTSTVYMELSSLRSEDTA |
| | | VYYCARSGGLPDWWGQGTTVTVSS |
| 160 | 77 VH | QVQLVQSGAEVKKPGASVKVSCKASGFDIQDTYMHWVRQAPGQGL |
| | | EWMGRIEPASGHIKYDPKFQGRATITRDTSTSTVYMELSSLRSEDTAV |
| | | YYCARSGGLPDWWGQGTTVTVSS |
| 161 | 92 VH | QVQLVQSGAEVKKPGASVKVSCKASGFDIQDTYMHWVRQAPGQGL |
| | | EWMGRIEPASGHIKYDPKFQGRVTMTRDTSTSTVYLELSSLRSEDTA |
| | | VYYCARSGGLPDWWGQGTTVTVSS |
| 162 | 93 VH | QVQLVQSGAEVKKPGASVKVSCKASGFDIQDTYMHWVRQAPGQGL |
| | | EWMGRIEPASGHIKYDPKFQGRVTMTRDTSTSTAYLELSSLRSEDTA |
| | | VYYCARSGGLPDWWGQGTTVTVSS |
| 163 | 121 VH | QVQLVQSGAEVKKPGASVKVSCKASGFDIQDTYMHWVRQRPGQGL |
| | | EWMGRIDPASGHTKYDPKFQVRATITTDTSTSTAYLELSSLRSEDTAV |
| | | YYCARSGGLPDVWGQGTTVTVSS |
| 164 | 124 VH | QVQLVQSGAEVKKPGASVKVSCKASGFDIQDTYMHWVRQRPGQGL |
| | | EWIGRIDPASGHTKYDPKFQVRVTITTDTSTSTAYLELSSLRSEDTAV |
| | | YYCARSGGLPDVWGQGTTVTVSS |
| 165 | 161 VH | QVQLVQSGAEVKKPGASVKVSCKASGFDIQDTYMHWVRQRPGQGL |
| | | EWMGRIDPASGHTKYDPKFQVRVTITTDTSTSTVYLELSSLRSEDTAV |
| | | YYCARSGGLPDVWGQGTTVTVSS |
| 166 | 163 VH | QVQLVQSGAEVKKPGASVKVSCKASGFDIQDTYMHWVRQRPGQGL |
| | | EWMGRIDPASGHTKYDPKFQVRVTITTDTSTSTAYMELSSLRSEDTA |
| | | VYYCARSGGLPDVWGQGTTVTVSS |
| 167 | 164 VH | QVQLVQSGAEVKKPGASVKVSCKASGFDIQDTYMHWVRQRPGQGL |
| | | EWMGRIDPASGHTKYDPKFQVRVTMTTDTSTSTAYLELSSLRSEDTA |
| | | VYYCARSGGLPDVWGQGTTVTVSS |
| 168 | 171 VH, 177 VH | QVQLVQSGAEVKKPGASVKVSCKASGFDIQDTYMHWVRQAPGQGL |
| | | EWMGRIDPASGHIKYDPKFQGRATITTDTSTSTVYMELSSLRSEDTAV |
| | | YYCARSGGLPDWWGQGTTVTVSS |
| 169 | 195 VH, 201 | EVQLVQSGAEVKKPGASVKVSCKASGFDIQDTYMHWVRQAPGQGL |
| | VH, 207 VH | EWMGRIEPASGHIKYDPKFQGRATITTDTSTSTVYMELSSLRSEDTAV |
| | | YYCARSGGLPDWWGQGTTVTVSS |
| 170 | 5C3D11 VH | EVQLQQSGAELVKPGASVKLSCTASGFDIQDTYMHWVKQRPEQGLE |
| | | WIGRIDPASGHTKYDPKFQVKATITTDTSSNTAYLQLSSLTSEDTAVY |
| | | YCSRSGGLPDVWGAGTTVTVSS |
| 171 | 9E12E5 VH | QVHLQQSGPELVKPGASVKLSCKASGYTFTKYDINWVRQRPEQGLE |
| | | WIGWIFPGDGRTDYNEKFKGKATLTTDKSSSTAYMEVSRLTSEDSAV |
| | | YFCARYGPAMDYWGQGTSVTVAS |
| 172 | AS12824 VH | QVQLVQSGAEVKKPGASVKVSCKASGFDICDTYMHWVKQRPGQGL |
| | | EWIGRIDPASGHTKYDPKFQVRATITTDTSTSTAYLELSSLRSEDTAV |
| | | YYCARSGGLPDVWGQGTTVTVSS |
| 173 | AS12823 VH | QVQLVQSGAEVKKPGASVKLSCKASGFDICDTYMHWVRQRPGQGL |
| | | EWIGRIDPASGHTKYDPKFQVRATMTTDTSTSTVYLELSSLRSEDTAV |
| | | YYCARSGGLPDVWGQGTTVTVSS |
| 174 | AS12819 VH | QVQLVQSGAEVVKPGASVKLSCKASGFDICDTYMHWVRQRPGQGL |
| | | EWMGRIDPASGHTKYDPKFQVRVTMTTDTSTSTVYLELSSLRSEDTA |
| | | VYYCARSGGLPDVWGQGTTVTVSS |
| 175 | AS12816 VH | QVQLVQSGAEVKKPGASVKVSCKASGFDICDTYMHWVKQRPGQGL |
| | | EWIGRIDPASGHTKYDPKFQVRATITRDTSTSTAYLELSSLRSEDTAV |
| | | YYCSRSGGLPDVWGQGTTVTVSS |
| 176 | AS12825 VH | QVQLVQSGAEVKKPGASVKVSCKASGFDICDTYMHWVKQAPGQGL |
| | | EWMGRIDPASGHTKYDPKFQVRATMTTDTSTSTAYLELSSLRSEDTA |
| | | VYYCSRSGGLPDVWGQGTTVTVSS |
| 177 | 12835 VH | QVQLVQSGAEVKKPGASVKLSCKASGFDIQDTYMHWVRQAPGQGL |
| | | EWMGRIDPASGHTKYDPKFQVRVTMTTDTSTSTVYMELSSLRSEDT |
| | | AVYYCSRSGGLPDVWGQGTTVTVSS |
| 178 | 18-7 VH | QVQLVQSGAEVKKPGASVKLSCKASGFDIQDTYMHWVRQAPGQGL |
| | | EWMGRIDPASGHTKYDPKFQVRVTMTRDTSTSTVYMELSSLRSEDT |
| | | AVYYCSRSGGLPDVWGQGTTVTVSS |
| 179 | 21-3 VH, L8 VH | QVQLVQSGAEVKKPGASVKVSCKASGFDIQDTYMHWVRQAPGQGL |
| | | EWMGRIDPASGHTKYDPKFQVRVTMTRDTSTSTVYMELSSLRSEDT |
| | | AVYYCARSGGLPDVWGQGTTVTVSS |
| 180 | 21-3 V102K VH | QVQLVQSGAEVKKPGASVKVSCKASGFDIQDTYMHWVRQAPGQGL |
| | | EWMGRIDPASGHTKYDPKFQVRVTMTRDTSTSTVYMELSSLRSEDT |
| | | AVYYCARSGGLPDKWGQGTTVTVSS |
| 181 | 21-3 V102M VH | QVQLVQSGAEVKKPGASVKVSCKASGFDIQDTYMHWVRQAPGQGL |
| | | EWMGRIDPASGHTKYDPKFQVRVTMTRDTSTSTVYMELSSLRSEDT |
| | | AVYYCARSGGLPDMWGQGTTVTVSS |
| 182 | 21-3 V102Q VH | QVQLVQSGAEVKKPGASVKVSCKASGFDIQDTYMHWVRQAPGQGL |
| | | EWMGRIDPASGHTKYDPKFQVRVTMTRDTSTSTVYMELSSLRSEDT |
| | | AVYYCARSGGLPDQWGQGTTVTVSS |
| 183 | 21-3 V102W VH | QVQLVQSGAEVKKPGASVKVSCKASGFDIQDTYMHWVRQAPGQGL |
| | | EWMGRIDPASGHTKYDPKFQVRVTMTRDTSTSTVYMELSSLRSEDT |
| | | AVYYCARSGGLPDWWGQGTTVTVSS |
| 184 | 21-3 CDRv VH | QVQLVQSGAEVKKPGASVKVSCKASGFX₁X₂X₃DTX₄X₅HWVRQAPG |
| | | QGLEWMGRIDPASGHTKYDPKFQVRVTMTRDTSTSTVYMELSSLRS |
| | | EDTAVYYCARSGGX₆PDX₇WGQGTTVTVSS |
| | | X₁ = D or E |
| | | X₂ = I, P, or V |
| | | X₃ = G, Q, S, or V |
| | | X₄ = F or Y |
| | | X₅ = I or M |
| | | X₆ = L or M |
| | | X₇ = E, I, K, L, M, Q, T, V, W, or Y |
| 185 | 21-3 CDRv VH | QVQLVQSGAEVKKPGASVKVSCKASGFX₁X₂X₃DTX₄X₅HWVRQAPG |
| | | QGLEWMGRIDPASGHTKYDPKFQVRVTMTRDTSTSTVYMELSSLRS |
| | | EDTAVYYCSRSGGX₆PDX₇WGQGTTVTVSS |
| | | X₁ = D or E |
| | | X₂ = I, P, or V |
| | | X₃ = G, Q, S, or V |
| | | X₄ = F or Y |
| | | X₅ = I or M |
| | | X₆ = L or M |
| | | X₇ = E, I, K, L, M, Q, T, V, W, or Y |
| 186 | Clone 2 VH, | QVQLVQSGAEVKKPGASVKVSCKASGFDIQDTYMHWVRQAPGQGL |
| | Clone 52 VH | EWMGRIEPASGHIKYSPKFQGRVTMTRDTSTSTVYMELSSLRSEDTA |
| | | VYYCARSGGLPDWWGQGTTVTVSS |
| 187 | Clone 46 VH | QVQLVQSGAEVKKPGASVKVSCKASGFDIQDTYMHWVRQAPGQGL |
| | | EWMGRIEPASGHVKYSPKFQVRVTMTRDTSTSTVYMELSSLRSEDTA |
| | | VYYCARSGGLPDWWGQGTTVTVSS |
| 188 | Clone 47 VH | QVQLVQSGAEVKKPGASVKVSCKASGFDIQDTYMHWVRQAPGQGL |
| | | EWMGRIEPASGHVKYDPKFQTRVTMTRDTSTSTVYMELSSLRSEDTA |
| | | VYYCARSGGLPDWWGQGTTVTVSS |
| 189 | Clone 14 VH | QVQLVQSGAEVKKPGASVKVSCKASGFDIQDTYMHWVRQAPGQGL |
| | | EWMGRIDPASGHIKYDPKFQKRVTMTRDTSTSTVYMELSSLRSEDTA |
| | | VYYCARSGGLPDMWGQGTTVTVSS |
| 190 | Clone 16L VH | QVQLVQSGAEVKKPGASVKVSCKASGFDIQDTYMHWVRQAPGQGL |
| | | EWMGRIDPASGHvKiDPKFQVRVTMTRDTSTSTVYMELSSLRSEDTA |
| | | VYYCARSGGLPDMWGQGTTVTVSS |
| 191 | Clone 17L VH | QVQLVQSGAEVKKPGASVKVSCKASGFDIQDTYMHWVRQAPGQGL |
| | | EWMGRIDPASGHLKYDPKFQVRVTMTRDTSTSTVYMELSSLRSEDT |
| | | AVYYCARSGGLPDMWGQGTTVTVSS |
| 192 | Clone 17L-1 VH | QVQLVQSGAEVKKPGASVKVSCKASGFDIQDTYMHWVRQAPGQGL |
| | | EWMGRIDPASGHLKYDPKFQRRVTMTRDTSTSTVYMELSSLRSEDT |
| | | AVYYCARSGGLPDMWGQGTTVTVSS |
| 193 | Clone 23 VH, | QVQLVQSGAEVKKPGASVKVSCKASGFDIQDTYMHWVRQAPGQGL |
| | clone A1 VH | EWMGRIDPASGHLKYDPKFQNRVTMTRDTSTSTVYMELSSLRSEDT |
| | | AVYYCARSGGLPDKWGQGTTVTVSS |
| 194 | Clone 53 VH, | QVQLVQSGAEVKKPGASVKVSCKASGFDIQDTYMHWVRQAPGQGL |
| | clone E1 VH | EWMGRIEPASGHLKYDPKFQERVTMTRDTSTSTVYMELSSLRSEDTA |
| | | VYYCARSGGLPDKWGQGTTVTVSS |
| 195 | Clone 3-17L V- | QVQLVQSGAEVKKPGASVKVSCKASGFDIQDTYMHWVRQAPGQGL |
| | A VH | EWMGRIDPASGHLKYDPKFQGRVTITRDTSASTAYMELSSLRSEDTA |
| | | VYYCARSGGLPDMWGQGTTVTVSS |
| 196 | Clone 3-17L VH | QVQLVQSGAEVKKPGASVKVSCKASGFDIQDTYMHWVRQAPGQGL |
| | | EWMGRIDPASGHLKYDPKFQGRVTITRDTSASTVYMELSSLRSEDTA |
| | | VYYCARSGGLPDMWGQGTTVTVSS |
| 197 | Clone L8mod | QVQLVQSGAEVKKPGASVKVSCKASGFDIQDTYMHWVRQAPGQGL |
| | VH | EWMGRIDPASGHTKYDPKFQGRATITTDTSASTAYLQLSSLRSEDTA |
| | | VYYCARSGGLPDVWGQGTTVTVSS |
| 198 | Clone X-V VH | QVQLVQSGAEVKKPGASVKVSCKASGFDIQDTYMHWVRQAPGQGL |
| | | EWMGRIDPASGHTKYDPKFQVRATITTDTSASTAYLQLSSLRSEDTA |
| | | VYYCARSGGLPDFWGQGTTVTVSS |
| 199 | Clone X VH, | QVQLVQSGAEVKKPGASVKVSCKASGFDIQDTYMHWVRQAPGQGL |
| | clone XL3-6 VH, | EWMGRIDPASGHTKYDPKFQGRATITTDTSASTAYLQLSSLRSEDTA |
| | clone XL3-10 | VYYCARSGGLPDFWGQGTTVTVSS |
| | VH, clone XL3- | |
| | 15 VH, clone L3- | |
| | 13 VH | |
| 200 | Clone H3-1 VH | QVQLVQSGAEVKKPGASVKVSCKASGFDIQDTYMHWVRQAPGQGL |
| | | EWMGRIDPASGHTKYDPKFQGRATITTDTSASTAYLQLSSLRSEDTA |
| | | VYYCARSGGLPDLWGQGTTVTVSS |
| 267 | Clone H2-2 VH | QVQLVQSGAEVKKPGASVKVSCKASGFDIQDTYMHWVRQAPGQGL |
| | | EWMGRIDPASGHSKYDPKFQVRATITIDTSASTAYLQLSSLRSEDTA |
| | | VYYCARSGGLPDFWGQGTTVTVSS |
| 268 | Clone H2-5 VH | QVQLVQSGAEVKKPGASVKVSCKASGFDIQDTYMHWVRQAPGQGL |
| | | EWMGRIDPASGHYKYDPKFQVRATITTDTSASTAYLQLSSLRSEDTA |
| | | VYYCARSGGLPDFWGQGTTVTVSS |
| 269 | M1 VH | EVQLVESGGGLVQPGGSLRLSCAVSGFTFSSYWMSWVRQAPGKGLE |
| | | WVANIKEDGSEKNYVDSVKGRFTLSSDNAKNSLYLQMNSLRAEDTA |
| | | VYYCAREDYDSYYKYGMDVWGQGTAVIVSS |
| 270 | M2 VH | QVQLQQWGAGLLKPSETLSLTCAVYGGSFTGFYWSWIRQPPGKGLE |
| | | WIGEINHRGNTNYNPSLKSRVTMSVDTSKNQFSLNMISVTAADTAM |
| | | YFCASPFYDFWSGSDYWGQGTLVTVSS |
| 271 | M3 VH | QVQLVQSGAEVKKPGASVKVSCKASGYDFTYYGISWVRQAPGQGLE |
| | | WMGWISTYNGNTHYARMLQGRVTMTTDTSTRTAYMELRSLRSDDT |
| | | AVYYCARENYYGSGAYRGGMDVWGQGTTVTVSS |
| 272 | M3 VH + | QVQLVQSGAEVKKPGASVKVSCKASGYDFTYYGISWVRQAPGQGLE |
| | constant | WMGWISTYNGNTHYARMLQGRVTMTTDTSTRTAYMELRSLRSDDT |
| | | AVYYCARENYYGSGAYRGGMDVWGQGTTVTVSSASTKGPSVFPLA |
| | | PSSKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDKT |
| | | HTCPPCPAPEAAGAPSVFLFPPKPKDTLMISRTPEVTCVVVDVSHEDP |
| | | EVKFNWYVDGVEVHNAKTKPREEQYNSTYRVVSVLTVLHQDWLNG |
| | | KEYKCKVSNKALPAPIEKTISKAKGQPREPQVYTLPPSREEMTKNQV |
| | | SLTCLVKGFYPSDIAVEWESNGQPENNYKTTPPVLDSDGSFFLYSKLT |
| | | VDKSRWQQGNVFSCSVMHEALHNHYTQKSLSLSPG |
| 273 | M4 VH | QLQLQESGPGLVKPSETLSLTCTVSGGSISSRSYYWGWIRQPPGKGLE |
| | | WIGSIYYNGRTYYNPSLKSRVTISVDTSKNQFSLKLSSVTAADTAVYY |
| | | CAREDYGDYGAFDIWGQGTMVTVSS |
| 274 | M5 VH | QVTLKESGPALVKPTQTLTLTCTFSGFSLSTSNMGVVWIRQPPGKALE |
| | | WLAHILWDDREYSNPALKSRLTISKDTSKNQVVLTMTNMDPVDTAT |
| | | YYCARMSRNYYGSSYVMDYWGQGTLVTVSS |
| 275 | M6 VH | QVQLVQSGSELKKPGASVKVSCKASGYTFTLYGMNWVRQAPGQGL |
| | | EWMGWINTYTGEPTYADDFKGRFVFSLDTSVSTAYLQISSLKAEDTA |
| | | VYYCARDTAMDYAMAYWGQGTLVTVSS |
| 276 | M6 VH | QVQLVQSGSELKKPGASVKVSCKASGYTFTLYGMNWVKQAPGKGL |
| | | KWMGWINTYTGEPTYADDFKGRFVFSLDTSVSTAYLQISSLKAEDTA |
| | | VYFCARDTAMDYAMAYWGQGTLVTVSS |
| 277 | M6 VH | QVQLVQSGSELKKPGASVKVSCKASGYTFTNYGMNWVRQAPGQGL |
| | | EWMGWINTYTGEPTYADDFKGRFVFSLDTSVSTAYLQISSLKAEDTA |
| | | VYYCARDYGKYGDYYAMDYWGQGTLVTVSS |
| 278 | M6 VH | QVQLVQSGSELKKPGASVKVSCKASGYTFTNYGMNWVRQAPGKGL |
| | | KWMGWINTYTGEPTYADDFKGRFVFSLDTSVSTAYLQISSLKAEDTA |
| | | VYFCARDYGKYGDYYAMDYWGQGTLVTVSS |
| 279 | M7 VH | QVQLQQPGSVLVRPGASVKVSCKASGYTFTSSWMHWAKQRPGQGL |
| | | EWIGEIHPNSGGTNYNEKFKGKATVDTSSSTAYVDLSSLTSEDSAVY |
| | | YCARGDYYGYVSWFAYWGQGTLVTVSS |
| 280 | M7 VH | QVQLVQSGAEVKKPGASVKVSCKASGYTFTSSWMHWARQAPGQGL |
| | | EWIGEIHPNSGGTNYAQKFQGRATLTVDTSSSTAYMELSRLRSDDTA |
| | | VYYCARGDYYGYVSWFAYWGQGTLVTVSS |
| 281 | M7 VH | QVQLVQSGAEVKKPGASVKVSCKASGYTFTSSWMHWARQAPGQGL |
| | | EWIGEIHPNSGGTNYAQKFQGRATMTVDTSISTAYMELSRLRSDDTA |
| | | VYYCARGDYYGYVSWFAYWGQGTLVTVSS |
| 282 | M7 VH | QVQLVQSGAEVKKPGASVKVSCKASGYTFTSSWMHWARQAPGQGL |
| | | EWIGEIHPNSGGTNYAQKFQGRVTMTVDTSISTAYMELSRLRSDDTA |
| | | VYYCARGDYYGYVSWFAYWGQGTLVTVSS |
| 283 | M7 VH | QVQLVQSGAEVKKPGASVKVSCKASGYTFTSSWMHWARQAPGQGL |
| | | EWMGEIHPNSGGTNYAQKFQGRVTMTVDTSISTAYMELSRLRSDDT |
| | | AVYYCARGDYYGYVSWFAYWGQGTLVTVSS |
| 284 | M8 VH | QVQLVQSGAEVKKPGASVKVSCKASGYTFTSYDINWVRQAPGQGLE |
| | | WMGWLNPNSGNTGYAQKFQGRVTMTADRSTSTAYMELSSLRSEDT |
| | | AVYYCAREVPETAAFEYWGQGTLVTVSS |
| 285 | M8 VH | QVQLVQSGAEVKKPGASVKVSCKASGYTFTSYDINWVRQAPGQGLE |
| | | WMGWLNPNSGYTGYAQKFQGRVTMTADRSTSTAYMELSSLRSEDT |
| | | AVYYCAREVPETAAFEYWGQGTLVTVSS |
| 286 | M9 VH | QVQLQESGPGLVKPSETLSLTCTVSGGSISSYFWSWIRQPPGKGLEWI |
| | | GYIYYSGNTKYNPSLKSRVTISIDTSKNQFSLKLSSVTAADTAVYYCA |
| | | RETGSYYGFDYWGQGTLVTVSS |
| 287 | M10 VH | QVQLQQWGAGLLKPSETLSLTCAVHGGSFSGYYWNWIRQPPGKGLE |
| | | WIGEINHAGNTNYNPSLKSRVTISLDTSKNQFSLTLTSVTAADTAVYY |
| | | CARGYCRSTTCYFDYWGQGTLVTVSS |
| 288 | M11 VH | EVQLLESGGGLVQPGKSLRLSCAVSGFTFSTYGMNWVRQAPGKGLE |
| | | WVSSISGTGRTTYHADSVQGRFTVSRDNSKNILYLQMNSLRADDTAV |
| | | YFCTKERGDYYYGVFDYWGQGTLVTVSS |
| 289 | M12 VH | QIQLVQSGPELKKPGETVKISCKASGYTFTTYGMSWVKQAPGKGLK |
| | | WMGWMNTYSGVTTYADDFKGRFAFSLETSASTAYMQIDNLKNEDT |
| | | ATYFCAREGYVFDDYYATDYWGQGTSVTVSS |

In certain embodiments, an anti-TL1A antibody comprises the light chain CDRs set forth in an antibody selected from Table 4.

TABLE 4
Example light chain variable region sequences

| SEQ | | |
|---|---|---|
| ID | | |
| NO | Description | Sequence |
| 201 | 217 VL, | EIVLTQSPGTLSLSPGERATLSCRASSSVSYMYWYQQKPGQA |
| | 219 VL, 221 VL, 200 VL, 213 | PRPLIYATSNLASGIPDRFSGSGSGTDFTLTISRLEPEDFAVYY |
| | VL, 212 VL, 211 VL, 199 VL, | CQQWEGNPRTFGGGTKLEIK |
| | 214 VL, 216 VL, 222 VL, 203 | |
| | VL, 147 VL, 218 VL, 179 VL, | |
| | 148 VL, 149 VL, 151 VL, 180 | |
| | VL, 175 VL, 178 VL, 145 VL, | |
| | 146 VL, 150 VL, 152 VL, 176 | |
| | VL, 177 VL, 201 VL, 202 VL, | |
| | 204 VL, 215 VL, 224 VL | |
| 202 | 223 VL, 107 VL, 205 VL, 181 | EIVLTQSPGTLSLSPGERATLSCRASSSVSYMYWYQQKPGQA |
| | VL, 188 VL, 64 VL, 67 VL, 68 | PRLLIYATSNLASGIPDRFSGSGSGTDFTLTISRLEPEDFAVYY |
| | VL, 94 VL, 33 VL, 57 VL, 58 | CQQWEGNPRTFGGGTKLEIK |
| | VL, 59 VL, 60 VL, 61 VL, 62 | |
| | VL, 63 VL, 65 VL, 66 VL, 69 | |
| | VL, 70 VL, 71 VL, 72 VL, 76 | |
| | VL, 77 VL, 78 VL, 91 VL, 92 | |
| | VL, 93 VL, 97 VL, 98 VL, 99 | |
| | VL, 140 VL, 142 VL, 143 VL, | |
| | 182 VL, 183 VL, 184 VL, 185 | |
| | VL, 186 VL, 187 VL, 189 VL, | |
| | 190 VL, 191 VL, 192 VL, 206 | |
| | VL, 207 VL, 208 VL, 209 VL, | |
| | 210 VL | |
| 203 | 15 VL | EIVLTQSPGTLSLSPGERATLSCRASSSVSYMYWYQQKPGQA |
| | | PRLLIYATSNLASGIPDRFSGSGSGTDFTLTISRLEPEDFAVYY |
| | | CQQWSGNPRTFGGGTKLEIK |
| 204 | 30 VL, 100 VL, 129 VL, 122 | EIVLTQSPGTLSLSPGERATLSCRASSSVSYMYWYQQKPGQA |
| | VL, 127 VL, 126 VL, 160 VL, | PRLLIYATSNLASGIPDRFSGSGSGTDFTLTISRLEPEDFAVYY |
| | 157 VL, 159 VL, 158 VL, 125 | CQQWKGNPRTFGGGTKLEIK |
| | VL, 103 VL, 101 VL, 102 VL, | |
| | 104 VL, 105 VL, 121 VL, 123 | |
| | VL, 124 VL, 128 VL, 144 VL, | |
| | 161 VL, 162 VL, 163 VL, 164 | |
| | VL | |
| 205 | 110 VL, 197 VL, 112 VL, 169 | EIVLTQSPGTLSLSPGERATLSCRASSSVSYMYWYQQK |
| | VL, 173 VL, 115 VL, 113 VL, | PGQAPRPWIYATSNLASGIPDRFSGSGSGTDFTLTISRL |
| | 96 VL, 196 VL, 172 VL, 75 | EPEDFAVYYCQQWEGNPRTFGGGTKLEIK |
| | VL, 174 VL, 109 VL, 198 VL, | |
| | 170 VL, 29 VL, 31 VL, 32 VL, | |
| | 73 VL, 74 VL, 95 VL, 108 VL, | |
| | 111 VL, 114 VL, 116 VL, 117 | |
| | VL, 118 VL, 119 VL, 120 VL, | |
| | 130 VL, 153 VL, 154 VL, 155 | |
| | VL, 156 VL, 165 VL, 166 VL, | |
| | 167 VL, 168 VL, 171 VL, 193 | |
| | VL, 194 VL, 195 VL, 220 VL | |
| 206 | 134 VL, 132 VL, 133 VL, 135 | EIVLTQSPGTLSLSPGERATLSCRASSSVSYMYWYQQK |
| | VL, 136 VL | PGQAPRPLIYATSNLASGIPDRFSGSGSGTDFTLTISRLE |
| | | PEDFAVYYCQQWKGNPRTFGGGTKLEIK |
| 207 | 138 VL, 137 VL, 139 VL, 141 | EIVLTQSPGTLSLSPGERATLSCRASSSVSYMYWYQQK |
| | VL | PGQAPRLLIYATSNLASGIPDRFSGSGSGTDFTLTISRLE |
| | | PEDFAVYYCQQWSRNPRTFGGGTKLEIK |
| 208 | 34 VL, 35 VL, 36 VL, 37 VL, | EIVLTQSPGTLSASPGERATMSCRASSSVSYMYWYQQ |
| | 38 VL, 39 VL, 40 VL, 41 VL, | KPGQAPRPWIYATSNLASGVPDRFSGSGSGTDYTLTIS |
| | 42 VL, 43 VL, 44 VL, 45 VL, | RVEPEDFAVYYCQQWSGNPRTFGGGTKLEIK |
| | 46 VL, 47 VL, 53 VL, 54 VL, | |
| | 55 VL, 79 VL, 81 VL, 82 VL, | |
| | 83 VL | |
| 209 | 85 VL | EIVLTQSPGTLSLSPGERATLSCRASSSVSYMYWYQQK |
| | | PGQAPRLLIYATSNLASGVPDRFSGSGSGTDFTLTISRL |
| | | EPEDFAVYYCQQWEGNPRTFGGGTKLEIK |
| 210 | 48 VL | EIVLTQSPGTLSASPGERATLSCRASSSVSYMYWYQQK |
| | | PGQAPRPWIYATSNLASGVPDRFSGSGSGTDYTLTISR |
| | | VEPEDFAVYYCQQWSGNPRTFGGGTKLEIK |
| 211 | 49 VL | EIVLTQSPGTLSASPGERATMSCRASSSVSYMYWYQQ |
| | | KPGQAPRLLIYATSNLASGVPDRFSGSGSGTDYTLTISR |
| | | VEPEDFAVYYCQQWSGNPRTFGGGTKLEIK |
| 212 | 50 VL | EIVLTQSPGTLSASPGERATMSCRASSSVSYMYWYQQ |
| | | KPGQAPRPWIYATSNLASGVPDRFSGSGSGTDFTLTISR |
| | | VEPEDFAVYYCQQWSGNPRTFGGGTKLEIK |
| 213 | 51 VL | EIVLTQSPGTLSASPGERATMSCRASSSVSYMYWYQQ |
| | | KPGQAPRPWIYATSNLASGVPDRFSGSGSGTDYTLTIS |
| | | RLEPEDFAVYYCQQWSGNPRTFGGGTKLEIK |
| 214 | 52 VL | EIVLTQSPGTLSASPGERATMSCRASSSVSYMYWYQQ |
| | | KPGQAPRPWIYATSNLASGVPDRFSGSGSGTDFTLTISR |
| | | LEPEDFAVYYCQQWSGNPRTFGGGTKLEIK |
| 215 | 56 VL | EIVLTQSPGTLSASPGERATMSCRASSSVSYMYWYQQ |
| | | KPGQAPRPWIYATSNLASGIPDRFSGSGSGTDYTLTISR |
| | | VEPEDFAVYYCQQWSGNPRTFGGGTKLEIK |
| 216 | 86 VL | EIVLTQSPGTLSLSPGERATLSCRASSSVSYMYWYQQK |
| | | PGQAPRLLIYATSNLASGIPDRFSGSGSGTDYTLTISRLE |
| | | PEDFAVYYCQQWEGNPRTFGGGTKLEIK |
| 217 | 87 VL | EIVLTQSPGTLSLSPGERATLSCRASSSVSYMYWYQQK |
| | | PGQAPRLLIYATSNLASGIPDRFSGSGSGTDFTLTISRVE |
| | | PEDFAVYYCQQWEGNPRTFGGGTKLEIK |
| 218 | 88 VL | EIVLTQSPGTLSLSPGERATLSCRASSSVSYMYWYQQK |
| | | PGQAPRLLIYATSNLASGIPDRFSGSGSGTDYTLTISRV |
| | | EPEDFAVYYCQQWEGNPRTFGGGTKLEIK |
| 219 | 89 VL | EIVLTQSPGTLSASPGERATLSCRASSSVSYMYWYQQK |
| | | PGQAPRLLIYATSNLASGIPDRFSGSGSGTDFTLTISRLE |
| | | PEDFAVYYCQQWEGNPRTFGGGTKLEIK |
| 220 | 90 VL | EIVLTQSPGTMSLSPGERATLSCRASSSVSYMYWYQQ |
| | | KPGQAPRLLIYATSNLASGIPDRFSGSGSGTDFTLTISRL |
| | | EPEDFAVYYCQQWEGNPRTFGGGTKLEIK |
| 221 | 5C3D11 VL | QIVLSQSPAILSASPGEKVTMTCRASSSVSYMYWYQQ |
| | | KPGSSPKPWIYATSNLASGVPDRFSGSGSGTSYSLTISR |
| | | VEAEDAATYYCQQWSGNPRTFGGGTKLEIK |
| 222 | 9E12E5 VL | MKLPVRLLVLMFWIPASSSDVLMTQTPLSLPVSLGDQ |
| | | ASISCRSSQTIVHSNGDTYLDWFLQKPGQSPKLLIYKVS |
| | | NRFSGVPDRFSGSGSGTDFTLKISRVEAEDLGVYYCFQ |
| | | GSHVPYTFGGGTKLEIK |
| 223 | AS12823 VL | EIVLTQSPGTLSLSPGERATMSCRASSSVSYMYWYQQ |
| | | KPGQAPRPWIYATSNLASGIPDRFSGSGSGTDFTLTISR |
| | | VEPEDFAVYYCQQWSGNPRTFGGGTKLEIK |
| 224 | AS12819 VL | EIVLTQSPGTLSLSPGERVTMSCRASSSVSYMYWYQQ |
| | | KPGQAPRPWIYATSNLASGVPDRFSGSGSGTDFTLTISR |
| | | VEPEDFAVYYCQQWSGNPRTFGGGTKVEIK |
| 225 | AS12816 VL | EIVLTQSPGTLSASPGERVTLSCRASSSVSYMYWYQQK |
| | | PGQAPRPWIYATSNLASGVPDRFSGSGSGTDFTLTISRL |
| | | EPEDFAVYYCQQWSGNPRTFGGGTKLEIK |
| 226 | AS12825 VL | EIVLTQSPGTLSASPGERVTMSCRASSSVSYMYWYQQ |
| | | KPGQAPRLLIYATSNLASGVPDRFSGSGSGTDFTLTISR |
| | | VEPEDFAVYYCQQWSGNPRTFGGGTKLEIK |
| 227 | 12835 VL | EIVLTQSPGTLSLSPGERVTMSCRASSSVSYMYWYQQ |
| | | KPGQAPRPWIYATSNLASGVPDRFSGSGSGTDYTLTIS |
| | | RLEPEDFAVYYCQQWSGNPRTFGGGTKLEIK |
| 228 | 21-3 VL | EIVLTQSPGTLSLSPGERATLSCRASSSVSYMYWYQQK |
| | | PGQAPRLLIYATSNLASGVPDRFSGSGSGTDYTLTISRL |
| | | EPEDFAVYYCQQWSGNPRTFGGGTKLEIK |
| 229 | 18-7 S92D VL | EIVLTQSPGTLSLSPGERATLSCRASSSVSYMYWYQQK |
| | | PGQAPRLLIYATSNLASGIPDRFSGSGSGTDFTLTISRLE |
| | | PEDFAVYYCQQWDGNPRTFGGGTKLEIK |
| 230 | 18-7 S92H VL | EIVLTQSPGTLSLSPGERATLSCRASSSVSYMYWYQQK |
| | | PGQAPRLLIYATSNLASGIPDRFSGSGSGTDFTLTISRLE |
| | | PEDFAVYYCQQWHGNPRTFGGGTKLEIK |
| 231 | 18-7 S92N VL | EIVLTQSPGTLSLSPGERATLSCRASSSVSYMYWYQQK |
| | | PGQAPRLLIYATSNLASGIPDRFSGSGSGTDFTLTISRLE |
| | | PEDFAVYYCQQWNGNPRTFGGGTKLEIK |
| 232 | 18-7 S92Q VL, clone 14 VL, | EIVLTQSPGTLSLSPGERATLSCRASSSVSYMYWYQQK |
| | clone 16L VL, clone 17L VL, | PGQAPRLLIYATSNLASGIPDRFSGSGSGTDFTLTISRLE |
| | clone 17L-1 VL, clone 3-17L | PEDFAVYYCQQWQGNPRTFGGGTKLEIK |
| | V-A VL, clone 3-17L VL | |
| 233 | 18-7 CDRv VL | EIVLTQSPGTLSLSPGERATLSCRASSSVSYMYWYQQKPGQA |
| | | PRLLIYATSNLASGIPDRFSGSGSGTDFTLTISRLEPEDFAVYY |
| | | CX₁QWX₂X₃X₄PRTFGGGTKLEIK |
| | | X₁ = Q or N |
| | | X₂ = D, E, H, N, Q, or S |
| | | X₃ = A or G |
| | | X₄ = D, F, K, N, R, S, or T |
| 234 | Clone 52 VL, clone Al VL, | EIVLTQSPGTLSLSPGERATLSCGASSSVSYMYWYQQK |
| | clone E1 VL | PGQAPRLLIYATSNLASGIPDRFSGSGSGTDFTLTISRLE |
| | | PEDFAVYYCQQWEGNPRTFGGGTKLEIK |
| 235 | Clone XL3-6 VL | EIVLTQSPGTLSLSPGERATLSCRASSSVSYMYWYQQK |
| | | PGQAPRLLIYATSNLASGIPDRFSGSGSGTDFTLTISRLE |
| | | PEDFAVYYCSQWSGNPRTFGGGTKLEIK |
| 236 | Clone XL3-10 VL | EIVLTQSPGTLSLSPGERATLSCRASSSVSYMYWYQQK |
| | | PGQAPRLLIYATSNLASGIPDRFSGSGSGTDFTLTISRLE |
| | | PEDFAVYYCQQWSGNPRSFGGGTKLEIK |
| 237 | M1 VL | DIVMTQSPDSLAVSLGERATINCKSSQSILYSSNNKNYL |
| | | AWYQQKPGQPPKLLIYWASTRESGVPDRFSGSGSGTD |
| | | FTLTISSLQAEDVSVYYCQQYYSTPFTFGPGTKVDIK |
| 238 | M2 VL | DIMLTQTPLTSPVTLGQPASISCKSSQSLVHSDGNTYLS |
| | | WLQQRPGQPPRLLFYKISNRFSGVPDRFSGSGAGTDFT |
| | | LKISRVEAEDVGVYYCMQATQFPLTFGGGTKVEIK |
| 239 | M3 VL | EIVLTQSPATLSLSPGERATLSCRASQSVSSYLAWYQQ |
| | | KPGQAPRLLIYDASNRATGIPARFSGSGSGTDFTLTISS |
| | | LEPEDFAVYYCQQRSNWPWTFGQGTKVEIK |
| 240 | M3 VL | EIVLTQSPATLSLSPGERATLSCRASQSVSSYLAWYQQ |
| | | KPGQAPRLLIYDASNRATGIPARFSGSGSGTDFTLTISS |
| | | LEPEDFAVYYCQQRSNWPWTFGQGTKVEIKRTVAAPS |
| | | VFIFPPSDEQLKSGTASVVCLLNNFYPREAKVQWKVD |
| | | NALQSGNSQESVTEQDSKDSTYSLSSTLTLSKADYEKH |
| | | KVYACEVTHQGLSSPVTKSFNRGEC |
| 241 | M4 VL | AIQLTQSPSSLSASVGDRVTITCRASQGISSALAWYQQ |
| | | KPGKAPKLLIYDASSLESGVPSRFSGSGSGTDFTLTISSL |
| | | QPEDFATYYCQQFNSYPLTFGGGTKVEIK |
| 242 | M5 VL | DIQLTQSPSFLSASVGDRVTITCSASSSVNYMHWYQQK |
| | | PGKAPKLLIYSTSNLASGVPSRFSGSGSGTEFTLTISSLQ |
| | | PEDFATYYCHQWNNYGTFGQGTKVEIKR |
| 243 | M6 VL | DVVMTQSPLSLPVTLGQPASISCKSSQNIVHSDGNTYL |
| | | EWFQQRPGQSPRRLIYKVSNRFSGVPDRFSGSGSGTDF |
| | | TLKISRVEAEDVGVYYCFQGSHVPLTFGGGTKVEIKR |
| 244 | M6 VL | DVVMTQSPLSLPVTLGQPASISCKSSQNIVHSDGNTYL |
| | | EWFQQRPGQSPRRLIYKVSNRFSGVPDRFSGSGSGTDF |
| | | TLKISRVEAEDVGVYYCFQGSHVPLTFGQGTKVEIKR |
| 245 | M6 VL | DVVMTQTPLSLPVTPGEPASISCKSSQNIVHSDGNTYL |
| | | EWYLQKPGQSPQLLIYKVSNRFSGVPDRFSGSGSGTDF |
| | | TLKISRVEAEDLGVYYCFQGSHVPLTFGGGTKVEIKR |
| 246 | M6 VL | DVVMTQTPLSLPVSLGDQASISCKSSQNIVHSDGNTYL |
| | | EWYLQKPGQSPKVLIYKVSNRFSGVPDRFSGSGSGTDF |
| | | TLKISRVEAEDLGVYYCFQGSHVPLTFGGGTKVEIKR |
| 247 | M6 VL | DVVMTQSPLSLPVTLGQPASISCRSSQSIVHSNGNTYL |
| | | DWFQQRPGQSPRRLIYKVSNRFSGVPDRFSGSGSGTDF |
| | | TLKISRVEAEDVGVYYCFQGSHVPLTFGGGTKVEIKR |
| 248 | M6 VL | DVVMTQSPLSLPVTLGQPASISCRSSQSIVHSNGNTYL |
| | | DWFQQRPGQSPRRLIYKVSNRFSGVPDRFSGSGSGTDF |
| | | TLKISRVEAEDVGVYYCFQGSHVPLTFGQGTKVEIKR |
| 249 | M6 VL | DVVMTQTPLSLPVTPGEPASISCRSSQSIVHSNGNTYLD |
| | | WYLQKPGQSPQLLIYKVSNRFSGVPDRFSGSGSGTDFT |
| | | LKISRVEAEDLGVYYCFQGSHVPLTFGGGTKVEIKR |
| 250 | M6 VL | DVVMTQTPLSLPVSLGDQASISCRSSQSIVHSNGNTYL |
| | | DWYLQKPGQSPKVLIYKVSNRFSGVPDRFSGSGSGTD |
| | | FTLKINRVEAEDLGVYFCFQGSHVPLTFGGGTKLEIKR |
| 251 | M7 VL | DIQMNQSPSSLSASLGDTITITCHASQNINVLLSWYQQ |
| | | KPGNIPKLLIYKASNLHTGVPSRFSGSGSGTGFTFTISSL |
| | | QPEDIATYYCQQGQSYPYTFGGGTKLEIK |
| 252 | M7 VL | DIQMTQSPSSLSASVGDRVTITCQASQDISNYLNWYQQ |
| | | KPGKAPKLLIYDASNLETGVPSRFSGSGSGTDFTFTISS |
| | | LQPEDIATYYCQQYDNLPYTFGQGTKLEIK |
| 253 | M7 VL | DIQMTQSPSSLSASVGDRVTITCQASQNINVLLNWYQQ |
| | | KPGKAPKLLIYKASNLHTGVPSRFSGSGSGTDFTFTISS |
| | | LQPEDIATYYCQQGQSYPYTFGQGTKLEIK |
| 254 | M7 VL | DIQMNQSPSSLSASVGDRVTITCQASQNINVLLSWYQQ |
| | | KPGKAPKLLIYKASNLHTGVPSRFSGSGSGTDFTFTISS |
| | | LQPEDIATYYCQQGQSYPYTFGQGTKLEIK |
| 255 | M8 VL | QSVLTQPPSVSGAPGQRVTISCTSSSSDIGAXXGVXWY |
| | | QQLPGTAPKLLIEGYYNRPSGVPDRFSGSKSGTSASLTI |
| | | TGLLPEDEGDYYCQSXDGTLSALFGGGTKLTVLG |
| 256 | M8 VL | QSVLTQPPSVSGAPGQRVTISCTSSSSDIGAGLGVHWY |
| | | QQLPGTAPKLLIEGYYNRPSGVPDRFSGSKSGTSASLTI |
| | | TGLLPEDEGDYYCQSWDGTLSALFGGGTKLTVLG |
| 257 | M8 VL | QSVLTQPPSVSGAPGQRVTISCTSSSSDIGAGLGVHWY |
| | | QQLPGTAPKLLIEGYYNRPSGVPDRFSGSKSGTSASLTI |
| | | TGLLPEDEGDYYCQSYDGTLSALFGGGTKLTVLG |
| 258 | M8 VL | QSVLTQPPSVSGAPGQRVTISCTSSSSDIGAALGVHWY |
| | | QQLPGTAPKLLIEGYYNRPSGVPDRFSGSKSGTSASLTI |
| | | TGLLPEDEGDYYCQSWDGTLSALFGGGTKLTVLG |
| 259 | M8 VL | QSVLTQPPSVSGAPGQRVTISCTSSSSDIGAGSGVHWY |
| | | QQLPGTAPKLLIEGYYNRPSGVPDRFSGSKSGTSASLTI |
| | | TGLLPEDEGDYYCQSWDGTLSALFGGGTKLTVLG |
| 260 | M8 VL | QSVLTQPPSVSGAPGQRVTISCTSSSSDIGAGQGVHWY |
| | | QQLPGTAPKLLIEGYYNRPSGVPDRFSGSKSGTSASLTI |
| | | TGLLPEDEGDYYCQSWDGTLSALFGGGTKLTVLG |
| 261 | M8 VL | QSVLTQPPSVSGAPGQRVTISCTSSSSDIGAGLGVLWY |
| | | QQLPGTAPKLLIEGYYNRPSGVPDRFSGSKSGTSASLTI |
| | | TGLLPEDEGDYYCQSWDGTLSALFGGGTKLTVLG |
| 262 | M8 VL | QSVLTQPPSVSGAPGQRVTISCTSSSSDIGAGLGVHWY |
| | | QQLPGTAPKLLIEGYYNRPSGVPDRFSGSKSGTSASLTI |
| | | TGLLPEDEGDYYCQSFDGTLSALFGGGTKLTVLG |
| 263 | M9 VL | DIQMTQSPSSLSASVGDRVTITCRASQSINNYLNWYQQ |
| | | RPGKAPKLLIYAASSLQSGVPSRFSGSGSGTDFTLTISSL |
| | | QPGDFATYYCQQSYSTPRTFGQGTKLEIK |
| 264 | M10 VL | EIVLTQSPGTLSLSPGERATLSCRASQSVRSSYLAWYQ |
| | | QKPGQAPRLLIYGASSRATGIPDRFSGSGSGTDFTLTIS |
| | | RLEPEDFAVYYCQQYGSSPTFGQGTRLEIK |
| 265 | M11 VL | DIQMTQSPSTLSASVGDRVTITCRASQTISSWLAWYQQ |
| | | TPEKAPKLLIYAASNLQSGVPSRFSGSGSGTEFTLTISSL |
| | | QPDDFATYYCQQYHRSWTFGQGTKVEIT |
| 266 | M12 VL | DVLMTQTPLSLPVSLGDQASISCRSSQNIVHSDGNTYL |
| | | EWYLQKPGQSPKLLIYKVSNRFSGVPDRFSGSGSGTDF |
| | | TLKISRVEAEDLGIYYCFQGSHVPLTFGAGTKLELK |

In certain embodiments, an anti-TL1A antibody comprises the CDRs set forth in any one of the antibodies of Table 7. For instance, an anti-TL1A antibody comprises the CDRs of antibody A15, A29, A30, A31, A32, A33, A34, A35, A36, A37, A38, A39, A40, A41, A42, A43, A44, A45, A46, A47, A48, A49, A50, A51, A52, A53, A54, A55, A56, A57, A58, A59, A60, A61, A62, A63, A64, A65, A66, A67, A68, A69, A70, A71, A72, A73, A74, A75, A76, A77, A78, A79, A81, A82, A83, A85, A86, A87, A88, A89, A90, A91, A92, A93, A94, A95, A96, A97, A98, A99, A100, A101, A102, A103, A104, A105, A107, A108, A109, A110, A111, A112, A113, A114, A115, A116, A117, A118, A119, A120, A121, A122, A123, A124, A125, A126, A127, A128, A129, A130, A132, A133, A134, A135, A136, A137, A138, A139, A140, A141, A142, A143, A144, A145, A146, A147, A148, A149, A150, A151, A152, A153, A154, A155, A156, A157, A158, A159, A160, A161, A162, A163, A164, A165, A166, A167, A168, A169, A170, A171, A172, A173, A174, A175, A176, A177, A178, A179, A180, A181, A182, A183, A184, A185, A186, A187, A188, A189, A190, A191, A192, A193, A194, A195, A196, A197, A198, A199, A200, A201, A202, A203, A204, A205, A206, A207, A208, A209, A210, A211, A212, A213, A214, A215, A216, A217, A218, A219, A220, A221, A222, A223, A224, A500, or A501. In a non-limiting example, an anti-TL1A antibody comprises the CDRs of antibody A219. In some instances, an anti-TL1A antibody comprises the CDRs of antibody 5C3D11, 9E12E5, AS12824, AS12823, AS12819, AS12816, AS12825, 12835, 18-7, 21-3, L8, 21-3 V102K, 21-3 V102M, 21-3 V102Q, 21-3 V102 W, 21-3 CDRv, 21-3 CDRv, Clone2, Clone 52, Clone 46, Clone 47, Clone 14, Clone 16L, Clone 17L, Clone 17L-1, Clone 23, Clone A1, Clone 53, Clone E1, Clone 3-17L V-A, Clone 3-17L, Clone L8mod, CloneX-V, Clone X, Clone XL3-6, Clone XL3-10, Clone XL3-15, Clone L3-13, Clone H3-1, Clone H2-2, or Clone H2-5. Table3 and Table 4 provide the variable region sequences comprising these CDRs. In some instances, an anti-TL1A antibody comprises the CDRs of antibody M1, M2, M3, M4, M5, M6, M7, M8, M9, M10, M11, or M12. Table 3 and Table 4 provide the variable region sequences comprising the CDRs of antibodies M1-M12.

Antibody CDRs may be defined by the Kabat, Chothia, or IMGT methods.

TABLE 7
Variable region sequences of select anti-TL1A Antibodies

| | HC Variable Region | LC Variable Region |
|---|---|---|
| Antibody | SEQ ID | SEQ ID |
| A15 | 108 | 203 |
| A29 | 108 | 205 |
| A30 | 108 | 204 |
| A31 | 136 | 205 |
| A32 | 137 | 205 |
| A33 | 137 | 202 |
| A34 | 107 | 208 |
| A35 | 138 | 208 |
| A36 | 139 | 208 |
| A37 | 140 | 208 |
| A38 | 141 | 208 |
| A39 | 142 | 208 |
| A40 | 143 | 208 |
| A41 | 115 | 208 |
| A42 | 144 | 208 |
| A43 | 145 | 208 |
| A44 | 146 | 208 |
| A45 | 120 | 208 |
| A46 | 147 | 208 |
| A47 | 148 | 208 |
| A48 | 108 | 210 |
| A49 | 108 | 211 |
| A50 | 108 | 212 |
| A51 | 108 | 213 |
| A52 | 108 | 214 |
| A53 | 146 | 208 |
| A54 | 149 | 208 |
| A55 | 109 | 208 |
| A56 | 108 | 215 |
| A57 | 150 | 202 |
| A58 | 125 | 202 |
| A59 | 117 | 202 |
| A60 | 151 | 202 |
| A61 | 152 | 202 |
| A62 | 153 | 202 |
| A63 | 154 | 202 |
| A64 | 121 | 202 |
| A65 | 128 | 202 |
| A66 | 155 | 202 |
| A67 | 122 | 202 |
| A68 | 123 | 202 |
| A69 | 156 | 202 |
| A70 | 157 | 202 |
| A71 | 158 | 202 |
| A72 | 131 | 202 |
| A73 | 157 | 205 |
| A74 | 158 | 205 |
| A75 | 131 | 205 |
| A76 | 159 | 202 |
| A77 | 160 | 202 |
| A78 | 124 | 202 |
| A79 | 107 | 208 |
| A81 | 139 | 208 |
| A82 | 140 | 208 |
| A83 | 144 | 208 |
| A85 | 136 | 209 |
| A86 | 136 | 216 |
| A87 | 136 | 217 |
| A88 | 136 | 218 |
| A89 | 136 | 219 |
| A90 | 136 | 220 |
| A91 | 133 | 202 |
| A92 | 161 | 202 |
| A93 | 162 | 202 |
| A94 | 124 | 202 |
| A95 | 131 | 205 |
| A96 | 128 | 205 |
| A97 | 121 | 202 |
| A98 | 122 | 202 |
| A99 | 123 | 202 |
| A100 | 107 | 204 |
| A101 | 140 | 204 |
| A102 | 115 | 204 |
| A103 | 120 | 204 |
| A104 | 139 | 204 |
| A105 | 143 | 204 |
| A107 | 108 | 202 |
| A108 | 156 | 205 |
| A109 | 133 | 205 |
| A110 | 125 | 205 |
| A111 | 150 | 205 |
| A112 | 117 | 205 |
| A113 | 124 | 205 |
| A114 | 121 | 205 |
| A115 | 122 | 205 |
| A116 | 123 | 205 |
| A117 | 151 | 205 |
| A118 | 153 | 205 |
| A119 | 159 | 205 |
| A120 | 154 | 205 |
| A121 | 163 | 204 |
| A122 | 113 | 204 |
| A123 | 112 | 204 |
| A124 | 164 | 204 |
| A125 | 105 | 204 |
| A126 | 114 | 204 |
| A127 | 118 | 204 |
| A128 | 111 | 204 |
| A129 | 110 | 204 |
| A130 | 121 | 205 |
| A132 | 128 | 206 |
| A133 | 121 | 206 |
| A134 | 122 | 206 |
| A135 | 133 | 206 |
| A136 | 125 | 206 |
| A137 | 121 | 207 |
| A138 | 122 | 207 |
| A139 | 110 | 207 |
| A140 | 110 | 202 |
| A141 | 111 | 207 |
| A142 | 111 | 202 |
| A143 | 136 | 202 |
| A144 | 111 | 204 |
| A145 | 133 | 201 |
| A146 | 125 | 201 |
| A147 | 117 | 201 |
| A148 | 121 | 201 |
| A149 | 122 | 201 |
| A150 | 128 | 201 |
| A151 | 124 | 201 |
| A152 | 131 | 201 |
| A153 | 133 | 205 |
| A154 | 125 | 205 |
| A155 | 121 | 205 |
| A156 | 122 | 205 |
| A157 | 104 | 204 |
| A158 | 101 | 204 |
| A159 | 119 | 204 |
| A160 | 102 | 204 |
| A161 | 165 | 204 |
| A162 | 106 | 204 |
| A163 | 166 | 204 |
| A164 | 167 | 204 |
| A165 | 139 | 205 |
| A166 | 146 | 205 |
| A167 | 120 | 205 |
| A168 | 147 | 205 |
| A169 | 126 | 205 |
| A170 | 135 | 205 |
| A171 | 168 | 205 |
| A172 | 130 | 205 |
| A173 | 127 | 205 |
| A174 | 132 | 205 |
| A175 | 126 | 201 |
| A176 | 135 | 201 |
| A177 | 168 | 201 |
| A178 | 130 | 201 |
| A179 | 127 | 201 |
| A180 | 132 | 201 |
| A181 | 107 | 202 |
| A182 | 138 | 202 |
| A183 | 140 | 202 |
| A184 | 145 | 202 |
| A185 | 147 | 202 |
| A186 | 144 | 202 |
| A187 | 120 | 202 |
| A188 | 115 | 202 |
| A189 | 146 | 202 |
| A190 | 141 | 202 |
| A191 | 142 | 202 |
| A192 | 143 | 202 |
| A193 | 109 | 205 |
| A194 | 103 | 205 |
| A195 | 169 | 205 |
| A196 | 129 | 205 |
| A197 | 116 | 205 |
| A198 | 134 | 205 |
| A199 | 109 | 201 |
| A200 | 103 | 201 |
| A201 | 169 | 201 |
| A202 | 129 | 201 |
| A203 | 116 | 201 |
| A204 | 134 | 201 |
| A205 | 109 | 202 |
| A206 | 103 | 202 |
| A207 | 169 | 202 |
| A208 | 129 | 202 |
| A209 | 116 | 202 |
| A210 | 134 | 202 |
| A211 | 108 | 201 |
| A212 | 107 | 201 |
| A213 | 106 | 201 |
| A214 | 111 | 201 |
| A215 | 110 | 201 |
| A216 | 112 | 201 |
| A217 | 101 | 201 |
| A218 | 119 | 201 |
| A219 | 104 | 201 |
| A220 | 102 | 205 |
| A221 | 105 | 201 |
| A222 | 114 | 201 |
| A223 | 103 | 202 |
| A224 | 116 | 201 |
| A500 | 301 | 303 |
| A501 | 302 | 303 |

As used herein, reference to A(number), refers to an antibody of this table. For instance, A15 used herein refers to A15 in Table 7

### Anti-TL1A Framework Regions

In certain embodiments, an anti-TL1A antibody comprises a heavy chain (HC) framework 1 (FR1) as set forth by SEQ ID NO: 304. In certain embodiments, an anti-TL1A antibody comprises a HC FR2 as set forth by any one of SEQ ID NOS: 305 or 313. In certain embodiments, an anti-TL1A antibody comprises a HC FR3 as set forth by any one of SEQ ID NOS: 306-307, 314-315. In certain embodiments, an anti-TL1A antibody comprises a HC FR4 as set forth by SEQ ID NO: 308. In certain embodiments, an anti-TL1A antibody comprises a LC FR1 as set forth by SEQ ID NO: 309. In certain embodiments, an anti-TL1A antibody comprises a LC FR2 as set forth by SEQ ID NO: 310. In certain embodiments, an anti-TL1A antibody comprises a LC FR3 as set forth by SEQ ID NO: 311. In certain embodiments, an anti-TL1A antibody comprises a LC FR4 as set forth by SEQ ID NO: 312. In a non-limiting example, an anti-TL1A antibody comprises a HC FR1 as set forth by SEQ ID NO: 304, a HC FR2 as set forth by SEQ ID NO: 305, a HC FR3 as set forth by SEQ ID NO: 306, a HC FR4 as set forth by SEQ ID NO: 308, a LC FR1 as set forth by SEQ ID NO: 309, a LC FR2 as set forth by SEQ ID NO: 310, a LC FR3 as set forth by SEQ ID NO: 311, and a LC FR4 as set forth by SEQ ID NO: 312. In a non-limiting example, an anti-TL1A antibody comprises a HC FR1 as set forth by SEQ ID NO: 304, a HC FR2 as set forth by SEQ ID NO: 305, a HC FR3 as set forth by SEQ ID NO: 307, a HC FR4 as set forth by SEQ ID NO: 308, a LC FR1 as set forth by SEQ ID NO: 309, a LC FR2 as set forth by SEQ ID NO: 310, a LC FR3 as set forth by SEQ ID NO: 311, and a LC FR4 as set forth by SEQ ID NO: 312. In certain embodiments, an anti-TL1A antibody comprises a HC FR2 as set forth by SEQ ID NO: 382. In certain embodiments, an anti-TL1A antibody comprises a HC FR3 as set forth by any one of SEQ ID NOS: 383-388. In certain embodiments, an anti-TL1A antibody comprises a LC FR2 as set forth by SEQ ID NO: 389. In certain embodiments, an anti-TL1A antibody comprises a LC FR3 as set forth by SEQ ID NO: 390.

In certain embodiments, an anti-TL1A antibody comprises the heavy chain framework regions set forth in an antibody selected from Table 3. In certain embodiments, an anti-TL1A antibody comprises the light chain framework regions set forth in an antibody selected from Table 4. In certain embodiments, an anti-TL1A antibody comprises the framework regions set forth in any one of the antibodies of Table 7. For instance, an anti-TL1A antibody comprises the framework regions of antibody A15, A29, A30, A31, A32, A33, A34, A35, A36, A37, A38, A39, A40, A41, A42, A43, A44, A45, A46, A47, A48, A49, A50, A51, A52, A53, A54, A55, A56, A57, A58, A59, A60, A61, A62, A63, A64, A65, A66, A67, A68, A69, A70, A71, A72, A73, A74, A75, A76, A77, A78, A79, A81, A82, A83, A85, A86, A87, A88, A89, A90, A91, A92, A93, A94, A95, A96, A97, A98, A99, A100, A101, A102, A103, A104, A105, A107, A108, A109, A110, A111, A112, A113, A114, A115, A116, A117, A118, A119, A120, A121, A122, A123, A124, A125, A126, A127, A128, A129, A130, A132, A133, A134, A135, A136, A137, A138, A139, A140, A141, A142, A143, A144, A145, A146, A147, A148, A149, A150, A151, A152, A153, A154, A155, A156, A157, A158, A159, A160, A161, A162, A163, A164, A165, A166, A167, A168, A169, A170, A171, A172, A173, A174, A175, A176, A177, A178, A179, A180, A181, A182, A183, A184, A185, A186, A187, A188, A189, A190, A191, A192, A193, A194, A195, A196, A197, A198, A199, A200, A201, A202, A203, A204, A205, A206, A207, A208, A209, A210, A211, A212, A213, A214, A215, A216, A217, A218, A219, A220, A221, A222, A223, A224, A500, or A501. In anon-limiting example, an anti-TL1A antibody comprises the framework region of antibody A219. In some instances, an anti-TL1A antibody comprises the framework regions of antibody 5C3D11, 9E12E5, AS12824, AS12823, AS12819, AS12816, AS12825, 12835, 18-7, 21-3, L8, 21-3 V102K, 21-3 V102M, 21-3 V102Q, 21-3 V102 W, 21-3 CDRv, 21-3 CDRv, Clone2, Clone 52, Clone 46, Clone 47, Clone 14, Clone 16L, Clone 17L, Clone 17L-1, Clone 23, Clone A1, Clone 53, Clone E1, Clone 3-17L V-A, Clone 3-17L, Clone L8mod, Clone X-V, Clone X, Clone XL3-6, Clone XL3-10, Clone XL3-15, Clone L3-13, Clone H3-1, Clone H2-2, or Clone H2-5. Table 3 and Table 4 provide the variable region sequences comprising these framework regions. In some instances, an anti-TL1A antibody comprises the framework regions of antibody M1, M2, M3, M4, M5, M6, M7, M8, M9, M10, M11, or M12. Table 3 and Table 4 provide the variable region sequences comprising the framework regions of antibodies M1-M12.

Antibody CDR and framework regions may be defined by the Kabat, Chothia, or IMGT methods.

In some embodiments, an anti-TL1A antibody comprises a heavy chain variable framework region comprising a human IGHV1-46*02 framework or a modified human IGHV1-46*02 framework, and a light chain variable framework region comprising a human IGKV3-20 framework or a modified human IGKV3-20 framework; wherein the heavy chain variable framework region and the light chain variable framework region collectively comprise no or fewer than nine amino acid modification(s) from the human IGHV1-46*02 framework and the human IGKV3-20 framework. In some embodiments, the amino acid modification(s) comprise: (a) a modification at amino acid position 45 in the heavy chain variable region; (b) a modification at amino acid position 47 in the heavy chain variable region; (c) a modification at amino acid position 55 in the heavy chain variable region; (d) a modification at amino acid position 78 in the heavy chain variable region; (e) a modification at amino acid position 80 in the heavy chain variable region; (f) a modification at amino acid position 82 in the heavy chain variable region; (g) a modification at amino acid position 89 in the heavy chain variable region; or (h) a modification at amino acid position 91 in the heavy chain variable region, per Kabat numbering; or a combination of two or more modifications selected from (a) to (h). In some embodiments, the amino acid modification(s) comprise (a) R45K, (b) A47R, (c) M55I, (d) V78A, (e) M80I, (f) R82T, (g) V89A, or (h) M91L in the heavy chain variable region, per Kabat numbering; or a combination of two or more modifications selected from (a) to (h). In some embodiments, the amino acid modification(s) comprise: A47R. In some embodiments, the amino acid modification(s) comprise: A47R, M55I, V78A, M80I, R82T, V89A, and M91L; A47R, M80I, and R82T; A47R, M80I, R82T, V89A, and M91L; or A47R, M55I, V78A, M80I, V89A, and M91L. In some embodiments, the amino acid modification(s) comprise: R45K and A47R. In some embodiments, the amino acid modification(s) comprise: R45K, A47R, V89A, and M91L. In some embodiments, the amino acid modification(s) comprise: R45K and A47R, and M80I. In some embodiments, the amino acid modification(s) comprise: R45K, A47R, M80I, and M91L; R45K, A47R, V78A, M80I, V89A, and M91L; R45K, A47R, M55I, V78A, M80I, R82T, V89A, and M91L; R45K, A47R, M80I, V89A, and M91L; R45K, A47R, M55I, M80I, R82T, V89A, and M91L; R45K, A47R, M80I, and V89A; R45K, A47R, M80I, R82T, V89A, M91L; or R45K, A47R, M55I, M80I, V89A, and M91L. In some embodiments, the amino acid modification(s) comprise: R45K. In some embodiments, the amino acid modification(s) comprise: R45K and V78A. In some embodiments, the amino acid modification(s) comprise: V78A. In some embodiments, the amino acid modification(s) comprise: V78A and V89A; V78A and M80I; or V78A, M80I, and R82T. In some embodiments, the amino acid modification(s) comprise: V89A. In some embodiments, the amino acid modification(s) comprise: M80I. In some embodiments, the amino acid modification(s) comprises: (a) a modification at amino acid position 54 in the light chain variable region; and/or (b) a modification at amino acid position 55 in the light chain variable region, per Kabat numbering. In some embodiments, the amino acid modification(s) comprises L54P in the light chain variable region, per Kabat numbering. In some embodiments, the amino acid modification(s) comprises L55 W in the light chain variable region, per Kabat numbering.

In some embodiments, an anti-TL1A antibody comprises a heavy chain framework comprising SEQ ID NO: 301 (X1VQLVQSGAEVKKPGASVKVSCKAS[HCDR1]WVX2QX3PGQGLEWX4G[HCDR2]RX 5TX6TX7DTSTSTX8YX9ELSSLRSEDTAVYYCAR[HCDR3]WGQGTTVTVSS) or SEQ ID NO: 302 (X1VQLVQSGAEVKKPGASVKVSCKAS[HCDR1]WVX2QX3PGQGLEWX4G[HCDR2]RX 5TX6TX7DTSTSTX8YX9ELSSLRSEDTAVYYC[HCDR3]WGQGTTVTVSS). In some cases, X1 is Q. In some cases, X1=E. In some cases, X2=R. In some cases, X2=K. In some cases, X3=A. In some cases, X3=R. In some cases, X4=M. In some cases, X4=I. In some cases, X5=V. In some cases, X5=A. In some cases, X6=M. In some cases, X6=I. In some cases, X7=R. In some cases, X7=T. In some cases, X8=V. In some cases, X8=A. In some cases, X9=M. In some cases, X9=L. In some embodiments, X1 is at position 1 of IGHV1-46*02 as determined by Kabat numbering. In some embodiments, X2 is at position 45 of IGHV1-46*02 as determined by Kabat numbering. In some embodiments, X3 is at position 47 of IGHV1-46*02 as determined by Kabat numbering. In some embodiments, X4 is at position 55 of IGHV1-46*02 as determined by Kabat numbering. In some embodiments, X5 is at position 78 of IGHV1-46*02 as determined by Kabat numbering. In some embodiments, X6 is at position 80 of IGHV1-46*02 as determined by Kabat numbering. In some embodiments, X7 is at position 82 of IGHV1-46*02 as determined by Kabat numbering. In some embodiments, X8 is at position 89 of IGHV1-46*02 as determined by Kabat numbering. In some embodiments, X9 is at position 91 of IGHV1-46*02 as determined by Kabat numbering.

In one aspect, provided herein is a first embodiment of an anti-TL1A antibody comprising a heavy chain framework comprising IGHV1-46*02, or a variant thereof, wherein the variant comprises between about 1 and about 9 amino acid substitutions, or between about 1 and about 20 amino acid substitutions, or about 1, 2, 3, 4, 5, 6, 7, 8, 9, 10, 11, 12, 13, 14, 15, 16, 17, 18, 19, or 20 amino acid substitutions from IGHV1-46*02 framework. Additional embodiments include: (2) The anti-TL1A of embodiment (1), wherein the heavy chain framework comprises SEQ ID NO: 301. (3) The anti-TL1A of embodiment 2, wherein X1=Q. (4) The anti-TL1A of embodiment 2, wherein X1=E. (5) The anti-TL1A of any one of embodiments 2-4, wherein X2=R. (6) The anti-TL1A of any one of embodiments 2-4, wherein X2=K. (7) The anti-TL1A of any one of embodiments 2-6, wherein X3=A. (8) The anti-TL1A of any one of embodiments 2-6, wherein X3=R. (9) The anti-TL1A of any one of embodiments 2-8, wherein X4=M. (10) The anti-TL1A of any one of embodiments 2-8, wherein X4=I. (11) The anti-TL1A of any one of embodiments 2-10, wherein X5=V. (12) The anti-TL1A of any one of embodiments 2-10, wherein X5=A. (13) The anti-TL1A of any one of embodiments 2-12, wherein X6=M. (14) The anti-TL1A of any one of embodiments 2-12, wherein X6=I. (15) The anti-TL1A of any one of embodiments 2-14, wherein X7=R. (16) The anti-TL1A of any one of embodiments 2-14, wherein X7=T. (17) The anti-TL1A of any one of embodiments 2-16, wherein X8=V. (18) The anti-TL1A of any one of embodiments 2-16, wherein X8=A. (19) The anti-TL1A of any one of embodiments 2-18, wherein X9=M. (20) The anti-TL1A of any one of embodiments 2-4, wherein X9=L. (21) The anti-TL1A of any one of embodiments 1-20, comprising antibody A. (22) The anti-TL1A of any one of embodiments 1-20, comprising antibody B. (23) The anti-TL1A of any one of embodiments 1-20, comprising antibody C. (24) The anti-TL1A of any one of embodiments 1-20, comprising antibody D. (25) The anti-TL1A of any one of embodiments 1-20, comprising antibody E. (26) The anti-TL1A of any one of embodiments 1-20, comprising antibody F. (27) The anti-TL1A of any one of embodiments 1-20, comprising antibody G or I. (28) The anti-TL1A of any one of embodiments 1-20, comprising antibody H. (34) The anti-TL1A of any one of embodiments 1-33, comprising a light chain comprising a light chain framework comprising IGKV3-20*01, or a variant thereof, wherein the variant comprises between about 1 and about 2 substitutions, or between about 1 and about 20 amino acid substitutions, or about 1, 2, 3, 4, 5, 6, 7, 8, 9, 10, 11, 12, 13, 14, 15, 16, 17, 18, 19, or 20 amino acid substitutions in the framework. (35) The anti-TL1A antibody of embodiment 34, wherein X10 is L. (36) The anti-TL1A antibody of embodiment 34, wherein X10 is P. (37) The anti-TL1A antibody of any one of embodiments 34-36, wherein X1I is L. (38) The anti-TL1A antibody of any one of embodiments 34-36, wherein X11 is W.

In some embodiments, an anti-TL1A antibody comprises a light chain framework comprising SEQ ID NO: 303 (EIVLTQSPGTLSLSPGERATLSC[LCDR1]WYQQKPGQAPRX10X11IY[LCDR2]GIPDRFS GSGSGTDFTLTISRLEPEDFAVYYC[LCDR3]FGGGTKLEIK). In some cases, X10 is L. In some cases, X10 is P. In some cases, X11 is L. In some cases, X11 is W. In some embodiments, X10 is at position 54 of IGKV3-20*01 as determined by Kabat numbering. In some embodiments, X11 is at position 55 of IGKV3-20*01 as determined by Kabat numbering.

In some embodiments, an anti-TL1A antibody comprises a heavy chain framework comprising IGHV1-46*02. In some embodiments, an anti-TL1A antibody comprises a heavy chain framework comprising a variant of IGHV1-46*02 comprising between about 1 and about 20 amino acid substitutions from SEQ ID NO: 316. In some embodiments, an anti-TL1A antibody comprises a heavy chain framework comprising a variant of IGHV1-46*02 comprising between about 1 and about 9 amino acid substitutions from SEQ ID NO: 316. In some embodiments, an anti-TL1A antibody comprises a heavy chain framework comprising a variant of IGHV1-46*02 comprising about 1, 2, 3, 4, 5, 6, 7, 8, 9, 10, 11, 12, 13, 14, 15, 16, 17, 18, 19, or 20 amino acid substitutions from SEQ ID NO: 316 in the framework. In some cases, the heavy chain framework substitution comprises Q1E, as determined by Kabat numbering. In some cases, the heavy chain framework substitution comprises R45K, as determined by Kabat numbering. In some cases, the heavy chain framework substitution comprises A47R, as determined by Kabat numbering. In some cases, the heavy chain framework substitution comprises M55I, as determined by Kabat numbering. In some cases, the heavy chain framework substitution comprises V78A, as determined by Kabat numbering. In some cases, the heavy chain framework substitution comprises M80I, as determined by Kabat numbering. In some cases, the heavy chain framework substitution comprises R82T, as determined by Kabat numbering. In some cases, the heavy chain framework substitution comprises V89A, as determined by Kabat numbering. In some cases, the heavy chain framework substitution comprises M91L, as determined by Kabat numbering.

In some embodiments, an anti-TL1A antibody comprises a light chain framework comprising IGKV3-20*01. In some embodiments, an anti-TL1A antibody comprises a variant of IGKV3-20*01 comprising between about 1 and about 20 amino acid substitutions from SEQ ID NO: 317. In some embodiments, an anti-TL1A antibody comprises a variant of IGKV3-20*01 comprising about 1 amino acid substitution from SEQ ID NO: 317. In some embodiments, an anti-TL1A antibody comprises a light chain framework comprising a variant of IGKV3-20*01 comprising about 2 amino acid substitutions from SEQ ID NO: 317. In some embodiments, an anti-TL1A antibody comprises a light chain framework comprising a variant of IGKV3-20*01 comprising about 1, 2, 3, 4, 5, 6, 7, 8, 9, 10, 11, 12, 13, 14, 15, 16, 17, 18, 19, or 20 amino acid substitutions from SEQ ID NO: 317 in the framework. In some cases, the light chain framework substitution comprises Q1E, as determined by Kabat numbering. In some cases, the light chain framework substitution comprises R45K, as determined by Kabat numbering.

In some embodiments, an anti-TL1A antibody comprises a framework region of Table 5.

TABLE 5
Example framework sequences

| SEQ | | |
|---|---|---|
| ID | | |
| NO | Description | Sequence |
| 301 | Variable Heavy 1 | X1VQLVQSGAEVKKPGASVKVSCKAS[HCDR1]WVX2QX3PGQG |
| | | LEWX4G[HCDR2]RX5TX6TX7DTSTSTX8YX9ELSSLRSEDTAVY |
| | | YCAR[HCDR3]WGQGTTVTVSS |
| | | wherein each of X1-X11 is independently selected from A, R, N, D, C, |
| | | Q E, G, H, I, L, K, M, F, P, S, T, W, Y, or V |
| | | In some cases, X1 = Q or E |
| | | In some cases, X2 = R or K |
| | | In some cases, X3 = A or R |
| | | In some cases, X4 = M or I |
| | | In some cases, X5 = V or A |
| | | In some cases, X6 = M or I |
| | | In some cases, X7 = R or T |
| | | In some cases, X8 = V or A |
| | | In some cases, X9 = M or L |
| 302 | Variable Heavy 2 | X1VQLVQSGAEVKKPGASVKVSCKAS[HCDR1]WVX2QX3PGQG |
| | | LEWX4G[HCDR2]RX5TX6TX7DTSTSTX8YX9ELSSLRSEDTAVY |
| | | YC[HCDR3]WGQGTTVTVSS |
| | | wherein each of X1-X11 is independently selected from A, R, N, D, C, |
| | | Q E, G, H, I, L, K, M, F, P, S, T, W, Y, or V |
| | | In some cases, X1 = Q or E |
| | | In some cases, X2 = R or K |
| | | In some cases, X3 = A or R |
| | | In some cases, X4 = M or I |
| | | In some cases, X5 = V or A |
| | | In some cases, X6 = M or I |
| | | In some cases, X7 = R or T |
| | | In some cases, X8 = V or A |
| | | In some cases, X9 = M or L |
| 303 | Variable Light | EIVLTQSPGTLSLSPGERATLSC[LCDR1]WYQQKPGQAPRX10X11 |
| | | IY[LCDR2]GIPDRFSGSGSGTDFTLTISRLEPEDFAVYYC[LCDR3]F |
| | | GGGTKLEIK |
| | | wherein each of X10 and X11 is independently selected from A, R, N, |
| | | D, C, Q E, G, H, I, L, K, M, F, P, S, T, W, Y, or V |
| | | In some cases, X10 = L or P |
| | | In some cases, X11 = L or W |
| 304 | 219 HC FR1, | QVQLVQSGAEVKKPGASVKVSCKAS |
| | 212 HC FR1 | |
| 305 | 219 HC FR2 | WVKQRPGQGLEWMG |
| 306 | 219 HC FR3a | RVTITRDTSTSTVYLELSSLRSEDTAVYYCAR |
| 307 | 219 HC FR3b | RVTITRDTSTSTVYLELSSLRSEDTAVYYC |
| 308 | 219 HC FR4, | WGQGTTVTVSS |
| | 212 HC FR4 | |
| 309 | 219 LC FR1, | EIVLTQSPGTLSLSPGERATLSC |
| | 212 LC FR1 | |
| 310 | 219 LC FR2, | WYQQKPGQAPRPLIY |
| | 212 LC FR2 | |
| 311 | 219 LC FR3, | GIPDRFSGSGSGTDFTLTISRLEPEDFAVYYC |
| | 212 LC FR3 | |
| 312 | 219 LC FR4, | FGGGTKLEIK |
| | 212 LC FR4 | |
| 313 | 212 HC FR2 | WVRQRPGQGLEWIG |
| 314 | 212 HC FR3a | RATITTDTSTSTAYLELSSLRSEDTAVYYCAR |
| 315 | 212 HC FR3b | RATITTDTSTSTAYLELSSLRSEDTAVYYC |
| 382 | HCFR2 | WVRQAPGQGLEWMG |
| 383 | HCFR3 | RVTMTRDTSTSTVYMELSSLRSEDTAVYYC |
| 384 | HCFR3 | RATITTDTSASTAYLQLSSLRSEDTAVYYC |
| 385 | HCFR3 | RVTITRDTSASTVYMELSSLRSEDTAVYYC |
| 386 | HCFR3 | RVTITRDTSASTAYMELSSLRSEDTAVYYC |
| 387 | HCFR3 | RVTMTRDTSTSTVYMELSSLRSEDTAVYYCSR |
| 388 | HCFR3 | RVTMTRDTSTSTVYMELSSLRSEDTAVYYCAR |
| 389 | LCFR2 | WYQQKPGQAPRLLIY |
| 390 | LCFR3 | GVPDRFSGSGSGTDYTLTISRLEPEDFAVYYC |
| 316 | IGHV1-46*02 | QVQLVQSGAEVKKPGASVKVSCKASGYTFNSYYMHWVRQAPG |
| | | QGLEWMGIINPSGGSTSYAQKFQGRVTMTRDTSTSTVYMELSSL |
| | | RSEDTAVYYCAR |
| 317 | IGKV3-20*01 | EIVLTQSPGTLSLSPGERATLSCRASQSVSSSYLAWYQQKPGQAP |
| | | RLLIYGASSRATGIPDRFSGSGSGTDFTLTISRLEPEDFAVYYCQQ |
| | | YGSSP |

### Anti-TL1A Variable Regions

In one aspect, provided herein is an anti-TL1A antibody comprising a heavy chain variable region comprising an amino acid sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 101-169; and a light chain variable region at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 201-220.

In some cases, an anti-TL1A antibody comprises heavy chain and light chain variable regions, each having a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to antibody A15, A29, A30, A31, A32, A33, A34, A35, A36, A37, A38, A39, A40, A41, A42, A43, A44, A45, A46, A47, A48, A49, A50, A51, A52, A53, A54, A55, A56, A57, A58, A59, A60, A61, A62, A63, A64, A65, A66, A67, A68, A69, A70, A71, A72, A73, A74, A75, A76, A77, A78, A79, A81, A82, A83, A85, A86, A87, A88, A89, A90, A91, A92, A93, A94, A95, A96, A97, A98, A99, A100, A101, A102, A103, A104, A105, A107, A108, A109, A110, A111, A112, A113, A114, A115, A116, A117, A118, A119, A120, A121, A122, A123, A124, A125, A126, A127, A128, A129, A130, A132, A133, A134, A135, A136, A137, A138, A139, A140, A141, A142, A143, A144, A145, A146, A147, A148, A149, A150, A151, A152, A153, A154, A155, A156, A157, A158, A159, A160, A161, A162, A163, A164, A165, A166, A167, A168, A169, A170, A171, A172, A173, A174, A175, A176, A177, A178, A179, A180, A181, A182, A183, A184, A185, A186, A187, A188, A189, A190, A191, A192, A193, A194, A195, A196, A197, A198, A199, A200, A201, A202, A203, A204, A205, A206, A207, A208, A209, A210, A211, A212, A213, A214, A215, A216, A217, A218, A219, A220, A221, A222, A223, A224, A500, or A501. In a non-limiting example, an anti-TL1A antibody comprises the variable regions of antibody A219. In some instances, an anti-TL1A antibody comprises heavy chain and light chain variable regions, each having a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to antibody 5C3D11, 9E12E5, AS12824, AS12823, AS12819, AS12816, AS12825, 12835, 18-7, 21-3, L8, 21-3 V102K, 21-3 V102M, 21-3 V102Q, 21-3 V102 W, 21-3 CDRv, 21-3 CDRv, Clone 2, Clone 52, Clone 46, Clone 47, Clone 14, Clone 16L, Clone 17L, Clone 17L-1, Clone 23, Clone A1, Clone 53, Clone E1, Clone 3-17L V-A, Clone 3-17L, Clone L8mod, Clone X-V, Clone X, Clone XL3-6, Clone XL3-10, Clone XL3-15, Clone L3-13, Clone H3-1, Clone H2-2, or Clone H2-5. Table 3 and Table 4 provide the variable region sequences. In some instances, an anti-TL1A antibody comprises heavy chain and light chain variable regions, each having a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to antibody M1, M2, M3, M4, M5, M6, M7, M8, M9, M10, M11, or M12. Table 3 and Table 4 provide the variable region sequences of antibodies M1-M12.

Further provided herein is an anti-TL1A antibody comprising a heavy chain variable region and a light chain variable region. In some embodiments, the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 101-169 or a sequence having about 1, 2, 3, 4, 5, 6, 7, 8, 9 or 10 amino acid substitutions or deletions as compared to any one of SEQ ID NOS: 101-169.

In some embodiments, the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 201-220 or the light chain variable region comprises a sequence having about 1, 2, 3, 4, 5, 6, 7, 8, 9 or 10 amino acid substitutions or deletions as compared to any one of SEQ ID NOS: 201-220.

In some embodiments, the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 170-200, 267, 268 or the heavy chain variable region comprises a sequence having about 1, 2, 3, 4, 5, 6, 7, 8, 9 or 10 amino acid substitutions or deletions as compared to any one of SEQ ID NOS: 170-200, 267, 268. In some embodiments, the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 221-236 or the light chain variable region comprises a sequence having about 1, 2, 3, 4, 5, 6, 7, 8, 9 or 10 amino acid substitutions or deletions as compared to any one of SEQ ID NOS: 221-236.

In some embodiments, the heavy chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 269-289 or the heavy chain variable region comprises a sequence having about 1, 2, 3, 4, 5, 6, 7, 8, 9 or 10 amino acid substitutions or deletions as compared to any one of SEQ ID NOS: 269-289. In some embodiments, the light chain variable region comprises a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 237-266 or the light chain variable region comprises a sequence having about 1, 2, 3, 4, 5, 6, 7, 8, 9 or 10 amino acid substitutions or deletions as compared to any one of SEQ ID NOS: 237-266.

### Anti-TL1A Constant Regions

In some embodiments, one or more amino acid modifications may be introduced into the Fragment crystallizable (Fc) region of a human or humanized antibody, thereby generating an Fc region variant. An Fc region may comprise a C-terminal region of an immunoglobulin heavy chain that comprises a hinge region, CH2 domain, CH3 domain, or any combination thereof. As used herein, an Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution, addition, or deletion) at one or more amino acid positions. In one embodiment, the Fc region comprises any one of SEQ ID NOS: 320-367. In some embodiments, the anti-TL1A antibody comprises a constant region comprising any one of SEQ ID NOS: 319, 368-381.

In some embodiments, antibodies of this disclosure have a reduced effector function as compared to a human IgG. Effector function refers to a biological event resulting from the interaction of an antibody Fc region with an Fc receptor or ligand. Non-limiting effector functions include C1q binding, complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g., B cell receptor), and B cell activation. In some cases, antibody-dependent cell-mediated cytotoxicity (ADCC) refers to a cell-mediated reaction in which nonspecific cytotoxic cells expressing Fc receptors (e.g., natural killer cells, neutrophils, macrophages) recognize bound antibody on a target cell, subsequently causing lysis of the target cell. In some cases, complement dependent cytotoxicity (CDC) refers to lysing of a target cells in the presence of complement, where the complement action pathway is initiated by the binding of C1q to antibody bound with the target.

Some Fc regions have a natural lack of effector function, and some Fc regions can comprise mutations that reduce effector functions. For instance, IgG4 has low ADCC and CDC activities and IgG2 has low ADCC activity.

The disclosure provides antibodies comprising Fc regions characterized by exhibiting ADCC that is reduced by at least about 30%, at least about 40%, at least about 50%, at least about 60%, at least about 70% or more as compared to an antibody comprising a non-variant Fc region, i.e., an antibody with the same sequence identity but for the substitution(s) that decrease ADCC (such as human IgG1, SEQ ID NO: 320). The disclosure provides antibodies comprising Fc regions characterized by exhibiting CDC that is reduced by at least about 30%, at least about 40%, at least about 50%, at least about 60%, at least about 70% or more as compared to an antibody comprising a non-variant Fc region, i.e., an antibody with the same sequence identity but for the substitution(s) that decrease CDC (such as human IgG1, SEQ ID NO: 320). In certain embodiments, the antibodies of this disclosure have reduced effector function as compared with human IgG1. In certain embodiments, antibodies herein have no detectable ADCC activity. In certain embodiments, the reduction and/or abatement of ADCC activity may be attributed to the reduced affinity antibodies described herein exhibit for Fc ligands and/or receptors. In certain embodiments, antibodies herein exhibit no detectable CDC activities. In some embodiments, the reduction and/or abatement of CDC activity may be attributed to the reduced affinity antibodies described herein exhibit for Fc ligands and/or receptors.

In some embodiments, antibodies comprising Fc regions described herein exhibit decreased affinities to C1q relative to an unmodified antibody (e.g., human IgG1 having SEQ ID NO: 320). In some embodiments, antibodies herein exhibit affinities for C1q receptor that are at least 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold less than an unmodified antibody. In some embodiments, antibodies herein exhibit affinities for C1q that are at least 90%, at least 80%, at least 70%, at least 60%, at least 50%, at least 40%, at least 30%, at least 20%, at least 10%, or at least 5% less than an unmodified antibody.

In some embodiments, the antibodies of this disclosure are variants that possess some but not all effector functions, which make it a desirable candidate for applications in which the half-life of the antibody in vivo is important yet certain effector functions (such as complement and ADCC) are unnecessary or deleterious.

In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity) but retains FcRn binding ability.

In some embodiments, antibodies are tested for binding to Fcγ receptors and complement C1q by ELISA. In some embodiments, antibodies are tested for the ability to activate primary human immune cells in vitro, for example, by assessing their ability to induce expression of activation markers.

In some embodiments, assessment of ADCC activity of an anti-TL1A antibody comprises adding the antibody to target cells in combination with immune effector cells, which may be activated by the antigen antibody complexes resulting in cytolysis of the target cell. Cytolysis may be detected by the release of label (e.g. radioactive substrates, fluorescent dyes or natural intracellular proteins) from the lysed cells. Useful effector cells for such assays include peripheral blood mononuclear cells (PBMC) and Natural Killer (NK) cells. Specific examples of in vitro ADCC assays are described in Wisecarver et al., 1985 79:277-282; Bruggemann et al., 1987, J Exp Med 166:1351-1361; Wilkinson et al., 2001, J Immunol Methods 258:183-191; Patel et al., 1995 J Immunol Methods 184:29-38. Alternatively, or additionally, ADCC activity of the antibody of interest may be assessed in vivo, e.g., in an animal model such as that disclosed in Clynes et al., 1998, PNAS USA 95:652-656.

In some embodiments, an assessment of complement activation, a CDC assay, may be performed as described in Gazzano-Santoro et al., 1996, J. Immunol. Methods, 202:163.

Non-limiting examples of Fc mutations in IgG1 that may reduce ADCC and/or CDC include substitutions at one or more of positions: 231, 232, 234, 235, 236, 237, 238, 239, 264, 265, 267, 269, 270, 297, 299, 318, 320, 322, 325, 327, 328, 329, 330, and 331 in IgG1, where the numbering system of the constant region is that of the EU index as set forth by Kabat. In certain embodiments, the antibodies of this disclosure have reduced effector function as compared with human IgG1.

In some embodiments, an antibody comprises an IgG1 Fc region comprising one or more of the following substitutions according to the Kabat numbering system: N297A, N297Q, N297D, D265A, S228P, L235A, L237A, L234A, E233P, L234V, C236 deletion, P238A, A327Q, P329A, P329G, L235E, P331S, L234F, 235G, 235Q, 235R, 235S, 236F, 236R, 237E, 237K, 237N, 237R, 238A, 238E, 238G, 238H, 238I, 238V, 238 W, 238Y, 248A, 254D, 254E, 254G, 254H, 254I, 254N, 254P, 254Q, 254T, 254V, 255N, 256H, 256K, 256R, 256V, 264S, 265H, 265K, 265S, 265Y, 267G, 267H, 267I, 267K, 268K, 269N, 269Q, 270A, 270G, 270M, 270N, 271T, 272N, 279F, 279K, 279L, 292E, 292F, 292G, 292I, 293 S, 301 W, 304E, 311E, 311G, 311S, 316F, 327T, 328V, 329Y, 330R, 339E, 339L, 343I, 343V, 373A, 373G, 373S, 376E, 376 W, 376Y, 380D, 382D, 382P, 385P, 424H, 424M, 424V, 434I, 438G, 439E, 439H, 439Q, 440A, 440D, 440E, 440F, 440M, 440T, 440V.

In some embodiments, an antibody comprises a Fc region selected from the representative sequences disclosed in Table 8, Table 6, or Table 9. In some embodiments, an antibody comprises an IgG1 Fc region comprising E233P, according to the Kabat numbering system. In some embodiments, an antibody comprises an IgG4 Fc region comprising S228P and L235E. In some embodiments, an antibody comprises an IgG1 Fc region comprising L235E, according to the Kabat numbering system. In some embodiments, an antibody comprises an IgG1 Fc region comprising L234A and L235A, according to the Kabat numbering system. In some embodiments, an antibody comprises an IgG1 Fc region comprising L234A, L235A, and G237A, according to the Kabat numbering system. In some embodiments, an antibody comprises an IgG1 Fc region comprising L234A, L235A, P329G, according to the Kabat numbering system. In some embodiments, an antibody comprises an IgG1 Fc region comprising L234F, L235E, and P331 S, according to the Kabat numbering system. In some embodiments, an antibody comprises an IgG1 Fc region comprising L234A, L235E, and G237A, according to the Kabat numbering system. In some embodiments, an antibody comprises an IgG1 Fc region comprising L234A, L235E, G237A, and P331S, according to the Kabat numbering system. In some embodiments, an antibody comprises an IgG1 Fc region comprising L234A, L235A, G237A, P238S, H268A, A330S, and P331S (IgG1σ), according to the Kabat numbering system. In some embodiments, an antibody comprises an IgG1 Fc region comprising L234A, L235A, and P329A, according to the Kabat numbering system. In some embodiments, an antibody comprises an IgG1 Fc region comprising G236R and L328R, according to the Kabat numbering system. In some embodiments, an antibody comprises an IgG1 Fc region comprising G237A, according to the Kabat numbering system. In some embodiments, an antibody comprises an IgG1 Fc region comprising F241A, according to the Kabat numbering system. In some embodiments, an antibody comprises an IgG1 Fc region comprising V264A, according to the Kabat numbering system. In some embodiments, an antibody comprises an IgG1 Fc region comprising D265A, according to the Kabat numbering system. In some embodiments, an antibody comprises an IgG1 Fc region comprising D265A and N297A, according to the Kabat numbering system. In some embodiments, an antibody comprises an IgG1 Fc region comprising D265A and N297G, according to the Kabat numbering system. In some embodiments, an antibody comprises an IgG1 Fc region comprising D270A, according to the Kabat numbering system. In some embodiments, an antibody comprises an IgG1 Fc region comprising N297A, according to the Kabat numbering system. In some embodiments, an antibody comprises an IgG1 Fc region comprising N297G, according to the Kabat numbering system. In some embodiments, an antibody comprises an IgG1 Fc region comprising N297D, according to the Kabat numbering system. In some embodiments, an antibody comprises an IgG1 Fc region comprising N297Q, according to the Kabat numbering system. In some embodiments, an antibody comprises an IgG1 Fc region comprising P329A, according to the Kabat numbering system. In some embodiments, an antibody comprises an IgG1 Fc region comprising P329G, according to the Kabat numbering system. In some embodiments, an antibody comprises an IgG1 Fc region comprising P329R, according to the Kabat numbering system. In some embodiments, an antibody comprises an IgG1 Fc region comprising A330L, according to the Kabat numbering system. In some embodiments, an antibody comprises an IgG1 Fc region comprising P331A, according to the Kabat numbering system. In some embodiments, an antibody comprises an IgG1 Fc region comprising P331 S, according to the Kabat numbering system. In some embodiments, an antibody comprises an IgG2 Fc region. In some embodiments, an antibody comprises an IgG4 Fc region. In some embodiments, an antibody comprises an IgG4 Fc region comprising S228P, according to the Kabat numbering system. In some embodiments, an antibody comprises an IgG4 Fc region comprising S228P, F234A, and L235A, according to the Kabat numbering system. In some embodiments, an antibody comprises an IgG2-IgG4 cross-subclass (IgG2/G4) Fc region. In some embodiments, an antibody comprises an IgG2-IgG3 cross-subclass Fc region. In some embodiments, an antibody comprises an IgG2 Fc region comprising H268Q, V309L, A330S, and P331S, according to the Kabat numbering system. In some embodiments, an antibody comprises an IgG2 Fc region comprising V234A, G237A, P238S, H268A, V309L, A330S, and P331S, according to the Kabat numbering system. In some embodiments, an antibody comprises a Fc region comprising high mannose glycosylation.

In some embodiments, an antibody comprises an IgG4 Fc region comprising a S228P substitution, according to the Kabat numbering system. In some embodiments, an antibody comprises an IgG4 Fc region comprising an A330S substitution, according to the Kabat numbering system. In some embodiments, an antibody comprises an IgG4 Fe region comprising a P331S substitution, according to the Kabat numbering system.

In some embodiments, an antibody comprises an IgG2 Fe region comprising an A330S substitution, according to the Kabat numbering system. In some embodiments, an antibody comprises an IgG2 Fe region comprising an P331 S substitution, according to the Kabat numbering system. In some embodiments, an antibody comprises an IgG2 Fe region comprising an 234A substitution, according to the Kabat numbering system. In some embodiments, an antibody comprises an IgG2 Fe region comprising an 237A substitution, according to the Kabat numbering system.

In certain embodiments, an anti-TL1A described herein comprises a Fe region as shown in Table 6.

TABLE 6
Fc Mutations

| | Constant Region (SEQ ID NO) | | |
|---|---|---|---|
| Mutations | K_DL | R_EM | K_EM |
| Wild-type IgG1 | 320 | 321 | 322 |
| L235E | 323 | 324 | 325 |
| L234A, L235A | 326 | 327 | 328 |
| L234A, L235A, G237A | 329 | 330 | 331 |
| L234A, L235A, P329G | 332 | 333 | 334 |
| L234F, L235E, P331S | 335 | 336 | 337 |
| L234A, L235E, G237A | 338 | 339 | 340 |
| L234A, L235E, G237A, P331S | 341 | 342 | 343 |
| L234A, L235A, P329A | 344 | 345 | 346 |
| D265A | 347 | 348 | 349 |
| N297G | 350 | 351 | 352 |
| D265A, N297A | 353 | 354 | 355 |
| D265A, N297G | 356 | 357 | 358 |
| L235A, G237A | 359 | 360 | 361 |
| Wild-type IgG4 | 362 | | |

TABLE 8
Representative approaches to abrogating effector function

| Mutation(s) | Effect |
|---|---|
| E233P | Decreases binding to FcγRI, II, III |
| S228P, L235E SPLE in IgG4 | Decreases binding to FcγRI |
| L235E | Decreases binding to FcγRs |
| L234A, L235A | Decreases binding to FcγRI, II, III |
| L234A, L235A, G237A | Decreases binding to FcγRI, II, III, C1q |
| L234A, L235A, P329G | Decreases binding to FcγRI, II, III, C1q |
| L234F, L235E, P331S | Decreases binding to FcγRI, II, III, C1q |
| L234A, L235E, G237A | Decreases binding to FcγRI, II, III, C1q |
| L234A, L235E, G237A, P331S | Decreases binding to FcγRI, II, III, C1q |
| L234A, L235A, G237A, P238S, H268A, | Decreases binding to FcγRI, IIa, IIb, IIIa |
| A330S, P331S (IgG1σ) | |
| L234A, L235A, P329A | Decreases binding to FcγRI, II, III, C1q |
| G236R, L328R | Decreases binding to FcγRI, II, III |
| G237A | Decreases binding to FcγRII |
| F241A | Decreases binding to C1q |
| V264A | Decreases binding to C1q |
| D265A | Decreases binding to FcγRI, II, III |
| D265A, N297A | Decreases binding to FcγRI, II, III, C1q |
| D265A, N297G | Decreases binding to FcγRI, II, III, C1q |
| D270A | Decreases binding to C1q |
| N297A, G, D, Q | Elimination of N-linked glycosylation |
| | Decreases binding to FcγRI, II, III, C1q |
| P329A, G, R | Decreases binding to C1q |
| A330L | Decreases binding to C1q |
| P331A, S | Diminished C1q binding |
| IgG2 | Decreases binding to FcγRs |
| IgG4 | Decreases binding to FcγRs; |
| | Does not activate complement system |
| S228P | Prevent IgG4 Fab arm exchange |
| S228P, F234A, L235A (IgG4) | Decreases binding to FcγRI, IIa, IIIa |
| IgG2-IgG4 cross-subclass (IgG2/G4) | Decreases binding to FcγRI, II, III, C1q |
| IgG2-IgG3 cross-subclass | Decreases binding to FcγRs; |
| | Decreases binding to C1q |
| H268Q, V309L, A330S, P331S (IgG2m4) | Decreases binding to FcγRI, II, III, C1q |
| V234A, G237A, P238S, H268A, V309L, | Decreases binding to FcγRI, IIa, IIb, IIIa, C1q |
| A330S, P331S (IgG2σ) | |
| High mannose glycosylation | Decreases binding to C1q |

TABLE 9
Fc and Constant Regions

SEQ ID NO: 319 Light Chain Constant
RTVAAPSVFIFPPSDEQLKSGTASVVCLLNNFYPREAKVQWKVDNALQSGNSQESVTEQDSKD
STYSLSSTLTLSKADYEKHKVYACEVTHQGLSSPVTKSFNRGEC
SEQ ID NO: 320 IgG1 Constant
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEALH
NHYTQKSLSLSPGK
SEQ ID NO: 321 IgG1 Constant
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCPAPELLGGPSVFLFPPK
PKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALH
NHYTQKSLSLSPGK
SEQ ID NO: 322 IgG1 Constant
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALH
NHYTQKSLSLSPGK
SEQ ID NO: 323 Fc1 (L235E)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELEGGPSVFLFPPK
PKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEALH
NHYTQKSLSLSPGK
SEQ ID NO: 324 Fc2 (L235E)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCPAPELEGGPSVFLFPPK
PKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALH
NHYTQKSLSLSPGK
SEQ ID NO: 325 Fc3 (L235E)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELEGGPSVFLFPPK
PKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALH
NHYTQKSLSLSPGK
SEQ ID NO: 326 Fc4 (L234A, L235A)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPEAAGGPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEAL
HNHYTQKSLSLSPGK
SEQ ID NO: 327 Fc5 (L234A, L235A)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCPAPEAAGGPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEAL
HNHYTQKSLSLSPGK
SEQ ID NO: 328 Fc6 (L234A, L235A)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPEAAGGPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEAL
HNHYTQKSLSLSPGK
SEQ ID NO: 329 Fc7 (L234A, L235A, G237A)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPEAAGAPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEAL
HNHYTQKSLSLSPGK
SEQ ID NO: 330 Fc8 (L234A, L235A, G237A)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCPAPEAAGAPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEAL
HNHYTQKSLSLSPG
SEQ ID NO: 331 Fc9 (L234A, L235A, G237A)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPEAAGAPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEAL
HNHYTQKSLSLSPG
SEQ ID NO: 332 Fc10 (L234A, L235A, P329G)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPEAAGGPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALGAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEAL
HNHYTQKSLSLSPGK
SEQ ID NO: 333 Fc11 (L234A, L235A, P329G)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCPAPEAAGGPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALGAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSVMHEA
LHNHYTQKSLSLSPGK
SEQ ID NO: 334 Fc12 (L234A, L235A, P329G)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPEAAGGPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALGAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSVMHEA
LHNHYTQKSLSLSPGK
SEQ ID NO: 335 Fc13 (L234F, L235E, P331S)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPEFEGGPSVFLFPPK
PKDTLMISRTPEVTCVVVDVSHEDPEVKFNWYVDGVEVHNAKTKPREEQYNSTYRVVSVLTV
LHQDWLNGKEYKCKVSNKALPASIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEALH
NHYTQKSLSLSPGK
SEQ ID NO: 336 Fc14 (L234F, L235E, P331S)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCPAPEFEGGPSVFLFPPK
PKDTLMISRTPEVTCVVVDVSHEDPEVKFNWYVDGVEVHNAKTKPREEQYNSTYRVVSVLTV
LHQDWLNGKEYKCKVSNKALPASIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALH
NHYTQKSLSLSPGK
SEQ ID NO: 337 Fc15 (L234F, L235E, P331S)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPEFEGGPSVFLFPPK
PKDTLMISRTPEVTCVVVDVSHEDPEVKFNWYVDGVEVHNAKTKPREEQYNSTYRVVSVLTV
LHQDWLNGKEYKCKVSNKALPASIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALH
NHYTQKSLSLSPGK
SEQ ID NO: 338 Fc16 (L234A, L235E, G237A)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPEAEGAPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEAL
HNHYTQKSLSLSPGK
SEQ ID NO: 339 Fc17 (L234A, L235E, G237A)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCPAPEAEGAPSVFLFPPK
PKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALH
NHYTQKSLSLSPG
SEQ ID NO: 340 Fc18 (L234A, L235E, G237A)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPEAEGAPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEAL
HNHYTQKSLSLSPG
SEQ ID NO: 341 Fc19 (L234A, L235E, G237A, P331S)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPEAEGAPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALPASIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEAL
HNHYTQKSLSLSPG
SEQ ID NO: 342 Fc20 (L234A, L235E, G237A, P331S)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCPAPEAEGAPSVFLFPPK
PKDTLMISRTPEVTCVVVDVSHEDPEVKFNWYVDGVEVHNAKTKPREEQYNSTYRVVSVLTV
LHQDWLNGKEYKCKVSNKALPASIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALH
NHYTQKSLSLSPG
SEQ ID NO: 343 Fc21 (L234A, L235E, G237A, P331S)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPEAEGAPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALPASIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEAL
HNHYTQKSLSLSPG
SEQ ID NO: 344 Fc22 (L234A, L235A, P329A)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPEAAGGPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALAAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEAL
HNHYTQKSLSLSPGK
SEQ ID NO: 345 Fc23 (L234A, L235A, P329A)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCPAPEAAGGPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALAAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSVMHEA
LHNHYTQKSLSLSPGK
SEQ ID NO: 346 Fc24 (L234A, L235A, P329A)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPEAAGGPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALAAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSVMHEA
LHNHYTQKSLSLSPGK
SEQ ID NO: 347 Fc25 (D265A)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLFPPK
PKDTLMISRTPEVTCVVVAVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEALH
NHYTQKSLSLSPGK
SEQ ID NO: 348 Fc26 (D265A)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCPAPELLGGPSVFLFPPK
PKDTLMISRTPEVTCVVVAVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALH
NHYTQKSLSLSPGK
SEQ ID NO: 349 Fc27 (D265A)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLFPPK
PKDTLMISRTPEVTCVVVAVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALH
NHYTQKSLSLSPGK
SEQ ID NO: 350 Fc28 (N297G)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYGSTYRVVSVLTV
LHQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEALH
NHYTQKSLSLSPGK
SEQ ID NO: 351 Fc29 (N297G)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCPAPELLGGPSVFLFPPK
PKDTLMISRTPEVTCVVVDVSHEDPEVKFNWYVDGVEVHNAKTKPREEQYGSTYRVVSVLTV
LHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALH
NHYTQKSLSLSPGK
SEQ ID NO: 352 Fc30 (N297G)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYGSTYRVVSVLTV
LHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALH
NHYTQKSLSLSPGK
SEQ ID NO: 353 Fc31 (D265A, N297A)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLFPPK
PKDTLMISRTPEVTCVVVAVSHEDPEVKFNWYVDGVEVHNAKTKPREEQYASTYRVVSVLTV
LHQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEALH
NHYTQKSLSLSPGK
SEQ ID NO: 354 Fc32 (D265A, N297A)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCPAPELLGGPSVFLFPPK
PKDTLMISRTPEVTCVVVAVSHEDPEVKFNWYVDGVEVHNAKTKPREEQYASTYRVVSVLTV
LHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALH
NHYTQKSLSLSPGK
SEQ ID NO: 355 Fc33 (D265A, N297A)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLFPPK
PKDTLMISRTPEVTCVVVAVSHEDPEVKFNWYVDGVEVHNAKTKPREEQYASTYRVVSVLTV
LHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALH
NHYTQKSLSLSPGK
SEQ ID NO: 356 Fc34 (D265A, N297G)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLFPPK
PKDTLMISRTPEVTCVVVAVSHEDPEVKFNWYVDGVEVHNAKTKPREEQYGSTYRVVSVLTV
LHQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEALH
NHYTQKSLSLSPGK
SEQ ID NO: 357 Fc35 (D265A, N297G)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCPAPELLGGPSVFLFPPK
PKDTLMISRTPEVTCVVVAVSHEDPEVKFNWYVDGVEVHNAKTKPREEQYGSTYRVVSVLTV
LHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALH
NHYTQKSLSLSPGK
SEQ ID NO: 358 Fc36 (D265A, N297G)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLFPPK
PKDTLMISRTPEVTCVVVAVSHEDPEVKFNWYVDGVEVHNAKTKPREEQYGSTYRVVSVLTV
LHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALH
NHYTQKSLSLSPGK
SEQ ID NO: 359 Fc37 (L235A, G237A)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELAGAPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEAL
HNHYTQKSLSLSPGK
SEQ ID NO: 360 Fc38 (L235A, G237A)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCPAPELAGAPSVFLFPPK
PKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALH
NHYTQKSLSLSPGK
SEQ ID NO: 361 Fc39 (L235A, G237A)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELAGAPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEAL
HNHYTQKSLSLSPGK
SEQ ID NO: 362 Fc40 (IgG4)
ASTKGPSVFPLAPCSRSTSESTAALGCLVKDYFPEPVTVSWNSGALTSGVHTFPAVLQSSGLYS
LSSVVTVPSSSLGTKTYTCNVDHKPSNTKVDKRVESKYGPPCPSCPAPEFLGGPSVFLFPPKPK
DTLMISRTPEVTCVVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVMHEALHNH
YTQKSLSLSLGK
SEQ ID NO: 363 (P329A)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALAAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEAL
HNHYTQKSLSLSPGK
SEQ ID NO: 364 (L234E, L235F, P331S)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPEEFGGPSVFLFPPK
PKDTLMISRTPEVTCVVVDVSHEDPEVKFNWYVDGVEVHNAKTKPREEQYNSTYRVVSVLTV
LHQDWLNGKEYKCKVSNKALPASIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEALH
NHYTQKSLSLSPGK
SEQ ID NO: 365 (S228P)
ASTKGPSVFPLAPCSRSTSESTAALGCLVKDYFPEPVTVSWNSGALTSGVHTFPAVLQSSGLYS
LSSVVTVPSSSLGTKTYTCNVDHKPSNTKVDKRVESKYGPPCPPCPAPEFLGGPSVFLFPPKPK
DTLMISRTPEVTCVVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVMHEALHNH
YTQKSLSLSLGK
SEQ ID NO: 366 (S228P, L235E)
ASTKGPSVFPLAPCSRSTSESTAALGCLVKDYFPEPVTVSWNSGALTSGVHTFPAVLQSSGLYS
LSSVVTVPSSSLGTKTYTCNVDHKPSNTKVDKRVESKYGPPCPPCPAPEFEGGPSVFLFPPKPK
DTLMISRTPEVTCVVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVMHEALHNH
YTQKSLSLSLGK
SEQ ID NO: 367 (S228P, F234A, L235A)
ASTKGPSVFPLAPCSRSTSESTAALGCLVKDYFPEPVTVSWNSGALTSGVHTFPAVLQSSGLYS
LSSVVTVPSSSLGTKTYTCNVDHKPSNTKVDKRVESKYGPPCPPCPAPEAAGGPSVFLFPPKPK
DTLMISRTPEVTCVVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVMHEALHNH
YTQKSLSLSLGK
SEQ ID NO: 368 (L234A, L235A)
QVQLVQSGAEVKKPGASVKVSCKASGFDIQDTYMHWVKQRPGQGLEWMGRIDPASGHTKY
DPKFQVRVTITRDTSTSTVYLELSSLRSEDTAVYYCARSGGLPDVWGQGTTVTVSSASTKGPS
VFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAGGPSVFLFPPKPKDTLM
ISRTPEVTCVVVDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHEALHNHYTQ
KSLSLSPGK
SEQ ID NO: 369 (L235E)
QVQLVQSGAEVKKPGASVKVSCKASGFDIQDTYMHWVKQRPGQGLEWMGRIDPASGHTKY
DPKFQVRVTITRDTSTSTVYLELSSLRSEDTAVYYCARSGGLPDVWGQGTTVTVSSASTKGPS
VFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELEGGPSVFLFPPKPKDTLMI
SRTPEVTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKS
LSLSPGK
SEQ ID NO: 370 (L234A, L235A, G237A)
QVQLVQSGAEVKKPGASVKVSCKASGFDIQDTYMHWVKQRPGQGLEWMGRIDPASGHTKY
DPKFQVRVTITRDTSTSTVYLELSSLRSEDTAVYYCARSGGLPDVWGQGTTVTVSSASTKGPS
VFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAGAPSVFLFPPKPKDTLM
ISRTPEVTCVVVDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHEALHNHYTQ
KSLSLSPGK
SEQ ID NO: 371 (L234A, L235E, G237A)
QVQLVQSGAEVKKPGASVKVSCKASGFDIQDTYMHWVKQRPGQGLEWMGRIDPASGHTKY
DPKFQVRVTITRDTSTSTVYLELSSLRSEDTAVYYCARSGGLPDVWGQGTTVTVSSASTKGPS
VFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAEGAPSVFLFPPKPKDTLM
ISRTPEVTCVVVDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHEALHNHYTQ
KSLSLSPGK
SEQ ID NO: 372 (L234A, L235A, P329A)
QVQLVQSGAEVKKPGASVKVSCKASGFDIQDTYMHWVKQRPGQGLEWMGRIDPASGHTKY
DPKFQVRVTITRDTSTSTVYLELSSLRSEDTAVYYCARSGGLPDVWGQGTTVTVSSASTKGPS
VFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAGGPSVFLFPPKPKDTLM
ISRTPEVTCVVVDVSHEDPEVKFNWYVDGVEVHNAKTKPREEQYNSTYRVVSVLTVLHQDW
LNGKEYKCKVSNKALAAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHEALHNHYTQ
KSLSLSPGK
SEQ ID NO: 373 (L234A, L235A, P329G)
QVQLVQSGAEVKKPGASVKVSCKASGFDIQDTYMHWVKQRPGQGLEWMGRIDPASGHTKY
DPKFQVRVTITRDTSTSTVYLELSSLRSEDTAVYYCARSGGLPDVWGQGTTVTVSSASTKGPS
VFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAGGPSVFLFPPKPKDTLM
ISRTPEVTCVVVDVSHEDPEVKFNWYVDGVEVHNAKTKPREEQYNSTYRVVSVLTVLHQDW
LNGKEYKCKVSNKALGAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHEALHNHYTQ
KSLSLSPGK
SEQ ID NO: 374 (P329A)
QVQLVQSGAEVKKPGASVKVSCKASGFDIQDTYMHWVKQRPGQGLEWMGRIDPASGHTKY
DPKFQVRVTITRDTSTSTVYLELSSLRSEDTAVYYCARSGGLPDVWGQGTTVTVSSASTKGPS
VFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGGPSVFLFPPKPKDTLMI
SRTPEVTCVVVDVSHEDPEVKFNWYVDGVEVHNAKTKPREEQYNSTYRVVSVLTVLHQDWL
NGKEYKCKVSNKALAAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKS
LSLSPGK
SEQ ID NO: 375 (L234E, L235F, P331S)
QVQLVQSGAEVKKPGASVKVSCKASGFDIQDTYMHWVKQRPGQGLEWMGRIDPASGHTKY
DPKFQVRVTITRDTSTSTVYLELSSLRSEDTAVYYCARSGGLPDVWGQGTTVTVSSASTKGPS
VFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEEFGGPSVFLFPPKPKDTLMI
SRTPEVTCVVVDVSHEDPEVKFNWYVDGVEVHNAKTKPREEQYNSTYRVVSVLTVLHQDWL
NGKEYKCKVSNKALPASIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKS
LSLSPGK
SEQ ID NO: 376 (D265A, N297G)
QVQLVQSGAEVKKPGASVKVSCKASGFDIQDTYMHWVKQRPGQGLEWMGRIDPASGHTKY
DPKFQVRVTITRDTSTSTVYLELSSLRSEDTAVYYCARSGGLPDVWGQGTTVTVSSASTKGPS
VFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGGPSVFLFPPKPKDTLMI
SRTPEVTCVVVAVSHEDPEVKFNWYVDGVEVHNAKTKPREEQYGSTYRVVSVLTVLHQDWL
NGKEYKCKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKS
LSLSPGK
SEQ ID NO: 377 (N297G)
QVQLVQSGAEVKKPGASVKVSCKASGFDIQDTYMHWVKQRPGQGLEWMGRIDPASGHTKY
DPKFQVRVTITRDTSTSTVYLELSSLRSEDTAVYYCARSGGLPDVWGQGTTVTVSSASTKGPS
VFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGGPSVFLFPPKPKDTLMI
SRTPEVTCVVVDVSHEDPEVKFNWYVDGVEVHNAKTKPREEQYGSTYRVVSVLTVLHQDWL
NGKEYKCKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKS
LSLSPGK
SEQ ID NO: 378 (S228P)
QVQLVQSGAEVKKPGASVKVSCKASGFDIQDTYMHWVKQRPGQGLEWMGRIDPASGHTKY
DPKFQVRVTITRDTSTSTVYLELSSLRSEDTAVYYCARSGGLPDVWGQGTTVTVSSASTKGPS
VFPLAPCSRSTSESTAALGCLVKDYFPEPVTVSWNSGALTSGVHTFPAVLQSSGLYSLSSVVTV
PSSSLGTKTYTCNVDHKPSNTKVDKRVESKYGPPCPPCPAPEFLGGPSVFLFPPKPKDTLMISRT
PEVTCVVVDVSQEDPEVQFNWYVDGVEVHNAKTKPREEQFNSTYRVVSVLTVLHQDWLNGK
EYKCKVSNKGLPSSIEKTISKAKGQPREPQVYTLPPSQEEMTKNQVSLTCLVKGFYPSDIAVEW
ESNGQPENNYKTTPPVLDSDGSFFLYSRLTVDKSRWQEGNVFSCSVMHEALHNHYTQKSLSLS
LGK
SEQ ID NO: 379 (S228P, L235E)
QVQLVQSGAEVKKPGASVKVSCKASGFDIQDTYMHWVKQRPGQGLEWMGRIDPASGHTKY
DPKFQVRVTITRDTSTSTVYLELSSLRSEDTAVYYCARSGGLPDVWGQGTTVTVSSASTKGPS
VFPLAPCSRSTSESTAALGCLVKDYFPEPVTVSWNSGALTSGVHTFPAVLQSSGLYSLSSVVTV
PSSSLGTKTYTCNVDHKPSNTKVDKRVESKYGPPCPPCPAPEFEGGPSVFLFPPKPKDTLMISRT
PEVTCVVVDVSQEDPEVQFNWYVDGVEVHNAKTKPREEQFNSTYRVVSVLTVLHQDWLNGK
EYKCKVSNKGLPSSIEKTISKAKGQPREPQVYTLPPSQEEMTKNQVSLTCLVKGFYPSDIAVEW
ESNGQPENNYKTTPPVLDSDGSFFLYSRLTVDKSRWQEGNVFSCSVMHEALHNHYTQKSLSLS
LGK
SEQ ID NO: 380 (S228P, F234A, L235A)
QVQLVQSGAEVKKPGASVKVSCKASGFDIQDTYMHWVKQRPGQGLEWMGRIDPASGHTKY
DPKFQVRVTITRDTSTSTVYLELSSLRSEDTAVYYCARSGGLPDVWGQGTTVTVSSASTKGPS
VFPLAPCSRSTSESTAALGCLVKDYFPEPVTVSWNSGALTSGVHTFPAVLQSSGLYSLSSVVTV
PSSSLGTKTYTCNVDHKPSNTKVDKRVESKYGPPCPPCPAPEAAGGPSVFLFPPKPKDTLMISR
TPEVTCVVVDVSQEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSLTCLVKGFYPSDIAVE
WESNGQPENNYKTTPPVLDSDGSFFLYSRLTVDKSRWQEGNVFSCSVMHEALHNHYTQKSLS
LSLGK
SEQ ID NO: 381
EIVLTQSPGTLSLSPGERATLSCRASSSVSYMYWYQQKPGQAPRPLIYATSNLASGIPDRFSGSG
SGTDFTLTISRLEPEDFAVYYCQQWEGNPRTFGGGTKLEIKRTVAAPSVFIFPPSDEQLKSGTAS
VVCLLNNFYPREAKVQWKVDNALQSGNSQESVTEQDSKDSTYSLSSTLTLSKADYEKHKVYA
CEVTHQGLSSPVTKSFNRGEC

In certain embodiments, an anti-TL1A antibody described herein comprises a Fc region comprising a sequence from Table 9. In certain embodiments, an anti-TL1A antibody described herein comprises a Fc region comprising any one of SEQ ID NOS: 320-367 or a sequence at least about 90%, 91%, 92%, 93%, 94%, 95%, 96%, 97%, 98%, or 99% identical to any one of SEQ ID NOS: 320-367.

In some embodiments, anti-TL1A described herein comprise a light chain constant region comprising SEQ ID NO: 319 or a sequence at least about 90%, 91%, 92%, 93%, 94%, 95%, 96%, 97%, 98%, or 99% identical to SEQ ID NO: 319.

### Additional Non-Limiting Example Anti-TL1A Antibody Embodiments

### CDR Embodiments

In one aspect, provided herein is a first embodiment of an anti-TL1A antibody. As used herein, an anti-TL1A antibody includes an anti-TL1A antigen binding fragment. Non-limiting additional embodiments include: (Embodiment 2) The anti-TL1A antibody of embodiment 1, comprising a heavy chain comprising a HCDR1, a HCDR2, and a HCDR3, and a light chain comprising a LCDR1, a LCDR2, and a LCDR3. (Embodiment 3) The anti-TL1A antibody of embodiment 1, comprising a HCDR1 comprising SEQ ID NO: 1. (Embodiment 4) The anti-TL1A antibody of embodiment 1 or embodiment 2, comprising a HCDR2 comprising SEQ ID NO: 2. (Embodiment 5) The anti-TL1A antibody of embodiment 1 or embodiment 2, comprising a HCDR2 comprising SEQ ID NO: 3. (Embodiment 6) The anti-TL1A antibody of embodiment 1 or embodiment 2, comprising a HCDR2 comprising SEQ ID NO: 4. (Embodiment 7) The anti-TL1A antibody of embodiment 1 or embodiment 2, comprising a HCDR2 comprising SEQ ID NO: 5. (Embodiment 8) The anti-TL1A antibody of any one of embodiments 1-6, comprising a HCDR3 comprising SEQ ID NO: 6. (Embodiment 9) The anti-TL1A antibody of any one of embodiments 1-6, comprising a HCDR3 comprising SEQ ID NO: 7. (Embodiment 10) The anti-TL1A antibody of any one of embodiments 1-6, comprising a HCDR3 comprising SEQ ID NO: 8. (Embodiment 11) The anti-TL1A antibody of any one of embodiments 1-6, comprising a HCDR3 comprising SEQ ID NO: 9. (Embodiment 12) The anti-TL1A antibody of any one of embodiments 1-10, comprising a LCDR1 comprising SEQ ID NO: 10. (Embodiment 13) The anti-TL1A antibody of any one of embodiments 1-11, comprising a LCDR2 comprising SEQ ID NO: 11. (Embodiment 14) The anti-TL1A antibody of any one of embodiments 1-12, comprising a LCDR3 comprising SEQ ID NO: 12. (Embodiment 15) The anti-TL1A antibody of any one of embodiments 1-12, comprising a LCDR3 comprising SEQ ID NO: 13. (Embodiment 16) The anti-TL1A antibody of any one of embodiments 1-12, comprising a LCDR3 comprising SEQ ID NO: 14 or 15. (Embodiment 17) the anti-TL1A antibody of embodiment 1, comprising the CDRs of antibody A, B, C, D, E, F, G, H, I, A2, B2, C2, D2, E2, F2, G2, H2, or I2 (Table 2), or antibody A15, A29, A30, A31, A32, A33, A34, A35, A36, A37, A38, A39, A40, A41, A42, A43, A44, A45, A46, A47, A48, A49, A50, A51, A52, A53, A54, A55, A56, A57, A58, A59, A60, A61, A62, A63, A64, A65, A66, A67, A68, A69, A70, A71, A72, A73, A74, A75, A76, A77, A78, A79, A81, A82, A83, A85, A86, A87, A88, A89, A90, A91, A92, A93, A94, A95, A96, A97, A98, A99, A100, A101, A102, A103, A104, A105, A107, A108, A109, A110, A111, A112, A113, A114, A115, A116, A117, A118, A119, A120, A121, A122, A123, A124, A125, A126, A127, A128, A129, A130, A132, A133, A134, A135, A136, A137, A138, A139, A140, A141, A142, A143, A144, A145, A146, A147, A148, A149, A150, A151, A152, A153, A154, A155, A156, A157, A158, A159, A160, A161, A162, A163, A164, A165, A166, A167, A168, A169, A170, A171, A172, A173, A174, A175, A176, A177, A178, A179, A180, A181, A182, A183, A184, A185, A186, A187, A188, A189, A190, A191, A192, A193, A194, A195, A196, A197, A198, A199, A200, A201, A202, A203, A204, A205, A206, A207, A208, A209, A210, A211, A212, A213, A214, A215, A216, A217, A218, A219, A220, A221, A222, A223, A224, A500, A501, 5C3D11, 9E12E5, AS12824, AS12823, AS12819, AS12816, AS12825, 12835, 18-7, 21-3, L8, 21-3 V102K, 21-3 V102M, 21-3 V102Q, 21-3 V102 W, 21-3 CDRv, 21-3 CDRv, Clone 2, Clone 52, Clone 46, Clone 47, Clone 14, Clone 16L, Clone 17L, Clone 17L-1, Clone 23, Clone A1, Clone 53, Clone E1, Clone 3-17L V-A, Clone 3-17L, Clone L8mod, Clone X-V, Clone X, Clone XL3-6, Clone XL3-10, Clone XL3-15, Clone L3-13, Clone H3-1, Clone H2-2, Clone H2-5, M1, M2, M3, M4, M5, M6, M7, M8, M9, M10, M11, or M12. (Embodiment 18) The anti-TL1A antibody of embodiment 1, comprising a heavy chain variable region comprising: (a) an HCDR1 comprising an amino acid sequence set forth by SEQ ID NO: 1; (b) an HCDR2 comprising an amino acid sequence set forth by any one of SEQ ID NOS: 2-5; and (c) an HCDR3 comprising an amino acid sequence set forth by any one of SEQ ID NOS: 6-9; and the light chain variable region comprises: (d) an LCDR1 comprising an amino acid sequence set forth by SEQ ID NO: 10; (e) an LCDR2 comprising an amino acid sequence set forth by SEQ ID NO: 11; and (f) an LCDR3 comprising an amino acid sequence set forth by any one of SEQ ID NOS: 12-15. (Embodiment 19) The anti-TL1A antibody of embodiment 1, comprising a HCDR1 as set forth by SEQ ID NO: 1, a HCDR2 as set forth by SEQ ID NO: 2, a HCDR3 as set forth by SEQ ID NO: 6, a LCDR1 as set forth by SEQ ID NO: 10, a LCDR2 as set forth by SEQ ID NO: 11, and a LCDR3 as set forth by SEQ ID NO: 12

### Framework Embodiments

(Embodiment 20) The anti-TL1A antibody of any one of embodiments 1-19, comprising a heavy chain framework comprising IGHV1-46*02. (Embodiment 21) The anti-TL1A antibody of any one of embodiments 1-19, comprising a heavy chain framework comprising a variant of IGHV1-46*02 comprising between about 1 and about 20 amino acid substitutions from SEQ ID NO: 316. (Embodiment 22) The anti-TL1A antibody of any one of embodiments 1-19, comprising a heavy chain framework comprising a variant of IGHV1-46*02 comprising between about 1 and about 9 amino acid substitutions from SEQ ID NO: 316. (Embodiment 23) The anti-TL1A antibody of any one of embodiments 1-19, comprising a heavy chain framework comprising a variant of IGHV1-46*02 comprising about 1, 2, 3, 4, 5, 6, 7, 8, 9, 10, 11, 12, 13, 14, 15, 16, 17, 18, 19, or 20 amino acid substitutions from SEQ ID NO: 316 in the framework. (Embodiment 24) The anti-TL1A antibody of any one of embodiments 21-23, wherein the heavy chain framework substitution comprises Q1E, as determined by Kabat numbering. (Embodiment 25) The anti-TL1A antibody of any one of embodiments 21-24, wherein the heavy chain framework substitution comprises R45K, as determined by Kabat numbering. (Embodiment 26) The anti-TL1A antibody of any one of embodiments 21-25, wherein the heavy chain framework substitution comprises A47R, as determined by Kabat numbering. (Embodiment 27) The anti-TL1A antibody of any one of embodiments 21-26, wherein the heavy chain framework substitution comprises M55I, as determined by Kabat numbering. (Embodiment 28) The anti-TL1A antibody of any one of embodiments 21-27, wherein the heavy chain framework substitution comprises V78A, as determined by Kabat numbering. (Embodiment 29) The anti-TL1A antibody of any one of embodiments 21-28, wherein the heavy chain framework substitution comprises M80I, as determined by Kabat numbering. (Embodiment 30) The anti-TL1A antibody of any one of embodiments 21-29, wherein the heavy chain framework substitution comprises R82T, as determined by Kabat numbering. (Embodiment 31) The anti-TL1A antibody of any one of embodiments 21-30, wherein the heavy chain framework substitution comprises V89A, as determined by Kabat numbering. (Embodiment 32) The anti-TL1A antibody of any one of embodiments 21-31, wherein the heavy chain framework substitution comprises M91L, as determined by Kabat numbering.

(Embodiment 33) The anti-TL1A antibody of any one of embodiments 1-19, comprising a heavy chain framework comprising SEQ ID NO: 301. (Embodiment 34) The anti-TL1A antibody of embodiment 33, wherein X1 is Q. (Embodiment 35) The anti-TL1A of embodiment 33, wherein X1=E. (Embodiment 36) The anti-TL1A of any one of embodiments 33-35, wherein X2=R. (Embodiment 37) The anti-TL1A of any one of embodiments 33-35, wherein X2=K. (Embodiment 38) The anti-TL1A of any one of embodiments 33-37, wherein X3=A. (Embodiment 39) The anti-TL1A of any one of embodiments 33-37, wherein X3=R. (Embodiment 40) The anti-TL1A of any one of embodiments 33-39, wherein X4=M. (Embodiment 41) The anti-TL1A of any one of embodiments 33-39, wherein X4=I. (Embodiment 42) The anti-TL1A of any one of embodiments 33-41, wherein X5=V. (Embodiment 43) The anti-TL1A of any one of embodiments 33-41, wherein X5=A. (Embodiment 44) The anti-TL1A of any one of embodiments 33-43, wherein X6=M. (Embodiment 45) The anti-TL1A of any one of embodiments 33-43, wherein X6=I. (Embodiment 46) The anti-TL1A of any one of embodiments 33-45, wherein X7=R. (Embodiment 47) The anti-TL1A of any one of embodiments 33-45, wherein X7=T. (Embodiment 48) The anti-TL1A of any one of embodiments 33-47, wherein X8=V. (Embodiment 49) The anti-TL1A of any one of embodiments 33-47, wherein X8=A. (Embodiment 50) The anti-TL1A of any one of embodiments 33-49, wherein X9=M. (Embodiment 51) The anti-TL1A of any one of embodiments 33-49, wherein X9=L.

(Embodiment 52) The anti-TL1A antibody of any one of embodiments 1-51, comprising a light chain framework comprising IGKV3-20*01. (Embodiment 53) The anti-TL1A antibody of any one of embodiments 1-51, comprising a light chain framework comprising a variant of IGKV3-20*01 comprising between about 1 and about 20 amino acid substitutions from SEQ ID NO: 317. (Embodiment 54) The anti-TL1A antibody of any one of embodiments 1-51, comprising a light chain framework comprising a variant of IGKV3-20*01 comprising about 1 amino acid substitution from SEQ ID NO: 317. (Embodiment 55) The anti-TL1A antibody of any one of embodiments 1-51, comprising a light chain framework comprising a variant of IGKV3-20*01 comprising about 2 amino acid substitutions from SEQ ID NO: 317. (Embodiment 56) The anti-TL1A antibody of any one of embodiments 1-51, comprising a light chain framework comprising a variant of IGKV3-20*01 comprising about 1, 2, 3, 4, 5, 6, 7, 8, 9, 10, 11, 12, 13, 14, 15, 16, 17, 18, 19, or 20 amino acid substitutions from SEQ ID NO: 317 in the framework. (Embodiment 57) The anti-TL1A antibody of any one of embodiments 53-56, wherein the light chain framework substitution comprises Q1E, as determined by Kabat numbering. (Embodiment 58) The anti-TL1A antibody of any one of embodiments 53-57, wherein the light chain framework substitution comprises R45K, as determined by Kabat numbering.

(Embodiment 59) The anti-TL1A antibody of any one of embodiments 1-51, comprising a light chain comprising a light chain framework comprising SEQ ID NO: 303. (Embodiment 60) The anti-TL1A antibody of embodiment 59, wherein X10 is L. (Embodiment 61) The anti-TL1A antibody of embodiment 59, wherein X10 is P. (Embodiment 62) The anti-TL1A antibody of any one of embodiments 59-61, wherein X1I is L. (Embodiment 63) The anti-TL1A antibody of any one of embodiments 59-61, wherein X1I is W.

(Embodiment 64) The anti-TL1A antibody of any one of embodiments 1-19, comprising a heavy chain variable framework region comprising a modified human IGHV1-46*02 framework, and a light chain variable framework region comprising a human IGKV3-20 framework or a modified human IGKV3-20 framework, wherein the heavy chain variable framework region and the light chain variable framework region collectively comprise at least one amino acid modification(s) as compared to the human IGHV1-46*02 framework and the human IGKV3-20 framework. (Embodiment 65) The antibody of embodiment 64, wherein the at least one amino acid modification(s) is no more than about 13, 12, 11, 10, 9, or 8 amino acid modifications. (Embodiment 66) The antibody of embodiment 64 or embodiment 65, wherein the amino acid modification(s) comprise: a modification at amino acid position 45 in the heavy chain variable region. (Embodiment 67) The antibody of any one of embodiments 64-66, wherein the amino acid modification(s) comprise a modification at amino acid position 47 in the heavy chain variable region. (Embodiment 68) The antibody of any one of embodiments 64-67, wherein the amino acid modification(s) comprise a modification at amino acid position 55 in the heavy chain variable region. (Embodiment 69) The antibody of any one of embodiments 64-68, wherein the amino acid modification(s) comprise a modification at amino acid position 78 in the heavy chain variable region. (Embodiment 70) The antibody of any one of embodiments 64-69, wherein the amino acid modification(s) comprise a modification at amino acid position 80 in the heavy chain variable region. (Embodiment 71) The antibody of any one of embodiments 64-70, wherein the amino acid modification(s) comprise a modification at amino acid position 82 in the heavy chain variable region. (Embodiment 72) The antibody of any one of embodiments 64-71, wherein the amino acid modification(s) comprise a modification at amino acid position 89 in the heavy chain variable region. (Embodiment 73) The antibody of any one of embodiments 64-72, wherein the amino acid modification(s) comprise a modification at amino acid position 91 in the heavy chain variable region, per Kabat numbering. (Embodiment 74) The antibody of any one of embodiments 64-65, wherein the amino acid modification(s) comprise (a) R45K, (b) A47R, (c) M55I, (d) V78A, (e) M80I, (f) R82T, (g) V89A, or (h) M91L in the heavy chain variable region, per Kabat numbering; or a combination of two or more modifications selected from (a) to (h). (Embodiment 75) The antibody of embodiment 74, wherein the amino acid modification(s) comprise: A47R. (Embodiment 76) The antibody of embodiment 74, wherein the amino acid modification(s) comprise: A47R, M55I, V78A, M80I, R82T, V89A, and M91L; A47R, M80I, and R82T; A47R, M80I, R82T, V89A, and M91L; or A47R, M55I, V78A, M80I, V89A, and M91L. (Embodiment 77) The antibody of embodiment 74, wherein the amino acid modification(s) comprise: R45K and A47R. (Embodiment 78) The antibody of embodiment 74, wherein the amino acid modification(s) comprise: R45K, A47R, V89A, and M91L. (Embodiment 79) The antibody of embodiment 74, wherein the amino acid modification(s) comprise: R45K and A47R, and M80I. (Embodiment 80) The antibody of embodiment 74, wherein the amino acid modification(s) comprise: R45K, A47R, M80I, and M91L; R45K, A47R, V78A, M80I, V89A, and M91L; R45K, A47R, M55I, V78A, M80I, R82T, V89A, and M91L; R45K, A47R, M80I, V89A, and M91L; R45K, A47R, M5SI, M80I, R82T, V89A, and M91L; R45K, A47R, M80I, and V89A; R45K, A47R, M80I, R82T, V89A, M91L; or R45K, A47R, M55I, M80I, V89A, and M91L. (Embodiment 81) The antibody of embodiment 74, wherein the amino acid modification(s) comprise: R45K. (Embodiment 82) The antibody of embodiment 74, wherein the amino acid modification(s) comprise: R45K and V78A. (Embodiment 83) The antibody of embodiment 74, wherein the amino acid modification(s) comprise: V78A. (Embodiment 84) The antibody of embodiment 74, wherein the amino acid modification(s) comprise: V78A and V89A; V78A and M80I; or V78A, M80I, and R82T. (Embodiment 85) The antibody of embodiment 74, wherein the amino acid modification(s) comprise: V89A. (Embodiment 86) The antibody of embodiment 74, wherein the amino acid modification(s) comprise: M80I. (Embodiment 87) The antibody of any one of embodiments 64-86, wherein the amino acid modification(s) comprises: (a) a modification at amino acid position 54 in the light chain variable region; and/or (b) a modification at amino acid position 55 in the light chain variable region, per Kabat numbering. (Embodiment 88) The antibody of embodiment 87, wherein the amino acid modification(s) comprises L54P in the light chain variable region, per Kabat numbering. (Embodiment 89) The antibody of embodiment 87 or 88, wherein the amino acid modification(s) comprises L55 W in the light chain variable region, per Kabat numbering.

(Embodiment 90) The antibody of any one of embodiments 1-19, comprising a heavy chain FR1 as set forth by SEQ ID NO: 304. (Embodiment 91) The antibody of any one of embodiments 1-19 or 90, comprising a heavy chain FR2 as set forth by SEQ ID NO: 305. (Embodiment 92) The antibody of any one of embodiments 1-19 or 90, comprising a heavy chain FR2 as set forth by SEQ ID NO: 313. (Embodiment 93) The antibody of any one of embodiments 1-19 or 90-92, comprising a heavy chain FR3 as set forth by SEQ ID NO: 306. (Embodiment 94) The antibody of any one of embodiments 1-19 or 90-92, comprising a heavy chain FR3 as set forth by SEQ ID NO: 307. (Embodiment 95) The antibody of any one of embodiments 1-19 or 90-92, comprising a heavy chain FR3 as set forth by SEQ ID NO: 314. (Embodiment 96) The antibody of any one of embodiments 1-19 or 90-92, comprising a heavy chain FR3 as set forth by SEQ ID NO: 315. (Embodiment 97) The antibody of any one of embodiments 1-19 or 90-96, comprising a heavy chain FR4 as set forth by SEQ ID NO: 308. (Embodiment 98) The antibody of any one of embodiments 1-19 or 90-97, comprising a light chain FR1 as set forth by SEQ ID NO: 309. (Embodiment 99) The antibody of any one of embodiments 1-19 or 90-98, comprising a light chain FR2 as set forth by SEQ ID NO: 310. (Embodiment 100) The antibody of any one of embodiments 1-19 or 90-99, comprising a light chain FR3 as set forth by SEQ ID NO: 311. (Embodiment 101) The antibody of any one of embodiments 1-19 or 90-100, comprising a light chain FR4 as set forth by SEQ ID NO: 312. (Embodiment 102) The antibody of any one of embodiments 1-19, comprising a HC FR1 as set forth by SEQ ID NO: 304, a HC FR2 as set forth by SEQ ID NO: 305, a HC FR3 as set forth by SEQ ID NO: 307, a HC FR4 as set forth by SEQ ID NO: 308, a LC FR1 as set forth by SEQ ID NO: 309, a LC FR2 as set forth by SEQ ID NO: 310, a LC FR3 as set forth by SEQ ID NO: 311, and a LC FR4 as set forth by SEQ ID NO: 312.

### Variable Region Embodiments

(Embodiment 103) The antibody of embodiment 1, comprising a heavy chain variable domain comprising an amino acid sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 101-169, 170-200, 267-268, 269-289, and a light chain variable domain comprising an amino acid sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 201-220, 221-236, 237-266. (Embodiment 104) The antibody of embodiment 103, comprising a heavy chain variable domain comprising an amino acid sequence at least 96% identical to SEQ ID NO: 104, and a light chain variable domain comprising an amino acid sequence at least 97% identical to SEQ ID NO: 201. (Embodiment 105) The antibody of embodiment 103, comprising an amino acid sequence at least 97% identical to SEQ ID NO: 104. (Embodiment 106) The antibody of embodiment 103, comprising an amino acid sequence at least 98% identical to SEQ ID NO: 104. (Embodiment 107) The antibody of embodiment 103, comprising an amino acid sequence at least 99% identical to SEQ ID NO: 104. (Embodiment 108) The antibody of embodiment 103, comprising SEQ ID NO: 104. (Embodiment 109) The antibody of any one of embodiments 103-108, comprising an amino acid sequence at least 98% identical to SEQ ID NO: 201. (Embodiment 110) The antibody of embodiment 109, comprising an amino acid sequence at least about 99% identical to SEQ ID NO: 201. (Embodiment 111) The antibody of embodiment 109, comprising SEQ ID NO: 201.

(Embodiment 112) The antibody of embodiment 103, comprising a heavy chain variable domain comprising an amino acid sequence at least about 97% identical to SEQ ID NO: 104, and a light chain variable domain comprising an amino acid sequence at least about 97% identical to SEQ ID NO: 201. (Embodiment 113) The antibody of embodiment 112, wherein the heavy chain variable domain comprises an amino acid sequence at least about 98% identical to SEQ ID NO: 104. (Embodiment 114) The antibody of embodiment 112, wherein the heavy chain variable domain comprises an amino acid sequence at least about 99% identical to SEQ ID NO: 104. (Embodiment 115) The antibody of embodiment 112, wherein the heavy chain variable domain comprises SEQ ID NO: 104. (Embodiment 116) The antibody of any one of embodiments 112-115, wherein the light chain variable domain comprises an amino acid sequence at least about 98% identical to SEQ ID NO: 201. (Embodiment 117) The antibody of any one of embodiments 112-116, wherein the light chain variable domain comprises an amino acid sequence at least about 99% identical to SEQ ID NO: 201. (Embodiment 118) The antibody of any one of embodiments 112-117, wherein the light chain variable domain comprises SEQ ID NO: 201.

### Fc Region Embodiments

(Embodiment 119) The antibody of any one of embodiments 1-118, comprising a fragment crystallizable (Fc) region. (Embodiment 120) The antibody of embodiment 119, comprising reduced antibody-dependent cell-mediated cytotoxicity (ADCC) function as compared to human IgG1 and/or reduced complement-dependent cytotoxicity (CDC) as compared to human IgG1. (Embodiment 121) The antibody of embodiment 120, wherein the human IgG1 comprises SEQ ID NO: 320. (Embodiment 122) The antibody of embodiment 120 or embodiment 121, wherein the ADCC function of the Fc region comprising reduced ADCC is at least about 50% reduced as compared to human IgG1. (Embodiment 123) The antibody of any one of embodiments 120-122, wherein the CDC function of the Fc region comprising reduced ADCC is at least about 50% reduced as compared to human IgG1. (Embodiment 124) The anti-TL1A antibody of any one of embodiments 119-123, comprising a human IgG1 Fc region comprising (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238 W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271 T, (y) 272N, (z) 292E, 292F, 292G, or 292I, (aa) 293 S, (bb) 301 W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376 W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S (IgG1σ), (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331 S, or (nnn) any combination of (a)-(uu), per Kabat numbering. (Embodiment 125) The anti-TL1A of any one of embodiments 119-123, comprising a (i) human IgG4 Fc region or (ii) a human IgG4 Fc region comprising (a) S228P, (b) S228P and L235E, or (c) S228P, F234A, and L235A, per Kabat numbering. (Embodiment 126) The anti-TL1A of any one of embodiments 119-123, comprising a human IgG2 Fc region; IgG2-IgG4 cross-subclass Fc region; IgG2-IgG3 cross-subclass Fc region; IgG2 comprising H268Q, V309L, A330S, P331S (IgG2m4); or IgG2 comprising V234A, G237A, P238S, H268A, V309L, A330S, P331 S (IgG1σ) (Embodiment 127) The antibody of any one of embodiments 119-123, comprising a human IgG1 comprising one or more substitutions selected from the group comprising 329A, 329G, 329Y, 331S, 236F, 236R, 238A, 238E, 238G, 238H, 238I, 238V, 238 W, 238Y, 248A, 254D, 254E, 254G, 254H, 254I, 254N, 254P, 254Q, 254T, 254V, 264S, 265H, 265K, 265S, 265Y, 265A, 267G, 267H, 267I, 267K, 434I, 438G, 439E, 439H, 439Q, 440A, 440D, 440E, 440F, 440M, 440T, and 440V, per Kabat numbering. (Embodiment 128) The anti-TL1A of any one of embodiments 119-123, comprising a heavy chain Fc region comprising a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 320-367. (Embodiment 129) The anti-TL1A of any one of embodiments 119-123, comprising a heavy chain constant region comprising a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 368-380. (Embodiment 130) The anti-TL1A of any one of embodiments 119-123, comprising a constant region comprising a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 381.

### Additional Antibody Features

(Embodiment 131) The anti-TL1A antibody of any one of embodiments 1-130, comprising a light chain constant region comprising a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 319.

(Embodiment 132) The anti-TL1A antibody of any one of embodiments 1-131, comprising at least about 80%, 81%, 82%, 83%, 84%, 85%, 86%, 87%, 88%, 89%, 90%, 91%, 92%, 93%, 94%, 95%, 96%, 97%, 98%, or 99% monomeric fraction as determined by size exclusion chromatography. (Embodiment 133), The antibody of embodiment 132, wherein the size exclusion chromatography comprises injecting purified antibody onto a size exclusion column, wherein the antibody is purified by protein A. (Embodiment 134) The antibody of embodiment 132 or 133, wherein the antibody is purified as described in Example 2. (Embodiment 135) The antibody of any one of embodiments 132-134, wherein the antibody is expressed under conditions described in Example 2. (Embodiment 136) The antibody of any one of embodiments 132-135, wherein the size exclusion chromatography column has an inner diameter of 4.6 mm. (Embodiment 137) The antibody of any one of embodiments 132-136, wherein the size exclusion chromatography column has a length of 150 mm. (Embodiment 138) The antibody of any one of embodiments 132-137, wherein the size exclusion chromatography column has a pore size of 200 Å. (Embodiment 139) The antibody of any one of embodiments 132-138, wherein the size exclusion chromatography column has a particle size of 1.7 micrometer. (Embodiment 140) The antibody of any one of embodiments 132-139, wherein the size exclusion chromatography column is ACQUITY UPLC BEH200 SEC column. (Embodiment 141) The antibody of any one of embodiments 132-140, wherein the antibody or antigen binding fragment is injected at a total volume of 15 μL. (Embodiment 142) The antibody of any one of embodiments 132-141, wherein the antibody is injected at a concentration of about 0.1 μg/μL to about 1.0 μg/μL. (Embodiment 143) The antibody of any one of embodiments 132-142, wherein the size exclusion chromatography is performed on a Shimadzu UPLC instrument. (Embodiment 144) The antibody of any one of embodiments 132-143, wherein the size exclusion chromatography is performed at a flow rate of 0.2 mL/min. (Embodiment 145) The antibody of any one of embodiments 132-144, wherein the size exclusion chromatography is performed at a column oven temperature of 30° C. (Embodiment 146) The antibody of any one of embodiments 132-145, wherein the percentage of monomer is calculated using Shimadzu software. (Embodiment 147) The antibody of any one of embodiments 132-146, wherein the size exclusion chromatography is performed as described in Example 2.

(Embodiment 148) The anti-TL1A antibody of any one of embodiments 1-147, wherein the anti-TL1A is expressed at a concentration of at least about 2 μg/mL, between about 2 μg/mL and about 60 μg/mL, between about 5 μg/mL and about 60 μg/mL, between about 10 μg/mL and about 60 μg/mL, at least about 5 μg/mL, at least about 10 μg/mL, at least about 15 μg/mL, at least about 20 μg/mL, between about 2 μg/mL and about 50 μg/mL, between about 2 μg/mL and about 40 μg/mL, between about 2 μg/mL and about 30 μg/mL, between about 2 μg/mL and about 20 μg/mL, between about 5 μg/mL and about 50 μg/mL, between about 5 μg/mL and about 40 μg/mL, between about 5 μg/mL and about 30 μg/mL, between about 10 μg/mL and about 50 μg/mL, between about 10 μg/mL and about 40 μg/mL, or between about 10 μg/mL and about 30 μg/mL, as determined by a method disclosed herein. (Embodiment 149) The anti-TL1A antibody of any one of embodiments 1-147, wherein the expression level is at least about 2, 3, 4, 5, 6, 7, 8, 9, 10, 11, 12, 13, 14, 15, 16, 17, 18, 19, 20, 21, 22, 23, 24, 25, 26, 27, 28, 29, or 30 μg/mL as determined by a method disclosed herein. (Embodiment 150) The antibody of embodiment 148 or embodiment 149, wherein the antibody is expressed in FreeStyle 293-F cells. (Embodiment 151) The antibody of any one of embodiments 148-150, wherein the antibody is expressed as described in Example 2. (Embodiment 152) The antibody of any one of embodiments 148-151, wherein the antibody expression level is quantified using Enzyme-Linked Immunosorbent assay (ELISA). (Embodiment 153) The antibody of embodiment 152, wherein the ELISA comprises coating a surface of a substrate with a capture antibody that binds to a human or humanized antibody, applying the anti-TL1A antibody to the substrate, and applying to the substrate a second antibody that binds to a human or humanized antibody. (Embodiment 154) The antibody of embodiment 153, where the capture antibody comprises an anti-kappa antibody. (Embodiment 155) The antibody of embodiment 153 or embodiment 154, where the second antibody comprises an anti-Fc antibody. (Embodiment 156) The antibody of any one of embodiments 152-155, where the ELISA is performed as described in Example 2.

(Embodiment 157) A method of treating a disease and/or condition of the skin in a subject in need thereof, the method comprising administering to the subject an antibody or antigen binding fragment of any one of embodiments 1-156. (Embodiment 158) The method of embodiment 157, wherein the disease and/or condition of the skin comprises systemic sclerosis or scleroderma, psoriasis, lupus, dermatomyositis, eczema, epidermolysis bullosa, or bullous pemphigoid, or a combination thereof. (Embodiment 159) A method of treating inflammation and/or fibrosis in a subject in need thereof, the method comprising administering to the subject an antibody or antigen binding fragment of any one of embodiments 1-156. (Embodiment 160) The method of embodiment 159, wherein the subject has inflammatory bowel disease. (Embodiment 161) A method of treating systemic sclerosis in a subject in need thereof, the method comprising administering to the subject an antibody or antigen binding fragment of any one of embodiments 1-156.

(Embodiment 162) A nucleic acid encoding the antibody of any one of embodiments 1-156. (Embodiment 163) A vector comprising the nucleic acid of embodiment 162. (Embodiment 164) A cell comprising the nucleic acid of embodiment 162. (Embodiment 165) A cell comprising the vector of embodiment 163.

### Antibody Properties

Anti-TL1A antibodies described herein bind to specific regions or epitopes of human TL1A. In various embodiments, an anti-TL1A antibody provided herein has a binding affinity to human TL1A of less than about IE⁻⁷, IE⁻⁸, IE⁻⁹, or IE⁻¹⁰ Kd. In some cases, the binding affinity is from about IE⁻⁹ to about IE⁻¹⁰ Kd. In some embodiments, an anti-TL1A antibody provided herein has a binding affinity to murine TL1A and/or rat TL1A of less than about IE⁻⁷, IE⁻⁸, IE⁻⁹, IE⁻¹⁰, or IE⁻¹¹ Kd. Methods for determining binding affinity are exemplified herein, including in Example 2.

In various embodiments, an anti-TL1A antibody provided herein is an antagonist of a TL1A receptor, such as, but not limited to, DR3 and TR6/DcR3. In certain embodiments, the antibody inhibits at least about 10%, at least about 20%, at least about 30%, at least about 50%, at least about 75%, at least about 90%, or about 100% of one or more activity of the bound TL1A receptor. In certain embodiments, the anti-TL1A antibody inhibits TL1A activation as measured by interferon gamma release in human blood. In certain embodiments, the antibody inhibits interferon gamma release in human blood at an IC₅₀ of between about 1 nanomolar and about 30 picomolar. In certain embodiments, the antibody inhibits interferon gamma release in human blood at an IC₅₀ of between about 500 picomolar and about 30 picomolar. In certain embodiments, the antibody inhibits interferon gamma release in human blood at an IC₅₀ of between about 200 picomolar and about 30 picomolar. In certain embodiments, the antibody inhibits interferon gamma release in human blood at an IC₅₀ of less than or equal to about 200 picomolar. In certain embodiments, the antibody inhibits interferon gamma release in human blood at an IC₅₀ of less than or equal to about 100 picomolar.

In various embodiments, an anti-TL1A antibody provided herein comprises at least about 80% monomeric fraction after expression and purification as described in Example 2 or elsewhere herein. In various embodiments, an anti-TL1A antibody provided herein comprises at least about 85% monomeric fraction after expression and purification as described in Example 2 or elsewhere herein. In various embodiments, an anti-TL1A antibody provided herein comprises at least about 90% monomeric fraction after expression and purification as described in Example 2 or elsewhere herein. In various embodiments, an anti-TL1A antibody provided herein comprises at least about 91%, 92%, 93%, 94%, 95%, 96%, 97%, 98%, 99%, or 100% monomeric fraction after expression and purification as described in Example 2 or elsewhere herein.

In various embodiments, an anti-TL1A antibody provided herein has at least about 2 μg/mL expression as determined by the method disclosed herein. In some embodiments, the anti-TL1A antibody has about 2 μg/mL to about 60 μg/mL expression as determined by the method disclosed herein. In some embodiments, the anti-TL1A antibody has about 5 μg/mL to about 60 μg/mL expression as determined by the method disclosed herein. In some embodiments, the anti-TL1A antibody has about 10 μg/mL to about 60 μg/mL expression as determined by the method disclosed herein. In some embodiments, the anti-TL1A antibody has at least about 5 μg/mL expression as determined by the method disclosed herein. In some embodiments, the anti-TL1A antibody has at least about 10 μg/mL expression as determined by the method disclosed herein. In some embodiments, the anti-TL1A antibody has at least about 15 μg/mL expression as determined by the method disclosed herein. In some embodiments, the anti-TL1A antibody has at least about 20 μg/mL expression as determined by the method disclosed herein. In some embodiments, the anti-TL1A antibody expresses between about 2 μg/mL and about 50 μg/mL, between about 2 μg/mL and about 40 μg/mL, between about 2 μg/mL and about 30 μg/mL expression, between about 2 μg/mL and about 20 μg/mL, between about 5 μg/mL and about 50 μg/mL, between about 5 μg/mL and about 40 μg/mL, between about 5 μg/mL and about 30 μg/mL, between about 10 μg/mL and about 50 μg/mL, between about 10 μg/mL and about 40 μg/mL, or between about 10 μg/mL and about 30 μg/mL as determined by the method disclosed herein. In some embodiments, the anti-TL1A antibody has about 2, 3, 4, 5, 6, 7, 8, 9, 10, 11, 12, 13, 14, 15, 16, 17, 18, 19, 20, 21, 22, 23, 24, 25, 26, 27, 28, 29, 30 μg/mL expression as determined by the method disclosed herein. Methods disclosed herein include those described in Example 2.

In various embodiments, an anti-TL1A antibody provided herein is humanized and has less than about 20% non-human sequence in the framework region of each of the heavy chain and light chain variable regions. For instance, the humanized antibody comprises less than about 20%, 19%, 18%, 17%, 16%, 15%, 14%, 13%, 12%, 11%, 10%, 9%, 8%, 7%, 6%, 5%, 4%, 3%, 2%, or 1% non-human sequence in the framework region of each of the heavy chain and light chain variable regions. As another example, the humanized antibody comprises about or less than about 15, 14, 13, 12, 11, 10, 9, 8, 7, 6, 5, 4, 3, 2, or 1 non-human sequences in the framework region of each of the heavy chain and light chain variable regions. The humanized heavy chain variable domain may comprise IGHV1-46*02 framework with no or fewer than about 10, 9, 8, 7, 6, 5, 4, 3, 2, or 1 non-human mutations. The humanized light chain variable domain may comprise IGKV3-20 framework with no or fewer than about 10, 9, 8, 7, 6, 5, 4, 3, 2, or 1 non-human mutations.

### Epitope

Various embodiments provide for an anti-TL1A antibody that binds to the same region of a TL1A protein or portion thereof as a reference antibody such as the anti-TL1A antibodies described herein. In some embodiments, the reference antibody comprises antibody A, B, C, D, E, F, G, H, A2, B2, C2, D2, E2, F2, G2, or H2, or a combination thereof. In some embodiments, provided herein is an anti-TL1A antibody that binds specifically to the same region of TL1A as a reference antibody comprising a heavy chain sequence at least about 90%, 92%, 93%, 94%, 95%, 96%, 97%, 98%, 99% or 100% identical to SEQ ID NO: 104, and a light chain comprising a sequence at least about 90%, 92%, 93%, 94%, 95%, 96%, 97%, 98%, 99% or 100% identical to SEQ ID NO: 201. In some embodiments, provided herein is an anti-TL1A antibody that binds specifically to the same region of TL1A as a reference antibody comprising a heavy chain sequence at least about 90%, 92%, 93%, 94%, 95%, 96%, 97%, 98%, 99% or 100% identical to SEQ ID NO: 107, and a light chain comprising a sequence at least about 90%, 92%, 93%, 94%, 95%, 96%, 97%, 98%, 99% or 100% identical to SEQ ID NO: 201.

Non-limiting methods for determining whether an anti-TL1A antibody (i.e. test antibody) binds to the same region of a TL1A protein or portion thereof as an antibody described herein are provided. In one embodiment, the method comprises a competition assay. For instance, the method comprises determining whether the test antibody can compete with binding between the reference antibody and the TL1A protein or portion thereof, or determining whether the reference antibody can compete with binding between the test antibody and the TL1A protein or portion thereof. In some embodiments, methods include use of surface plasmon resonance to evaluate whether an anti-TL1A antibody can compete with the binding between TL1A and another anti-TL1A antibody. In some cases, surface plasmon resonance is utilized in the competition assay. Non-limiting methods are described in the examples.

In certain embodiments, disclosed herein are antibodies that compete for binding TL1A with the antibodies described herein. In certain embodiments, disclosed herein are antibodies that bind a discrete epitope that overlaps with an epitope of TL1A bound by an antibody described herein. In certain embodiments, disclosed herein are antibodies that bind the same epitope of TL1A, overlap with the an epitope of TL1A by one or more amino acid residues, or that compete for binding to an epitope of TL1A with an antibody or fragment thereof that comprises a heavy chain variable region comprising the amino acid sequence of SEQ ID NO: 104; and a light chain variable region comprising the amino acid of SEQ ID NO: 201. In certain embodiments, disclosed herein are antibodies that bind the same epitope of TL1A, overlap with the an epitope of TL1A by one or more amino acid residues, or that compete for binding to an epitope of TL1A with an antibody or fragment thereof that comprises a heavy chain variable region comprising the amino acid sequence of SEQ ID NO: 107; and a light chain variable region comprising the amino acid of SEQ ID NO: 201.

### Assays

In one embodiment, a screening paradigm for identification of antibody variants that express well in mammalian cells and preserve TL1A binding activity while minimizing the propensity of the antibody to aggregate comprises a five-step process. This screen was performed as detailed in the examples. Briefly, (1) variants were cloned and transiently expressed as intact Ig in 293 cells using small-scale (3 mL, 6-well culture plates) transfections, (2) the expression level of the antibody was assessed in the culture supernatant 96-120 hours after transfection using an antibody quantitation ELISA, (3) the binding of the supernatant antibody variants to human TL1A was assessed by ELISA, (4) the antibody was purified in a single step using Protein A and (5) the material was analyzed by analytical SEC to assess monomer/aggregate content. This approach enabled identification of variants that expressed well, preserved binding to TL1A, and displayed high monomer content.

Further provided herein are methods for analyzing antibody solubility based on percentage of monomeric fraction. For example, as described in Example 2.

Further provided herein are assays for quantifying antibody expression. For example, as described in Example 2.

Further provided herein are assays for quantifying immunogenicity of an antibody.

The antibodies described herein can be assayed for specific binding. The immunoassays which can be used include, but are not limited to, competitive and non-competitive assay systems using techniques such as BIAcore analysis, FACS analysis, immunofluorescence, immunocytochemistry, Western blots, radioimmunoassays, ELISA, “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, and protein A immunoassays. Such assays are provided in for e.g., Ausubel et al., eds, 1994, Current Protocols in Molecular Biology, Vol. 1, John Wiley & Sons, Inc., New York.

### Methods of Generating Antibodies

In various embodiments, monoclonal antibodies are prepared using methods, such as, but not limited to the hybridoma method, where a host animal is immunized to elicit the production by lymphocytes of antibodies that will specifically bind to an immunizing antigen (Kohler and Milstein (1975) Nature 256:495). Hybridomas produce monoclonal antibodies directed specifically against a chosen antigen. The monoclonal antibodies are purified from the culture medium or ascites fluid, when propagated either in vitro or in vivo.

In some embodiments, monoclonal antibodies are made using recombinant DNA methods. The polynucleotides encoding a monoclonal antibody are isolated from mature B-cells or hybridoma cells. The isolated polynucleotides encoding the heavy and light chains are then cloned into suitable expression vectors, which when transfected into host cells (e.g., E. coli cells, simian COS cells, Chinese hamster ovary (CHO) cells, or myeloma cells) generate monoclonal antibodies. The polynucleotide(s) encoding a monoclonal antibody can further be modified in a number of different manners using recombinant DNA technology to generate alternative antibodies.

In various embodiments, a chimeric antibody, a molecule in which different portions are derived from different animal species, such as those having a variable region derived from a murine monoclonal antibody and a human immunoglobulin constant region (e.g., humanized antibodies) can be generated.

In some embodiments, the anti-TL1A monoclonal antibody is a humanized antibody, to reduce antigenicity and HAMA (human anti-mouse antibody) responses when administered to a human subject. Humanized antibodies can be produced using various techniques. For example, an antibody is humanized by (1) determining the nucleotide and predicted amino acid sequence of the starting antibody light and heavy variable domains; (2) designing the humanized antibody, e.g., deciding which antibody framework region to use during the humanizing process; (3) the actual humanizing methodologies/techniques; and (4) the transfection and expression of the humanized antibody. In various embodiments, a humanized antibody can be further optimized to decrease potential immunogenicity, while maintaining functional activity, for therapy in humans.

Humanized antibodies can also be made in transgenic mice containing human immunoglobulin loci that are capable, upon immunization, of producing the full repertoire of human antibodies in the absence of endogenous immunoglobulin production. A humanized antibody may also be obtained by a genetic engineering approach that enables production of affinity-matured human-like polyclonal antibodies in large animals.

A fully humanized antibody may be created by first designing a variable region amino acid sequence that contains non-human, e.g., rodent-derived CDRs, embedded in human-derived framework sequences. The non-human CDRs provide the desired specificity. Accordingly, in some cases these residues are included in the design of the reshaped variable region essentially unchanged. In some cases, modifications may therefore be restricted to a minimum and closely watched for changes in the specificity and affinity of the antibody. On the other hand, framework residues in theory can be derived from any human variable region. A human framework sequences may be chosen, which is equally suitable for creating a reshaped variable region and for retaining antibody affinity, in order to create a reshaped antibody which shows an acceptable or an even improved affinity. The human framework may be of germline origin, or may be derived from non-germline (e.g., mutated or affinity matured) sequences. Genetic engineering techniques, for example, but not limited to, phage display of libraries of human antibodies, transgenic mice, human-human hybridoma, hybrid hybridoma, B cell immortalization and cloning, single-cell RT-PCR or HuRAb Technology, may be used to generate a humanized antibody with a hybrid DNA sequence containing a human framework and a non-human CDR.

In certain embodiments, the anti-TL1A antibody is a human antibody. Human antibodies can be directly prepared using various techniques. Immortalized human B lymphocytes immunized in vitro or isolated from an immunized individual that produce an antibody directed against a target antigen can be generated.

Chimeric, humanized and human antibodies may be produced by recombinant expression. Recombinant polynucleotide constructs typically include an expression control sequence operably linked to the coding sequences of antibody chains, including naturally associated or heterologous promoter regions. In certain embodiments, it may be desirable to generate amino acid sequence variants of these humanized antibodies, particularly where these improve the binding affinity or other biological properties of the antibody.

In certain embodiments, an antibody fragment is used to treat and/or ameliorate inflammation and/or fibrosis. In certain embodiments, an antibody fragment is used to treat and/or ameliorate a disease and/or condition of the skin and/or systemic sclerosis. Various techniques may be used for the production of antibody fragments. Generally, these fragments are derived via proteolytic digestion of intact antibodies (for example Morimoto et al., 1993, Journal of Biochemical and Biophysical Methods 24:107-117; Brennan et al., 1985, Science, 229:81). Fab, Fv, and scFv antibody fragments can all be expressed in and secreted from E. coli or other host cells, thus allowing the production of large amounts of these fragments. Other techniques for the production of antibody fragments will be apparent to the skilled practitioner.

According to the present disclosure, techniques can be adapted for the production of single-chain antibodies specific to TL1A. In addition, methods can be adapted for the construction of Fab expression libraries to allow rapid and effective identification of monoclonal Fab fragments with the desired specificity for TL1A, or derivatives, fragments, analogs or homologs thereof. Antibody fragments may be produced by techniques in the art including, but not limited to: (a) a F(ab′)2 fragment produced by pepsin digestion of an antibody molecule; (b) a Fab fragment generated by reducing the disulfide bridges of an F(ab′)2 fragment, (c) a Fab fragment generated by the treatment of the antibody molecule with papain and a reducing agent, and (d) Fv fragments.

Also provided herein are modified antibodies comprising any type of variable region that provides for the association of the antibody with TL1A. Those skilled in the art will appreciate that the modified antibodies may comprise antibodies (e.g., full-length antibodies or immunoreactive fragments thereof) in which at least a fraction of one or more of the constant region domains has been deleted or otherwise altered so as to provide desired biochemical characteristics such as decreasing TL1A. In certain embodiments, the variable regions in both the heavy and light chains are altered by at least partial replacement of one or more CDRs and, if necessary, by partial framework region replacement and sequence changing. In some embodiments, the replaced CDRs may be derived from an antibody of the same class, subclass, from an antibody of a different class, for instance, from an antibody from a different species and/or a combination thereof. In some embodiments, the constant region of the modified antibodies will comprise a human constant region. Modifications to the constant region compatible with this disclosure comprise additions, deletions or substitutions of one or more amino acids in one or more domains.

In various embodiments, the expression of an antibody or antigen-binding fragment thereof as described herein can occur in either prokaryotic or eukaryotic cells. Suitable hosts include bacterial or eukaryotic hosts, including yeast, insects, fungi, bird and mammalian cells either in vivo, or in situ, or host cells of mammalian, insect, bird or yeast origin. The mammalian cell or tissue can be of human, primate, hamster, rabbit, rodent, cow, pig, sheep, horse, goat, dog or cat origin, but any other mammalian cell may be used. In other embodiments, the antibody or antigen-fragment thereof as described herein may be transfected into the host.

In some embodiments, the expression vectors are transfected into the recipient cell line for the production of the chimeric, humanized, or composite human antibodies described herein. In various embodiments, mammalian cells can be useful as hosts for the production of antibody proteins, which can include, but are not limited to cells of fibroblast origin, such as Vero (ATCC CRL 81) or CHO-K1 (ATCC CRL 61) cells, HeLa cells and L cells. In one embodiment, eukaryotic cells that can be used to express polypeptides include, but are not limited to, COS cells, including COS 7 cells; 293 cells, including 293-6E cells; CHO cells, including CHO S and DG44 cells; PER.C6™ cells (Crucell); and NSO cells. In some embodiments, a particular eukaryotic host cell is selected based on its ability to make desired post-translational modifications to the heavy chains and/or light chains.

A number of suitable host cell lines capable of secreting intact heterologous proteins have been developed in the art, and include, but are not limited to CHO cell lines, various COS cell lines, HeLa cells, L cells and multiple myeloma cell lines.

An expression vector carrying a chimeric, humanized, or composite human antibody construct, antibody or antigen-binding fragment thereof as described herein can be introduced into an appropriate host cell by any of a variety of suitable means, depending on the type of cellular host including, but not limited to transformation, transfection, lipofection, conjugation, electroporation, direct microinjection, and microprojectile bombardment. Expression vectors for these cells can include expression control sequences, such as an origin of replication sites, a promoter, an enhancer and necessary processing information sites, such as ribosome binding sites, RNA splice sites, polyadenylation sites, and transcriptional terminator sequences.

In various embodiments, yeast can also be utilized as hosts for the production of the antibody molecules or peptides described herein. In various other embodiments, bacterial strains can also be utilized as hosts for the production of the antibody molecules or peptides described herein. Examples of bacterial strains include, but are not limited to E. coli, Bacillus species, enterobacteria, and various Pseudomonas species.

In some embodiments, one or more antibodies or antigen-binding fragments thereof as described herein can be produced in vivo in an animal that has been engineered (transgenic) or transfected with one or more nucleic acid molecules encoding the polypeptides, according to any suitable method. For production of transgenic animals, transgenes can be microinjected into fertilized oocytes, or can be incorporated into the genome of embryonic stem cells, and the nuclei of such cells transferred into enucleated oocytes. Once expressed, antibodies can be purified according to standard procedures of the art, including HPLC purification, column chromatography, gel electrophoresis and the like (see generally, Scopes, Protein Purification (Springer-Verlag, NY, 1982)).

Once expressed in the host, the whole antibodies, antibody-fragments (e.g., individual light and heavy chains), or other immunoglobulin forms of the present disclosure can be recovered and purified by various techniques, e.g., immunoabsorption or immunoaffinity chromatography, chromatographic methods such as HPLC (high performance liquid chromatography), ammonium sulfate precipitation, gel electrophoresis, or any combination of these. See generally, Scopes, PROTEIN PURIF. (Springer-Verlag, N Y, 1982). Substantially pure immunoglobulins of at least about 90% to 95% homogeneity are advantageous, as are those with 98% to 99% or more homogeneity, particularly for pharmaceutical uses. Once purified, partially or to homogeneity as desired, a humanized or composite human antibody can then be used therapeutically or in developing and performing assay procedures, immunofluorescent stainings, etc. See generally, Vols. I & II Immunol. Meth. (Lefkovits & Pernis, eds., Acad. Press, N Y, 1979 and 1981).

Various embodiments provide for a genetic construct comprising a nucleic acid encoding an anti-TL1A antibody or fragment provided herein. Genetic constructs of the antibody can be in the form of expression cassettes, which can be suitable for expression of the encoded anti-TL1A antibody or fragment. The genetic construct may be introduced into a host cell with or without being incorporated in a vector. For example, the genetic construct can be incorporated within a liposome or a virus particle. Alternatively, a purified nucleic acid molecule can be inserted directly into a host cell by various methods. The genetic construct can be introduced directly into cells of a host subject by transfection, infection, electroporation, cell fusion, protoplast fusion, microinjection or ballistic bombardment.

Various embodiments provide a recombinant vector comprising the genetic construct of an antibody provided herein. The recombinant vector can be a plasmid, cosmid or phage. The recombinant vectors can include other functional elements; for example, a suitable promoter to initiate gene expression.

Various embodiments provide a host cell comprising a genetic construct and/or recombinant vector described herein.

Various host systems are also advantageously employed to express recombinant protein. Examples of suitable mammalian host cell lines include the COS-7 lines of monkey kidney cells, and other cell lines capable of expressing an appropriate vector including, for example, L cells, C127, 3 T3, Chinese hamster ovary (CHO), HeLa and BHK cell lines. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

The proteins produced by a transformed host can be purified according to any suitable method. Such standard methods include chromatography (e.g., ion exchange, affinity and sizing column chromatography), centrifugation, differential solubility, or by any other standard technique for protein purification. Affinity tags such as hexahistidine (SEQ ID NO: 391), maltose binding domain, influenza coat sequence and glutathione-S-transferase can be attached to the protein to allow easy purification by passage over an appropriate affinity column. Isolated proteins can also be physically characterized using such techniques as proteolysis, nuclear magnetic resonance and x-ray crystallography. Recombinant protein produced in bacterial culture can be isolated.

One of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retain the ability to specifically bind the target antigen. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as He, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, may be used. Polypeptides comprising conservative amino acid substitutions can be tested in any one of the assays described herein to confirm that a desired activity, e.g. antigen-binding activity and specificity of a native or reference polypeptide is retained.

Particular conservative substitutions include, for example; Ala into Gly or into Ser; Arg into Lys; Asn into Gin or into H is; Asp into Glu; Cys into Ser; Gin into Asn; Glu into Asp; Gly into Ala or into Pro; His into Asn or into Gin; lie into Leu or into Val; Leu into lie or into Val; Lys into Arg, into Gin or into Glu; Met into Leu, into Tyr or into lie; Phe into Met, into Leu or into Tyr; Ser into Thr; Thr into Ser; Trp into Tyr; Tyr into Trp; and/or Phe into Val, into lie or into Leu.

In some embodiments, the antibody and/or antigen-binding fragment thereof described herein can be a variant of a sequence described herein, e.g., a conservative substitution variant of an antibody polypeptide. In some embodiments, the variant is a conservatively modified variant. A variant may refer to a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity, e.g., antigen-specific binding activity for the relevant target polypeptide.

Alterations of the native amino acid sequence can be accomplished by any of a number of techniques. Mutations can be introduced at particular loci or by oligonucleotide-directed site-specific mutagenesis procedures. Techniques for making such alterations are very well established and include, for example, those disclosed by Walder et al. (Gene 42: 133, 1986); Bauer et al. (Gene 37:73, 1985); Craik (BioTechniques, Jan. 1985, 12-19); Smith et al. (Genetic Engineering: Principles and Methods, Plenum Press, 1981).

Nucleic acid molecules encoding amino acid sequence variants of antibodies are prepared by a variety of methods. These methods include, but are not limited to, preparation by oligonucleotide-mediated (or site-directed) mutagenesis, PCR mutagenesis, and cassette mutagenesis of an earlier prepared variant or a non-variant version of the antibody. A nucleic acid sequence encoding at least one antibody, portion or polypeptide as described herein can be recombined with vector DNA in accordance with conventional techniques, including but not limited to, blunt-ended or staggered-ended termini for ligation and restriction enzyme digestion. Techniques for such manipulations are disclosed, e.g., by Maniatis et al., Molecular Cloning, Lab. Manual (Cold Spring Harbor Lab. Press, N Y, 1982 and 1989), and can be used to construct nucleic acid sequences which encode a monoclonal antibody molecule or antigen-binding region.

In some embodiments, a nucleic acid encoding an antibody or antigen-binding fragment thereof as described herein is comprised by a vector. In some of the aspects described herein, a nucleic acid sequence encoding an antibody or antigen-binding fragment thereof as described herein, or any module thereof, is operably linked to a vector. The term “vector,” as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding an antibody or antigen-binding portion thereof as described herein in place of non-essential viral genes. The vector and/or particle may be utilized for the purpose of transferring any nucleic acids into cells either in vitro or in vivo.

By “recombinant vector,” it is meant that the vector includes a heterologous nucleic acid sequence, or “transgene” that is capable of expression in vivo.

### Pharmaceutical Compositions

In one aspect, anti-TL1A antibodies provided herein are formulated into pharmaceutical compositions that are useful in a variety of applications including, but not limited to, therapeutic methods.

In various embodiments, the pharmaceutical compositions are formulated for delivery via any route of administration. “Route of administration” includes any administration pathway, including but not limited to intravenous, subcutaneous, aerosol, nasal, oral, transmucosal, transdermal and parenteral. In example embodiment, the route of administration is subcutaneous.

The pharmaceutical compositions may contain any pharmaceutically acceptable carrier. “Pharmaceutically acceptable carrier” refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must b e “pharmaceutically acceptable” in that it must be compatible with the other ingredients of the formulation. It must also be suitable for use in contact with any tissues or organs with which it may come in contact, meaning that does not carry a risk of toxicity, irritation, allergic response, immunogenicity, or any other complication that excessively outweighs its therapeutic benefits.

In various embodiments, provided are pharmaceutical compositions including a pharmaceutically acceptable excipient along with a therapeutically effective amount of an anti-TL1A antibody. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. The active ingredient can be mixed with excipients which are pharmaceutically acceptable and compatible with the active ingredient and in amounts suitable for use in therapeutic methods described herein. Such excipients may be solid, liquid, semisolid, or, in the case of an aerosol composition, gaseous. Suitable excipients are, for example, starch, glucose, lactose, sucrose, gelatin, malt, rice, flour, chalk, silica gel, sodium stearate, glycerol monostearate, talc, sodium chloride, dried skim milk, water, saline, dextrose, propylene glycol, glycerol, ethanol, mannitol, polysorbate or the like and combinations thereof. In addition, if desired, the composition can contain auxiliary substances such as wetting or emulsifying agents, pH buffering agents and the like which enhance or maintain the effectiveness of the active ingredient. Therapeutic compositions as described herein can include pharmaceutically acceptable salts. Pharmaceutically acceptable salts include the acid addition salts formed with inorganic acids such as, for example, hydrochloric or phosphoric acids, organic acids, for example, acetic, tartaric or mandelic, salts formed from inorganic bases such as, for example, sodium, potassium, ammonium, calcium or ferric hydroxides, and salts formed from organic bases such as isopropylamine, trimethylamine, 2-ethylamino ethanol, histidine, procaine and the like. Liquid compositions can contain liquid phases in addition to and in the exclusion of water, for example, glycerin, vegetable oils such as cottonseed oil, and water-oil emulsions. In some embodiments the composition comprises physiologically tolerable carriers. The amount of antibody used that will be effective in the treatment of a particular disorder or condition will depend on the nature of the disorder or condition and can be determined with standard clinical techniques.

### Methods of Monitoring Treatment

In certain embodiments, described herein are methods for evaluating an effect of a treatment described herein. In some embodiments, the treatment comprises administration with an inhibitor of TL1A activity or expression and optionally, one or more additional therapeutic agents. In some embodiments, the treatment is monitored by evaluating the quantity of TL1A in the subject prior to and/or after administration of a therapeutic agent.

In some embodiments, the methods described herein comprise treating a subject with an inhibitor of TL1A activity or expression, wherein a genetic risk score is calculated for the subject. In some embodiments, the genetic risk score is correlated with a high TL1A fold-change. In some embodiments, the dose of the inhibitor of TL1A activity or expression is increased based on the calculated genetic risk score. In some embodiments, the genetic risk score is a polygenic risk score. In some embodiments, the dose of the inhibitor of TL1A activity or expression is increased if the polygenic risk score is in the 75^{th} percentile compared to a reference population. In some embodiments, the dose of the inhibitor of TL1A activity or expression is maintained or decreased if the polygenic risk score is lower than the 75^{th} percentile compared to a reference population.

In some embodiments, the methods described herein comprise treating a subject with an inhibitor of TL1A activity or expression, followed by monitoring the level of a biomarker. In some embodiments, the biomarker is the level of TL1A fold-change. In some embodiments, the methods further comprise treating the subject with a second dose of the inhibitor of TL1A activity or expression, wherein the second dose is increased compared to the first dose, provided the TL1A fold-change is high. In some embodiments, the methods further comprise administering a second dose of the inhibitor of TL1A activity or expression, wherein the second dose is the same as the first dose provided the TL1A fold-change is not high. In some embodiments, the methods further comprise administering a second dose of TL1A activity or expression, provided the TL1A fold-change is not high. In certain embodiments, a high TL1A fold-change comprises a TL1A fold-change that is at least equal to the mean plus two standard deviations relative to an index or control population.

### Kits and Compositions

### Compositions

Disclosed herein are compositions useful for the detection of a genotype or biomarker in a sample obtained from a subject according to the methods described herein. Aspects disclosed herein provide compositions comprises a polynucleotide sequence comprising at least 10 but less than 50 contiguous nucleotides of any one of SEQ ID NOS: 401-409 or reverse complements thereof, wherein the contiguous polynucleotide sequence comprises a detectable molecule. In various embodiments, the detectable molecule comprises a fluorophore. In other embodiments, the polynucleotide sequences further comprise a quencher.

Also disclosed herein are compositions comprising an antibody or antigen-binding fragment that specifically binds to a target protein described herein (e.g., TL1A) wherein the antibody or antigen-binding fragment comprises a detectable molecule. In various embodiments, the antibody comprises a monoclonal antibody, a chimeric antibody, a CDR-grafted antibody, a Fab, a Fab′, a F(ab′)2, a Fv, a disulfide linked Fv, a scFv, a single domain antibody, a diabody, a multispecific antibody, a dual specific antibody, an anti-idiotypic antibody, or a bispecific antibody. In some embodiments, the antibody or antigen-binding fragment comprises an IgG antibody, an IgM antibody, and/or an IgE antibody. In some embodiments, the detectable molecule comprises a fluorophore. In some embodiments, the antibody or antigen-binding fragment is conjugated to a paramagnetic particle (e.g., bead).

### Kits

Disclosed herein, are kits useful for to detect the genotypes and/or biomarkers disclosed herein. In some embodiments, the kits disclosed herein may be used to diagnose and/or treat a disease or condition in a subject; or select a patient for treatment and/or monitor a treatment disclosed herein. In some embodiments, the kit comprises the compositions described herein, which can be used to perform the methods described herein. Kits comprise an assemblage of materials or components, including at least one of the compositions. Thus, in some embodiments the kit contains a composition including of the pharmaceutical composition, for the treatment of IBD. In other embodiments, the kits contains all of the components necessary and/or sufficient to perform an assay for detecting and measuring IBD markers, including all controls, directions for performing assays, and any necessary software for analysis and presentation of results.

In some embodiments, the kits described herein comprise components for detecting the presence, absence, and/or quantity of a target nucleic acid and/or protein described herein. In some embodiments, the kit further comprises components for detecting the presence, absence, and/or quantity of a serological marker described herein. In some embodiments, the kit comprises the compositions (e.g., primers, probes, antibodies) described herein. The disclosure provides kits suitable for assays such as enzyme-linked immunosorbent assay (ELISA), single-molecular array (Simoa), PCR, and qPCR. The exact nature of the components configured in the kit depends on its intended purpose.

In some embodiments, the kits described herein are configured for the purpose of treating and/or characterizing a disease or condition (e.g., Crohn's disease), or subclinical phenotype thereof (e.g., stricturing, penetrating, or stricturing and penetrating disease phenotypes) in a subject. In some embodiments, the kits described herein are configured for the purpose of identifying a subject suitable for treatment with an inhibitor of TL1A activity or expression (e.g., anti-TL1A antibody). In some embodiments, the kit is configured particularly for the purpose of treating mammalian subjects. In some embodiments, the kit is configured particularly for the purpose of treating human subjects. In further embodiments, the kit is configured for veterinary applications, treating subjects such as, but not limited to, farm animals, domestic animals, and laboratory animals. In some embodiments, the kit is configured to select a subject for a therapeutic agent, such as those disclosed herein. In some embodiments, the kit is configured to select a subject for treatment with a therapeutic agent disclosed herein. In one embodiment, a therapeutic agent is an anti-TL1A antibody.

Instructions for use may be included in the kit. Instructions may comprise instructions for calculating a polygenic risk score, instructions for treatment of a subject, instructions for selection of a subject, instructions for selection of a therapeutic agent, or instructions for a diagnosis of a subject. Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a glass vial or prefilled syringes used to contain suitable quantities of the pharmaceutical composition. The packaging material has an external label which indicates the contents and/or purpose of the kit and its components.

### Systems

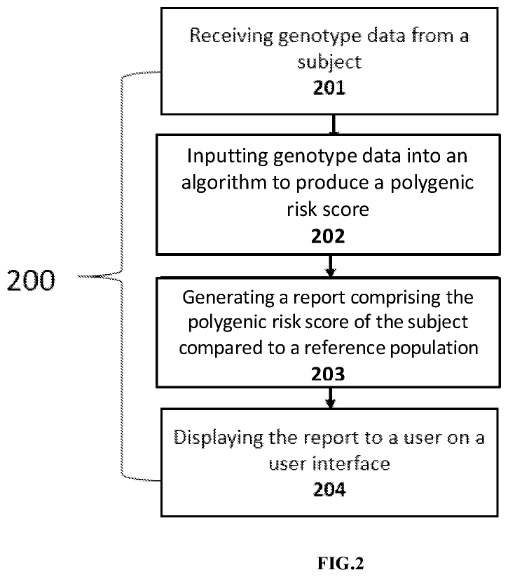

Disclosed herein are systems for identifying a subject as being suitable for treatment with an inhibitor of TL1A activity or expression (e.g., anti-TL1A antibody). In some embodiments, the systems described herein comprise kits and compositions for detecting the genotypes described herein in a biological sample of a subject. The system may comprise a computer system for implementing one or more methods of the disclosure, such as for example, receiving genotype data of a subject 201, inputting the genotype data into an algorithm to produce a polygenic risk score (PRS) 202, and generating a report comprising the PRS of the subject compared to a reference population 203, and displaying the report to a user on a graphical user interface 204, as shown in FIG. 2. A “genetic risk profile” as used herein refers to a profile of expression of one or more genotypes described herein in a subject that is detected in a biological sample obtained from the subject. In some embodiments, a genetic risk profile comprises a positive, a negative, or an indeterminate result (e.g., therapeutic response to treatment with an inhibitor of TL1A activity or expression).

### Computer Systems

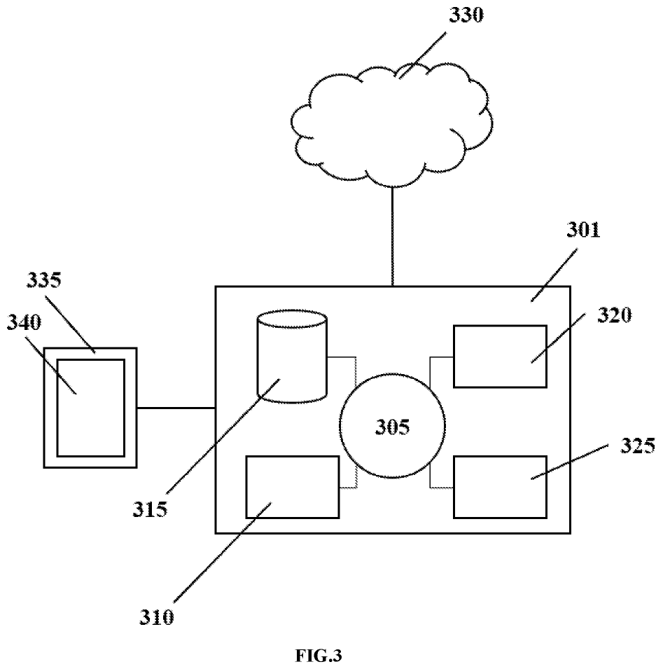

FIG. 3 shows a computer system 301 that is programmed or otherwise configured to generate a genetic risk profile for a subject in need thereof. The computer system 301 can regulate various aspects of producing the genetic risk profile (e.g., receiving genotype data, generating a report with the genetic risk profile of the biological sample, and displaying the report to a user), of the present disclosure, such as, for example, by including permissions or encryption of genotype data and/or genetic risk profile of the subject to ensure patient privacy. In some embodiments, the computer system selects one or more genetic variants. In some embodiments, the one of more genetic variants comprise one or more genotypes of the subject or a predetermined genetic variant in a linkage disequilibrium (LD) therewith. In some embodiments, the gene or genetic locus is selected from the group consisting of ETS Proto-oncogene 1 (ETS1), Interferon Gamma (IFNG), TNF Receptor Superfamily Member 6b (TNFRSF6B), TNF Superfamily Member 15 (TNFSF15), TNF Superfamily Member 8 (TNFSF8), U2 (novel transcript AF111167.2), Fos Proto-oncogene (FOS), Interferon Gamma Receptor 2 (IFNGR2), Zinc Finger CCH-Type and G-Patch Domain Containing (ZGPAT), Solute Carrier Family 19 Member 3 (SLC19A3), and C—C Motif Chemokine Ligand 20 (CC120). In some embodiments, the one or more genetic variants comprises rs11221332, rs7134599, rs6062496, rs4246905, rs7468800, rs1569328, rs2284553, rs6062504, and rs7556897. In some embodiments, the computer system calculates a genetic risk score for the subject, based, at least in part on the one or more genetic variants.

In some embodiments, the computer system predicts high TL1A fold-change in the subject. In some embodiments, the computer system calculates a positive predictive value for the genetic risk score to predict a high TL1A fold-change in the subject. In some embodiments, the computer system calculates a negative predictive value for the genetic risk score to predict a high TL1A fold-change in the subject. In some embodiments, the computer system calculates a specificity f for the genetic risk score to predict a high TL1A fold-change in the subject. In some embodiments, the computer system calculates a sensitivity for the genetic risk score to predict a high TL1A fold-change in the subject.

In some embodiments, the computer system selects a subject for treatment with a TL1A inhibitor described herein. In some embodiments, the computer system calculates a positive predictive value for the genetic risk score to predict the ability of the subject to respond to a treatment with an inhibitor of TL1A. In some embodiments, the computer system calculates a negative predictive value for the genetic risk score to predict the ability of the subject to respond to a treatment with an inhibitor of TL1A. In some embodiments, the computer system calculates a specificity for the genetic risk score to predict the ability of the subject to respond to a treatment with an inhibitor of TL1A. In some embodiments, the computer system calculates a sensitivity for the genetic risk score to predict the ability of the subject to respond to a treatment with an inhibitor of TL1A.

The computer system 301 can be an electronic device of a user or a computer system that is remotely located with respect to the electronic device. The electronic device can be a mobile electronic device, such as a mobile electronic device belonging to a physician.

The computer system 301 includes a central processing unit (CPU, also “processor” and “computer processor” herein) 305, which can be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 can be a data storage unit (or data repository) for storing data. The computer system 301 can be operatively coupled to a computer network (“network”) 330 with the aid of the communication interface 320. The network 330 can be the Internet, an internet and/or extranet, or an intranet and/or extranet that is in communication with the Internet. The network 330 in some cases is a telecommunication and/or data network. The network 330 can include one or more computer servers, which can enable distributed computing, such as cloud computing. The network 330, in some cases with the aid of the computer system 301, can implement a peer-to-peer network, which may enable devices coupled to the computer system 301 to behave as a client or a server.

The CPU 305 can execute a sequence of machine-readable instructions, which can be embodied in a program or software. The instructions may be stored in a memory location, such as the memory 310. The instructions can be directed to the CPU 305, which can subsequently program or otherwise configure the CPU 305 to implement methods of the present disclosure. Examples of operations performed by the CPU 305 can include fetch, decode, execute, and writeback.

The CPU 305 can be part of a circuit, such as an integrated circuit. One or more other components of the system 301 can be included in the circuit. In some cases, the circuit is an application specific integrated circuit (ASIC).

The storage unit 315 can store files, such as drivers, libraries and saved programs. The storage unit 315 can store user data, e.g., user preferences and user programs. The computer system 301 in some cases can include one or more additional data storage units that are external to the computer system 301, such as located on a remote server that is in communication with the computer system 301 through an intranet or the Internet.

The computer system 301 can communicate with one or more remote computer systems through the network 330. For instance, the computer system 301 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 301 via the network 330.

Methods as described herein can be implemented byway of machine (e.g., computer processor) executable code stored on an electronic storage location of the computer system 301, such as, for example, on the memory 310 or electronic storage unit 315. The machine executable or machine readable code can be provided in the form of software. During use, the code can be executed by the processor 305. In some cases, the code can be retrieved from the storage unit 315 and stored on the memory 310 for ready access by the processor 305. In some situations, the electronic storage unit 315 can be precluded, and machine-executable instructions are stored on memory 310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 301 can include or be in communication with an electronic display 335 that comprises a user interface (UI) 340 for providing, for example, a report comprising the genetic risk profile of the subject or other relevant clinical information for purposes of informing a selection of a therapeutic agent (e.g., anti-TL1A antibody) to treat a disease or condition of the subject described herein. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 305. The algorithm can, for example, perform: (a) receiving genotype data of a subject, (b) determining whether the genotypes are heterozygous or homozygous for nine polymorphisms, (c) generating an outcome using predetermined parameters, and (d) displaying the outcome to a user (e.g., physician) on a user interface of an electronic device. In some embodiments, the outcome is positive, negative or indeterminant. In some embodiments, the predetermined parameters are genotype combinations known to be predictive of a therapeutic response to a treatment, such as with an inhibitor of TL1A activity or expression.

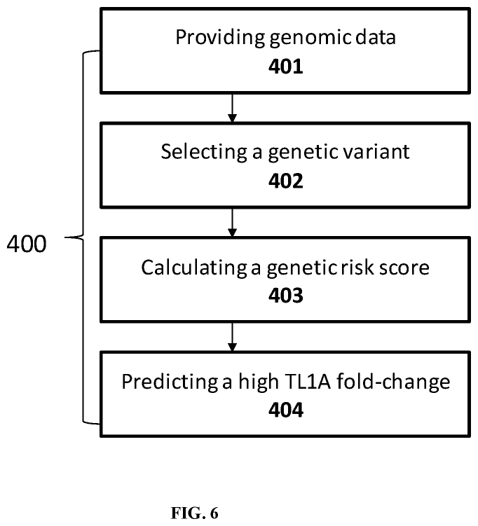

In some embodiments, the computer-implemented method 400 is depicted in FIG. 6. In some embodiments, the method comprises (a) providing genomic data 401, (b) selecting a genetic variant 402, (c) calculating a genetic risk score 403 and (d) predicting a high TL1A fold-change 404. In some embodiments, the genomic data comprises one or more genotypes of the subject as described herein.

### Web Application

In some embodiments, the computer system comprises software for a web application. In light of the disclosure provided herein, in some embodiments, a web application may utilize one or more software frameworks and one or more database systems. A web application, for example, is created upon a software framework such as Microsoft®.NET or Ruby on Rails (RoR). A web application, In some embodiments, utilizes one or more database systems including, by way of non-limiting examples, relational, non-relational, feature oriented, associative, and XML database systems. Suitable relational database systems include, by way of non-limiting examples, Microsoft® SQL Server, mySQL™, and Oracle®. In some embodiments, a web application may be written in one or more versions of one or more languages. In some embodiments, a web application is written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PUP), Python™, Ruby, Tcl, Smalltalk, WebDNA®, or Groovy. In some embodiments, a web application is written to some extent in a database query language such as Structured Query Language (SQL). A web application may integrate enterprise server products such as IBM® Lotus Domino®. A web application may include a media player element. A media player element may utilize one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, the computer system comprises software for a mobile application. The mobile application may be provided to a mobile digital processing device at the time it is manufactured. The mobile application may be provided to a mobile digital processing device via the computer network described herein.

A mobile application is created by using hardware, languages, and development environments. In some embodiments, the mobile applications may be written in several languages. Suitable programming languages include, by way of non-limiting examples, C, C++, C#, Featureive-C, Java™, Javascript, Pascal, Feature Pascal, Python™, Ruby, VB .NET, WML, and XHTML/HTML with or without CSS, or combinations thereof.

Suitable mobile application development environments are available from several sources. Commercially available development environments include, by way of non-limiting examples, Airplay SDK, alcheMo, Appcelerator®, Celsius, Bedrock, Flash Lite, .NET Compact Framework, Rhomobile, and WorkLight Mobile Platform. Other development environments may be available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, the computer system comprises software a standalone application, which is a program that may be run as an independent computer process, not an add-on to an existing process, e.g., not a plug-in. In some embodiments, standalone applications are sometimes compiled. In some embodiments, a compiler is a computer program(s) that transforms source code written in a programming language into binary feature code such as assembly language or machine code. Suitable compiled programming languages include, by way of non-limiting examples, C, C++, Featureive-C, COBOL, Delphi, Eiffel, Java™, Lisp, Python™ Visual Basic, and VB .NET, or combinations thereof. Compilation may be often performed, at least in part, to create an executable program. In some embodiments, a computer program includes one or more executable complied applications.

### Web Browser Plug-In

In some embodiments, the computer system comprises software that comprises a web browser plug-in. In computing, a plug-in, In some embodiments, is one or more software components that add specific functionality to a larger software application. Makers of software applications may support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Examples of web browser plug-ins include, without limitations, Adobe® Flash® Player, Microsoft® Silverlight®, and Apple® QuickTime®. The toolbar may comprise one or more web browser extensions, add-ins, or add-ons. The toolbar may comprise one or more explorer bars, tool bands, or desk bands.

In view of the disclosure provided herein, plug-in frameworks are available that enable development of plug-ins in various programming languages, include, by way of non-limiting examples, C++, Delphi, Java™, PHP, Python™, and VB .NET, or combinations thereof.

In some embodiments, Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. The web browser, In some embodiments, is a mobile web browser. Mobile web browsers (also called microbrowsers, mini-browsers, and wireless browsers) may be designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

The medium, method, and system disclosed herein comprise one or more softwares, servers, and database modules, or use of the same. In view of the disclosure provided herein, software modules may be created by using machines, software, and languages. The software modules disclosed herein may be implemented in a multitude of ways. In some embodiments, a software module comprises a file, a section of code, a programming feature, a programming structure, or combinations thereof. A software module may comprise a plurality of files, a plurality of sections of code, a plurality of programming features, a plurality of programming structures, or combinations thereof. By way of non-limiting examples, the one or more software modules comprise a web application, a mobile application, and/or a standalone application. Software modules may be in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

### Databases

The medium, method, and system disclosed herein comprise one or more databases, or use of the same. In view of the disclosure provided herein, some databases are suitable for storage and retrieval of geologic profile, operator activities, division of interest, and/or contact information of royalty owners may be used. Suitable databases include, by way of non-limiting examples, relational databases, non-relational databases, feature oriented databases, feature databases, entity-relationship model databases, associative databases, and XML databases. In some embodiments, a database is internet-based. In some embodiments, a database is web-based. In some embodiments, a database is cloud computing-based. A database may be based on one or more local computer storage devices.

### Data Transmission

The subject matter described herein, including methods for producing a genetic risk profile are configured to be performed in one or more facilities at one or more locations. Facility locations are not limited by country and include any country or territory. In some embodiments, one or more steps are performed in a different country than another step of the method. In some embodiments, one or more steps for obtaining a sample are performed in a different country than one or more steps for detecting the presence or absence of a genotype in a biological sample. In some embodiments, one or more method steps involving a computer system are performed in a different country than another step of the methods provided herein. In some embodiments, data processing and analyses are performed in a different country or location than one or more steps of the methods described herein. In some embodiments, one or more articles, products, or data are transferred from one or more of the facilities to one or more different facilities for analysis or further analysis. An article includes, but is not limited to, one or more components obtained from a subject, e.g., processed cellular material. Processed cellular material includes, but is not limited to, cDNA reverse transcribed from RNA, amplified RNA, amplified cDNA, sequenced DNA, isolated and/or purified RNA, isolated and/or purified DNA, and isolated and/or purified polypeptide. Data includes, but is not limited to, information regarding the stratification of a subject, and any data produced by the methods disclosed herein. In some embodiments of the methods and systems described herein, the analysis is performed and a subsequent data transmission step will convey or transmit the results of the analysis.

In some embodiments, any step of any method described herein is performed by a software program or module on a computer. In additional or further embodiments, data from any step of any method described herein is transferred to and from facilities located within the same or different countries, including analysis performed in one facility in a particular location and the data shipped to another location or directly to an individual in the same or a different country. In additional or further embodiments, data from any step of any method described herein is transferred to and/or received from a facility located within the same or different countries, including analysis of a data input, such as genetic or processed cellular material, performed in one facility in a particular location and corresponding data transmitted to another location, or directly to an individual, such as data related to the diagnosis, prognosis, responsiveness to therapy (e.g., anti-TL1A therapy), or the like, in the same or different location or country.

### Business Methods Utilizing a Computer

The methods described herein may utilize one or more computers. The computer may be used for managing customer and biological sample information such as sample or customer tracking, database management, analyzing molecular profiling data, analyzing cytological data, storing data, billing, marketing, reporting results, storing results, or a combination thereof. The computer may include a monitor or other user interface for displaying data, results, billing information, marketing information (e.g. demographics), customer information, or sample information. The computer may also include means for data or information input. The computer may include a processing unit and fixed or removable media or a combination thereof. The computer may be accessed by a user in physical proximity to the computer, for example via a keyboard and/or mouse, or by a user that does not necessarily have access to the physical computer through a communication medium such as a modem, an internet connection, a telephone connection, or a wired or wireless communication signal carrier wave. In some cases, the computer may be connected to a server or other communication device for relaying information from a user to the computer or from the computer to a user. In some cases, the user may store data or information obtained from the computer through a communication medium on media, such as removable media. It is envisioned that data relating to the methods can be transmitted over such networks or connections for reception and/or review by a party. The receiving party can be but is not limited to an individual, a health care provider (e.g., physician) or a health care manager. In one embodiment, a computer-readable medium includes a medium suitable for transmission of a result of an analysis of a biological sample, such as exosome bio-signatures. The medium can include a result regarding an exosome bio-signature of a subject, wherein such a result is derived using the methods described herein.

The entity obtaining a report with the genetic risk profile may enter biological sample information into a database for the purpose of one or more of the following: inventory tracking, assay result tracking, order tracking, customer management, customer service, billing, and sales. Sample information may include, but is not limited to: customer name, unique customer identification, customer associated medical professional, indicated assay or assays, assay results, adequacy status, indicated adequacy tests, medical history of the individual, preliminary diagnosis, suspected diagnosis, sample history, insurance provider, medical provider, third party testing center or any information suitable for storage in a database. Sample history may include but is not limited to: age of the sample, type of sample, method of acquisition, method of storage, or method of transport.

The database may be accessible by a customer, medical professional, insurance provider, or other third party. Database access may take the form of electronic communication such as a computer or telephone. The database may be accessed through an intermediary such as a customer service representative, business representative, consultant, independent testing center, or medical professional. The availability or degree of database access or sample information, such as assay results, may change upon payment of a fee for products and services rendered or to be rendered. The degree of database access or sample information may be restricted to comply with generally accepted or legal requirements for patient or customer confidentiality.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J Mol Biol. 1990 Oct. 5; 215(3):403-10; Nucleic Acids Res. 1997 Sep. 1; 25(17):3389-402). Percent homology of sequences can be determined using the most recent version of BLAST, as of the filing date of this application. Percent identity of sequences can be determined using the most recent version of BLAST, as of the filing date of this application.

The terms “increased,” or “increase” are used herein to generally mean an increase by a statically significant amount. In some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level. An increase can be an absolute amount (e.g., level of protein expression), or a rate of production (e.g., rate of protein expression between two points in time).

The terms, “decreased” or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. Other examples of“increase” include an increase of at least 2-fold, at least 5-fold, at least 10-fold, at least 20-fold, at least 50-fold, at least 100-fold, at least 1000-fold or more as compared to a reference level. An increase can be an absolute amount (e.g., level of protein expression), or a rate of production (e.g., rate of protein expression between two points in time). In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The terms “subject” encompass mammals. Non-limiting examples of mammal include, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human. The term “animal” as used herein comprises human beings and non-human animals. In one embodiment, a “non-human animal” is a mammal, for example a rodent such as rat or a mouse. In some embodiments, a human subject is a “patient,” which as used herein, refers to a subject who has or may be diagnosed with a disease or condition disclosed herein.

The term “gene,” as used herein, refers to a segment of nucleic acid that encodes an individual protein or RNA (also referred to as a “coding sequence” or “coding region”), optionally together with associated regulatory region such as promoter, operator, terminator and the like, which may be located upstream or downstream of the coding sequence. A “genetic locus” referred to herein, is a particular location within a gene.

The term, “genotype” as disclosed herein, refers to the chemical composition of a polynucleotide sequence within the genome of an individual. In some embodiments, the genotype comprises a single nucleotide polymorphism (SNP) or and indel (insertion or deletion, of a nucleobase within a polynucleotide sequence). In some embodiments, a genotype for a particular SNP, or indel is heterozygous. In some embodiments, a genotype for a particular SNP, or indel is homozygous.

A “polymorphism” as used herein refers to an aberration in (e.g., a mutation), or of (e.g., insertion/deletion), a nucleic acid sequence, as compared to the nucleic acid sequence in a reference population. In some embodiments, the polymorphism is common in the reference population. In some embodiments, the polymorphism is rare in the reference population. In some embodiments, the polymorphism is a single nucleotide polymorphism.

The term, “single nucleotide polymorphism” or SNP as disclosed herein, refers to a variation in a single nucleotide within a polynucleotide sequence. The term should not be interpreted as placing a restriction on a frequency of the SNP in a given population. The variation of an SNP may have multiple different forms. A single form of an SNP is referred to as an “allele.” An SNP can be mono-, bi-, tri, or tetra-allelic. A SNP may include a “risk allele,” a “protective allele,” or neither. By way of example, a reference polynucleotide sequence reading 5′ to 3′ is TTACG. A SNP at allele position 3 (of 5′-TTACG-3′) comprise a substitution of the reference allele, “A” to a non-reference allele, “C.” If the “C” allele of the SNP is associated with an increased probability of developing a phenotypic trait, the allele is considered a “risk” allele. However, the same SNP may also comprise a substitution of the “A” allele to a “T” allele at position 3. If the T allele of the SNP is associated with a decreased probability of developing a phenotypic trait, the allele is considered a “protective” allele. The SNP may be observed in at least 1% of a given population. In some embodiments, the SNP is represented by an “rs” number, which refers to the accession of reference cluster of one more submitted SNPs in the db SNP bioinformatics database as of the filing date of this patent application, and which is included within a sequence that comprises the total number of nucleobases from 5′ to 3′. In some embodiments, a SNP may be further defined by the position of the SNP (nucleobase) within the dbSNP sequence, the position of which is always with reference to 5′ length of the sequence plus 1. In some embodiments, a SNP is defined as the genomic position in a reference genome and the allele change (e.g. chromosome 7 at position 234,123,567 from G allele to A allele in the reference human genome build 37). In some embodiments, the SNV is defined as the genomic position identified with [brackets] or an “N” in a sequence disclosed herein.

The term, “indel,” as disclosed herein, refers to an insertion, or a deletion, of a nucleobase within a polynucleotide sequence. An indel can be mono-, bi-, tri, or tetra-allelic. An indel may be “risk,” a “protective,” or neither, for a phenotypic trait. In some embodiments, the indel is represented by an “rs” number, which refers to the accession of reference cluster of one more submitted indels in the db SNP bioinformatics database as of the filing date of this patent application, and which is included in a sequence that comprises the total number of nucleobases from 5′ to 3′. In some embodiments, an indel may be further defined by the position of the insertion/deletion within the dbSNP sequence, the position of which is always with reference to the 5′ length of the sequence plus 1. In some embodiments, an indel is defined as the genomic position in a reference genome and the allele change. In some embodiments, the indel is defined as the genomic position identified with [brackets] or an “N” in a sequence disclosed herein.

“Haplotype” as used herein, encompasses a group of one or more genotypes, which tend to be inherited together in a reference population. In some embodiments, a haplotype comprises particular polymorphism or another polymorphism in linkage disequilibrium (LD) therewith.

“Linkage disequilibrium,” or “LD,” as used herein refers to the non-random association of alleles or indels in different gene loci in a given population. LD may be defined by a D′ value corresponding to the difference between an observed and expected allele or indel frequencies in the population (D=Pab−PaPb), which is scaled by the theoretical maximum value of D. LD may be defined by an r2 value corresponding to the difference between an observed and expected unit of risk frequencies in the population (D=Pab−PaPb), which is scaled by the individual frequencies of the different loci. In some embodiments, D′ comprises at least 0.20. In some embodiments, r2 comprises at least 0.70.

The term “medically refractory,” or “refractory,” as used herein, refers to the failure of a standard treatment to induce remission of a disease. In some embodiments, the disease comprises an inflammatory disease disclosed herein. A non-limiting example of refractory inflammatory disease includes refractory Crohn's disease, and refractory ulcerative colitis (e.g., mrUC). Non-limiting examples of standard treatment include glucocorticosteriods, anti-TNF therapy, anti-a4-b7 therapy (vedolizumab), anti-IL12p40 therapy (ustekinumab), Thalidomide, and Cytoxin.

The terms “treat,” “treating,” and “treatment” as used herein refers to alleviating or abrogating a disorder, disease, or condition; or one or more of the symptoms associated with the disorder, disease, or condition; or alleviating or eradicating a cause of the disorder, disease, or condition itself. Desirable effects of treatment can include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The term “therapeutically effective amount” refers to the amount of a compound or therapy that, when administered, is sufficient to prevent development of, or alleviate to some extent, one or more of the symptoms of a disorder, disease, or condition of the disease; or the amount of a compound that is sufficient to elicit biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, P A, 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, F L, 2004).

The term “pharmaceutical composition” refers to a mixture of a compound disclosed herein with other chemical components, such as diluents or carriers. The pharmaceutical composition can facilitate administration of the compound to an organism. Multiple techniques of administering a compound exist in the art including, but not limited to, oral, injection, aerosol, parenteral, and topical administration.

The term “inflammatory bowel disease” or “IBD” as used herein refers to gastrointestinal disorders of the gastrointestinal tract. Non-limiting examples of IBD include, Crohn's disease (CD), ulcerative colitis (UC), indeterminate colitis (IC), microscopic colitis, diversion colitis, Behcet's disease, and other inconclusive forms of IBD. In some embodiments, IBD comprises fibrosis, fibrostenosis, stricturing and/or penetrating disease, obstructive disease, or a disease that is refractory (e.g., mrUC, refractory CD), perianal CD, or other complicated forms of IBD.

Non-limiting examples of “sample” include any material from which nucleic acids and/or proteins can be obtained. As non-limiting examples, this includes whole blood, peripheral blood, plasma, serum, saliva, mucus, urine, semen, lymph, fecal extract, cheek swab, cells or other bodily fluid or tissue, including but not limited to tissue obtained through surgical biopsy or surgical resection. In various embodiments, the sample comprises tissue from the large and/or small intestine. In various embodiments, the large intestine sample comprises the cecum, colon (the ascending colon, the transverse colon, the descending colon, and the sigmoid colon), rectum and/or the anal canal. In some embodiments, the small intestine sample comprises the duodenum, jejunum, and/or the ileum. Alternatively, a sample can be obtained through primary patient derived cell lines, or archived patient samples in the form of preserved samples, or fresh frozen samples.

The term “biomarker” comprises a measurable substance in a subject whose presence, level, or activity, is indicative of a phenomenon (e.g., phenotypic expression or activity; disease, condition, subclinical phenotype of a disease or condition, infection; or environmental stimuli). In some embodiments, a biomarker comprises a gene, gene expression product (e.g., RNA or protein), or a cell-type (e.g., immune cell).

The term “serological marker,” as used herein refers to a type of biomarker representing an antigenic response in a subject that may be detected in the serum of the subject. In some embodiments, a serological comprises an antibody against various fungal antigens. Non-limiting examples of a serological marker comprise anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globasa antibody, anti-Cladosporium albicans antibody, anti-laminaribiose antibody (ALCA), anti-chitobioside antibody (ACCA), anti-laminarin antibody, anti-chitin antibody, pANCA antibody, anti-I2 antibody, and anti-Cbir1 flagellin antibody.

The term “microbiome” and its variation used herein describe the populations and interactions of the bacteria, fungi, protists, and virus that align the gastrointestinal tract of a subject. A subject afflicted with IBD may possess presence, absence, excess, diminished, or a combination thereof of a microbiome s compared to a healthy subject.

The terms “non-response,” or “loss-of-response,” as used herein, refer to phenomena in which a subject or a patient does not respond to the induction of a standard treatment (e.g., anti-TNF therapy), or experiences a loss of response to the standard treatment after a successful induction of the therapy. The induction of the standard treatment may include 1, 2, 3, 4, or 5, doses of the therapy. A “successful induction” of the therapy may be an initial therapeutic response or benefit provided by the therapy. The loss of response may be characterized by a reappearance of symptoms consistent with a flare after a successful induction of the therapy.

The section headings used herein are for organizational purposes only and are not to be construed as limiting the subject matter described.

### EXAMPLES

The following examples are included for illustrative purposes only and are not intended to limit the scope of the invention.

### Example 1: Calculating a Crohn's Disease Polygenic Risk Score

A polygenic risk score (PRS) based on polymorphisms within multiple genes of interest (e.g., involved in the TL1A-mediated inflammatory pathways) and their associated weights in each respective reference population was calculated. The PRS is referred to herein as the “PRS.” The polymorphisms were selected from multiple GWAS based on a defined distances from the transcription start and stop sites for the gene(s) of interest (e.g., 250 kilobases upstream and downstream). Each GWAS was to define the individual weights for contribution of a polymorphism to the total score. The GWAS used include, but are not limited to, Jostins et al., 2012. Nature. 491:119-124, Liu et al., 2015. Nat Genet. 47:979-986, Ellinghaus et al., 2016. Nat Genet. 48:510-518, Huang et al., 2017. Nat Genet. 49:256-261, and de Lange et al., 2017. Nat Genet. 48:256-261. In some cases, the weights were calculated using the beta value for genetic associations to disease.

To confirm the relevance of inclusion of a polymorphism within the PRS, the polymorphisms were cross-checked by (i) evidence of cis-QTL, where the SNP is directly associated with target gene (e.g., TNFSF15) expression in tissues and (ii) sensitivity analysis, where selected polymorphisms are removed from the PRS and a regression analysis is run against disease susceptibility and subclinical phenotypes, thus highlighting relevant polymorphisms to disease risk. In some cases, the polymorphisms were subjected to sensitivity analysis, and in other cases, they were not. For example, in some cases, only those polymorphisms with questionable association to the pathway PRS (e.g., no eQTL or multiple genes within the loci) are subjected to sensitivity analysis.

Patients with Crohn's disease (CD) were recruited. The diagnosis of each patient was based on standard endoscopic, histologic, and radiographic features. Blood samples were collected from patients at the time of enrollment. Genotyping was performed using Immunochip (ICHIP) per manufacturer's protocol on all samples collected.

A PRS was calculated for Caucasian patients within a Cedars-Sinai CD, based on the defined set of polymorphisms provided in Table 10. An example of calculation of PRS is outlined in Li et al., 2018. Inflamm Bowel Dis. 12; 24(11):2413-2422. The PRS is calculated as the weighted sum of the number of risk alleles carried by each patient (in the Cedars-Sinai CD cohort) (0, 1, or 2) at each loci for the genes described, divided by a total number of genetic variants used in the model. The same calculations were performed for each individual belonging to a reference group, thereby generating a range of raw scores (observed range). The resulting CD PRS is generated by comparing the score of each patient with the observed range observed in the reference group.

TABLE 10
SNPs used to generate the CD PRS

| rsID | SNP | minor.allele2 | maf | major.allele | chr | pos_hg19 | Gene |
|---|---|---|---|---|---|---|---|
| rs11221332 | imm_11_127886184 | A | 0.232475 | G | chr11 | 128380974 | ETS1 |
| rs7134599 | imm_12_66786342 | A | 0.381954 | G | chr12 | 68500075 | IFNG |
| rs6062496 | imm_20_61799543 | G | 0.406514 | A | chr20 | 62329099 | TNFRSF6B |
| rs4246905 | imm_9_116593070 | A | 0.270925 | G | chr9 | 117553249 | TNFSF15 |
| rs7468800 | imm_9_116631826 | A | 0.124622 | C | chr9 | 117592005 | TNFSF15; TNFSF8 |
| rs1569328 | rs1569328 | A | 0.14869 | G | chr14 | 75741751 | U2; FOS |
| rs2284553 | rs2284553 | A | 0.386244 | G | chr21 | 34776695 | IFNGR2 |
| rs6062504 | rs6062504 | A | 0.27128 | G | chr20 | 62348907 | ZGPAT |
| rs7556897 | rs7556897 | G | 0.334936 | A | chr2 | 228660112 | SLC19A3; CCL20 |

TABLE 11
Sequences

| SEQ ID | | |
|---|---|---|
| NO | rsID | SEQUENCE |
| 401 | rs11221332 | CTAAGATGAC ATTAATCTCT GTATG[A/G]CTCCTTGGTA |
| | | CAGTTCTTTT CCACA |
| 402 | rs7134599 | ACATAAGTGG GAAGTAGATC |
| | | AAGAC[A/G]TGCTTATAAT TACATTCTTG TGGTT |
| 403 | rs6062496 | TGAGCCAGGG CACAGCCTCC |
| | | CCTGG[G/A]GAGCTCTGGG AAAGTGGGCA GCAAT |
| 404 | rs4246905 | GAAACTGTAG ACTTTGCTTA |
| | | AAAAG[A/G]GTCTCATATC ATTTTCAAAA TAGAC |
| 405 | rs7468800 | ATTCTAGAAT GACTGGAATA |
| | | CCCAG[A/C]AAACTGGGTA AGAATCATGT GTTCT |
| 406 | rs1569328 | CTGTGTGCGG CAGCATTTAC |
| | | GTAGG[A/G]GGTACAGACA GCAGCCTTTC ACTTA |
| 407 | rs2284553 | CAGGGCTCAG AACTGTCCGG |
| | | GTCCC[A/G]TCAGTGTTGG GGCGGAAGAG GAAGA |
| 408 | rs6062504 | TCCCTCTAAA CACTGCTTTC ACTGC[A/G]TTCCATAAGA |
| | | TTGTTTTTTG TTGTT |
| 409 | rs7556897 | GATTATAAGT AACATGTGTT TATTG[G/A]TTTTTTTTAA |
| | | AAAAGGGTAA ATATC |

### Example 2: Calculating an Inflammatory Bowel Disease Polygenic Risk Score

A polygenic risk score (PRS) based on polymorphisms within multiple genes of interest (e.g., involved in the TL1A-mediated inflammatory pathways) and their associated weights in each respective reference population was calculated. The PRS is referred to herein as the “PRS.” The polymorphisms were selected from multiple GWAS based on a defined distances from the transcription start and stop sites for the gene(s) of interest (e.g., 250 kilobases upstream and downstream). Each GWAS was to define the individual weights for contribution of a polymorphism to the total score. The GWAS used include, but are not limited to, Jostins et al., 2012. Nature. 491:119-124, Liu et al., 2015. Nat Genet. 47:979-986, Ellinghaus et al., 2016. Nat Genet. 48:510-518, Huang et al., 2017. Nat Genet. 49:256-261, and de Lange et al., 2017. Nat Genet. 48:256-261.

To confirm the relevance of inclusion of a polymorphism within the PRS, the polymorphisms were cross-checked by (i) evidence of cis-QTL, where the SNP is directly associated with target gene expression in tissues and (ii) sensitivity analysis, where selected polymorphisms are removed from the PRS and a regression analysis is run against disease susceptibility and subclinical phenotypes, thus highlighting relevant polymorphisms to disease risk. In some cases, the polymorphisms were subjected to sensitivity analysis, and in other cases, they were not. For example, in some cases, only those polymorphisms with questionable association to the pathway PRS (e.g., no eQTL or multiple genes within the loci) are subjected to sensitivity analysis.

Patients with Inflammatory Bowel Disease were recruited. The diagnosis of each patient was based on standard endoscopic, histologic, and radiographic features. Blood samples were collected from patients at the time of enrollment. Genotyping was performed using Immunochip (ICHIP) per manufacturer's protocol on all samples collected.

A PRS was calculated for Caucasian patients within a Cedars-Sinai IBD, based on the defined set of polymorphisms provided in Table 10. An example of calculation of PRS is outlined in Li et al., 2018. Inflamm Bowel Dis. 12; 24(11):2413-2422. The PRS is calculated as the weighted sum of the number of risk alleles carried by each patient (in the Cedars-Sinai IBD cohort) (0, 1, or 2) at each loci for the genes described, divided by a total number of genetic variants used in the model. The same calculations were performed for each individual belonging to a reference group, thereby generating a range of raw scores (observed range). The resulting IBD PRS is generated by comparing the score of each patient with the observed range observed in the reference group.

### Example 3: Measuring TL1A Fold-Change

Peripheral blood mononuclear cells (PBMCs) were isolated from patients with Crohn's disease. The patient sera was annotated with the following clinical parameters: presence of perianal Crohn's Disease, presence of males vs females, presence of subject with active disease, that is Harvey Bradshaw index (HBI) >=5, subject is taking systemic steroid versus none, subject is taking thiopurine versus none, subject is taking methotrexate versus none, subject taking any of the biologics (Cimzia/Remicade/Stelara/Humira) versus not taking any, subject is taking any medication (biologic or methotrexate or thiopurine) versus none, subject had any prior surgery (1 or more) versus none before collection, age at collection, age at diagnosis, Harvey Bradshaw index, disease location (L1,L2,L3,L4), 5 disease classifications (B1,B2,B2a,B2b,B3), CRP levels in subjects, presence of abnormal CRP (>=5) versus normal, clinical measurement SED, presence of abnormal SED (>=20) versus normal, clinical measurement MPV, mean corpuscular volume, duration of disease (age at draw-age at diagnosis), platelet count within 30 days of collection (units x10e3/ul), and the presence of 75% non-Jewish ancestry vs 75% Jewish ancestry.

The PBMCs were treated with immune complex. TL1A levels were measured at 6, 18, 24, and 72 hours after immune complex stimulation. A fold-change of TL1A was calculated by comparing TL1A levels at 18, 24, and 72 hours with TL1A levels at 6 hours.

The clinical parameters were compared to the changes in TL1A fold-change. Univariate correlations are shown in Table 12. Higher TL1A fold-changes levels at 24 hours are associated with the presence of abnormal SED, elevated SED rate, and increased age at diagnosis. At 72 hours, higher TL1A fold-change levels were associated with higher raw Harvey Bradshaw Index indicating increasing CD disease severity, subjects not taking methotrexate immunosuppressant, lower mean corpuscular volume (which may indicate anemia), and elevated SED rate. Multivariate association with disease duration as covariate are shown in Table 13. A score of 8 to 16 on the Harvey Bradshaw index indicates moderate disease while a score greater than 16 indicates severe disease.

TABLE 12
Clinical associations from a univariate analysis

| response (Clinical | | | | | | |
|---|---|---|---|---|---|---|
| parameter)_x(predictor) | TL1A FC | Association | | | | |
| combination | timepoint | type | pvalue | beta | N | OR |
| presence of abnormal SED (>=20) | 24 | logistic | 0.01953 | 0.09576 | 28 | 1.1005 |
| versus normal | | | | | | |
| Clinical measurement SED | 24 | linear | 0.03137 | 0.44916 | 28 | — |
| Age at diagnosis | 24 | linear | 0.04164 | 0.17864 | 97 | — |
| Age at collection | 24 | linear | 0.06693 | 0.16865 | — | — |
| Harvey Bradshaw Index | 72 | linear | 0.01430 | 0.02623 | 93 | — |
| Methotrexate | 72 | logistic | 0.03553 | −0.03904 | 95 | 0.9617 |
| Mean corpuscular volume | 72 | linear | 0.03763 | −0.07629 | 48 | — |
| Clinical measurement SED | 72 | linear | 0.04870 | 0.30273 | 28 | — |
| Age at diagnosis | 72 | linear | 0.09194 | 0.08128 | — | — |
| presence of abnormal SED (>=20) | 72 | logistic | 0.11962 | 0.03509 | — | 1.0357 |
| versus normal | | | | | | |

TABLE 13
Clinical associations from a multivariate analysis using disease duration (dz_duration) as covariate.

| response (Clinical | | | | | | | |
|---|---|---|---|---|---|---|---|
| parameter)_x(predictor) | TL1A FC | Association | | | | | |
| combination | timepoint | type | covariate | pvalue | beta | N | OR |
| presence of abnormal SED (>=20) | 24 | logistic | dz_duration | 0.02155 | 0.09617 | 28 | 1.10095 |
| versus normal | | | | | | | |
| Age at collection | 24 | linear | dz_duration | 0.03202 | 0.17430 | 97 | — |
| Age at diagnosis | 24 | linear | dz_duration | 0.03202 | 0.17430 | 97 | — |
| Clinical measurement SED | 24 | linear | dz_duration | 0.03486 | 0.44896 | 28 | — |
| Harvey Bradshaw Index | 72 | linear | dz_duration | 0.01272 | 0.02671 | 93 | — |
| Methotrexate | 72 | logistic | dz_duration | 0.03547 | −0.03869 | 95 | 0.96205 |
| Mean corpuscular volume | 72 | linear | dz_duration | 0.04685 | −0.07337 | 48 | — |
| Clinical measurement SED | 72 | linear | dz_duration | 0.05324 | 0.30261 | — | — |
| Age at collection | 72 | linear | dz_duration | 0.11663 | 0.07044 | — | — |
| Age at diagnosis | 72 | linear | dz_duration | 0.11663 | 0.07044 | — | — |

### Example 4: Correlation of TL1A Fold-Change with the CD PRS

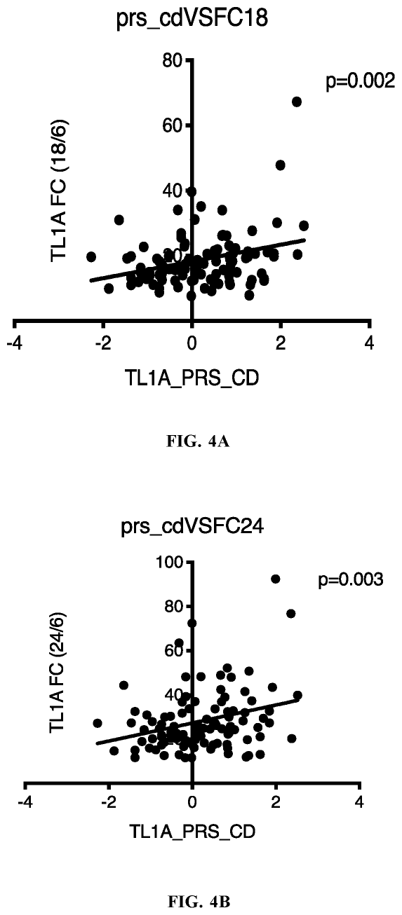

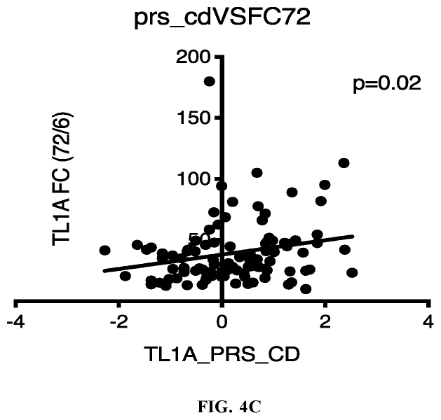

The levels of TL1A fold-change at 18, 24, and 72 hours were also correlated with CD PRS, as depicted in FIGS. 4A-4C. All associations indicated that higher TL1A fold-change levels were associated with higher levels of PRS. A high TL1A fold-change is a TL1A fold-change at least 2 standard deviations about average. A high PRS is a PRS in the 75 percentile or higher. This indicates that an increased TL1A fold-change is correlated with increasing disease severity. This also indicates that patients suitable for treatment with an anti-TL1A therapeutic have a higher risk of severe disease which may not respond to traditional IBD therapies. Hence, an anti-TL1A therapeutic may be a suitable second-line biologic for these individuals.

### Example 5: Correlation of TL1A Fold-Change with the IBD PRS

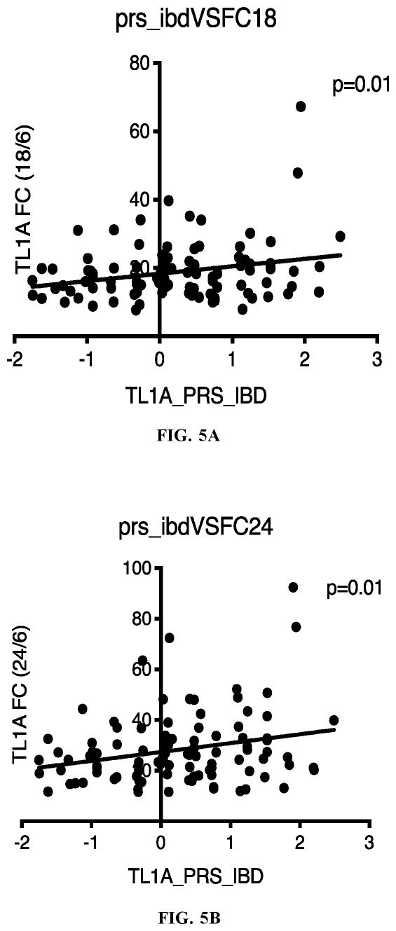

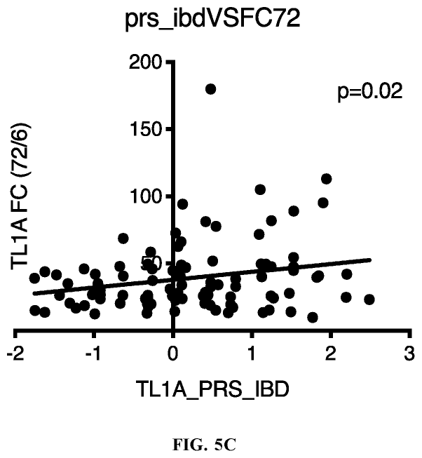

The levels of TL1A fold-change at 18, 24, and 72 hours were also correlated with the IBD PRS, as depicted in FIGS. 5A-5C. All associations indicated that higher TL1A fold-change levels were associated with higher levels of PRS. A high TL1A fold-change is a TL1A fold-change at least 2 standard deviations about average. A high PRS is a PRS in the 75 percentile or higher. This indicates that an increased TL1A fold-change is correlated with increasing disease severity. This also indicates that patients suitable for treatment with an anti-TL1A therapeutic have a higher risk of severe disease which may not respond to traditional IBD therapies. Hence, an anti-TL1A therapeutic may be a suitable second-line biologic for these individuals.

### Example 6: A Clinical Trial to Treat CD and IBD

A phase 1b clinical trial is performed to evaluate the efficacy of an anti-TL1A antibody on subjects having Crohn's disease (CD).

### Inclusion Criteria

Subjects with Crohn's disease will be included in this trial.

### Protocol

A CD PRS will be calculated for each patient as described in Example 1. The patients will be administered anti-TL1A antibody, Patients are monitored in real time. Central ready of endoscopy and biopsy is employed, with readers blinded to point of time of treatment and endpoints.

Primary Outcome Measures: Simple Endoscopic Score for Crohn's Disease (SESCD), Crohn's Disease Activity Index (CDAI), and Patient Reported Outcome (PRO). If risk either positive group shows 50% reduction from baseline, a Phase 2a clinical trial is performed.

Inclusion Criteria: PRO entry criteria: Abdominal pain score of 2 or more and/or stool frequency score of 4 or more. Primary outcome would be pain core of 0 or 1 and stool frequency score of 3 or less with no worsening from baseline. Endoscopy entry criteria: SESCD ileum only entry at score of 4 and 6 if colon is involved. Primary endoscopic outcome is 40-50% delta of mean SESCD.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of treating moderate to severely active Crohn's disease (CD) or ulcerative colitis (UC) in a subject, the method comprising: administering a therapeutically effective amount of an inhibitor of Tumor necrosis factor-like cytokine 1A (TL1A) activity or expression to a subject with moderately to severely active CD or UC that has been determined have a polygenetic risk score (PRS) in the 75^{th} percentile, which is indicative of high fold-change of TL1A expression relative to a cut-off fold-change value.

2. The method of claim 1, wherein a high TL1A fold-change is at least about 2 standard deviations above the mean of the index or control.

3. The method of claim 1, wherein calculating a PRS comprises providing genomic data comprising one or more genotypes of the subject, wherein the one or more genotypes is associated with high TL1A fold-change relative to an index or a control.

4. The method of claim 3, further comprising selecting, from a database, one or more genetic variants corresponding to:
(a) the one or more genotypes of the subject, or
(b) a predetermined genetic variant in a linkage disequilibrium (LD) therewith, wherein the one or more genetic variants comprises rs11221332, rs7134599, rs6062496, rs4246905, rs7468800, rs1569328, rs2284553, rs6062504, and rs7556897;

5. The method of claim 4, further comprising calculating a genetic risk score for the subject, based, at least in part, on the one or more selected genetic variants.

6. The method of any one of claims 1-5, wherein LD is defined by (i) a D′ value of at least 0.80 or (ii) a D′ value of 0 and an R² value of at least about 0.90.

7. The method of any one of claims 1-6, wherein the predetermined genetic variant is selected in (b) based least in part, on an association with case versus control.

8. The method of any one of claims 1-6, wherein the association with case comprises association with inflammatory bowel disease, Crohn's disease, or ulcerative colitis.

9. The method of any one of claims 1-8, wherein the index or control is a population of subjects with inflammatory disease, a fibrostenotic disease, or a fibrotic disease.

10. The method of any one of claims 1-8, wherein the index or control is a population of subjects without inflammatory disease, a fibrostenotic disease, or a fibrotic disease.

11. The method of any one of claims 1-10, wherein the anti-TL1A inhibitor is an anti-TL1A antibody.

12. The method of claim 11, wherein the anti-TL1A antibody is selected from the group consisting of
an anti-TL1A antibody comprising a HCDR1 as set forth by SEQ ID NOS: 27 or 28, a HCDR2 as set forth by SEQ ID NOS: 41 or 42, a HCDR3 as set forth by SEQ ID NOS: 54 or 55, a LCDR1 as set forth by SEQ ID NO: 68, a LCDR2 as set forth by SEQ ID NO: 81, and a LCDR3 as set forth by SEQ ID NO: 92;
an anti-TL1A antibody comprises a HCDR1 as set forth by SEQ ID NO: 1, a HCDR2 as set forth by SEQ ID NO: 2, a HCDR3 as set forth by SEQ ID NO: 6, a LCDR1 as set forth by SEQ ID NO: 10, a LCDR2 as set forth by SEQ ID NO: 11, and a LCDR3 as set forth by SEQ ID NO: 12;
an anti-TL1A antibody comprises a HCDR1 as set forth by SEQ ID NO: 16 or 17, a HCDR2 as set forth by SEQ ID NO: 18, a HCDR3 as set forth by SEQ ID NO: 19 or 20, a LCDR1 as set forth by SEQ ID NO: 21, a LCDR2 as set forth by SEQ ID NO: 22, and a LCDR3 as set forth by SEQ ID NO: 23 or 24;
an anti-TL1A antibody comprises a HCDR1 as set forth by SEQ ID NO: 25, a HCDR2 as set forth by SEQ ID NO: 39, a HCDR3 as set forth by SEQ ID NO: 52, a LCDR1 as set forth by SEQ ID NO: 66, a LCDR2 as set forth by SEQ ID NO: 79, and a LCDR3 as set forth by SEQ ID NO: 90; and
an anti-TL1A antibody comprises a HCDR1 as set forth by SEQ ID NO: 30, a HCDR2 as set forth by SEQ ID NO: 44, a HCDR3 as set forth by SEQ ID NO: 57, a LCDR1 as set forth by SEQ ID NO: 70, a LCDR2 as set forth by SEQ ID NO: 83, and a LCDR3 as set forth by SEQ ID NO: 94.

13. A computer-implemented method of analyzing genomic data, the method comprising:
(a) providing genomic data comprising one or more genotypes of the subject, wherein the one or more genotypes is associated with high Tumor necrosis factor-like cytokine 1A (TL1A) fold-change relative to an index or a control;
(b) selecting one or more genetic variants corresponding to:
(i) the one or more genotypes of the subject, or
(ii) a predetermined genetic variant in a linkage disequilibrium (LD) therewith, wherein the one or more genetic variants comprises rs11221332, rs7134599, rs6062496, rs4246905, rs7468800, rs1569328, rs2284553, rs6062504, and rs7556897;
(c) calculating a genetic risk score for the subject, based, at least in part, on the one or more genetic variants selected in (b);
(d) predicting high TL1A fold-change in the subject based, least in part, on the genetic risk score calculated in (c).

14. The method of claim 13, wherein LD is defined by (i) a D′ value of at least 0.80 or (ii) a D′ value of 0 and an R² value of at least about 0.80.

15. The method of claim 13, wherein the predetermined genetic variant is selected in (b) based least in part, on an association with case versus control.

16. The method of claim 15, wherein the association with case comprises association with inflammatory bowel disease, Crohn's disease, or ulcerative colitis.

17. The method of claim 15, wherein the index or control is a population of subjects with inflammatory disease, a fibrostenotic disease, or a fibrotic disease.

18. The method of claim 15, wherein the index or control is a population of subjects without inflammatory disease, a fibrostenotic disease, or a fibrotic disease.

19. The method of any one of claims 13-18, wherein a high TL1A fold-change is at least about 2 standard deviations above the mean of the index or control.

20. The method of any one of claims 13-19, further comprising predetermining that the predetermined genetic variant is associated with TL1A fold-change.

21. The method of any one of claims 13-20, further comprising predetermining that the predetermined genetic variant is implicated in the TL1A pathway.

22. The method of any one of claims 13-21, wherein the predetermined genetic variant is selected based on phasing unphased genotype data from the subject to generate a subject-specific phased haplotypes based on the clinical condition of the subject.

23. The method of any one of claims 13-22, wherein selecting the predetermined genetic variant comprises:
(a) imputing subject-specific genotypes not present in the phased subject-specific phased haplotypes using phased haplotype data from a reference group that has the same clinical phenotypes as the subject; and
(b) selecting the predetermined genetic variant from the imputed subject-specific genotypes.

24. The method of any one of claims 13-23, wherein the clinical phenotype comprises fibrosis, fibrostenosis, stricturing and/or penetrating disease, obstructive disease, mrUC, refractory CD, or perianal CD.

25. The method of any one of claims 13-24, wherein the clinical phenotype comprises the presence of perianal Crohn's disease.

26. The method of any one of claims 13-25, wherein the treatment is selected from the list consisting of administration of thiopurine, administration of methotrexate, administration of a biologic, and a surgery.

27. The method of any one of claims 13-26, wherein high TL1A fold-change is associated with the subject being at risk for developing, a non-response or loss-of-response to a standard therapy comprising glucocorticosteriods, anti-TNF therapy, anti-a4-b7 therapy, anti-IL12p40 therapy, or a combination thereof.

28. A method of treating moderate to severely active Crohn's disease (CD) or ulcerative colitis (UC) in a subject, the method comprising: administering a therapeutically effective amount of an inhibitor of Tumor necrosis factor-like cytokine 1A (TL1A) activity or expression to a subject with moderately to severely active CD or UC that has been determined to have high fold-change of TL1A expression relative to a cut-off fold-change value, when the high fold-change of the TL1A expression is measured by:
(a) stimulating peripheral blood mononuclear cells (PBMCs) obtained from the subject with immune complex;
(b) measuring a TL1A expression level at a first time point and a second time point, wherein the second time point is later than the first time point; and
(c) calculating the fold-change of the TL1A expression by dividing the TL1A expression level at the second time point by the TL1A expression level at the first time point.

29. The method of claim 28, wherein the cut-off value is derived from a control sample obtained from a subject that does not have the moderately to severely active CD or UC.

30. The method of claim 28, wherein the cut-off value is derived from the mean plus two times standard deviation for a population.

31. The method of any one of claims 28-30, wherein the first time point is 6 hours after treating the sample of PMBCs with sera derived from the subject.

32. The method of any one of claims 28-31, wherein the second time point is 24 hours after treating the sample of PMBCs with sera derived from the subject.

33. The method of any one of claims 28-32, wherein the second time point is 48 hours after treating the sample of PMBCs with sera derived from the subject.

34. The method of any one of claims 28-33, wherein the second time point is 72 hours after treating the sample of PMBCs with sera derived from the subject.

35. The method of any one of claims 28-34, wherein the subject has an inflammatory disease or condition.

36. The method of claim 35, wherein the inflammatory disease or condition comprises inflammatory bowel disease, Crohn's disease, obstructive Crohn's disease, or ulcerative colitis.

37. The method of claim 36, wherein the Crohn's disease is ileal, ileocolonic, or colonic Crohn's disease.

38. The method of any one of claims 28-37, wherein the subject has, or is at risk for developing, a non-response or loss-of-response to a standard therapy comprising glucocorticosteriods, anti-TNF therapy, anti-a4-b7 therapy, anti-IL12p40 therapy, or a combination thereof.

39. The method of any one of claims 28-38, further comprising administering an anti-TL1A antibody.

40. The method of claim 39, wherein the anti-TL1A inhibitor is an anti-TL1A antibody.

41. The method of claim 40, wherein the anti-TL1A antibody is selected from the group consisting of
an anti-TL1A antibody comprising a HCDR1 as set forth by SEQ ID NOS: 27 or 28, a HCDR2 as set forth by SEQ ID NOS: 41 or 42, a HCDR3 as set forth by SEQ ID NOS: 54 or 55, a LCDR1 as set forth by SEQ ID NO: 68, a LCDR2 as set forth by SEQ ID NO: 81, and a LCDR3 as set forth by SEQ ID NO: 92;
an anti-TL1A antibody comprises a HCDR1 as set forth by SEQ ID NO: 1, a HCDR2 as set forth by SEQ ID NO: 2, a HCDR3 as set forth by SEQ ID NO: 6, a LCDR1 as set forth by SEQ ID NO: 10, a LCDR2 as set forth by SEQ ID NO: 11, and a LCDR3 as set forth by SEQ ID NO: 12;
an anti-TL1A antibody comprises a HCDR1 as set forth by SEQ ID NO: 16 or 17, a HCDR2 as set forth by SEQ ID NO: 18, a HCDR3 as set forth by SEQ ID NO: 19 or 20, a LCDR1 as set forth by SEQ ID NO: 21, a LCDR2 as set forth by SEQ ID NO: 22, and a LCDR3 as set forth by SEQ ID NO: 23 or 24;
an anti-TL1A antibody comprises a HCDR1 as set forth by SEQ ID NO: 25, a HCDR2 as set forth by SEQ ID NO: 39, a HCDR3 as set forth by SEQ ID NO: 52, a LCDR1 as set forth by SEQ ID NO: 66, a LCDR2 as set forth by SEQ ID NO: 79, and a LCDR3 as set forth by SEQ ID NO: 90; and
an anti-TL1A antibody comprises a HCDR1 as set forth by SEQ ID NO: 30, a HCDR2 as set forth by SEQ ID NO: 44, a HCDR3 as set forth by SEQ ID NO: 57, a LCDR1 as set forth by SEQ ID NO: 70, a LCDR2 as set forth by SEQ ID NO: 83, and a LCDR3 as set forth by SEQ ID NO: 94.

42. A method of inhibiting or reducing Tumor necrosis factor-like cytokine 1A (TL1A) activity or expression in a subject, the method comprising:
(a) obtaining peripheral blood mononuclear cells (PBMCs) from the subject;
(b) bringing the PMBCs into contact with immune-complex under conditions sufficient to produce TL1A by the PBMCs;
(c) measuring TL1A expression at a first time point;
(d) measuring TL1A expression at a second time point, wherein the second time point is later in time than the first time point;
(e) calculating the fold-change of TL1A expression between the first and the second time point; and
(f) administering to the subject a therapeutically effective amount of an inhibitor of TL1A activity or expression.

43. The method of claim 42 wherein measuring the TL1A expression at the first time point in (c) is performed 6 hours after the contacting in (b).

44. The method of claim 42, wherein measuring the TL1A expression at the second time point in (d) is performed 24 hours after the contacting in (b).

45. The method of claim 42, wherein measuring the TL1A expression at the second time point in (d) is performed 48 hours after the contacting in (b).

46. The method of claim 42, wherein measuring the TL1A expression at the second time point in (d) is performed 72 hours after the contacting in (b).

47. The method of any one of claims 42-46, wherein the subject has an inflammatory disease, a fibrostenotic disease, or a fibrotic disease.

48. The method of claim 47, wherein the inflammatory disease, a fibrostenotic disease, or a fibrotic disease comprises inflammatory bowel disease, Crohn's disease, obstructive Crohn's disease, or ulcerative colitis.

49. The method of claim 48, wherein the Crohn's disease is ileal, ileocolonic, or colonic Crohn's disease.

50. The method of any one of claims 42-49, wherein the subject has, or is at risk for developing, a non-response or loss-of-response to a standard therapy comprising glucocorticosteriods, anti-TNF therapy, anti-a4-b7 therapy, anti-IL12p40 therapy, or a combination thereof.

51. The method of any one of claims 42-50, wherein the inhibitor of TL1A expression or activity comprises an anti-TL1A antibody.

52. The method of claim 51, wherein the anti-TL1A antibody is selected from the group consisting of
an anti-TL1A antibody comprising a HCDR1 as set forth by SEQ ID NOS: 27 or 28, a HCDR2 as set forth by SEQ ID NOS: 41 or 42, a HCDR3 as set forth by SEQ ID NOS: 54 or 55, a LCDR1 as set forth by SEQ ID NO: 68, a LCDR2 as set forth by SEQ ID NO: 81, and a LCDR3 as set forth by SEQ ID NO: 92;
an anti-TL1A antibody comprises a HCDR1 as set forth by SEQ ID NO: 1, a HCDR2 as set forth by SEQ ID NO: 2, a HCDR3 as set forth by SEQ ID NO: 6, a LCDR1 as set forth by SEQ ID NO: 10, a LCDR2 as set forth by SEQ ID NO: 11, and a LCDR3 as set forth by SEQ ID NO: 12;
an anti-TL1A antibody comprises a HCDR1 as set forth by SEQ ID NO: 16 or 17, a HCDR2 as set forth by SEQ ID NO: 18, a HCDR3 as set forth by SEQ ID NO: 19 or 20, a LCDR1 as set forth by SEQ ID NO: 21, a LCDR2 as set forth by SEQ ID NO: 22, and a LCDR3 as set forth by SEQ ID NO: 23 or 24;
an anti-TL1A antibody comprises a HCDR1 as set forth by SEQ ID NO: 25, a HCDR2 as set forth by SEQ ID NO: 39, a HCDR3 as set forth by SEQ ID NO: 52, a LCDR1 as set forth by SEQ ID NO: 66, a LCDR2 as set forth by SEQ ID NO: 79, and a LCDR3 as set forth by SEQ ID NO: 90; and
an anti-TL1A antibody comprises a HCDR1 as set forth by SEQ ID NO: 30, a HCDR2 as set forth by SEQ ID NO: 44, a HCDR3 as set forth by SEQ ID NO: 57, a LCDR1 as set forth by SEQ ID NO: 70, a LCDR2 as set forth by SEQ ID NO: 83, and a LCDR3 as set forth by SEQ ID NO: 94.

53. A method of selecting a subject for treatment, the method comprising
(a) obtaining peripheral blood mononuclear cells (PBMCs) from the subject
(b) bringing the PMBCs into contact with immune-complex under conditions sufficient to produce Tumor necrosis factor-like cytokine 1A (TL1A) by the PBMCs;
(c) measuring TL1A expression at a first time point;
(d) measuring TL1A expression at a second time point;
(e) calculating the fold-change of TL1A expression between the first and the second time point; and
(f) selecting the subject for treatment with an inhibitor of TL1A activity or expression, provided the fold-change of TL1A is high relative to an index or a control.

54. The method of claim 53, wherein measuring the TL1A expression at the first time point in (c) is performed 6 hours after the contacting in (b).

55. The method of claim 53, wherein measuring the TL1A expression at the second time point in (d) is performed 24 hours after the contacting in (b).

56. The method of claim 53, wherein measuring the TL1A expression at the second time point in (d) is performed 48 hours after the contacting in (b).

57. The method of claim 53, wherein measuring the TL1A expression at the second time point in (d) is performed 72 hours after the contacting in (b).

58. The method of any one of claims 53-57, wherein the subject has an inflammatory disease, a fibrostenotic disease, or a fibrotic disease.

59. The method of claim 58, wherein the inflammatory disease, a fibrostenotic disease, or a fibrotic disease comprises inflammatory bowel disease, Crohn's disease, obstructive Crohn's disease, or ulcerative colitis.

60. The method of claim 59, wherein the Crohn's disease is ileal, ileocolonic, or colonic Crohn's disease.

61. The method of any one of claims 53-60, wherein the subject has, or is at risk for developing, a non-response or loss-of-response to a standard therapy comprising glucocorticosteriods, anti-TNF therapy, anti-a4-b7 therapy, anti-IL12p40 therapy, or a combination thereof.

62. The method of any one of claims 53-61, wherein the inhibitor of TL1A expression or activity comprises an anti-TL1A antibody.

63. The method of claim 62, wherein the anti-TL1A antibody is selected from the group consisting of
an anti-TL1A antibody comprising a HCDR1 as set forth by SEQ ID NOS: 27 or 28, a HCDR2 as set forth by SEQ ID NOS: 41 or 42, a HCDR3 as set forth by SEQ ID NOS: 54 or 55, a LCDR1 as set forth by SEQ ID NO: 68, a LCDR2 as set forth by SEQ ID NO: 81, and a LCDR3 as set forth by SEQ ID NO: 92;
an anti-TL1A antibody comprises a HCDR1 as set forth by SEQ ID NO: 1, a HCDR2 as set forth by SEQ ID NO: 2, a HCDR3 as set forth by SEQ ID NO: 6, a LCDR1 as set forth by SEQ ID NO: 10, a LCDR2 as set forth by SEQ ID NO: 11, and a LCDR3 as set forth by SEQ ID NO: 12;
an anti-TL1A antibody comprises a HCDR1 as set forth by SEQ ID NO: 16 or 17, a HCDR2 as set forth by SEQ ID NO: 18, a HCDR3 as set forth by SEQ ID NO: 19 or 20, a LCDR1 as set forth by SEQ ID NO: 21, a LCDR2 as set forth by SEQ ID NO: 22, and a LCDR3 as set forth by SEQ ID NO: 23 or 24;
an anti-TL1A antibody comprises a HCDR1 as set forth by SEQ ID NO: 25, a HCDR2 as set forth by SEQ ID NO: 39, a HCDR3 as set forth by SEQ ID NO: 52, a LCDR1 as set forth by SEQ ID NO: 66, a LCDR2 as set forth by SEQ ID NO: 79, and a LCDR3 as set forth by SEQ ID NO: 90; and
an anti-TL1A antibody comprises a HCDR1 as set forth by SEQ ID NO: 30, a HCDR2 as set forth by SEQ ID NO: 44, a HCDR3 as set forth by SEQ ID NO: 57, a LCDR1 as set forth by SEQ ID NO: 70, a LCDR2 as set forth by SEQ ID NO: 83, and a LCDR3 as set forth by SEQ ID NO: 94.

64. A method of sample preparation, the method comprising:
(a) obtaining peripheral blood mononuclear cells (PBMCs) from the subject
(b) bringing the PMBCs into contact with immune-complex under conditions sufficient to produce Tumor necrosis factor-like cytokine 1A (TL1A) by the PBMCs;
(c) measuring TL1A expression at a first time point;
(d) measuring TL1A expression at a second time point; and
(e) calculating the fold-change of TL1A between the first and the second time point;
wherein a high fold-change of TL1A relative to an index or control indicates that the subject has a moderate to severely active form of CD or UC.

65. The method of claim 64, wherein measuring the TL1A expression at the first time point in (c) is performed 6 hours after the contacting in (b).

66. The method of claim 64, wherein measuring the TL1A expression at the second time point in (d) is performed 24 hours after the contacting in (b).

67. The method of claim 64, wherein measuring the TL1A expression at the second time point in (d) is performed 48 hours after the contacting in (b).

68. The method of claim 64, wherein measuring the TL1A expression at the second time point in (d) is performed 72 hours after the contacting in (b).

69. The method of claim 64-68, wherein the Crohn's disease is ileal, ileocolonic, or colonic Crohn's disease.
